(19) United States
(12) Patent Application Publication
(10) Pub. No.: US 2019/0015379 A1
(43) Pub. Date: Jan. 17, 2019

(54) USE OF DIANHYDROGALACTITOL AND ANALOGS AND DERIVATIVES THEREOF, TOGETHER WITH RADIATION, TO TREAT NON-SMALL-CELL CARCINOMA OF THE LUNG AND GLIOBLASTOMA MULTIFORME AND SUPPRESS PROLIFERATION OF CANCER STEM CELLS

(71) Applicant: DelMar Pharmaceuticals, Inc., Vancouver (CA)

(72) Inventors: Jeffrey A. BACHA, Vancouver (CA); Dennis M. BROWN, Menlo Park, CA (US); Anne STEINØ, Vancouver (CA); Shaun FOUSE, San Francisco, CA (US)

(21) Appl. No.: 15/525,933

(22) PCT Filed: Nov. 10, 2015
(86) PCT No.: PCT/US2015/059814

§ 371 Date: May 10, 2017

Related U.S. Application Data
(60) Provisional application No. 62/077,712, filed on Nov. 10, 2014.

(51) Int. Cl. A61K 31/336; A61K 38/38; A61K 45/06

(52) U.S. Cl. CPC A61K 31/336; A61K 45/06; A61K 38/38

(57) ABSTRACT

The use of dianhydrogalactitol provides a novel therapeutic modality for the treatment of non-small-cell lung carcinoma (NSCLC) and for the treatment of glioblastoma multiforme (GBM). Dianhydrogalactitol acts as an alkylating agent on DNA that creates N7 methylation. Dianhydrogalactitol is effective in suppressing the growth of cancer stem cells and is active against tumors that are refractory to temozolomide; the drug acts independently of the MGMT repair mechanism.

### CROSS-REFERENCES TO RELATED APPLICATIONS

This application is the United States National Stage application of International Application No. PCT/US2015/059814, filed Nov. 10, 2015, which claims the benefit of U.S. Provisional Patent Application No. 62/077,712, filed Nov. 10, 2014, the contents of which are hereby incorporated by reference in their entirety.

### FIELD OF THE INVENTION

The present invention relates to the general field of hyperproliferative diseases including oncology with a focus on novel methods and compositions for the improved utility of chemical agents, compounds, and dosage forms previously limited by suboptimal human therapeutic performance including substituted hexitols such as dianhydrogalactitol and diacetyldianhydrogalactitol, as well as other classes of chemical agents. In particular, the present invention relates to the treatment of non-small-cell carcinoma of the lung with dianhydrogalactitol, diacetyldianhydrogalactitol, or derivatives or analogs thereof.

### BACKGROUND OF THE INVENTION

The search for and identification of cures for many life-threatening diseases that plague humans still remains an empirical and sometimes serendipitous process. While many advances have been made from basic scientific research to improvements in practical patient management, there still remains tremendous frustration in the rational and successful discovery of useful therapies particularly for life-threatening diseases such as cancer, inflammatory conditions, infection, and other conditions.

Since the “War on Cancer” began in the early 1970's by the United States National Cancer Institute (NCI) of the National Institutes of Health (NIH), a wide variety of strategies and programs have been created and implemented to prevent, diagnose, treat and cure cancer. One of the oldest and arguably most successful programs has been the synthesis and screening of small chemical entities (<1500 MW) for biological activity against cancer. This program was organized to improve and streamline the progression of events from chemical synthesis and biological screening to preclinical studies for the logical progression into human clinical trials with the hope of finding cures for the many types of life-threatening malignant tumors. The synthesis and screening of hundreds of thousands of chemical compounds from academic and industrial sources, in addition to the screening of natural products and extracts from prokaryotes, invertebrate animals, plant collections, and other sources from all over the world has been and continues to be a major approach for the identification of novel lead structures as potential new and useful medicines. This is in addition to other programs including biotherapeutics designed to stimulate the human immune system with vaccines, therapeutic antibodies, cytokines, lymphokines, inhibitors of tumor blood vessel development (angiogenesis) or gene and antisense therapies to alter the genetic make-up of cancer cells, and other biological response modifiers.

The work supported by the NCI, other governmental agencies both domestic and foreign in academic or industrial research and development laboratories has resulted in an extraordinary body of biological, chemical and clinical information. In addition, large chemical libraries have been created, as well as highly characterized in vitro and in vivo biological screening systems that have been successfully used. However, from the tens of billions of dollars spent over the past thirty years supporting these programs both preclinically and clinically, only a small number of compounds have been identified or discovered that have resulted in the successful development of useful therapeutic products. Nevertheless, the biological systems both in vitro and in vivo and the “decision trees” used to warrant further animal studies leading to clinical studies have been validated. These programs, biological models, clinical trial protocols, and other information developed by this work remain critical for the discovery and development of any new therapeutic agent.

Unfortunately, many of the compounds that have successfully met the preclinical testing and federal regulatory requirements for clinical evaluation were either unsuccessful or disappointing in human clinical trials. Many compounds were found to have untoward or idiosyncratic side-effects that were discovered during human clinical Phase I dose-escalation studies used to determine the maximum tolerated dose (MTD) and side-effect profile. In some cases, these toxicities or the magnitude of their toxicity were not identified or predicted in preclinical toxicology studies. In other cases, chemical agents where in vitro and in vivo studies suggested a potentially unique activity against a particular tumor type, molecular target or biological pathway were not successful in human Phase II clinical trials where specific examination of particular cancer indications/types were evaluated in government sanctioned (e.g., U.S. FDA), IRB approved clinical trials. In addition, there are those cases where potential new agents were evaluated in randomized Phase III clinical trials where a significant clinical benefit could not be demonstrated; such cases have also been the cause of great frustration and disappointment. Finally, a number of compounds have reached commercialization but their ultimate clinical utility has been limited by poor efficacy as monotherapy (<25% response rates) and untoward dose-limiting side-effects (Grade III and IV) (e.g., myelosuppression, neurotoxicity, cardiotoxicity, gastrointestinal toxicities, or other significant side effects).

In many cases, after the great time and expense of developing and moving an investigational compound into human clinical trials and where clinical failure has occurred, the tendency has been to return to the laboratory to create a better analog, look for agents with different structures but potentially related mechanisms of action, or try other modifications of the drug. In some cases, efforts have been made to try additional Phase I or II clinical trials in an attempt to make some improvement with the side-effect profile or therapeutic effect in selected patients or cancer indications. In many of those cases, the results did not realize a significant enough improvement to warrant further clinical development toward product registration. Even for commercialized products, their ultimate use is still limited by suboptimal performance.

With so few therapeutics approved for cancer patients and the realization that cancer is a collection of diseases with a multitude of etiologies and that a patient's response and survival from therapeutic intervention is complex with many factors playing a role in the success or failure of treatment including disease indication, stage of invasion and metastatic spread, patient gender, age, health conditions, previous therapies or other illnesses, genetic markers that can either promote or retard therapeutic efficacy, and other factors, the opportunity for cures in the near term remains elusive. Moreover, the incidence of cancer continues to rise with an approximate 4% increase predicted for 2003 in the United States by the American Cancer Society such that over 1.3 million new cancer cases are estimated. In addition, with advances in diagnosis such as mammography for breast cancer and PSA tests for prostate cancer, more patients are being diagnosed at a younger age. For difficult to treat cancers, a patient's treatment options are often exhausted quickly resulting in a desperate need for additional treatment regimens. Even for the most limited of patient populations, any additional treatment opportunities would be of considerable value. This invention focuses on inventive compositions and methods for improving the therapeutic benefit of suboptimally administered chemical compounds including substituted hexitols such as dianhydrogalactitol.

Non-small-cell lung carcinoma (NSCLC) includes several types of lung cancer, including squamous cell carcinoma, large cell carcinoma, and adenocarcinoma, as well as other types of lung cancer. Although smoking is apparently the most frequent cause of squamous cell carcinoma, when lung cancer occurs in patients without any history of prior tobacco smoking, it is frequently adenocarcinoma. In many cases, NSCLC is refractory to chemotherapy, so surgical resection of the tumor mass is typically the treatment of choice, particularly if the malignancy is diagnosed early. However, chemotherapy and radiation therapy are frequently attempted, particularly if the diagnosis cannot be made at an early stage of the malignancy. Other treatments include radiofrequency ablation and chemoembolization. A wide variety of chemotherapeutic treatments has been tried for advanced or metastatic NSCLC. Some patients with particular mutations in the EGFR gene respond to EGFR tyrosine kinase inhibitors such as gefitinib (M. G. Kris, “How Today's Developments in the Treatment of Non-Small Cell Lung Cancer Will Change Tomorrow's Standards of Care,” Oncologist 10 (Suppl. 2): 23-29 (2005), incorporated herein by this reference). Cisplatin has frequently been used as ancillary therapy together with surgery. Erlotinib, pemetrexed, About 7% of NSCLC have EML4-ALK translocations, and such patients may benefit from ALK inhibitors such as crizotinib. Other therapies, including the vaccine TG4010, motesanib diphosphate, tivantinib, belotecan, eribulin mesylate, ramucirumab, necitumumab, the vaccine GSK1572932A, custirsen sodium, the liposome-based vaccine BLP25, nivolumab, EMD531444, dacomitinib, and genetespib, are being evaluated, particularly for advanced or metastatic NSCLC.

However, there is still a need for effective therapies against NSCLC, especially against advanced or metastatic NSCLC. Preferably, such therapies should be well-tolerated and with side effects, if any, that could be easily controlled. Also, preferably, such therapies should be compatible with other chemotherapeutic approaches and with surgery or radiation. Additionally, and preferably, such therapies should be able to exert a synergistic effect on other treatment modalities. Additionally, there is a need for effective treatments for glioblastoma multiforme.

In particular, there is a need for therapies against NSCLC and glioblastoma multiforme that can be used to suppress or prevent the growth of cancer stem cells (CSC). Additionally, there is a need for therapies against CSC that can be used together with radiation.

### SUMMARY OF THE INVENTION

The use of a substituted hexitol derivative to treat non-small-cell lung carcinoma (NSCLC) and glioblastoma multiforme (GBM) provides an improved therapy for NSCLC and GBM that yields increased survival and is substantially free of side effects. In general, the substituted hexitols usable in methods and compositions according to the present invention include galactitols, substituted galactitols, dulcitols, and substituted dulcitols. Typically, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. A particularly preferred substituted hexitol derivative is dianhydrogalactitol (DAG). The substituted hexitol derivative can be employed together with other therapeutic modalities for these malignancies. Dianhydrogalactitol is particularly suited for the treatment of these malignancies because it can suppress the growth of cancer stem cells (CSC), and because it is resistant to drug inactivation by O⁶-methylguanine-DNA methyltransferase (MGMT). The substituted hexitol derivative yields increased response rates and improved quality of life for patients with NSCLC and GBM.

Dianhydrogalactitol is a novel alkylating agent that creates N⁷-methylation in DNA. Specifically, the principal mechanism of action of dianhydrogalactitol is attributed to bi-functional N⁷ DNA alkylation, via actual or derived epoxide groups, which cross-links across DNA strands.

Accordingly, one aspect of the present invention is a method to improve the efficacy and/or reduce the side effects of the administration of a substituted hexitol derivative for treatment of NSCLC and GBM comprising the steps of:

(1) identifying at least one factor or parameter associated with the efficacy and/or occurrence of side effects of the administration of the substituted hexitol derivative for treatment of NSCLC or GBM; and

(2) modifying the factor or parameter to improve the efficacy and/or reduce the side effects of the administration of the substituted hexitol derivative for treatment of NSCLC or GBM.

Typically, the factor or parameter is selected from the group consisting of:

(1) dose modification;

(2) route of administration;

(3) schedule of administration;

(4) indications for use;

(5) selection of disease stage;

(6) other indications;

(7) patient selection;

(8) patient/disease phenotype;

(9) patient/disease genotype;

(10) pre/post-treatment preparation;

(11) toxicity management;

(12) pharmacokinetic/pharmacodynamic monitoring;

(13) drug combinations;

(14) chemosensitization;

(15) chemopotentiation;

(16) post-treatment patient management;

(17) alternative medicine/therapeutic support;

(18) bulk drug product improvements;

(19) diluent systems;

(20) solvent systems;

(21) excipients;

(22) dosage forms;

(23) dosage kits and packaging;

(24) drug delivery systems;

(25) drug conjugate forms;

(26) compound analogs;

(27) prodrugs;

(28) multiple drug systems;

(29) biotherapeutic enhancement;

(30) biotherapeutic resistance modulation;

(31) radiation therapy enhancement;

(32) novel mechanisms of action;

(33) selective target cell population therapeutics;

(34) use with ionizing radiation;

(35) use with an agent that counteracts myelosuppression;

(36) use with an agent that increases the ability of the substituted hexitol to pass through the blood-brain barrier to treat brain metastases of NSCLC; and

(37) use with an agent that suppresses proliferation of cancer stem cells (CSC).

As detailed above, typically, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the substituted hexitol derivative is dianhydrogalactitol.

Another aspect of the present invention is a composition to improve the efficacy and/or reduce the side effects of suboptimally administered drug therapy employing a substituted hexitol derivative for the treatment of NSCLC comprising an alternative selected from the group consisting of:

(i) a therapeutically effective quantity of a modified substituted hexitol derivative or a derivative, analog, or prodrug of a substituted hexitol derivative or a modified substituted hexitol derivative, wherein the modified substituted hexitol derivative or the derivative, analog or prodrug of the substituted hexitol derivative or modified substituted hexitol derivative possesses increased therapeutic efficacy or reduced side effects for treatment of NSCLC or GBM as compared with an unmodified substituted hexitol derivative;

(ii) a composition comprising:
(a) a therapeutically effective quantity of a substituted hexitol derivative, a modified substituted hexitol derivative, or a derivative, analog, or prodrug of a substituted hexitol derivative or a modified substituted hexitol derivative; and
(b) at least one additional therapeutic agent, therapeutic agent subject to chemosensitization, therapeutic agent subject to chemopotentiation, diluent, excipient, solvent system, drug delivery system, or agent to counteract myelosuppression, wherein the composition possesses increased therapeutic efficacy or reduced side effects for treatment of NSCLC or GBM as compared with an unmodified substituted hexitol derivative;

(iii) a therapeutically effective quantity of a substituted hexitol derivative, a modified substituted hexitol derivative or a derivative, analog, or prodrug of a substituted hexitol derivative or a modified substituted hexitol derivative that is incorporated into a dosage form, wherein the substituted hexitol derivative, the modified substituted hexitol derivative or the derivative, analog, or prodrug of a substituted hexitol derivative or a modified substituted hexitol derivative incorporated into the dosage form possesses increased therapeutic efficacy or reduced side effects for treatment of NSCLC or GBM as compared with an unmodified substituted hexitol derivative;

(iv) a therapeutically effective quantity of a substituted hexitol derivative, a modified substituted hexitol derivative or a derivative, analog, or prodrug of a substituted hexitol derivative or a modified substituted hexitol derivative that is incorporated into a dosage kit and packaging, wherein the substituted hexitol derivative, the modified substituted hexitol derivative or the derivative, analog, or prodrug of a substituted hexitol derivative or a modified substituted hexitol derivative incorporated into the dosage kit and packaging possesses increased therapeutic efficacy or reduced side effects for treatment of NSCLC or GBM as compared with an unmodified substituted hexitol derivative; and

(v) a therapeutically effective quantity of a substituted hexitol derivative, a modified substituted hexitol derivative or a derivative, analog, or prodrug of a substituted hexitol derivative or a modified substituted hexitol derivative that is subjected to a bulk drug product improvement, wherein substituted hexitol derivative, a modified substituted hexitol derivative or a derivative, analog, or prodrug of a substituted hexitol derivative or a modified substituted hexitol derivative subjected to the bulk drug product improvement possesses increased therapeutic efficacy or reduced side effects for treatment of NSCLC or GBM as compared with an unmodified substituted hexitol derivative.

As detailed above, typically the unmodified substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the unmodified substituted hexitol derivative is dianhydrogalactitol.

Another aspect of the present invention is a method of treating NSCLC or GBM comprising the step of administering a therapeutically effective quantity of a substituted hexitol derivative to a patient suffering from NSCLC or GBM. As detailed above, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the substituted hexitol derivative is dianhydrogalactitol. The method can be used to treat patients who have developed resistance to tyrosine kinase inhibitors (TKI) or platinum-based chemotherapeutic agents such as cisplatin. The method can also be used together with TKI or platinum-based chemotherapeutic agents. Additionally, the method can also be used together with ionizing radiation or with agents that suppress the proliferation of cancer stem cells.

### BRIEF DESCRIPTION OF THE DRAWINGS

The following invention will become better understood with reference to the specification, appended claims, and accompanying drawings, where:

FIG. 1 is a graph that shows body weight on the y-axis versus days post-inoculation on the x-axis for the results of the Example. In FIGS. 1-2 of the Example, • is the untreated control; ▪ is the cisplatin control; ▴ is dianhydrogalactitol at 1.5 mg/kg; ▴ is dianhydrogalactitol at 3.0 mg/kg; and □ is dianhydrogalactitol at 6.0 mg/kg.

FIG. 2 is a graph that shows the tumor volume (means±S.E.M.) for the A549 tumor-bearing female Rag2 mice with tumor volume on the y axis versus days post-inoculation on the x-axis for the results of the Example. The top panel of FIG. 2 represents all mice for the complete duration of the study. The bottom panel of FIG. 2 represents all mice until day 70 (last day for untreated control group).

FIG. 3 shows the mechanism of action for dianhydrogalactitol.

FIG. 4 shows the MGMT status of the cultures. “GAPDH” refers to glyceraldehyde-3-phosphate dehydrogenase as a control. For the cell cultures, CSCs were cultured in NSA media supplemented with B27, EGF and bFGF. Non-CSCs were grown in DMEM:F12 with 10% FBS. MGMT methylation and protein expression analysis of each culture was characterized. TMZ or VAL-083 was added to the cultures in the indicated concentrations. Depending on the experiment, cells were also irradiated with 2 Gy in a cesium irradiator. For assays, cell cycle analysis was performed with Propidium Iodide staining and FACs analysis. Cell viability was analyzed with CellTiter-Glo™ and read on a Promega GloMax™. In FIG. 4, Panel C shows the methylation status of MGMT for cell lines SF7996, SF8161, SF8279, and SF8565; “U” refers to unmethylated and “M” refers to methylated. In FIG. 4, “1° GBM” refers to primary glioblastoma multiforme cell cultures. FIG. 4 shows MGMT western blot analysis of protein extracts from 4 pairs of CSC and non-CSC cultures derived from primary GBM tissue.

FIG. 5 shows that dianhydrogalactitol (“VAL-083”) was better than TMZ for inhibiting tumor cell growth and that this occurred in an MGMT-independent manner.

FIG. 6 shows schematics of various treatment regimens for temozolomide (“TMZ”) or dianhydrogalactitol (“VAL”), with or without radiation (“XRT”).

FIG. 7 shows cell cycle analyses for cancer stem cells (CSC) treated with TMZ or dianhydrogalactitol (“VAL-083”), for 7996 CSC, 8161 CSC, 8565 CSC, and 8279 CSC. In these cell cycle analyses, G2 is shown at the top, S in the middle, and G1 at the bottom.

FIG. 8 shows cell cycle analyses for non-stem-cell cultures treated with TMZ or dianhydrogalactitol (“VAL-083”), for 7996 non-CSC, 8161 non-CSC, 8565 non-CSC, and U251. In these cell cycle analyses, G2 is shown at the top, S in the middle, and G1 at the bottom.

FIG. 9 shows examples of FACS profiles for 7996 non-CSC dianhydrogalactitol (“VAL”) treatment.

FIG. 10 shows a schematic of the treatment regimen using either temozolomide (“TMZ”) or dianhydrogalactitol (“VAL”) and radiation (“XRT”).

FIG. 11 shows results for 7996 CSC for TMZ only, VAL only, and TMZ or VAL with XRT. In FIG. 11, for TMZ “-D/-” indicates DMSO only (vehicle), “-T/-” indicates TMZ only, and “-D/X” or “-T/X” indicate DMSO or TMZ with XRT. Similarly, for VAL, “—P/-” indicates phosphate buffered saline (PBS) only (vehicle), “—V/-” indicates VAL only, and “—P/X” or “—V/X” indicate PBS or VAL with XRT. The left side of FIG. 11 shows cell cycle analysis where G2 is shown at the top, S in the middle, and G1 at the bottom; both 4- and 6-day results are shown, with the 4-day results (“D4”) presented to the left of the 6-day results (“D6”). The right side of FIG. 11 shows the results for cell viability as a percentage of control for D4 and D6.

FIG. 12 shows results for 8161 CSC depicted as in FIG. 11.

FIG. 13 shows results for 8565 CSC depicted as in FIG. 11.

FIG. 14 shows results for 7996 non-CSC depicted as in FIG. 11.

FIG. 15 shows results for U251 depicted as in FIG. 11.

FIG. 16 shows that dianhydrogalactitol causes cell cycle arrest in TMZ-resistant cultures. In FIG. 16, cells were treated with either increasing doses of TMZ (5, 50 100 and 200 μM) or dianhydrogalactitol (“VAL-083”) (1, 5, 25 and 100 μM) and cell cycle analysis was performed 4 days post treatment. TMZ resistant cultures (A, B, D) exhibited sensitivity to VAL-083, even at single-micromolar doses. Furthermore, this response was not dependent on culture type as paired CSC (A) and non-CSC (B) both exhibit sensitivity to VAL-083.

FIG. 17 shows that dianhydrogalactitol decreases cell viability in TMZ-resistant cultures. In FIG. 17, TMZ (50 μM) or dianhydrogalactitol (“VAL-083”) (5 μM) were added to primary CSC cultures at various doses with or without irradiation (2 Gy). Shown are cell cycle profile analysis at day 4 post treatment (A,C) and cell viability analysis at day 6 post treatment (B,D) for the paired CSC (A,B) and non-CSC (C,D) 7996 culture. Whereas these cultures are not very sensitive to TMZ, they are to VAL-083. However, the addition of radiation (XRT) in both cases does not result in increased sensitivity (D=DMSO, T=TMZ, X=XRT, P=PBS).

FIG. 18 shows that dianhydrogalactitol acts as a radiosensitizer in primary CSC cultures. In FIG. 18, dianhydrogalactitol (“VAL-083”) was added to primary CSC cultures at various doses (1, 2.5 and 5 μM) with or without irradiation (2 Gy). Shown are cell cycle profile analysis at day 4 post treatment (A,C) and cell viability analysis at day 6 post treatment (B,D) for two different patient-derived CSC cultures, 7996 (A,B) and 8565 (C,D).

FIG. 19 shows the treatment regimens with a wash or no wash for both dianhydrogalactitol and temozolomide.

FIG. 20 shows the results for 7996 GNS, showing cell cycle analysis where G2 is shown at the top, S in the middle, and G1 at the bottom. Results for TMZ are shown on the top and results for dianhydrogalactitol on the bottom. Results with a wash are shown on the left and results without a wash are shown on the right.

FIG. 21 shows the results for 8279 GNS, depicted as in FIG. 20.

FIG. 22 shows the results for 7996 ML, depicted as in FIG. 20.

FIG. 23 shows the results for 8565 ML, depicted as in FIG. 20.

FIG. 24 shows the treatment regimens for combining dianhydrogalactitol (“VAL”) and radiation (“XRT”).

FIG. 25 shows the results for 7996 GNS (CSC) when dianhydrogalactitol is combined with radiation. Results are shown at day 4 (“D4”) on the top and day 6 (“D6”) on the bottom. The left side shows cell cycle analysis where G2 is shown at the top, S in the middle, and G1 at the bottom. The right side shows cell viability at D4 and D6.

FIG. 26 shows the results for 8565 GNS (CSC) as depicted in FIG. 25.

FIG. 27 shows the results for 7996 ML (non-CSC) as depicted in FIG. 25.

FIG. 28 shows the results for 8565 ML (non-CSC) as depicted in FIG. 25.

FIG. 29 shows the activity of dianhydrogalactitol (VAL-083) and temozolomide (TMZ) in MGMT negative pediatric human GBM cell line SF188 (first panel), MGMT negative human GBM cell line U251 (second panel) and MGMT positive human GBM cell line T98G (third panel); immunoblots showing detection of MGMT and actin (as a control) in the individual cell lines are shown under the table providing the properties of the cell lines.

FIG. 30 shows the plasma concentration-time profiles of dianhydrogalactitol showing dose-dependent systemic exposure (mean of 3 subjects per cohort).

FIG. 31 shows the results from MRI scans from a human subject after two cycles dianhydrogalactitol treatment. Thick confluent regions of abnormal enhancement have diminished, now appearing more heterogeneous (left two scans, T=0; right two scans, T=64 days).

### DETAILED DESCRIPTION OF THE INVENTION

The compound dianhydrogalactitol (DAG) has been shown to have substantial efficacy in inhibiting the growth of non-small-cell lung carcinoma (NSCLC) cells. In the case of GBM, DAG has proven to be more effective in suppressing the growth of NSCLC cells in a mouse model than cisplatin (TMZ), the current chemotherapy of choice for NSCLC. As detailed below, DAG can effectively suppress the growth of cancer stem cells (CSCs). DAG acts independently of the MGMT repair mechanism. Therefore, DAG and derivatives or analogs thereof can be used to treat NSCLC or GBM.

The structure of dianhydrogalactitol (DAG) is shown in Formula (I), below.

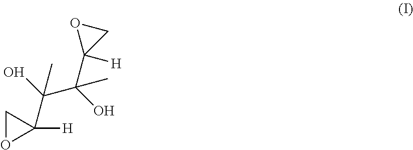

As detailed below, other substituted hexitols can be used in methods and compositions according to the present invention. In general, the substituted hexitols usable in methods and compositions according to the present invention include galactitols, substituted galacitols, dulcitols, and substituted dulcitols, including dianhydrogalactitol, diacetyldianhydrogalactitol, dibromodulcitol, and derivatives and analogs thereof. Typically, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the substituted hexitol derivative is dianhydrogalactitol.

These galactitols, substituted galacitols, dulcitols, and substituted dulcitols are either alkylating agents or prodrugs of alkylating agents, as discussed further below.

Also within the scope of the invention are derivatives of dianhydrogalactitol that, for example, have one or both hydrogens of the two hydroxyl groups of dianhydrogalactitol replaced with lower alkyl, have one or more of the hydrogens attached to the two epoxide rings replaced with lower alkyl, or have the methyl groups present in dianhydrogalactitol and that are attached to the same carbons that bear the hydroxyl groups replaced with C₂-C₆ lower alkyl or substituted with, for example, halo groups by replacing a hydrogen of the methyl group with, for example a halo group. As used herein, the term “halo group,” without further limitation, refers to one of fluoro, chloro, bromo, or iodo. As used herein, the term “lower alkyl,” without further limitation, refers to C₁-C₆ groups and includes methyl. The term “lower alkyl” can be further limited, such as “C₂-C₆ lower alkyl,” which excludes methyl. The term “lower alkyl”, unless further limited, refers to both straight-chain and branched alkyl groups. These groups can, optionally, be further substituted, as described below.

The structure of diacetyldianhydrogalactitol is shown in Formula (II), below.

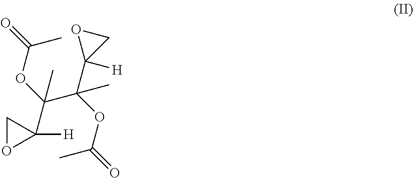 [SMILES: CC(=O)OC(C)(C1CO1)C(C)(OC(C)=O)C1CO1]

Also within the scope of the invention are derivatives of diacetyldianhydrogalactitol that, for example, have one or both of the methyl groups that are part of the acetyl moieties replaced with C₂-C₆ lower alkyl, have one or both of the hydrogens attached to the epoxide ring replaced with lower alkyl, or have the methyl groups attached to the same carbons that bear the acetyl groups replaced with lower alkyl or substituted with, for example, halo groups by replacing a hydrogen with, for example, a halo group.

The structure of dibromodulcitol is shown in Formula (III), below. Dibromodulcitol can be produced by the reaction of dulcitol with hydrobromic acid at elevated temperatures, followed by crystallization of the dibromodulcitol. Some of the properties of dibromodulcitol are described in N. E. Mischler et al., “Dibromoducitol,” Cancer Treat. Rev. 6: 191-204 (1979), incorporated herein by this reference. In particular, dibromodulcitol, as an α, ω-dibrominated hexitol, dibromodulcitol shares many of the biochemical and biological properties of similar drugs such as dibromomannitol and mannitol myleran. Activation of dibromodulcitol to the diepoxide dianhydrogalactitol occurs in vivo, and dianhydrogalactitol may represent a major active form of the drug; this means that dibromogalactitol has many of the properties of a prodrug. Absorption of dibromodulcitol by the oral route is rapid and fairly complete. Dibromodulcitol has known activity in melanoma, breast lymphoma (both Hodgkins and non-Hodgkins), colorectal cancer, acute lymphoblastic leukemia and has been shown to lower the incidence of central nervous system leukemia, non-small cell lung cancer, cervical carcinoma, bladder carcinoma, and metastatic hemangiopericytoma.

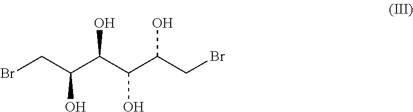 [SMILES: O[C@H]([C@H](O)[C@@H](O)CBr)[C@H](O)CBr]

Also within the scope of the invention are derivatives of dibromodulcitol that, for example, have one or more hydrogens of the hydroxyl groups replaced with lower alkyl, or have one or both of the bromo groups replaced with another halo group such as chloro, fluoro, or iodo.

In general, for optional substituents at saturated carbon atoms such as those that are part of the structures of dianhydrogalactitol, derivatives of dianhydrogalactitol, diacetyldianhydrogalactitol, derivatives of diacetyldianhydrogalactitol, dibromodulcitol, and derivatives of dibromodulcitol, the following substituents can be employed: C₆-C₁₀ aryl, heteroaryl containing 1-4 heteroatoms selected from N, O, and S, C₁-C₁₀ alkyl, C₁-C₁₀ alkoxy, cycloalkyl, F, amino (NR¹R²), nitro, —SR, —S(O)R, —S(O₂)R, —S(O₂)NR¹R², and —CONR¹R², which can in turn be optionally substituted. Further descriptions of potential optional substituents are provided below.

Optional substituents as described above that are within the scope of the present invention do not substantially affect the activity of the derivative or the stability of the derivative, particularly the stability of the derivative in aqueous solution. Definitions for a number of common groups that can be used as optional substituents are provided below; however, the omission of any group from these definitions cannot be taken to mean that such a group cannot be used as an optional substituent as long as the chemical and pharmacological requirements for an optional substituent are satisfied.

As used herein, the term “alkyl” refers to an unbranched, branched, or cyclic saturated hydrocarbyl residue, or a combination thereof, of from 1 to 12 carbon atoms that can be optionally substituted; the alkyl residues contain only C and H when unsubstituted. Typically, the unbranched or branched saturated hydrocarbyl residue is from 1 to 6 carbon atoms, which is referred to herein as “lower alkyl.” When the alkyl residue is cyclic and includes a ring, it is understood that the hydrocarbyl residue includes at least three carbon atoms, which is the minimum number to form a ring. As used herein, the term “alkenyl” refers to an unbranched, branched or cyclic hydrocarbyl residue having one or more carbon-carbon double bonds. As used herein, the term “alkynyl” refers to an unbranched, branched, or cyclic hydrocarbyl residue having one or more carbon-carbon triple bonds; the residue can also include one or more double bonds. With respect to the use of “alkenyl” or “alkynyl,” the presence of multiple double bonds cannot produce an aromatic ring. As used herein, the terms “hydroxyalkyl,” “hydroxyalkenyl,” and “hydroxyalkynyl,” respectively, refer to an alkyl, alkenyl, or alkynyl group including one or more hydroxyl groups as substituents; as detailed below, further substituents can be optionally included. As used herein, the term “aryl” refers to a monocyclic or fused bicyclic moiety having the well-known characteristics of aromaticity; examples include phenyl and naphthyl, which can be optionally substituted. As used herein, the term “hydroxyaryl” refers to an aryl group including one or more hydroxyl groups as substituents; as further detailed below, further substituents can be optionally included. As used herein, the term “heteroaryl” refers to monocyclic or fused bicyclic ring systems that have the characteristics of aromaticity and include one or more heteroatoms selected from O, S, and N. The inclusion of a heteroatom permits aromaticity in 5-membered rings as well as in 6-membered rings. Typical heteroaromatic systems include monocyclic C₅-C₆ heteroaromatic groups such as pyridyl, pyrimidyl, pyrazinyl, thienyl, furanyl, pyrrolyl, pyrazolyl, thiazolyl, oxazolyl, triazolyl, triazinyl, tetrazolyl, tetrazinyl, and imidazolyl, as well as the fused bicyclic moieties formed by fusing one of these monocyclic heteroaromatic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolylpyridyl, quinazolinyl, quinoxalinyl, cinnolinyl, and other ring systems known in the art. Any monocyclic or fused ring bicyclic system that has the characteristics of aromaticity in terms of delocalized electron distribution throughout the ring system is included in this definition. This definition also includes bicyclic groups where at least the ring that is directly attached to the remainder of the molecule has the characteristics of aromaticity, including the delocalized electron distribution that is characteristic of aromaticity. Typically the ring systems contain 5 to 12 ring member atoms and up to four heteroatoms, wherein the heteroatoms are selected from the group consisting of N, O, and S. Frequently, the monocyclic heteroaryls contain 5 to 6 ring members and up to three heteroatoms selected from the group consisting of N, O, and S; frequently, the bicyclic heteroaryls contain 8 to 10 ring members and up to four heteroatoms selected from the group consisting of N, O, and S. The number and placement of heteroatoms in heteroaryl ring structures is in accordance with the well-known limitations of aromaticity and stability, where stability requires the heteroaromatic group to be stable enough to be exposed to water at physiological temperatures without rapid degradation. As used herein, the term “hydroxheteroaryl” refers to a heteroaryl group including one or more hydroxyl groups as substituents; as further detailed below, further substituents can be optionally included. As used herein, the terms “haloaryl” and “haloheteroaryl” refer to aryl and heteroaryl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. As used herein, the terms “haloalkyl,” “haloalkenyl,” and “haloalkynyl” refer to alkyl, alkenyl, and alkynyl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included.

As used herein, the term “optionally substituted” indicates that the particular group or groups referred to as optionally substituted may have no non-hydrogen substituents, or the group or groups may have one or more non-hydrogen substituents consistent with the chemistry and pharmacological activity of the resulting molecule. If not otherwise specified, the total number of such substituents that may be present is equal to the total number of hydrogen atoms present on the unsubstituted form of the group being described; fewer than the maximum number of such substituents may be present. Where an optional substituent is attached via a double bond, such as a carbonyl oxygen (C═O), the group takes up two available valences on the carbon atom to which the optional substituent is attached, so the total number of substituents that may be included is reduced according to the number of available valiences. As used herein, the term “substituted,” whether used as part of “optionally substituted” or otherwise, when used to modify a specific group, moiety, or radical, means that one or more hydrogen atoms are, each, independently of each other, replaced with the same or different substituent or substituents.

Substituent groups useful for substituting saturated carbon atoms in the specified group, moiety, or radical include, but are not limited to, —Z^{a}, ═O, —OZ^{b}, —SZ^{b}, ═S⁻, —NZ^{c}Z^{c}, ═NZ^{b}, ═N—OZ^{b}, trihalomethyl, —CF₃, —CN, —OCN, —SCN, —NO, —NO₂, ═N₂, —N₃, —S(O)₂Z^{b}, —S(O)₂NZ^{b}, —S(O₂)O⁻, —S(O₂)OZ^{b}, —OS(O₂)OZ^{b}, —OS(O₂)O⁻, —OS(O₂)OZ^{b}, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c},—C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)Z^{b}, —NZ^{b}C(S)Z^{b}, —NZ^{b}C(O)O⁻, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, —NZ^{b}C(NZ^{b})NZ^{c}Z^{c}, wherein Z^{a} is selected from the group consisting of alkyl, cycloalkyl, heteroalkyl, cycloheteroalkyl, aryl, arylalkyl, heteroaryl and heteroarylalkyl; each Z^{b} is independently hydrogen or Z^{a}; and each Z^{c} is independently Z^{b} or, alternatively, the two Z^{c}'s may be taken together with the nitrogen atom to which they are bonded to form a 4-, 5-, 6-, or 7-membered cycloheteroalkyl ring structure which may optionally include from 1 to 4 of the same or different heteroatoms selected from the group consisting of N, O, and S. As specific examples, —NZ^{c}Z^{c} is meant to include —NH₂, —NH-alkyl, —N-pyrrolidinyl, and —N-morpholinyl, but is not limited to those specific alternatives and includes other alternatives known in the art. Similarly, as another specific example, a substituted alkyl is meant to include -alkylene-O-alkyl, -alkylene-heteroaryl, -alkylene-cycloheteroaryl, -alkylene-C(O)OZ^{b}, -alkylene-C(O)NZ^{b}Z^{b}, and —CH₂—CH₂—C(O)—CH₃, but is not limited to those specific alternatives and includes other alternatives known in the art. The one or more substituent groups, together with the atoms to which they are bonded, may form a cyclic ring, including, but not limited to, cycloalkyl and cycloheteroalkyl.

Similarly, substituent groups useful for substituting unsaturated carbon atoms in the specified group, moiety, or radical include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —N₃, —S(O)₂Z^{b}, —S(O₂)O⁻, —S(O₂)OZ^{b}, —OS(O₂)OZ^{b}, —OS(O₂)O⁻, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

Similarly, substituent groups useful for substituting nitrogen atoms in heteroalkyl and cycloheteroalkyl groups include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —S(O)₂Z^{b}, —S(O₂)O⁻, —S(O₂)OZ^{b}, —OS(O₂)OZ^{b}, —OS(O₂)O⁻, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)OZ^{b}, —OC(S)OZ^{b}, —NZ^{b}C(O)Z^{b}, —NZ^{b}C(S)Z^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

The compounds described herein may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms (such as the enantiomerically pure isomers, the E and Z isomers, and other alternatives for stereoisomers) as well as mixtures of stereoisomers in varying degrees of chiral purity or percentage of E and Z, including racemic mixtures, mixtures of diastereomers, and mixtures of E and Z isomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the invention to the depicted isomer. When the chemical name does not specify the isomeric form of the compound, it denotes any one of the possible isomeric forms or mixtures of those isomeric forms of the compound.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium; the equilibrium may strongly favor one of the tautomers, depending on stability considerations. For example, ketone and enol are two tautomeric forms of one compound.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate.” Examples of hydrate include, but are not limited to, hemihydrate, monohydrate, dihydrate, trihydrate, hexahydrate, and other water-containing species. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

As used herein, the term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolysable esters of the present compounds are the compounds whose carboxyls are present in the form of hydrolysable ester groups. That is, these esters are pharmaceutically acceptable and can be hydrolyzed to the corresponding carboxyl acid in vivo.

In addition to the substituents described above, alkyl, alkenyl and alkynyl groups can alternatively or in addition be substituted by C₁-C₈ acyl, C₂-C₈ heteroacyl, C₆-C₁₀ aryl, C₃-C₈ cycloalkyl, C₃-C₈ heterocyclyl, or C₅-C₁₀ heteroaryl, each of which can be optionally substituted. Also, in addition, when two groups capable of forming a ring having 5 to 8 ring members are present on the same or adjacent atoms, the two groups can optionally be taken together with the atom or atoms in the substituent groups to which they are attached to form such a ring.

“Heteroalkyl,” “heteroalkenyl,” and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker.

Similarly, “heterocyclyl” may be used to describe a non-aromatic cyclic group that contains at least one heteroatom (typically selected from N, O and S) as a ring member and that is connected to the molecule via a ring atom, which may be C (carbon-linked) or N (nitrogen-linked); and “heterocyclylalkyl” may be used to describe such a group that is connected to another molecule through a linker. The heterocyclyl can be fully saturated or partially saturated, but non-aromatic. The sizes and substituents that are suitable for the cycloalkyl, cycloalkylalkyl, heterocyclyl, and heterocyclylalkyl groups are the same as those described above for alkyl groups. The heterocyclyl groups typically contain 1, 2 or 3 heteroatoms, selected from N, O and S as ring members; and the N or S can be substituted with the groups commonly found on these atoms in heterocyclic systems. As used herein, these terms also include rings that contain a double bond or two, as long as the ring that is attached is not aromatic. The substituted cycloalkyl and heterocyclyl groups also include cycloalkyl or heterocyclic rings fused to an aromatic ring or heteroaromatic ring, provided the point of attachment of the group is to the cycloalkyl or heterocyclyl ring rather than to the aromatic/heteroaromatic ring.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain.

In general, any alkyl, alkenyl, alkynyl, acyl, or aryl or arylalkyl group that is contained in a substituent may itself optionally be substituted by additional substituents. The nature of these substituents is similar to those recited with regard to the primary substituents themselves if the substituents are not otherwise described.

“Amino” as used herein refers to —NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle,” “carbocyclyl,” or “carbocyclic” refers to a cyclic ring containing only carbon atoms in the ring, whereas the term “heterocycle” or “heterocyclic” refers to a ring comprising a heteroatom. The carbocyclyl can be fully saturated or partially saturated, but non-aromatic. For example, the carbocyclyl encompasses cycloalkyl. The carbocyclic and heterocyclic structures encompass compounds having monocyclic, bicyclic or multiple ring systems; and such systems may mix aromatic, heterocyclic, and carbocyclic rings. Mixed ring systems are described according to the ring that is attached to the rest of the compound being described.

As used herein, the term “heteroatom” refers to any atom that is not carbon or hydrogen, such as nitrogen, oxygen or sulfur. When it is part of the backbone or skeleton of a chain or ring, a heteroatom must be at least divalent, and will typically be selected from N, O, P, and S.

As used herein, the term “alkanoyl” refers to an alkyl group covalently linked to a carbonyl (C═O) group. The term “lower alkanoyl” refers to an alkanoyl group in which the alkyl portion of the alkanoyl group is C₁-C₆. The alkyl portion of the alkanoyl group can be optionally substituted as described above. The term “alkylcarbonyl” can alternatively be used. Similarly, the terms “alkenylcarbonyl” and “alkynylcarbonyl” refer to an alkenyl or alkynyl group, respectively, linked to a carbonyl group.

As used herein, the term “alkoxy” refers to an alkyl group covalently linked to an oxygen atom; the alkyl group can be considered as replacing the hydrogen atom of a hydroxyl group. The term “lower alkoxy” refers to an alkoxy group in which the alkyl portion of the alkoxy group is C₁-C₆. The alkyl portion of the alkoxy group can be optionally substituted as described above. As used herein, the term “haloalkoxy” refers to an alkoxy group in which the alkyl portion is substituted with one or more halo groups.

As used herein, the term “sulfo” refers to a sulfonic acid (—SO₃H) substituent.

As used herein, the term “sulfamoyl” refers to a substituent with the structure —S(O₂)NH₂, wherein the nitrogen of the NH₂ portion of the group can be optionally substituted as described above.

As used herein, the term “carboxyl” refers to a group of the structure —C(O₂)H.

As used herein, the term “carbamyl” refers to a group of the structure —C(O₂)NH₂, wherein the nitrogen of the NH₂ portion of the group can be optionally substituted as described above.

As used herein, the terms “monoalkylaminoalkyl” and “dialkylaminoalkyl” refer to groups of the structure -Alk₁-NH-Alk₂ and -Alk₁-N(Alk₂)(Alk₃), wherein Alk₁, Alk₂, and Alk₃ refer to alkyl groups as described above.

As used herein, the term “alkylsulfonyl” refers to a group of the structure —S(O)₂-Alk wherein Alk refers to an alkyl group as described above. The terms “alkenylsulfonyl” and “alkynylsulfonyl” refer analogously to sulfonyl groups covalently bound to alkenyl and alkynyl groups, respectively. The term “arylsulfonyl” refers to a group of the structure —S(O)₂—Ar wherein Ar refers to an aryl group as described above. The term “aryloxyalkylsulfonyl” refers to a group of the structure —S(O)₂-Alk-O—Ar, where Alk is an alkyl group as described above and Ar is an aryl group as described above. The term “arylalkylsulfonyl” refers to a group of the structure —S(O)₂-AlkAr, where Alk is an alkyl group as described above and Ar is an aryl group as described above.

As used herein, the term “alkyloxycarbonyl” refers to an ester substituent including an alkyl group wherein the carbonyl carbon is the point of attachment to the molecule. An example is ethoxycarbonyl, which is CH₃CH₂OC(O)—. Similarly, the terms “alkenyloxycarbonyl,” “alkynyloxycarbonyl,” and “cycloalkylcarbonyl” refer to similar ester substituents including an alkenyl group, alkenyl group, or cycloalkyl group respectively. Similarly, the term “aryloxycarbonyl” refers to an ester substituent including an aryl group wherein the carbonyl carbon is the point of attachment to the molecule. Similarly, the term “aryloxyalkylcarbonyl” refers to an ester substituent including an alkyl group wherein the alkyl group is itself substituted by an aryloxy group.

Other combinations of substituents are known in the art and, are described, for example, in U.S. Pat. No. 8,344,162 to Jung et al., incorporated herein by this reference. For example, the term “thiocarbonyl” and combinations of substituents including “thiocarbonyl” include a carbonyl group in which a double-bonded sulfur replaces the normal double-bonded oxygen in the group. The term “alkylidene” and similar terminology refer to an alkyl group, alkenyl group, alkynyl group, or cycloalkyl group, as specified, that has two hydrogen atoms removed from a single carbon atom so that the group is double-bonded to the remainder of the structure.

For the aspects described below relating to improvement in the therapeutic employment of a substituted hexitol derivative, typically, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol, unless otherwise specified. Preferably, the substituted hexitol derivative is dianhydrogalactitol, unless otherwise specified. In some cases, derivatives of dianhydrogalactitol such as compound analogs or prodrugs are preferred, as stated below.

As used herein, unless further defined or limited, the term “antibody” encompasses both polyclonal and monoclonal antibodies, as well as genetically engineered antibodies such as chimeric, humanized or fully human antibodies of the appropriate binding specificity. As used herein, unless further defined, the term “antibody” also encompasses antibody fragments such as sFv, Fv, Fab, Fab′ and F(ab)′₂ fragments. In many cases, it is preferred to use monoclonal antibodies. In some contexts, antibodies can include fusion proteins comprising an antigen-binding site of an antibody, and any other modified immunoglobulin molecule comprising an antigen recognition site (i.e., antigen-binding site) as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins, antineoplastic agents, antimetabolites, or radioisotopes; in some cases, conjugation occurs through a linker or through noncovalent interactions such as an avidin-biotin or streptavidin-biotin linkage.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises an antigen-binding site or epitope-binding site. The term “variable region” of an antibody refers to the variable region of an antibody light chain, or the variable region of an antibody heavy chain, either alone or in combination. The variable regions of the heavy and light chains each consist of four framework regions (FR) connected by three complementarity determining regions (CDRs), also known as “hypervariable regions.” The CDRs in each chain are held together in close proximity by the framework regions and, with the CDRs from the other chain, contribute to the formation of the antigen-binding site of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda, Md.), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs. The term “monoclonal antibody” as used herein refers to a homogeneous antibody population involved in the highly specific recognition and binding of a single antigenic determinant or epitope. This is in contrast to polyclonal antibodies that typically include a mixture of different antibodies directed against a variety of different antigenic determinants. The term “monoclonal antibody” encompasses both intact and full-length monoclonal antibodies as well as antibody fragments (e.g., Fab, Fab′, F(ab′)2, Fv), single chain (sFv) antibodies, fusion proteins comprising an antibody portion, and any other modified immunoglobulin molecule comprising an antigen recognition site (antigen-binding site). Furthermore, “monoclonal antibody” refers to such antibodies made by any number of techniques, including but not limited to, hybridoma production, phage selection, recombinant expression, and expression in transgenic animals. The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability (Jones et al., 1986, Nature, 321:522-525; Riechmann et al., 1988, Nature, 332:323-327; Verhoeyen et al., 1988, Science, 239:1534-1536). In some instances, the Fv framework region residues of a human immunoglobulin are replaced with the corresponding residues in an antibody from a non-human species that has the desired specificity, affinity, and/or binding capability. The humanized antibody can be further modified by the substitution of additional residues either in the Fv framework region and/or within the replaced non-human residues to refine and optimize antibody specificity, affinity, and/or binding capability. In general, the humanized antibody will comprise substantially all of at least one, and typically two or three, variable domains containing all or substantially all of the CDRs that correspond to the non-human immunoglobulin whereas all or substantially all of the framework regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in, for example, U.S. Pat. No. 5,225,539. The term “human antibody” as used herein refers to an antibody produced by a human or an antibody having an amino acid sequence corresponding to an antibody produced by a human. A human antibody may be made using any of the techniques known in the art. This definition of a human antibody specifically excludes a humanized antibody comprising non-human CDRs. The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, or other antibody producing mammal) with the desired specificity, affinity, and/or binding capability, while the constant regions correspond to sequences in antibodies derived from another species (usually human). The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5 or 8-10 amino acids in a unique spatial conformation.

The terms “antagonist” and “antagonistic” as used herein refer to any molecule that partially or fully blocks, inhibits, reduces, or neutralizes a biological activity of a target and/or signaling pathway, or that partially or fully blocks, inhibits, reduces, or neutralizes the activity of a protein. Suitable antagonist molecules specifically include, but are not limited to, antagonist antibodies or antibody fragments. Similarly, the term “agonist” as used herein refers to any molecule that partially or fully promotes, activates, or accelerates a biological activity of a target and/or signaling pathway or the activity of a protein, or that overcomes antagonism. The terms “modulation” and “modulate” as used herein refer to a change or an alteration in a biological activity. Modulation includes, but is not limited to, stimulating or inhibiting an activity. Modulation may be an increase or a decrease in activity, a change in binding characteristics, or any other change in the biological, functional, or immunological properties associated with the activity of a protein, pathway, or other biological point of interest. The terms “selectively binds” or “specifically binds” mean that a binding agent or an antibody reacts or associates more frequently, more rapidly, with greater duration, with greater affinity, or with some combination of the above to the epitope, protein, or target molecule than with alternative substances, including unrelated proteins. In certain embodiments “specifically binds” means, for instance, that an antibody binds a protein with a K_{D} of about 0.1 mM or less, but more usually less than about 1 μM. In certain embodiments, “specifically binds” means that an antibody binds a target at times with a K_{D} of at least about 0.1 μM or less, at other times at least about 0.01 μM or less, and at other times at least about 1 nM or less. Because of the sequence identity between homologous proteins in different species, specific binding can include an antibody that recognizes a protein in more than one species. Likewise, because of homology within certain regions of polypeptide sequences of different proteins, specific binding can include an antibody (or other polypeptide or binding agent) that recognizes more than one protein. It is understood that, in certain embodiments, an antibody or binding moiety that specifically binds a first target may or may not specifically bind a second target. As such, “specific binding” does not necessarily require (although it can include) exclusive binding, i.e. binding to a single target. Thus, an antibody may, in certain embodiments, specifically bind more than one target. In certain embodiments, multiple targets may be bound by the same antigen-binding site on the antibody. For example, an antibody may, in certain instances, comprise two identical antigen-binding sites, each of which specifically binds the same epitope on two or more proteins. In certain alternative embodiments, an antibody may be multispecific and comprise at least two antigen-binding sites with differing specificities. By way of non-limiting example, a bispecific antibody may comprise one antigen-binding site that recognizes an epitope on one protein and further comprise a second, different antigen-binding site that recognizes a different epitope on a second protein. Generally, but not necessarily, reference to binding means specific binding.

As used herein, “analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or hydrophobic or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring variant of the original compound. Other types of analogues include isomers (enantiomers, diastereomers, and the like) and other types of chiral variants of a compound, as well as structural isomers.

As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have different chemical or physical properties of the parent compound. For example, the derivative may be more hydrophilic or hydrophobic or it may have altered reactivity as compared to the parent compound. Derivatization (i.e., modification) may involve substitution of one or more moieties within the molecule (e.g., a change in functional group). The term “derivative” also includes conjugates and prodrugs of a parent compound (i.e., chemically modified derivatives which can be converted into the original compound under physiological conditions).

In general, a description of a compound includes salts and solvates, including hydrates, of the compound unless specifically excluded.

One aspect of the present invention is an improvement in the therapeutic employment of a substituted hexitol derivative such as dianhydrogalactitol for treatment of NSCLC or GBM made by alterations to the time that the compound is administered, the use of dose-modifying agents that control the rate of metabolism of the compound, normal tissue protective agents, and other alterations. General examples include: variations of infusion schedules (e.g., bolus i.v. versus continuous infusion), the use of lymphokines (e.g., G-CSF, GM-CSF, EPO) to increase leukocyte count for improved immune response or for preventing anemia caused by myelosuppressive agents, or the use of rescue agents such as leucovorin for 5-FU or thiosulfate for cisplatin treatment. Specific inventive examples for a substituted hexitol derivative such as dianhydrogalactitol for treatment of NSCLC or GBM include: continuous i.v. infusion for hours to days; biweekly administration; doses greater than 5 mg/m²/day; progressive escalation of dosing from 1 mg/m²/day based on patient tolerance; doses less than 1 mg/m² for greater than 14 days; use of caffeine to modulate metabolism; use of isoniazid to modulate metabolism; single and multiple doses escalating from 5 mg/m²/day via bolus; oral doses below 30 or above 130 mg/m²; oral dosages up to 40 mg/m² for 3 days and then a nadir/recovery period of 18-21 days; dosing at a lower level for an extended period (e.g., 21 days); dosing at a higher level; dosing with a nadir/recovery period longer than 21 days; the use of a substituted hexitol derivative such as dianhydrogalactitol as a single cytotoxic agent, typically at 30 mg/m²/day×5 days, repeated monthly; dosing at 3 mg/kg; the use of a substituted hexitol derivative such as dianhydrogalactitol in combination therapy, typically at 30 mg/m²/day×5 days; or dosing at 40 mg/day×5 days in adult patients, repeated every two weeks.

Another aspect of the invention is an improvement in the therapeutic employment of a substituted hexitol derivative such as dianhydrogalactitol for treatment of NSCLC or GBM made by alterations in the route by which the compound is administered. General examples include: changing route from oral to intravenous administration and vice versa; or the use of specialized routes such as subcutaneous, intramuscular, intraarterial, intraperitoneal, intralesional, intralymphatic, intratumoral, intrathecal, intravesicular, intracranial. Specific inventive examples for a substituted hexitol derivative such as dianhydrogalactitol for treatment of NSCLC include: topical administration; oral administration; slow-release oral delivery; intrathecal administration; intraarterial administration; continuous infusion; intermittent infusion; intravenous administration; or administration through a longer infusion; or administration through IV push.

Yet another aspect of the invention is an improvement in the therapeutic employment of a substituted hexitol derivative such as dianhydrogalactitol made by changes in the schedule of administration. General examples include: daily administration, biweekly administration, or weekly administration. Specific inventive examples for a substituted hexitol derivative such as dianhydrogalactitol for treatment of NSCLC or GBM include: daily administration; weekly administration; weekly administration for three weeks; biweekly administration; biweekly administration for three weeks with a 1-2 week rest period; intermittent boost dose administration; or daily administration for one week for multiple weeks.

Yet another aspect of the invention is an improvement in the therapeutic employment of a substituted hexitol derivative such as dianhydrogalactitol for treatment of NSCLC or GBM made by alterations in the stage of disease at diagnosis/progression that the compound is administered. General examples include: the use of chemotherapy for non-resectable local disease, prophylactic use to prevent metastatic spread or inhibit disease progression or conversion to more malignant stages. Specific inventive examples for a substituted hexitol derivative such as dianhydrogalactitol for treatment of NSCLC or GBM include: use in an appropriate disease stage for NSCLC; use of the substituted hexitol derivative such as dianhydrogalactitol with angiogenesis inhibitors such as Avastin™, a VEGF inhibitor, to prevent or limit metastatic spread; the use of a substituted hexitol derivative such as dianhydrogalactitol for newly diagnosed disease; the use of a substituted hexitol derivative such as dianhydrogalactitol for recurrent disease; or the use of a substituted hexitol derivative such as dianhydrogalactitol for resistant or refractory disease.

Yet another aspect of the invention is an improvement in the therapeutic employment of a substituted hexitol derivative such as dianhydrogalactitol for treatment of NSCLC or GBM made by alterations to the type of patient that would best tolerate or benefit from the use of the compound. General examples include: use of pediatric doses for elderly patients, altered doses for obese patients; exploitation of co-morbid disease conditions such as diabetes, cirrhosis, or other conditions that may uniquely exploit a feature of the compound. Specific inventive examples for a substituted hexitol derivative such as dianhydrogalactitol for treatment of NSCLC or GBM include: patients with a disease condition characterized by a high level of a metabolic enzyme selected from the group consisting of histone deacetylase and ornithine decarboxylase; patients with a low or high susceptibility to a condition selected from the group consisting of thrombocytopenia and neutropenia; patients intolerant of GI toxicities; patients characterized by over- or under-expression of a gene selected from the group consisting of c-Jun, a GPCR, a signal transduction protein, VEGF, a prostate-specific gene, and a protein kinase; prostate-specific gene, and a protein kinase; patients characterized by a mutation in EGFR including, but not limited to, EGFR Variant III; patients being administered a platinum-based drug as combination therapy; patients who do not have EGFR mutations and thus are less likely to respond to tyrosine kinase inhibitors (TKI); patients who have become resistant to TKI treatment; patients who have the BIM co-deletion mutation and thus are less likely to respond to TKI treatment; patients who have become resistant to platinum-based drug treatment; or patients with brain metastases.

Yet another aspect of the invention is an improvement in the therapeutic employment of a substituted hexitol derivative such as dianhydrogalactitol for treatment of NSCLC or GBM made by more precise identification of a patient's ability to tolerate, metabolize and exploit the use of the compound as associated with a particular phenotype of the patient. General examples include: use of diagnostic tools and kits to better characterize a patient's ability to process/metabolize a chemotherapeutic agent or the susceptibility of the patient to toxicity caused by potential specialized cellular, metabolic, or organ system phenotypes. Specific inventive examples for a substituted hexitol derivative such as dianhydrogalactitol for treatment of NSCLC or GBM include: use of a diagnostic tool, a diagnostic technique, a diagnostic kit, or a diagnostic assay to confirm a patient's particular phenotype; use of a method for measurement of a marker selected from the group consisting of histone deacetylase, ornithine decarboxylase, VEGF, a protein that is a gene product of jun, and a protein kinase; surrogate compound testing; or low dose pre-testing for enzymatic status.

Yet another aspect of the invention is an improvement in the therapeutic employment of a substituted hexitol derivative such as dianhydrogalactitol for treatment of NSCLC or GBM made by more precise identification of a patient's ability to tolerate, metabolize and exploit the use of the compound as associated with a particular genotype of the patient. General examples include: biopsy samples of tumors or normal tissues (e.g., glial cells or other cells of the central nervous system) that may also be taken and analyzed to specifically tailor or monitor the use of a particular drug against a gene target; studies of unique tumor gene expression patterns; or analysis of SNP's (single nucleotide polymorphisms), to enhance efficacy or to avoid particular drug-sensitive normal tissue toxicities. Specific inventive examples for a substituted hexitol derivative such as dianhydrogalactitol for treatment of NSCLC or GBM include: diagnostic tools, techniques, kits and assays to confirm a patient's particular genotype; gene/protein expression chips and analysis; Single Nucleotide Polymorphisms (SNP's) assessment; SNP's for histone deacetylase, ornithine decarboxylase, GPCR's, protein kinases, telomerase, or jun; identification and measurement of metabolism enzymes and metabolites; determination of mutation of PDGFRA gene; determination of mutation of IDH1 gene; determination of mutation of NF1 gene; determination of copy number of the EGFR gene; determination of status of methylation of promoter of MGMT gene; use for disease characterized by an unmethylated promoter region of the MGMT gene; use for disease characterized by a methylated promoter region of the MGMT gene; use for disease characterized by high expression of MGMT; use for disease characterized by low expression of MGMT; or use for disease characterized by EML4-ALK translocations.

Yet another aspect of the invention is an improvement in the therapeutic employment of a substituted hexitol derivative such as dianhydrogalactitol for treatment of NSCLC or GBM made by specialized preparation of a patient prior to or after the use of a chemotherapeutic agent. General examples include: induction or inhibition of metabolizing enzymes, specific protection of sensitive normal tissues or organ systems. Specific inventive examples for a substituted hexitol derivative such as dianhydrogalactitol for treatment of NSCLC or GBM include: the use of colchicine or analogs; use of diuretics such as probenecid; use of a uricosuric; use of uricase; non-oral use of nicotinamide; sustained release forms of nicotinamide; use of inhibitors of poly (ADP ribose) polymerase; use of caffeine; leucovorin rescue; infection control; antihypertensives.

Yet another aspect of the invention is an improvement in the therapeutic employment of a substituted hexitol derivative such as dianhydrogalactitol for treatment of NSCLC or GBM made by use of additional drugs or procedures to prevent or reduce potential side-effects or toxicities. General examples include: the use of anti-emetics, anti-nausea, hematological support agents to limit or prevent neutropenia, anemia, thrombocytopenia, vitamins, antidepressants, treatments for sexual dysfunction, and other supportive techniques. Specific inventive examples for a substituted hexitol derivative such as dianhydrogalactitol for treatment of NSCLC or GBM include: the use of colchicine or analogs; use of diuretics such as probenecid; use of a uricosuric; use of uricase; non-oral use of nicotinamide; use of sustained release forms of nicotinamide; use of inhibitors of poly ADP-ribose polymerase; use of caffeine; leucovorin rescue; use of sustained release allopurinol; non-oral use of allopurinol; use of bone marrow transplants; use of a blood cell stimulant; use of blood or platelet infusions; use of filgrastim, G-CSF, or GM-CSF; use of pain management techniques; use of anti-inflammatories; use of fluids; use of corticosteroids; use of insulin control medications; use of antipyretics; use of anti-nausea treatments; use of anti-diarrheal treatment; use of N-acetylcysteine; or use of antihistamines.

Yet another aspect of the invention is an improvement in the therapeutic employment of a substituted hexitol derivative such as dianhydrogalactitol for treatment of NSCLC or GBM made by the use of monitoring drug levels after dosing in an effort to maximize a patient's drug plasma level, to monitor the generation of toxic metabolites, monitoring of ancillary medicines that could be beneficial or harmful in terms of drug-drug interactions. General examples include: the monitoring of drug plasma protein binding, and monitoring of other pharmacokinetic or pharmacodynamic variables. Specific inventive examples for a substituted hexitol derivative such as dianhydrogalactitol for treatment of NSCLC or GBM include: multiple determinations of drug plasma levels; or multiple determinations of metabolites in the blood or urine.

Yet another aspect of the invention is an improvement in the therapeutic employment of a substituted hexitol derivative such as dianhydrogalactitol for treatment of NSCLC or GBM made by exploiting unique drug combinations that may provide a more than additive or synergistic improvement in efficacy or side-effect management. Specific inventive examples for a substituted hexitol derivative such as dianhydrogalactitol for treatment of NSCLC or GBM include: use with topoisomerase inhibitors; use with fraudulent nucleosides; use with fraudulent nucleotides; use with thymidylate synthetase inhibitors; use with signal transduction inhibitors; use with cisplatin or platinum analogs; use with alkylating agents such as the nitrosoureas (BCNU, Gliadel™ wafers, CCNU, nimustine (ACNU), bendamustine (Treanda™)); use with alkylating agents that damage DNA at a different place than does DAG (TMZ, BCNU, CCNU, and other alkylating agents all damage DNA at O⁶ of guanine, whereas DAG cross-links at N⁷); use with a monofunctional alkylating agent; use with a bifunctional alkylating agent; use with anti-tubulin agents; use with antimetabolites; use with berberine; use with apigenin; use with amonafide; use with colchicine or analogs; use with genistein; use with etoposide; use with cytarabine; use with camptothecins; use with vinca alkaloids; use with topoisomerase inhibitors; use with 5-fluorouracil; use with curcumin; use with NF-κB inhibitors; use with rosmarinic acid; use with mitoguazone; use with tetrandrine; use with temozolomide (TMZ); use with biological therapies such as antibodies such as Avastin™ (a VEGF inhibitor), Rituxan™, Herceptin™, Erbitux™; use with epidermal growth factor receptor (EGFR) inhibitors; use with tyrosine kinase inhibitors; use with poly (ADP-ribose) polymerase (PARP) inhibitors; or use with cancer vaccine therapy. The ability to be more than additive or synergistic is particularly significant with respect to the combination of a substituted hexitol derivative such as dianhydrogalactitol with cisplatin or other platinum-containing chemotherapeutic agents.

Yet another aspect of the invention is an improvement in the therapeutic employment of a substituted hexitol derivative such as dianhydrogalactitol for treatment of NSCLC or GBM made by exploiting the substituted hexitol derivative such as dianhydrogalactitol as a chemosensitizer where no measurable activity is observed when used alone but in combination with other therapeutics a more than additive or synergistic improvement in efficacy is observed. Specific inventive examples for a substituted hexitol derivative such as dianhydrogalactitol for treatment of NSCLC or GBM include: as a chemosensitizer in combination with topoisomerase inhibitors; as a chemosensitizer in combination with fraudulent nucleosides; as a chemosensitizer in combination with fraudulent nucleotides; as a chemosensitizer in combination with thymidylate synthetase inhibitors; as a chemosensitizer in combination with signal transduction inhibitors; as a chemosensitizer in combination with cisplatin or platinum analogs; as a chemosensitizer in combination with alkylating agents such as BCNU, BCNU wafers, Gliadel™, CCNU, bendamustine (Treanda™), or Temozolomide (Temodar™); as a chemosensitizer in combination with anti-tubulin agents; as a chemosensitizer in combination with antimetabolites; as a chemosensitizer in combination with berberine; as a chemosensitizer in combination with apigenin; as a chemosensitizer in combination with amonafide; as a chemosensitizer in combination with colchicine or analogs; as a chemosensitizer in combination with genistein; as a chemosensitizer in combination with etoposide; as a chemosensitizer in combination with cytarabine; as a chemosensitizer in combination with camptothecins; as a chemosensitizer in combination with vinca alkaloids; as a chemosensitizer in combination with topoisomerase inhibitors; as a chemosensitizer in combination with 5-fluorouracil; as a chemosensitizer in combination with curcumin; as a chemosensitizer in combination with NF-κB inhibitors; as a chemosensitizer in combination with rosmarinic acid; as a chemosensitizer in combination with mitoguazone; as a chemosensitizer in combination with tetrandrine; as a chemosensitizer in combination with a tyrosine kinase inhibitor; as a chemosensitizer in combination with an EGFR inhibitor; or as a chemosensitizer in combination with an inhibitor of poly (ADP-ribose) polymerase (PARP).

Yet another aspect of the invention is an improvement in the therapeutic employment of a substituted hexitol derivative such as dianhydrogalactitol for treatment of NSCLC or GBM made by exploiting the substituted hexitol derivative such as dianhydrogalactitol as a chemopotentiator where minimal therapeutic activity is observed alone but in combination with other therapeutics a more than additive or synergistic improvement in efficacy is observed. Specific inventive examples for a substituted hexitol derivative such as dianhydrogalactitol for treatment of NSCLC or GBM include: as a chemopotentiator in combination with topoisomerase inhibitors; as a chemopotentiator in combination with fraudulent nucleosides; as a chemopotentiator in combination with thymidylate synthetase inhibitors; as a chemopotentiator in combination with signal transduction inhibitors; as a chemopotentiator in combination with cisplatin or platinum analogs; as a chemopotentiator in combination with use with alkylating agents such as BCNU, BCNU wafers, Gliadel™, or bendamustine (Treanda™); as a chemopotentiator in combination with anti-tubulin agents; as a chemopotentiator in combination with antimetabolites; as a chemopotentiator in combination with berberine; as a chemopotentiator in combination with apigenin; as a chemopotentiator in combination with amonafide; as a chemopotentiator in combination with colchicine or analogs; as a chemopotentiator in combination with genistein; as a chemopotentiator in combination with etoposide; as a chemopotentiator in combination with cytarabine; as a chemopotentiator in combination with camptothecins; as a chemopotentiator in combination with vinca alkaloids; as a chemopotentiator in combination with topoisomerase inhibitors; as a chemopotentiator in combination with 5-fluorouracil; as a chemopotentiator in combination with curcumin; as a chemopotentiator in combination with NF-κB inhibitors; as a chemopotentiator in combination with rosmarinic acid; as a chemopotentiator in combination with mitoguazone; as a chemopotentiator in combination with tetrandrine; as a chemopotentiator in combination with a tyrosine kinase inhibitor; as a chemopotentiator in combination with an EGFR inhibitor; or as a chemopotentiator in combination with an inhibitor of poly (ADP-ribose) polymerase (PARP).

Yet another aspect of the invention is an improvement in the therapeutic employment of a substituted hexitol derivative such as dianhydrogalactitol for treatment of NSCLC or GBM made by drugs, treatments and diagnostics to allow for the maximum benefit to patients treated with a compound. General examples include: pain management, nutritional support, anti-emetics, anti-nausea therapies, anti-anemia therapy, anti-inflammatories. Specific inventive examples for a substituted hexitol derivative such as dianhydrogalactitol for treatment of NSCLC or GBM include: use with therapies associated with pain management; nutritional support; anti-emetics; anti-nausea therapies; anti-anemia therapy; anti-inflammatories: antipyretics; immune stimulants.

Yet another aspect of the invention is an improvement in the therapeutic employment of a substituted hexitol derivative such as dianhydrogalactitol for treatment of NSCLC or GBM made by the use of complementary therapeutics or methods to enhance effectiveness or reduce side effects. Specific inventive examples for a substituted hexitol derivative such as dianhydrogalactitol for treatment of NSCLC or GBM include: hypnosis; acupuncture; meditation; herbal medications created either synthetically or through extraction including NF-κB inhibitors (such as parthenolide, curcumin, rosmarinic acid); natural anti-inflammatories (including rhein, parthenolide); immunostimulants (such as those found in Echinacea); antimicrobials (such as berberine); flavonoids, isoflavones, and flavones (such as apigenenin, genistein, genistin, 6″-O-malonylgenistin, 6″-O-acetylgenistin, daidzein, daidzin, 6″-O-malonyldaidzin, 6″-O-acetylgenistin, glycitein, glycitin, 6″-O-malonylglycitin, and 6-O-acetylglycitin); applied kinesiology.

Yet another aspect of the invention is an improvement in the therapeutic employment of a substituted hexitol derivative such as dianhydrogalactitol for treatment of NSCLC or GBM made by alterations in the pharmaceutical bulk substance. General examples include: salt formation, homogeneous crystalline structure, pure isomers. Specific inventive examples for a substituted hexitol derivative such as dianhydrogalactitol for treatment of NSCLC or GBM include: salt formation; homogeneous crystalline structure; pure isomers; increased purity; lower residual solvents; or lower heavy metals.

Yet another aspect of the invention is an improvement in the therapeutic employment of a substituted hexitol derivative such as dianhydrogalactitol for treatment of NSCLC or GBM made by alterations in the diluents used to solubilize and deliver/present the compound for administration. General examples include: Cremophor-EL™, cyclodextrins for poorly water soluble compounds. Specific inventive examples for a substituted hexitol derivative such as dianhydrogalactitol for treatment of NSCLC or GBM include: use of emulsions; dimethyl sulfoxide (DMSO); N-methylformamide (NMF); dimethylformamide (DMF); dimethylacetamide (DMA); ethanol; benzyl alcohol; dextrose containing water for injection; Cremophor™; cyclodextrins; PEG.

Yet another aspect of the invention is an improvement in the therapeutic employment of a substituted hexitol derivative such as dianhydrogalactitol for treatment of NSCLC or GBM made by alterations in the solvents used or required to solubilize a compound for administration or for further dilution. General examples include: ethanol, dimethylacetamide (DMA). Specific inventive examples for a substituted hexitol derivative such as dianhydrogalactitol for treatment of NSCLC or GBM include: the use of emulsions; DMSO; NMF; DMF; DMA; ethanol; benzyl alcohol; dextrose containing water for injection; Cremophor™; cyclodextrin; or PEG.

Yet another aspect of the invention is an improvement in the therapeutic employment of a substituted hexitol derivative such as dianhydrogalactitol for treatment of NSCLC or GBM made by alterations in the materials/excipients, buffering agents, or preservatives required to stabilize and present a chemical compound for proper administration. General examples include: mannitol, albumin, EDTA, sodium bisulfite, benzyl alcohol. Specific inventive examples for a substituted hexitol derivative such as dianhydrogalactitol for treatment of NSCLC or GBM include: the use of mannitol; albumin; EDTA; sodium bisulfite; benzyl alcohol; carbonate buffers; phosphate buffers.

Yet another aspect of the invention is an improvement in the therapeutic employment of a substituted hexitol derivative such as dianhydrogalactitol for treatment of NSCLC or GBM made by alterations in the potential dosage forms of the compound dependent on the route of administration, duration of effect, plasma levels required, exposure to side effects in normal tissues and metabolizing enzymes. General examples include: tablets, capsules, topical gels, creams, patches, suppositories. Specific inventive examples for a substituted hexitol derivative such as dianhydrogalactitol for treatment of NSCLC or GBM include: the use of tablets; capsules; topical gels; topical creams; patches; suppositories; lyophilized dosage fills.

Yet another aspect of the invention is an improvement in the therapeutic employment of a substituted hexitol derivative such as dianhydrogalactitol for treatment of NSCLC or GBM made by alterations in the dosage forms, container/closure systems, accuracy of mixing and dosage preparation and presentation. General examples include: amber vials to protect from light, stoppers with specialized coatings. Specific inventive examples for a substituted hexitol derivative such as dianhydrogalactitol for treatment of NSCLC or GBM include: the use of amber vials to protect from light; stoppers with specialized coatings to improve shelf-life stability.

Yet another aspect of the invention is an improvement in the therapeutic employment of a substituted hexitol derivative such as dianhydrogalactitol for treatment of NSCLC or GBM made by the use of delivery systems to improve the potential attributes of a pharmaceutical product such as convenience, duration of effect, reduction of toxicities. General examples include: nanocrystals, bioerodible polymers, liposomes, slow release injectable gels, microspheres. Specific inventive examples for a substituted hexitol derivative such as dianhydrogalactitol for treatment of NSCLC or GBM include: the use of nanocrystals; bioerodible polymers; liposomes; slow release injectable gels; microspheres.

Yet another aspect of the invention is an improvement in the therapeutic employment of a substituted hexitol derivative such as dianhydrogalactitol for treatment of NSCLC or GBM made by alterations to the parent molecule with covalent, ionic, or hydrogen bonded moieties to alter the efficacy, toxicity, pharmacokinetics, metabolism, or route of administration. General examples include: polymer systems such as polyethylene glycols, polylactides, polyglycolides, amino acids, peptides, or multivalent linkers. Specific inventive examples for a substituted hexitol derivative such as dianhydrogalactitol for treatment of NSCLC or GBM include: the use of polymer systems such as polyethylene glycols; polylactides; polyglycolides; amino acids; peptides; multivalent linkers.

Yet another aspect of the invention is an improvement in the therapeutic employment of a substituted hexitol derivative such as dianhydrogalactitol for treatment of NSCLC or GBM made by alterations to the molecule such that improved pharmaceutical performance is gained with a variant of the active molecule in that after introduction into the body a portion of the molecule is cleaved to reveal the preferred active molecule. General examples include: enzyme sensitive esters, dimers, Schiff bases. Specific inventive examples for a substituted hexitol derivative such as dianhydrogalactitol for treatment of NSCLC or GBM include: the use of enzyme sensitive esters; dimers; Schiff bases; pyridoxal complexes; caffeine complexes.

Yet another aspect of the invention is an improvement in the therapeutic employment of a substituted hexitol derivative such as dianhydrogalactitol for treatment of NSCLC or GBM made by the use of additional compounds, biological agents that, when administered in the proper fashion, a unique and beneficial effect can be realized. General examples include: inhibitors of multi-drug resistance, specific drug resistance inhibitors, specific inhibitors of selective enzymes, signal transduction inhibitors, repair inhibition. Specific inventive examples for a substituted hexitol derivative such as dianhydrogalactitol for treatment of NSCLC or GBM include: the use of inhibitors of multi-drug resistance; specific drug resistance inhibitors; specific inhibitors of selective enzymes; signal transduction inhibitors; repair inhibition; topoisomerase inhibitors with non-overlapping side effects.

Yet another aspect of the invention is an improvement in the therapeutic employment of a substituted hexitol derivative such as dianhydrogalactitol for treatment of NSCLC or GBM made by the use of the substituted hexitol derivative such as dianhydrogalactitol in combination as sensitizers/potentiators with biological response modifiers. General examples include: use in combination as sensitizers/potentiators with biological response modifiers, cytokines, lymphokines, therapeutic antibodies, antisense therapies, gene therapies. Specific inventive examples for a substituted hexitol derivative such as dianhydrogalactitol for treatment of NSCLC or GBM include: use in combination as sensitizers/potentiators with biological response modifiers; cytokines; lymphokines; therapeutic antibodies such as Avastin™, Herceptin™, Rituxan™, and Erbitux™; antisense therapies; gene therapies; ribozymes; RNA interference; or vaccines.

Yet another aspect of the invention is an improvement in the therapeutic employment of a substituted hexitol derivative such as dianhydrogalactitol for treatment of NSCLC or GBM made by exploiting the selective use of the substituted hexitol derivative such as dianhydrogalactitol to overcome developing or complete resistance to the efficient use of biotherapeutics. General examples include: tumors resistant to the effects of biological response modifiers, cytokines, lymphokines, therapeutic antibodies, antisense therapies, gene therapies. Specific inventive examples for a substituted hexitol derivative such as dianhydrogalactitol for treatment of NSCLC or GBM include: the use against tumors resistant to the effects of biological response modifiers; cytokines; lymphokines; therapeutic antibodies; antisense therapies; therapies such as Avastin™, Rituxan™, Herceptin™, Erbitux™; gene therapies; ribozymes; RNA interference; and vaccines.

Yet another aspect of the invention is an improvement in the therapeutic employment of a substituted hexitol derivative such as dianhydrogalactitol for treatment of NSCLC or GBM made by exploiting their use in combination with ionizing radiation, phototherapies, heat therapies, or radio-frequency generated therapies. General examples include: hypoxic cell sensitizers, radiation sensitizers/protectors, photosensitizers, radiation repair inhibitors. Specific inventive examples for a substituted hexitol derivative such as dianhydrogalactitol for treatment of NSCLC or GBM include: use in combination with ionizing radiation; use in combination with hypoxic cell sensitizers; use in combination with radiation sensitizers/protectors; use in combination with photosensitizers; use in combination with radiation repair inhibitors; use in combination with thiol depletion; use in combination with vaso-targeted agents; use in combination with use with radioactive seeds; use in combination with radionuclides; use in combination with radiolabeled antibodies; use in combination with brachytherapy. This is useful because radiation therapy is frequently employed in the treatment of NSCLC or GBM, especially for advanced disease, and improvements in the efficacy of such radiation therapy or the ability to exert a synergistic effect by combining radiation therapy with the administration of a substituted hexitol derivative such as dianhydrogalactitol is significant for these malignancies.

Radiotherapy can be used for treatment of non-small-cell lung carcinoma (NSCLC), either alone or together with chemotherapy. The use of radiotherapy for the treatment of NSCLC has been described in M. Provencio et al., “Inoperable Stage III Non-Small Cell Lung Cancer: Current Treatment and Role of Vinorelbine,” J. Thoracic Dis. 3:197-204 (2011), incorporated herein by this reference. Various dosage protocols can be used, and radiation can be administered either concurrently or separately with chemotherapy when both radiation and chemotherapy are used. Radiation can be administered in either a single dose, or in fractionated doses. A typical single dose is 60 Gy, but when radiation is administered in fractionated doses, a somewhat higher dosage can be administered in toto. Total doses can range from about 40 Gy to about 79.2 Gy. Radiation can be administered as high-energy X-rays or high-energy electrons from linear accelerator units; in some cases, gamma rays can be administered from a cobalt-60-based device. Other radiotherapy methods are known in the art. For GBM, radiotherapy is also frequently used; the use of radiotherapy for the treatment of GBM is described in T. N. Showalter et al., “Multifocal Glioblastoma Multiforme: Prognostic Factors and Patterns of Progression,” Int. J. Radiation Oncol. Biol. Phys. 69:820-824 (2007), incorporated herein by this reference. A dose of about 60 Gy is generally considered optimal, and three-dimensional conformal radiotherapy is frequently used. As GBM tumors frequently include regions with hypoxia that are resistant to radiotherapy, in one alternative, a radiosensitizer such as trans sodium crocetinate can be used.

Yet another aspect of the invention is an improvement in the therapeutic employment of a substituted hexitol derivative such as dianhydrogalactitol for treatment of NSCLC or GBM made by optimizing its utility by determining the various mechanisms of action, biological targets of a compound for greater understanding and precision to better exploit the utility of the molecule. Specific inventive examples for a substituted hexitol derivative such as dianhydrogalactitol for treatment of NSCLC or GBM include: the use with inhibitors of poly-ADP ribose polymerase; agents that effect vasculature or vasodilation; oncogenic targeted agents; signal transduction inhibitors; EGFR inhibition; Protein Kinase C inhibition; Phospholipase C downregulation; Jun downregulation; histone genes; VEGF; ornithine decarboxylase; ubiquitin C; jun D; v-jun; GPCRs; protein kinase A; telomerase, prostate specific genes; protein kinases other than protein kinase A; histone deacetylase; and tyrosine kinase inhibitors.

Yet another aspect of the invention is an improvement in the therapeutic employment of a substituted hexitol derivative such as dianhydrogalactitol for treatment of NSCLC or GBM made by more precise identification and exposure of the compound to those select cell populations where the compound's effect can be maximally exploited, particularly NSCLC tumor cells or GBM tumor cells. Specific inventive examples for a substituted hexitol derivative such as dianhydrogalactitol for treatment of NSCLC or GBM include: use against radiation sensitive cells; use against radiation resistant cells; or use against energy depleted cells.

Yet another aspect of the invention is an improvement in the therapeutic employment of a substituted hexitol derivative such as dianhydrogalactitol for treatment of NSCLC or GBM made by use of an agent that counteracts myelosuppression. Specific inventive examples for a substituted hexitol derivative such as dianhydrogalactitol for treatment of NSCLC or GBM include use of dithiocarbamates to counteract myelosuppression.

Yet another aspect of the invention is an improvement in the therapeutic employment of a substituted hexitol derivative such as dianhydrogalactitol for treatment of brain metastases of NSCLC made by use of an agent that increases the ability of the substituted hexitol to pass through the blood-brain barrier. This can also be employed for GBM, which is a central nervous system malignancy. Specific examples for a substituted hexitol derivative such as dianhydrogalactitol for treatment of brain metastases of NSCLC or for GBM include chimeric peptides; compositions comprising either avidin or an avidin fusion protein bonded to a biotinylated substituted hexitol derivative; neutral liposomes that are pegylated and that incorporate the substituted hexitol derivative and wherein the polyethylene glycol strands are conjugated to at least one transportable peptide or targeting agent; a humanized murine antibody that binds to the human insulin receptor linked to the substituted hexitol derivative through an avidin-biotin linkage; and a fusion protein linked to the hexitol through an avidin-biotin linkage.

Yet another aspect of the invention is an improvement in the therapeutic employment of a substituted hexitol derivative such as dianhydrogalactitol for treatment of brain metastases of NSCLC or GBM made by use of an agent that suppresses the growth of cancer stem cells (CSCs). Specific examples for a substituted hexitol derivative such as dianhydrogalactitol for treatment of brain metastases of NSCLC or for GBM include: (1) naphthoquinones; (2) VEGF-DLLA bispecific antibodies; (3) farnesyl transferase inhibitors; (4) gamma-secretase inhibitors; (5) anti-TIM3 antibodies; (6) tankyrase inhibitors; (7) Wnt pathway inhibitors other than tankyrase inhibitors; (8) camptothecin-binding moiety conjugates; (9) Notch1 binding agents, including antibodies; (10) oxabicycloheptanes and oxabicycloheptenes; (11) inhibitors of the mitochondrial electron transport chains or the mitochondrial tricarboxylic acid cycle; (12) Axl inhibitors; (13) dopamine receptor antagonists; (14) anti-RSPO1 antibodies; (15) inhibitors or modulators of the Hedgehog pathway; (16) caffeic acid analogs and derivatives; (17) Stat3 inhibitors; (18) GRP-94-binding antibodies; (19) Frizzled receptor polypeptides; (20) immunoconjugates with cleavable linkages; (21) human prolactin, growth hormone, or placental lactogen; (22) anti-prominin-1 antibody; (23) antibodies specifically binding N-cadherin; (24) DR5 agonists; (25) anti-DLL4 antibodies or binding fragments thereof; (26) antibodies specifically binding GPR49; (27) DDR1 binding agents; (28) LGR5 binding agents; (29) telomerase-activating compounds; (30) fingolimod plus anti-CD74 antibodies or fragments thereof; (31) an antibody that prevents the binding of CD47 to SIPRα or a CD47 mimetic; (32) thienopyranone kinase inhibitors for inhibition of PI-3 kinases; (33) cancer-stem-cell-binding peptides; (34) diphtheria toxin-interleukin 3 conjugates; (35) inhibitors of histone deacetylase; (36) progesterone or analogs thereof; (37) antibodies binding the negative regulatory region (NRR) of Notch2; (38) inhibitors of HGFIN; (39) immunotherapeutic peptides; (40) inhibitors of CSCPK or related kinases; (41) imidazo[1,2-a]pyrazine derivatives as α-helix mimetics; (42) antibodies directed to an epitope of variant Heterogeneous Ribonucleoprotein G (HnRNPG); (43) antibodies binding TES7 antigen; (44) antibodies binding the ILR3α subunit; (45) ifenprodil tartrate and other compounds with a similar activity; (46) antibodies binding SALL4; (47) antibodies binding Notch4; (48) bispecific antibodies binding both NBR1 and Cep55; (49) Smo inhibitors; (50) peptides blocking or inhibiting interleukin-1 receptor 1; (51) antibodies specific for CD47 or CD19; (52) histone methyltransferase inhibitors; (53) antibodies specifically binding Lg5; (54) antibodies specifically binding EFNA1; (55) phenothiazine derivatives; (56) HDAC inhibitors plus AKT inhibitors; (57) ligands binding to cancer-stem-line-specific cell surface antigen stem cell markers; (58) Notch receptor agonists; (59) binding agents binding human MET; (60) PDGFR-β inhibitors; (61) pyrazolo compounds with histone demethylase activity; (62) heterocyclic substituted 3-heteroaryidenyl-2-indolinone derivatives; (63) albumin-binding arginine deiminase fusion proteins; (64) hydrogen-bond surrogate peptides and peptidomimetics that reactivate p53; (65) prodrugs of 2-pyrrolinodoxorubicin conjugated to antibodies; (66) targeted cargo proteins; (67) bisacodyl and analogs thereof; (68) N¹-cyclic amine-N⁵-substituted phenyl biguanide derivative; (69) fibulin-3 protein; (70) modulators of SCFSkp2; (71) inhibitors of Slingshot-2; (72) monoclonal antibodies specifically binding DCLK1 protein; (73) antibodies or soluble receptors that modulate the Hippo pathway; (74) selective inhibitors of CDK8 and CDK19; (75) antibodies and antibody fragments specifically binding IL-17; (76) antibodies specifically binding FRMD4A; (77) monoclonal antibodies specifically binding the ErbB-3 receptor; (78) antibodies that specifically bind human RSPO3 and modulate β-catenin activity; (79) esters of 4,9-dihydroxy-naphtho[2,3-b]furans; (80) CCR5 antagonists; (81) antibodies that specifically bind the extracellular domain of human C-type lectin-like molecule (CLL-1); (82) anti-hypertension compounds; (83) anthraquinone radiosensitizer agents plus ionizing radiation; (84) CDK inhibiting pyrrolopyrimidinone derivatives; (85) analogs of CC-1065 and conjugates thereof; (86) antibodies specifically binding to the protein Notum; (87) CDK8 antagonists; (88) bHLH proteins and nucleic acids encoding them; (89) inhibitors of the histone methyltransferase EZH2; (90) sulfonamides inhibiting carbonic anhydrase isoforms; (91) antibodies specifically binding DEspR; (92) antibodies specifically binding human leukemia inhibitory factor (LIF); (93) doxovir; (94) inhibitors of mTOR; (95) antibodies specifically binding FZD10; (96) napthofurans; (97) death receptor agonists; (98) tigecycline; (99) strigolactones and strigolactone analogs; and (100) compounds inducing methuosis.

Accordingly, one aspect of the present invention is a method to improve the efficacy and/or reduce the side effects of the administration of a substituted hexitol derivative such as dianhydrogalactitol for treatment of NSCLC or GBM comprising the steps of:

(1) identifying at least one factor or parameter associated with the efficacy and/or occurrence of side effects of the administration of the substituted hexitol derivative such as dianhydrogalactitol for treatment of NSCLC or GBM; and

(2) modifying the factor or parameter to improve the efficacy and/or reduce the side effects of the administration of the substituted hexitol derivative such as dianhydrogalactitol for treatment of NSCLC or GBM.

Typically, the factor or parameter is selected from the group consisting of:

(1) dose modification;

(2) route of administration;

(3) schedule of administration;

(4) indications for use;

(5) selection of disease stage;

(6) other indications;

(7) patient selection;

(8) patient/disease phenotype;

(9) patient/disease genotype;

(10) pre/post-treatment preparation;

(11) toxicity management;

(12) pharmacokinetic/pharmacodynamic monitoring;

(13) drug combinations;

(14) chemosensitization;

(15) chemopotentiation;

(16) post-treatment patient management;

(17) alternative medicine/therapeutic support;

(18) bulk drug product improvements;

(19) diluent systems;

(20) solvent systems;

(21) excipients;

(22) dosage forms;

(23) dosage kits and packaging;

(24) drug delivery systems;

(25) drug conjugate forms;

(26) compound analogs;

(27) prodrugs;

(28) multiple drug systems;

(29) biotherapeutic enhancement;

(30) biotherapeutic resistance modulation;

(31) radiation therapy enhancement;

(32) novel mechanisms of action;

(33) selective target cell population therapeutics;

(34) use with ionizing radiation;

(35) use with an agent that counteracts myelosuppression;

(36) use with an agent that increases the ability of the substituted hexitol to pass through the blood-brain barrier to treat brain metastases of NSCLC or to treat GBM; and

(37) use with an agent that suppresses proliferation of cancer stem cells (CSC).

As detailed above, in general, the substituted hexitol derivative usable in methods and compositions according to the present invention include galactitols, substituted galacitols, dulcitols, and substituted dulcitols, including dianhydrogalactitol, diacetyldianhydrogalactitol, dibromodulcitol, and derivatives and analogs thereof. Typically, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the substituted hexitol derivative is dianhydrogalactitol.

When the improvement made is by dose modification, the dose modification can be, but is not limited to, at least one dose modification selected from the group consisting of:
(a) continuous i.v. infusion for hours to days;
(b) biweekly administration;
(c) doses greater than 5 mg/m²/day;
(d) progressive escalation of dosing from 1 mg/m²/day based on patient tolerance;
(e) use of caffeine to modulate metabolism;
(f) use of isoniazid to modulate metabolism;
(g) selected and intermittent boosting of dosage administration;
(h) administration of single and multiple doses escalating from 5 mg/m²/day via bolus;
(i) oral dosages of below 30 mg/m²;
(j) oral dosages of above 130 mg/m²;
(k) oral dosages up to 40 mg/m² for 3 days and then a nadir/recovery period of 18-21 days;
(l) dosing at a lower level for an extended period (e.g., 21 days);
(m) dosing at a higher level;
(n) dosing with a nadir/recovery period longer than 21 days;
(o) the use of a substituted hexitol derivative such as dianhydrogalactitol as a single cytotoxic agent, typically at 30 mg/m²/day×5 days, repeated monthly;
(p) dosing at 3 mg/kg;
(q) the use of a substituted hexitol derivative such as dianhydrogalactitol in combination therapy, typically at 30 mg/m²/day×5 days; and
(r) dosing at 40 mg/day×5 days in adult patients, repeated every two weeks.

When the improvement is made by route of administration, the route of administration can be, but is not limited to, at least one route of administration selected from the group consisting of:
(a) topical administration;
(b) oral administration;
(c) slow release oral delivery;
(d) intrathecal administration;
(e) intraarterial administration;
(f) continuous infusion;
(g) intermittent infusion;
(h) intravenous administration, such as intravenous administration for 30 minutes;
(i) administration through a longer infusion; and
(j) administration through IV push.

When the improvement is made by schedule of administration, the schedule of administration can be, but is not limited to, at least one schedule of administration selected from the group consisting of:
(a) daily administration;
(b) weekly administration;
(c) weekly administration for three weeks;
(d) biweekly administration;
(e) biweekly administration for three weeks with a 1-2 week rest period;
(f) intermittent boost dose administration; and
(g) daily administration for one week for multiple weeks.

When the improvement is made by selection of disease stage, the selection of disease stage can be, but is not limited to, at least one selection of disease stage selected from the group consisting of:
(a) use in an appropriate disease stage for NSCLC;
(b) use with an angiogenesis inhibitor to prevent or limit metastatic spread;
(c) use for newly diagnosed disease;
(d) use for recurrent disease; and
(e) use for resistant or refractory disease.

When the improvement is made by patient selection, the patient selection can be, but is not limited to, a patient selection carried out by a criterion selected from the group consisting of:
(a) selecting patients with a disease condition characterized by a high level of a metabolic enzyme selected from the group consisting of histone deacetylase and ornithine decarboxylase;
(b) selecting patients with a low or high susceptibility to a condition selected from the group consisting of thrombocytopenia and neutropenia;
(c) selecting patients intolerant of GI toxicities;
(d) selecting patients characterized by over- or under-expression of a gene selected from the group consisting of c-Jun, a GPCR, a signal transduction protein, VEGF, a prostate-specific gene, and a protein kinase.
(e) selecting patients characterized by carrying extra copies of the EGFR gene for NSCLC;
(f) selecting patients characterized by methylation or lack of methylation of the promoter of the MGMT gene;
(g) selecting patients characterized by an unmethylated promoter region of MGMT (O⁶-methylguanine methyltransferase);
(h) selecting patients characterized by a methylated promoter region of MGMT;
(i) selecting patients characterized by a high expression of MGMT;
(j) selecting patients characterized by a low expression of MGMT;
(k) selecting patients characterized by a mutation in EGFR, including, but not limited to EGFR Variant III;
(l) selecting patients being administered a platinum-based drug as combination therapy;
(m) selecting patients who do not have EGFR mutations and thus are less likely to respond to tyrosine kinase inhibitors (TKI);
(n) selecting patients who have become resistant to TKI treatment;
(o) selecting patients who have the BIM co-deletion mutation and thus are less likely to respond to TKI treatment;
(p) selecting patients who have become resistant to platinum-based drug treatment; and
(q) selecting patients with brain metastases secondary to NSCLC.

The cellular proto-oncogene c-Jun encodes a protein that, in combination with c-Fos, forms the AP-1 early response transcription factor. This proto-oncogene plays a key role in transcription and interacts with a large number of proteins affecting transcription and gene expression. It is also involved in proliferation and apoptosis of cells that form part of a number of tissues, including cells of the endometrium and glandular epithelial cells. G-protein coupled receptors (GPCRs) are important signal transducing receptors. The superfamily of G protein coupled receptors includes a large number of receptors. These receptors are integral membrane proteins characterized by amino acid sequences that contain seven hydrophobic domains, predicted to represent the transmembrane spanning regions of the proteins. They are found in a wide range of organisms and are involved in the transmission of signals to the interior of cells as a result of their interaction with heterotrimeric G proteins. They respond to a diverse range of agents including lipid analogues, amino acid derivatives, small molecules such as epinephrine and dopamine, and various sensory stimuli. The properties of many known GPCR are summarized in S. Watson & S. Arkinstall, “The G-Protein Linked Receptor Facts Book” (Academic Press, London, 1994), incorporated herein by this reference. GPCR receptors include, but are not limited to, acetylcholine receptors, β-adrenergic receptors, β₃-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors, parathyroid hormone receptors, platelet activating factor receptors, prostanoid (prostaglandin) receptors, somatostatin receptors, thyrotropin-releasing hormone receptors, vasopressin and oxytocin receptors.

EGFR mutations can be associated with sensitivity to therapeutic agents such as gefitinib, as described in J. G. Paez et al., “EGFR Mutations in Lung Cancer: Correlation with Clinical Response to Gefitinib,” Science 304:1497-1500 (2004), incorporated herein by this reference. One specific mutation in EGFR that is associated with resistance to tyrosine kinase inhibitors is known as EGFR Variant III, which is described in C. A. Learn et al., “Resistance to Tyrosine Kinase Inhibition by Mutant Epidermal Growth Factor Variant III Contributes to the Neoplastic Phenotype of Glioblastoma Multiforme,” Clin. Cancer Res. 10:3216-3224 (2004), incorporated herein by this reference. EGFR Variant III is characterized by a consistent and tumor-specific in-frame deletion of 801 bp from the extracellular domain that splits a codon and produces a novel glycine at the fusion junction. This mutation encodes a protein with a constituently active thymidine kinase that enhances the tumorigenicity of the cells carrying this mutation. This mutated protein sequence is absent from normal tissues.

Recent work has established that resistance to TKI chemotherapy is at least partially due to genetic polymorphisms that affect the apoptotic response to TKI.

Specifically, these polymorphisms include, but are not necessarily limited to, polymorphisms in the gene BCL2L11 (also known as BIM), which encodes a BH3-only protein that is a BCL-2 family member. The BH3-only proteins activate cell death by either opposing the prosurvival members of the BCL2 family (BCL2, BCL2-like 1 (BCL-XL, also known as BCL2L1), myeloid cell leukemia sequence 1 (MCL1) and BCL2-related protein A1 (BCL2A1)) or by binding to the pro-apoptotic BCL2 family members (BCL2-associated X protein (BAX) and BCL2-antagonist/killer 1 (BAK1)) and directly activating their pro-apoptotic functions; the activation of pro-apoptotic functions would result in cell death (R. J. Youle & A. Strasser, “The BCL-2 Protein Family: Opposing Activities that Mediate Cell Death,” Nat. Rev. Mol. Cell. Biol. 9:47-59 (2008), incorporated herein by this reference.

It also has been previously shown that several kinase-driven cancers, such as CML and EGFR NSCLC, can maintain a survival advantage by suppressing BIM transcription and also by targeting BIM protein for proteasomal degradation through mitogen-activated protein kinase 1 (MAPK-1)-dependent phosphorylation. In all of these malignancies, BIM upregulation is required for TKIs to induce apoptosis of cancer cells, and suppression of BIM expression is sufficient to confer in vitro resistance to TKIs (J. Kuroda et al., “Bim and Bad Mediate Imatinib-Induced Killing of Bcr/Abl⁺ Leukemic Cells, and Resistance Due to Their Loss is Overcome by a BH3 Mimetic,” Proc. Natl. Acad. Sci. USA 103:14907-14912 (2006); K. J. Aichberger et al., “Low-Level Expression of Proapoptotic Bcl-2-Interacting Mediator in Leukemic Cells in Patients with Chronic Myeloid Leukemia: Role of BCR/ABL, Characterization of Underlying Signaling Pathways, and Reexpression by Novel Pharmacologic Compounds,” Cancer Res. 65: 9436-9444 (2005); R. Kuribara et al., “Roles of Bim in Apoptosis of Normal and Bcl Abr Expressing Hematopoietic Progenitors,” Mol. Cell. Biol. 24:6172-6183 (2004); M. S. Cragg et al., “Gefitinib-Induced Killing of NSCLC Cell Lines Expressing Mutant EGFR Requires BIM and Can Be Enhanced by BH3 Mimetics,” PLoS Med. 4:1681-1689 (2007); Y. Gong et al., “Induction of BIM Is Essential for Apoptosis Triggered by EGFR Kinase Inhibitors in Mutant EGFR-Dependent Lung Adenocarcinomas,” PLoS Med. 4:e294 (2007); D. B. Costa et al., “BIM Mediates EGFR Tyrosine Kinase Inhibitor-Induced Apoptosis in Lung Cancers with Oncogenic EGFR Mutations,” PLoS Med. 4:1669-1679 (2007), all of which are incorporated herein by this reference).

One recent finding has been the discovery of a deletion polymorphism in the BIM gene that results in the generation of alternatively spliced isoforms of BIM that lack the crucial BH3 domain that is involved in the promotion of apoptosis. This polymorphism has a profound effect on the TKI sensitivity of CML and EGFR NSCLC cells, such that one copy of the deleted allele is sufficient to render cells intrinsically TKI resistant. This polymorphism therefore functions in a dominant manner to render such cells resistant to TKI chemotherapy. This finding also includes the result that individuals with the polymorphism have markedly inferior responses to TKI than do individuals without the polymorphism. In particular, the presence of the polymorphism was correlated with a lesser degree of response to imatinib, a TKI, in CML, as well as a shorter progression-free survival (PFS) with EGFR TKI therapy in EGFR NSCLC (K. P. Ng et al., “A Common BIM Deletion Polymorphism Mediates Intrinsic Resistance and Inferior Responses to Tyrosine Kinase Inhibitors in Cancer,” Nature Med. doi 10.138/nm.2713 (Mar. 18, 2012), incorporated herein by this reference).

When the improvement is made by analysis of patient or disease phenotype, the analysis of patient or disease phenotype can be, but is not limited to, a method of analysis of patient or disease phenotype carried out by a method selected from the group consisting of:
(a) use of a diagnostic tool, a diagnostic technique, a diagnostic kit, or a diagnostic assay to confirm a patient's particular phenotype;
(b) use of a method for measurement of a marker selected from the group consisting of histone deacetylase, ornithine decarboxylase, VEGF, a protein that is a gene product of jun, and a protein kinase;
(c) surrogate compound dosing; and
(d) low dose pre-testing for enzymatic status.

When the improvement is made by analysis of patient or disease genotype, the analysis of patient or disease genotype can be, but is not limited to, a method of analysis of patient or disease genotype carried out by a method selected from the group consisting of:
(a) use of a diagnostic tool, a diagnostic technique, a diagnostic kit, or a diagnostic assay to confirm a patient's particular genotype;
(b) use of a gene chip;
(c) use of gene expression analysis;
(d) use of single nucleotide polymorphism (SNP) analysis;
(e) measurement of the level of a metabolite or a metabolic enzyme;
(f) determination of copy number of the EGFR gene;
(g) determination of status of methylation of promoter of MGMT gene;
(h) determination of the existence of an unmethylated promoter region of the MGMT gene;
(i) determination of the existence of a methylated promoter region of the MGMT gene;
(j) determination of the existence of high expression of MGMT; and
(k) determination of the existence of low expression of MGMT.

The use of gene chips is described in A. J. Lee & S. Ramaswamy, “DNA Microarrays in Biological Discovery and Patient Care” in Essentials of Genomic and Personalized Medicine (G. S. Ginsburg & H. F. Willard, eds., Academic Press, Amsterdam, 2010), ch. 7, pp. 73-88, incorporated herein by this reference.

When the method is the use of single nucleotide polymorphism (SNP) analysis, the SNP analysis can be carried out on a gene selected from the group consisting of histone deacetylase, ornithine decarboxylase, VEGF, a prostate specific gene, c-Jun, and a protein kinase. The use of SNP analysis is described in S. Levy and Y.-H. Rogers, “DNA Sequencing for the Detection of Human Genome Variation” in Essentials of Genomic and Personalized Medicine (G. S. Ginsburg & H. F. Willard, eds., Academic Press, Amsterdam, 2010), ch. 3, pp. 27-37, incorporated herein by this reference.

Still other genomic techniques such as copy number variation analysis and analysis of DNA methylation can be employed. Copy number variation analysis is described in C. Lee et al., “Copy Number Variation and Human Health” in Essentials of Genomic and Personalized Medicine (G. S. Ginsburg & H. F. Willard, eds., Academic Press, Amsterdam, 2010), ch. 5, pp. 46-59, incorporated herein by this reference. This is particularly significant for GBM as an increase in copy number of EGFR is associated with particular subtypes of GBM. DNA methylation analysis is described in S. Cottrell et al., “DNA Methylation Analysis: Providing New Insight into Human Disease” in Essentials of Genomic and Personalized Medicine (G. S. Ginsburg & H. F. Willard, eds., Academic Press, Amsterdam, 2010), ch. 6, pp. 60-72, incorporated herein by this reference. This is particularly significant for NSCLC in that the prognosis for NSCLC can vary with the degree of methylation of the promoter of the MGMT gene because of the role of the MGMT gene in promoting drug resistance, and is also relevant for GBM.

When the improvement is made by pre/post-treatment preparation, the pre/post-treatment preparation can be, but is not limited to, a method of pre/post treatment preparation selected from the group consisting of:
(a) the use of colchicine or an analog thereof;
(b) the use of a diuretic;
(c) the use of a uricosuric;
(d) the use of uricase;
(e) the non-oral use of nicotinamide;
(f) the use of a sustained-release form of nicotinamide;
(g) the use of an inhibitor of poly-ADP ribose polymerase;
(h) the use of caffeine;
(i) the use of leucovorin rescue;
(j) infection control; and
(k) the use of an anti-hypertensive agent.

Uricosurics include, but are not limited to, probenecid, benzbromarone, and sulfinpyrazone. A particularly preferred uricosuric is probenecid. Uricosurics, including probenecid, may also have diuretic activity. Other diuretics are well known in the art, and include, but are not limited to, hydrochlorothiazide, carbonic anhydrase inhibitors, furosemide, ethacrynic acid, amiloride, and spironolactone.

Poly-ADP ribose polymerase inhibitors are described in G. J. Southan & C. Szabó, “Poly(ADP-Ribose) Inhibitors,” Curr. Med. Chem. 10:321-240 (2003), incorporated herein by this reference, and include nicotinamide, 3-aminobenzamide, substituted 3,4-dihydroisoquinolin-1(2H)-ones and isoquinolin-1(2H)-ones, benzimidazoles, indoles, phthalazin-1(2H)-ones, quinazolinones, isoindolinones, phenanthridinones, and other compounds.

Leucovorin rescue comprises administration of folinic acid (leucovorin) to patients in which methotrexate has been administered. Leucovorin is a reduced form of folic acid that bypasses dihydrofolate reductase and restores hematopoietic function. Leucovorin can be administered either intravenously or orally.

In one alternative, wherein the pre/post treatment is the use of a uricosuric, the uricosuric is probenecid or an analog thereof.

When the improvement is made by toxicity management, the toxicity management can be, but is not limited to, a method of toxicity management selected from the group consisting of:
(a) the use of colchicine or an analog thereof;
(b) the use of a diuretic;
(c) the use of a uricosuric;
(d) the use of uricase;
(e) the non-oral use of nicotinamide;
(f) the use of a sustained-release form of nicotinamide;
(g) the use of an inhibitor of poly-ADP ribose polymerase;
(h) the use of caffeine;
(i) the use of leucovorin rescue;
(j) the use of sustained-release allopurinol;
(k) the non-oral use of allopurinol;
(l) the use of bone marrow transplants;
(m) the use of a blood cell stimulant;
(n) the use of blood or platelet infusions;
(o) the administration of an agent selected from the group consisting of filgrastim, G-CSF, and GM-CSF;
(p) the application of a pain management technique;
(q) the administration of an anti-inflammatory agent;
(r) the administration of fluids;
(s) the administration of a corticosteroid;
(t) the administration of an insulin control medication;
(u) the administration of an antipyretic;
(v) the administration of an anti-nausea treatment;
(w) the administration of an anti-diarrheal treatment;
(x) the administration of N-acetylcysteine; and
(y) the administration of an antihistamine.

Filgrastim is a granulocytic colony-stimulating factor (G-CSF) analog produced by recombinant DNA technology that is used to stimulate the proliferation and differentiation of granulocytes and is used to treat neutropenia; G-CSF can be used in a similar manner. GM-CSF is granulocyte macrophage colony-stimulating factor and stimulates stem cells to produce granulocytes (eosinophils, neutrophils, and basophils) and monocytes; its administration is useful to prevent or treat infection.

Anti-inflammatory agents are well known in the art and include corticosteroids and non-steroidal anti-inflammatory agents (NSAIDs). Corticosteroids with anti-inflammatory activity include, but are not limited to, hydrocortisone, cortisone, beclomethasone dipropionate, betamethasone, dexamethasone, prednisone, methylprednisolone, triamcinolone, fluocinolone acetonide, and fludrocortisone. Non-steroidal anti-inflammatory agents include, but are not limited to, acetylsalicylic acid (aspirin), sodium salicylate, choline magnesium trisalicylate, salsalate, diflunisal, sulfasalazine, olsalazine, acetaminophen, indomethacin, sulindac, tolmetin, diclofenac, ketorolac, ibuprofen, naproxen, flurbiprofen, ketoprofen, fenoprofin, oxaprozin, mefenamic acid, meclofenamic acid, piroxicam, meloxicam, nabumetone, rofecoxib, celecoxib, etodolac, nimesulide, aceclofenac, alclofenac, alminoprofen, amfenac, ampiroxicam, apazone, araprofen, azapropazone, bendazac, benoxaprofen, benzydamine, bermoprofen, benzpiperylon, bromfenac, bucloxic acid, bumadizone, butibufen, carprofen, cimicoxib, cinmetacin, cinnoxicam, clidanac, clofezone, clonixin, clopirac, darbufelone, deracoxib, droxicam, eltenac, enfenamic acid, epirizole, esflurbiprofen, ethenzamide, etofenamate, etoricoxib, felbinac, fenbufen, fenclofenac, fenclozic acid, fenclozine, fendosal, fentiazac, feprazone, filenadol, flobufen, florifenine, flosulide, flubichin methanesulfonate, flufenamic acid, flufenisal, flunixin, flunoxaprofen, fluprofen, fluproquazone, furofenac, ibufenac, imrecoxib, indoprofen, isofezolac, isoxepac, isoxicam, licofelone, lobuprofen, lomoxicam, lonazolac, loxaprofen, lumaricoxib, mabuprofen, miroprofen, mofebutazone, mofezolac, morazone, nepafanac, niflumic acid, nitrofenac, nitroflurbiprofen, nitronaproxen, orpanoxin, oxaceprol, oxindanac, oxpinac, oxyphenbutazone, pamicogrel, parcetasal, parecoxib, parsalmide, pelubiprofen, pemedolac, phenylbutazone, pirazolac, pirprofen, pranoprofen, salicin, salicylamide, salicylsalicylic acid, satigrel, sudoxicam, suprofen, talmetacin, talniflumate, tazofelone, tebufelone, tenidap, tenoxicam, tepoxalin, tiaprofenic acid, tiaramide, tilmacoxib, tinoridine, tiopinac, tioxaprofen, tolfenamic acid, triflusal, tropesin, ursolic acid, valdecoxib, ximoprofen, zaltoprofen, zidometacin, and zomepirac, and the salts, solvates, analogues, congeners, bioisosteres, hydrolysis products, metabolites, precursors, and prodrugs thereof.

The clinical use of corticosteroids is described in B. P. Schimmer & K. L. Parker, “Adrenocorticotropic Hormone; Adrenocortical Steroids and Their Synthetic Analogs; Inhibitors of the Synthesis and Actions of Adrenocortical Hormones” in Goodman & Gilman's The Pharmacological Basis of Therapeutics (L. L. Brunton, ed., 11^{th} ed., McGraw-Hill, New York, 2006), ch. 59, pp. 1587-1612, incorporated herein by this reference.

Anti-nausea treatments include, but are not limited to, ondansetron, metoclopramide, promethazine, cyclizine, hyoscine, dronabinol, dimenhydrinate, diphenhydramine, hydroxyzine, medizine, dolasetron, granisetron, palonosetron, ramosetron, domperidone, haloperidol, chlorpromazine, fluphenazine, perphenazine, prochlorperazine, betamethasone, dexamethasone, lorazepam, and thiethylperazine.

Anti-diarrheal treatments include, but are not limited to, diphenoxylate, difenoxin, loperamide, codeine, racecadotril, octreoside, and berberine.

N-acetylcysteine is an antioxidant and mucolytic that also provides biologically accessible sulfur.

Poly-ADP ribose polymerase (PARP) inhibitors include, but are not limited to: (1) derivatives of tetracycline as described in U.S. Pat. No. 8,338,477 to Duncan et al.; (2) 3,4-dihydro-5-methyl-1(2H)-isoquinoline, 3-aminobenzamide, 6-aminonicotinamide, and 8-hydroxy-2-methyl-4(3H)-quinazolinone, as described in U.S. Pat. No. 8,324,282 by Gerson et al.; (3) 6-(5H)-phenanthridinone and 1,5-isoquinolinediol, as described in U.S. Pat. No. 8,324,262 by Yuan et al.; (4) (R)-3-[2-(2-hydroxymethylpyrrolidin-1-yl)ethyl]-5-methyl-2H-isoquinolin-1-one, as described in U.S. Pat. No. 8,309,573 to Fujio et al.; (5) 6-alkenyl-substituted 2-quinolinones, 6-phenylalkyl-substituted quinolinones, 6-alkenyl-substituted 2-quinoxalinones, 6-phenylalkyl-substituted 2-quinoxalinones, substituted 6-cyclohexylalkyl substituted 2-quinolinones, 6-cyclohexylalkyl substituted 2-quinoxalinones, substituted pyridones, quinazolinone derivatives, phthalazine derivatives, quinazolinedione derivatives, and substituted 2-alkyl quinazolinone derivatives, as described in U.S. Pat. No. 8,299,256 to Vialard et al.; (6) 5-bromoisoquinoline, as described in U.S. Pat. No. 8,299,088 to Mateucci et al.; (7) 5-bis-(2-chloroethyl)amino]-1-methyl-2-benzimidazolebutyric acid, 4-iodo-3-nitrobenzamide, 8-fluoro-5-(4-((methylamino)methyl)phenyl)-3,4-dihydro-2H-azepino[5,4,3-cd]indol-1(6H)-one phosphoric acid, and N-[3-(3,4-dihydro-4-oxo-1-phthalazinyl)phenyl]-4-morpholinebutanamide methanesulfonate, as described in U.S. Pat. No. 8,227,807 to Gallagher et al.; (8) pyridazinone derivatives, as described in U.S. Pat. No. 8,268,827 to Branca et al.; (9) 4-[3-(4-cyclopropanecarbonyl-piperazine-1-carbonyl)-4-fluorobenzyl]-2H-phthalazin-1-one, as described in U.S. Pat. No. 8,247,416 to Menear et al.; (10) tetraaza phenalen-3-one compounds, as described in U.S. Pat. No. 8,236,802 to Xu et al.; (11) 2-substituted-1H-benzimidazole-4-carboxamides, as described in U.S. Pat. No. 8,217,070 to Zhu et al.; (12) substituted 2-alkyl quinazolinones, as described in U.S. Pat. No. 8,188,103 to Van der Aa et al.; (13) 1H-benzimidazole-4-carboxamides, as described in U.S. Pat. No. 8,183,250 to Penning et al.; (14) indenoisoquinolinone analogs, as described in U.S. Pat. No. 8,119,654 to Jagtap et al.; (15) benzoxazole carboxamides, described in U.S. Pat. No. 8,088,760 to Chu et al; (16) diazabenzo[de] anthracen-3-one compounds, described in U.S. Pat. No. 8,058,075 to Xu et al.; (17) dihydropyridophthalazinones, described in U.S. Pat. No. 8,012,976 to Wang et al., (18) substituted azaindoles, described in U.S. Pat. No. 8,008,491 to Jiang et al.; (19) fused tricyclic compounds, described in U.S. Pat. No. 7,956,064 to Chua et al.; (20) substituted 6a,7,8,9-tetrahydropyrido[3,2-e]pyrrolo[1,2-a]pyrazin-6(5H)-ones, described in U.S. Pat. No. 7,928,105 to Gangloff et al.; and (21) thieno[2,3-c] isoquinolines, described in U.S. Pat. No. 7,825,129, all of which patents are incorporated herein by this reference. Other PARP inhibitors are known in the art.

When the improvement is made by pharmacokinetic/pharmacodynamic monitoring, the pharmacokinetic/pharmacodynamic monitoring can be, but is not limited to a method selected from the group consisting of:
(a) multiple determinations of blood plasma levels; and
(b) multiple determinations of at least one metabolite in blood or urine.

Typically, determination of blood plasma levels or determination of at least one metabolite in blood or urine is carried out by immunoassays. Methods for performing immunoassays are well known in the art, and include radioimmunoassay, ELISA (enzyme-linked immunosorbent assay), competitive immunoassay, immunoassay employing lateral flow test strips, and other assay methods.

When the improvement is made by drug combination, the drug combination can be, but is not limited to, a drug combination selected from the group consisting of:
(a) use with topoisomerase inhibitors;
(b) use with fraudulent nucleosides;
(c) use with fraudulent nucleotides;
(d) use with thymidylate synthetase inhibitors;
(e) use with signal transduction inhibitors;
(f) use with cisplatin or platinum analogs;
(g) use with monofunctional alkylating agents;
(h) use with bifunctional alkylating agents;
(i) use with alkylating agents that damage DNA at a different place than does dianhydrogalactitol;
(j) use with anti-tubulin agents;
(k) use with antimetabolites;
(l) use with berberine;
(m) use with apigenin;
(n) use with amonafide;
(o) use with colchicine or analogs;
(p) use with genistein;
(q) use with etoposide;
(r) use with cytarabine;
(s) use with camptothecins
(t) use with vinca alkaloids;
(u) use with 5-fluorouracil;
(v) use with curcumin;
(w) use with NF-κB inhibitors;
(x) use with rosmarinic acid;
(y) use with mitoguazone;
(z) use with tetrandrine;
(aa) use with temozolomide;
(ab) use with VEGF inhibitors;
(ac) use with cancer vaccines;
(ad) use with EGFR inhibitors;
(ae) use with tyrosine kinase inhibitors;
(af) use with poly (ADP-ribose) polymerase (PARP) inhibitors; and
(ag) use with ALK inhibitors.

Topoisomerase inhibitors include, but are not limited to, irinotecan, topotecan, camptothecin, lamellarin D, amsacrine, etoposide, etoposide phosphate, teniposide, doxorubicin, and ICRF-193.

Fraudulent nucleosides include, but are not limited to, cytosine arabinoside, gemcitabine, and fludarabine; other fraudulent nucleosides are known in the art.

Fraudulent nucleotides include, but are not limited to, tenofovir disoproxil fumarate and adefovir dipivoxil; other fraudulent nucleotides are known in the art.

Thymidylate synthetase inhibitors include, but are not limited to, raltitrexed, pemetrexed, nolatrexed, ZD9331, GS7094L, fluorouracil, and BGC 945.

Signal transduction inhibitors are described in A. V. Lee et al., “New Mechanisms of Signal Transduction Inhibitor Action: Receptor Tyrosine Kinase Down-Regulation and Blockade of Signal Transactivation,” Clin. Cancer Res. 9:516s (2003), incorporated herein in its entirety by this reference.

Alkylating agents include, but are not limited to, Shionogi 254-S, aldo-phosphamide analogues, altretamine, anaxirone, Boehringer Mannheim BBR-2207, bendamustine, bestrabucil, budotitane, Wakunaga CA-102, carboplatin, carmustine (BCNU), Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)₂, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsulfam, ifosfamide, iproplatin, lomustine (CCNU), mafosfamide, melphalan, mitolactol, nimustine (ACNU), Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromustine, Tanabe Seiyaku TA-077, tauromustine, temozolomide, teroxirone, tetraplatin and trimelamol, as described in U.S. Pat. No. 7,446,122 by Chao et al., incorporated herein by this reference. Temozolomide, BCNU, CCNU, and ACNU all damage DNA at O⁶ of guanine, whereas DAG cross-links at N⁷); one alternative is therefore to use DAG in combination with an alkylating agent that damages DNA at a different place than DAG. The alkylating agent can be a monofunctional alkylating agent or a bifunctional alkylating agent. Monofunctional alkylating agents include, but are not limited to, carmustine lomustine, temozolomide, and dacarbazine, as described in N. Kondo et al., “DNA Damage Induced by Alkylating Agents and Repair Pathways,” J. Nucl. Acids doi:10.4061/2010/543531 (2010), incorporated herein by this reference; monofunctional alkylating agents also include such agents as methyl methanesulfonate, ethylmethanesulfonate, and N-methyl-N-nitrosoguanidine, as described in J. M. Walling & I. J. Stratford, “Chemosensitization by Monofunctional Alkylating Agents,” Int. J. Radiat. Oncol. Biol. Phys. 12:1397-1400 (1986), incorporated herein by this reference. Bifunctional alkylating agents include, but are not limited to, mechlorethamine, chlorambucil, cyclophosphamide, busulfan, nimustine, carmustine, lomustine, fotemustine, and bis-(2-chloroethyl) sulfide (N. Kondo et al. (2010), supra). One significant class of bifunctional alkylating agents includes alkylating agents that target O⁶ of guanine in DNA. Another significant class of alkylating agents comprises cisplatin and other platinum-containing agents, including, but not limited to, carboplatin, iproplatin, oxaliplatin, tetraplatin, satraplatin, picoplatin, nedaplatin, and triplatin. These agents cause cross-linking of DNA, which then induces apoptosis. The combination with cisplatin or other platinum-containing agents is a potential component of standard platinum doublet therapy. Additionally, the ability to be more than additive or synergistic is particularly significant with respect to the combination of a substituted hexitol derivative such as dianhydrogalactitol with cisplatin or other platinum-containing chemotherapeutic agents, as well as other chemotherapeutic agents recited herein.

Anti-tubulin agents include, but are not limited to, vinca alkaloids, taxanes, podophyllotoxin, halichondrin B, and homohalichondrin B.

Antimetabolites include, but are not limited to: methotrexate, pemetrexed, 5-fluorouracil, capecitabine, cytarabine, gemcitabine, 6-mercaptopurine, and pentostatin, alanosine, AG2037 (Pfizer), 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrill-Dow DDFC, deazaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, piritrexim, plicamycin, Asahi Chemical PL-AC, Takeda TAC-788, thioguanine, tiazofurin, Erbamont TIF, trimetrexate, tyrosine kinase inhibitors, tyrosine protein kinase inhibitors, Taiho UFT and uricytin.

Berberine has antibiotic activity and prevents and suppresses the expression of pro-inflammatory cytokines and E-selectin, as well as increasing adiponectin expression.

Apigenin is a flavone that can reverse the adverse effects of cyclosporine and has chemoprotective activity, either alone or derivatized with a sugar.

Amonafide is a topoisomerase inhibitor and DNA intercalator that has anti-neoplastic activity.

Curcumin is believed to have anti-neoplastic, anti-inflammatory, antioxidant, anti-ischemic, anti-arthritic, and anti-amyloid properties and also has hepatoprotective activity.

NF-κB inhibitors include, but are not limited to, bortezomib.

Rosmarinic acid is a naturally-occurring phenolic antioxidant that also has anti-inflammatory activity.

Mitoguazone is an inhibitor of polyamine biosynthesis through competitive inhibition of S-adenosylmethionine decarboxylase.

Tetrandrine has the chemical structure 6,6′,7,12-tetramethoxy-2,2′-dimethyl-1 β-berbaman and is a calcium channel blocker that has anti-inflammatory, immunologic, and antiallergenic effects, as well as an anti-arrhythmic effect similar to that of quinidine. It has been isolated from Stephania tetranda and other Asian herbs.

VEGF inhibitors include bevacizumab (Avastin™), which is a monoclonal antibody against VEGF, itraconazole, and suramin, as well as batimastat and marimastat, which are matrix metalloproteinase inhibitors, and cannabinoids and derivatives thereof.

Cancer vaccines are being developed. Typically, cancer vaccines are based on an immune response to a protein or proteins occurring in cancer cells that does not occur in normal cells. Cancer vaccines include Provenge™ for metastatic hormone-refractory prostate cancer, Oncophage™ for kidney cancer, CimaVax-EGF™ for lung cancer, MOBILAN, Neuvenge for Her2/neu expressing cancers such as breast cancer, colon cancer, bladder cancer, and ovarian cancer, Stimuvax™ for breast cancer, and others. Cancer vaccines are described in S. Pejawar-Gaddy & O. Finn, “Cancer Vaccines: Accomplishments and Challenges,” Crit. Rev. Oncol. Hematol. 67:93-102 (2008), incorporated herein by this reference.

The epidermal growth factor receptor (EGFR) exists on the cell surface of mammalian cells and is activated by binding of the receptor to its specific ligands, including, but not limited to epidermal growth factor and transforming growth factor α. Upon activation by binding to its growth factor ligands, EGFR undergoes a transition from an inactive monomeric form to an active homodimer, although preformed active dimers may exist before ligand binding. In addition to forming active homodimers after ligand binding, EGFR may pair with another member of the ErbB receptor family, such as ErbB2/Her2/neu, to create an activated heterodimer. There is also evidence that clusters of activated EGFRs form, although it is uncertain whether such clustering is important for activation itself or occurs subsequent to activation of individual dimers. EGFR dimerization stimulates its intracellular intrinsic protein-tyrosine kinase activity. As a result, autophosphorylation of several tyrosine residues in the carboxyl-terminal domain of EGFR occurs. These residues include Y992, Y1045, Y1068, Y1148, and Y1171. Such autophosphorylation elicits downstream activation and signaling by several other proteins that associate with the phosphorylated tyrosine residues through their own phosphotyrosine-binding SH2 domains. The signaling of these proteins that associate with the phosphorylated tyrosine residues through their own phosphotyrosine-binding SH2 domains can then initiate several signal transduction cascades and lead to DNA synthesis and cell proliferation. The kinase domain of EGFR can also cross-phosphorylate tyrosine residues of other receptors that it is aggregated with, and can itself be activated in that manner. EGFR is encoded by the c-erbB1 proto-oncogene and has a molecular mass of 170 kDa. It is a transmembrane glycoprotein with a cysteine-rich extracellular region, an intracellular domain containing an uninterrupted tyrosine kinase site, and multiple autophosphorylation sites clustered at the carboxyl-terminal tail as described above. The extracellular portion has been subdivided into four domains: domains I and III, which have 37% sequence identity, are cysteine-poor and conformationally contain the site for ligand (EGF and transforming growing factor α (TGFα) binding. Cysteine-rich domains II and IV contain N-linked glycosylation sites and disulfide bonds, which determine the tertiary conformation of the external domain of the protein molecule. In many human cell lines, TGFα expression has a strong correlation with EGFR overexpression, and therefore TGFα was considered to act in an autocrine manner, stimulating proliferation of the cells in which it is produced via activation of EGFR. Binding of a stimulatory ligand to the EGFR extracellular domain results in receptor dimerization and initiation of intracellular signal transduction, the first step of which is activation of the tyrosine kinase. The earliest consequence of kinase activation is the phosphorylation of its own tyrosine residues (autophosphorylation) as described above. This is followed by association with activation of signal transducers leading to mitogenesis. Mutations that lead to EGFR expression or overactivity have been associated with a number of malignancies, including glioblastoma multiforme. A specific mutation of EGFR known as EGFR Variant III has frequently been observed in glioblastoma (C. T. Kuan et al., “EGF Mutant Receptor VIII as a Molecular Target in Cancer Therapy,” Endocr. Relat. Cancer 8:83-96 (2001), incorporated herein by this reference). EGFR is considered an oncogene. Inhibitors of EGFR include, but are not limited to, erlotinib, gefitinib, lapatinib, lapatinib ditosylate, afatinib, canertinib, neratinib, CP-724714, WHI-P154, TAK-285, AST-1306, ARRY-334543, ARRY-380, AG-1478, tyrphostin 9, dacomitinib, desmethylerlotinib, OSI-420, AZD8931, AEE788, pelitinib, CUDC-101, WZ8040, WZ4002, WZ3146, AG-490, XL647, PD153035 HCl, BMS-599626, BIBW 2992, CI 1033, CP 724714, OSI 420, and vandetinib. Particularly preferred EGFR inhibitors include erlotinib, afatinib, and lapatinib.

Tyrosine kinase inhibitors include, but are not limited to, imatinib, gefitinib, erlotinib, sunitinib, sorafenib, foretinib, cederinib, axitinib, carbozantinib, BIBF1120, golvatinib, dovitinib, ZM 306416, ZM 323881 HCl, SAR 131675, semaxinib, telatinib, pazopanib, ponatinib, crenolanib, tivanitib, mubritinib, danusertib, brivanib, fingolimod, saracatinib, rebastinib, quizartinib, tandutinib, amuvatinib, ibrutinib, fostamatinib, crizotinib, and linsitinib. Such tyrosine kinase inhibitors can inhibit tyrosine kinases associated with one or more of the following receptors: VEGFR, EGFR, PDGFR, c-Kit, c-Met, Her-2, FGFR, FLT-3, IGF-1R, ALK, c-RET, and Tie-2. As the activity of epidermal growth factor receptor (EGFR) involves the activity of a tyrosine kinase, the category of tyrosine kinase inhibitors overlaps with the category of EGFR inhibitors. A number of tyrosine kinase inhibitors inhibit the activity of both EGFR and at least one other tyrosine kinase. In general, tyrosine kinase inhibitors can operate by four different mechanisms: competition with adenosine triphosphate (ATP), used by the tyrosine kinase to carry out the phosphorylation reaction; competition with the substrate; competition with both ATP and the substrate; or allosteric inhibition. The activity of these inhibitors is disclosed in P. Yaish et al., “Blocking of EGF-Dependent Cell Proliferation by EGF Receptor Kinase Inhibitors,” Science 242:933-935 (1988); A. Gazit et al., “Tyrphostins. 2. Heterocyclic and α-Substituted Benzylidenemalononitrile Tyrphostins as Potent Inhibitors of EGF Receptor and ErbB2/neu Tyrosine Kinases,” J. Med. Chem. 34:1896-1907 (1991); N. Osherov et al., “Selective Inhibition of the Epidermal Growth Factor and HER2/neu Receptors by Tyrphostins,” J. Biol. Chem. 268: 11134-11142 (1993); and A. Levitzki & E. Mishani, “Tyrphostins and Other Tyrosine Kinase Inhibitors,” Annu. Rev. Biochem. 75:93-109 (2006), all of which are incorporated herein by this reference.

ALK inhibitors act on tumors with variations of anaplastic lymphoma kinase (ALK) such as an EML4-ALK translocation. ALK inhibitors include, but are not limited to: crizotinib (3-[(1R)-1-(2,6-dichloro-3-fluorophenyl)ethoxy]-5-(1-piperidin-4-ylpyrazol-4-yl)pyridin-2-amine); AP26113 ((2-((5-chloro-2-((4-(4-(dimethylamino)piperidin-1-yl)-2-methoxyphenyl)amino)pyrimidin-4-yl)amino)phenyl)dimethylphosphine oxide); ASP-3026 (N2-[2-methoxy-4-[4-(4-methyl-1-piperazinyl)-1-piperidinyl]phenyl]-N4-[2-[(1-methylethyl)sulfonyl]phenyl]-1,3,5-triazine-2,4-diamine); alectinib (9-ethyl-6,6-dimethyl-8-(4-morpholin-4-ylpiperidin-1-yl)-11-oxo-5H-benzo[b]carbazole-3-carbonitrile); NMS-E628 (N-(5-(3,5-difluorobenzyl)-1H-indazol-3-yl)-4-(4-methylpiperazin-1-yl)-2-((tetrahydro-2H-pyran-4-yl)amino)benzamide); ceritinib; PF-06363922; TSR-011; CEP-37440 (2-[[5-Chloro-2-[[(6S)-6-[4-(2-hydroxyethyl)piperazin-1-yl]-1-methoxy-6,7,8,9-tetrahydro-5H-benzo[7]annulen-2-yl]amino]pyrimidin-4-yl]amino]-N-methyl-benzamide); and X-396 (R)-6-amino-5-(1-(2,6-dichloro-3-fluorophenyl)ethoxy)-N-(4-(4-methylpiperazine-1-carbonyl)phenyl)pyridazine-3-carboxamide).

When the improvement is made by chemosensitization, the chemosensitization can comprise, but is not limited to, the use of a substituted hexitol derivative as a chemosensitizer in combination with an agent selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(j) berberine;
(k) apigenin;
(l) amonafide;
(m) colchicine or analogs;
(n) genistein;
(o) etoposide;
(p) cytarabine;
(q) camptothecins;
(r) vinca alkaloids;
(s) topoisomerase inhibitors;
(t) 5-fluorouracil;
(u) curcumin;
(v) NF-κB inhibitors;
(w) rosmarinic acid;
(x) mitoguazone;
(y) tetrandrine;
(z) a tyrosine kinase inhibitor;
(aa) an inhibitor of EGFR; and
(ab) an inhibitor of PARP.

When the improvement is made by chemopotentiation, the chemopotentiation can comprise, but is not limited to, the use of a substituted hexitol derivative as a chemopotentiator in combination with an agent selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(j) berberine;
(k) apigenin;
(l) amonafide;
(m) colchicine or analogs;
(n) genistein;
(o) etoposide;
(p) cytarabine;
(q) camptothecins;
(r) vinca alkaloids;
(s) 5-fluorouracil;
(t) curcumin;
(u) NF-κB inhibitors;
(v) rosmarinic acid;
(w) mitoguazone;
(x) tetrandrine;
(y) a tyrosine kinase inhibitor;
(z) an inhibitor of EGFR; and
(aa) an inhibitor of PARP.

When the improvement is made by post-treatment management, the post-treatment management can be, but is not limited to, a method selected from the group consisting of:
(a) a therapy associated with pain management;
(b) administration of an anti-emetic;
(c) an anti-nausea therapy;
(d) administration of an anti-inflammatory agent;
(e) administration of an anti-pyretic agent; and
(f) administration of an immune stimulant.

When the improvement is made by alternative medicine/post-treatment support, the alternative medicine/post-treatment support can be, but is not limited to, a method selected from the group consisting of:
(a) hypnosis;
(b) acupuncture;
(c) meditation;
(d) a herbal medication created either synthetically or through extraction; and
(e) applied kinesiology.

In one alternative, when the method is a herbal medication created either synthetically or through extraction, the herbal medication created either synthetically or through extraction can be selected from the group consisting of:
(a) a NF-κB inhibitor;
(b) a natural anti-inflammatory;
(c) an immunostimulant;
(d) an antimicrobial; and
(e) a flavonoid, isoflavone, or flavone.

When the herbal medication created either synthetically or through extraction is a NF-κB inhibitor, the NF-κB inhibitor can be selected from the group consisting of parthenolide, curcumin, and rosmarinic acid. When the herbal medication created either synthetically or through extraction is a natural anti-inflammatory, the natural anti-inflammatory can be selected from the group consisting of rhein and parthenolide. When the herbal medication created either synthetically or through extraction is an immunostimulant, the immunostimulant can be a product found in or isolated from Echinacea. When the herbal medication created either synthetically or through extraction is an anti-microbial, the anti-microbial can be berberine. When the herbal medication created either synthetically or through extraction is a flavonoid or flavone, the flavonoid, isoflavone, or flavone can be selected from the group consisting of apigenin, genistein, apigenenin, genistein, genistin, 6″-O-malonylgenistin, 6″-O-acetylgenistin, daidzein, daidzin, 6″-O-malonyldaidzin, 6″-O-acetylgenistin, glycitein, glycitin, 6″-O-malonylglycitin, and 6-O-acetylglycitin.

When the improvement is made by a bulk drug product improvement, the bulk drug product improvement can be, but is not limited to, a bulk drug product improvement selected from the group consisting of:
(a) salt formation;
(b) preparation as a homogeneous crystal structure;
(c) preparation as a pure isomer;
(d) increased purity;
(e) preparation with lower residual solvent content; and
(f) preparation with lower residual heavy metal content.

When the improvement is made by use of a diluent, the diluent can be, but is not limited to, a diluent selected from the group consisting of:
(a) an emulsion;
(b) dimethylsulfoxide (DMSO);
(c) N-methylformamide (NMF)
(d) DMF;
(e) ethanol;
(f) benzyl alcohol;
(g) dextrose-containing water for injection;
(h) Cremophor™;
(i) cyclodextrin; and
(j) PEG.

When the improvement is made by use of a solvent system, the solvent system can be, but is not limited to, a solvent system selected from the group consisting of:
(a) an emulsion;
(b) dimethylsulfoxide (DMSO);
(c) N-methylformamide (NMF)
(d) DMF;
(e) ethanol;
(f) benzyl alcohol;
(g) dextrose-containing water for injection;
(h) Cremophor™;
(i) cyclodextrin; and
(j) PEG.

When the improvement is made by use of an excipient, the excipient can be, but is not limited to, an excipient selected from the group consisting of:
(a) mannitol;
(b) albumin;
(c) EDTA;
(d) sodium bisulfite;
(e) benzyl alcohol;
(f) a carbonate buffer; and
(g) a phosphate buffer.

When the improvement is made by use of a dosage form, the dosage form can be, but is not limited to, a dosage form selected from the group consisting of:
(a) tablets;
(b) capsules;
(c) topical gels;
(d) topical creams;
(e) patches;
(f) suppositories; and
(g) lyophilized dosage fills.

Formulation of pharmaceutical compositions in tablets, capsules, and topical gels, topical creams or suppositories is well known in the art and is described, for example, in United States Patent Application Publication No. 2004/0023290 by Griffin et al., incorporated herein by this reference.

Formulation of pharmaceutical compositions as patches such as transdermal patches is well known in the art and is described, for example, in U.S. Pat. No. 7,728,042 to Eros et al., incorporated herein by this reference.

Lyophilized dosage fills are also well known in the art. One general method for the preparation of such lyophilized dosage fills, applicable to dianhydrogalactitol and derivatives thereof and to diacetyldianhydrogalactitol and derivatives thereof, comprises the following steps:

(1) Dissolve the drug in water for injection precooled to below 10° C. Dilute to final volume with cold water for injection to yield a 40 mg/mL solution.

(2) Filter the bulk solution through an 0.2-μm filter into a receiving container under aseptic conditions. The formulation and filtration should be completed in 1 hour.

(3) Fill nominal 1.0 mL filtered solution into sterilized glass vials in a controlled target range under aseptic conditions.

(4) After the filling, all vials are placed with rubber stoppers inserted in the “lyophilization position” and loaded in the prechilled lyophilizer. For the lyophilizer, shelf temperature is set at +5° C. and held for 1 hour; shelf temperature is then adjusted to −5° C. and held for one hour, and the condenser, set to −60° C., turned on.

(5) The vials are then frozen to 30° C. or below and held for no less than 3 hours, typically 4 hours.

(6) Vacuum is then turned on, the shelf temperature is adjusted to −5° C., and primary drying is performed for 8 hours; the shelf temperature is again adjusted to −5° C. and drying is carried out for at least 5 hours.

(7) Secondary drying is started after the condenser (set at −60° C.) and vacuum are turned on. In secondary drying, the shelf temperature is controlled at +5° C. for 1 to 3 hours, typically 1.5 hours, then at 25° C. for 1 to 3 hours, typically 1.5 hours, and finally at 35-40° C. for at least 5 hours, typically for 9 hours, or until the product is completely dried.

(8) Break the vacuum with filtered inert gas (e.g., nitrogen). Stopper the vials in the lyophilizer.

(9) Vials are removed from the lyophilizer chamber and sealed with aluminum flip-off seals. All vials are visually inspected and labeled with approved labels.

When the improvement is made by use of dosage kits and packaging, the dosage kits and packaging can be, but are not limited to, dosage kits and packaging selected from the group consisting of the use of amber vials to protect from light and the use of stoppers with specialized coatings to improve shelf-life stability. The dosage kits can be labeled to indicate details of use and may contain one or more than one therapeutically active agent; if more than one therapeutic agent is included, the two or more therapeutic agents can be combined or separately packaged.

When the improvement is made by use of a drug delivery system, the drug delivery system can be, but is not limited to, a drug delivery system selected from the group consisting of:
(a) nanocrystals;
(b) bioerodible polymers;
(c) liposomes;
(d) slow release injectable gels; and
(e) microspheres.

Nanocrystals are described in U.S. Pat. No. 7,101,576 to Hovey et al., incorporated herein by this reference.

Bioerodible polymers are described in U.S. Pat. No. 7,318,931 to Okumu et al., incorporated herein by this reference. A bioerodible polymer decomposes when placed inside an organism, as measured by a decline in the molecular weight of the polymer over time. Polymer molecular weights can be determined by a variety of methods including size exclusion chromatography (SEC), and are generally expressed as weight averages or number averages. A polymer is bioerodible if, when in phosphate buffered saline (PBS) of pH 7.4 and a temperature of 37° C., its weight-average molecular weight is reduced by at least 25% over a period of 6 months as measured by SEC. Useful bioerodible polymers include polyesters, such as poly(caprolactone), poly(glycolic acid), poly(lactic acid), and poly(hydroxybutyrate); polyanhydrides, such as poly(adipic anhydride) and poly(maleic anhydride); polydioxanone; polyamines; polyamides; polyurethanes; polyesteramides; polyorthoesters; polyacetals; polyketals; polycarbonates; polyorthocarbonates; polyphosphazenes; poly(malic acid); poly(amino acids); polyvinylpyrrolidone; poly(methyl vinyl ether); poly(alkylene oxalate); poly(alkylene succinate); polyhydroxycellulose; chitin; chitosan; and copolymers and mixtures thereof.

Liposomes are well known as drug delivery vehicles. Liposome preparation is described in European Patent Application Publication No. EP 1332755 by Weng et al., incorporated herein by this reference.

Slow release injectable gels are known in the art and are described, for example, in B. Jeong et al., “Drug Release from Biodegradable Injectable Thermosensitive Hydrogel of PEG-PLGA-PEG Triblock Copolymers,” J. Controlled Release 63:155-163 (2000), incorporated herein by this reference.

The use of microspheres for drug delivery is known in the art and is described, for example, in H. Okada & H. Taguchi, “Biodegradable Microspheres in Drug Delivery,” Crit. Rev. Ther. Drug Carrier Sys. 12:1-99 (1995), incorporated herein by this reference.

When the improvement is made by use of a drug conjugate form, the drug conjugate form can be, but is not limited to, a drug conjugate form selected from the group consisting of:
(a) a polymer system;
(b) polylactides;
(c) polyglycolides;
(d) amino acids;
(e) peptides; and
(f) multivalent linkers.

Polylactide conjugates are well known in the art and are described, for example, in R. Tong & C. Cheng, “Controlled Synthesis of Camptothecin-Polylactide Conjugates and Nanoconjugates,” Bioconjugate Chem. 21:111-121 (2010), incorporated by this reference.

Polyglycolide conjugates are also well known in the art and are described, for example, in PCT Patent Application Publication No. WO 2003/070823 by Elmaleh et al., incorporated herein by this reference.

Multivalent linkers are known in the art and are described, for example, in United States Patent Application Publication No. 2007/0207952 by Silva et al., incorporated herein by this reference. For example, multivalent linkers can contain a thiophilic group for reaction with a reactive cysteine, and multiple nucleophilic groups (such as NH or OH) or electrophilic groups (such as activated esters) that permit attachment of a plurality of biologically active moieties to the linker.

Suitable reagents for cross-linking many combinations of functional groups are known in the art. For example, electrophilic groups can react with many functional groups, including those present in proteins or polypeptides. Various combinations of reactive amino acids and electrophiles are known in the art and can be used. For example, N-terminal cysteines, containing thiol groups, can be reacted with halogens or maleimides. Thiol groups are known to have reactivity with a large number of coupling agents, such as alkyl halides, haloacetyl derivatives, maleimides, aziridines, acryloyl derivatives, arylating agents such as aryl halides, and others. These are described in G. T. Hermanson, “Bioconjugate Techniques” (Academic Press, San Diego, 1996), pp. 146-150, incorporated herein by this reference. The reactivity of the cysteine residues can be optimized by appropriate selection of the neighboring amino acid residues. For example, a histidine residue adjacent to the cysteine residue will increase the reactivity of the cysteine residue. Other combinations of reactive amino acids and electrophilic reagents are known in the art. For example, maleimides can react with amino groups, such as the ε-amino group of the side chain of lysine, particularly at higher pH ranges. Aryl halides can also react with such amino groups. Haloacetyl derivatives can react with the imidazolyl side chain nitrogens of histidine, the thioether group of the side chain of methionine, and the ε-amino group of the side chain of lysine. Many other electrophilic reagents are known that will react with the ε-amino group of the side chain of lysine, including, but not limited to, isothiocyanates, isocyanates, acyl azides, N-hydroxysuccinimide esters, sulfonyl chlorides, epoxides, oxiranes, carbonates, imidoesters, carbodiimides, and anhydrides. These are described in G. T. Hermanson, “Bioconjugate Techniques” (Academic Press, San Diego, 1996), pp. 137-146, incorporated herein by this reference. Additionally, electrophilic reagents are known that will react with carboxylate side chains such as those of aspartate and glutamate, such as diazoalkanes and diazoacetyl compounds, carbonydilmidazole, and carbodiimides. These are described in G. T. Hermanson, “Bioconjugate Techniques” (Academic Press, San Diego, 1996), pp. 152-154, incorporated herein by this reference. Furthermore, electrophilic reagents are known that will react with hydroxyl groups such as those in the side chains of serine and threonine, including reactive haloalkane derivatives. These are described in G. T. Hermanson, “Bioconjugate Techniques” (Academic Press, San Diego, 1996), pp. 154-158, incorporated herein by this reference. In another alternative embodiment, the relative positions of electrophile and nucleophile (i.e., a molecule reactive with an electrophile) are reversed so that the protein has an amino acid residue with an electrophilic group that is reactive with a nucleophile and the targeting molecule includes therein a nucleophilic group. This includes the reaction of aldehydes (the electrophile) with hydroxylamine (the nucleophile), described above, but is more general than that reaction; other groups can be used as electrophile and nucleophile. Suitable groups are well known in organic chemistry and need not be described further in detail.

Additional combinations of reactive groups for cross-linking are known in the art. For example, amino groups can be reacted with isothiocyanates, isocyanates, acyl azides, N-hydroxysuccinimide (NHS) esters, sulfonyl chlorides, aldehydes, glyoxals, epoxides, oxiranes, carbonates, alkylating agents, imidoesters, carbodiimides, and anhydrides. Thiol groups can be reacted with haloacetyl or alkyl halide derivatives, maleimides, aziridines, acryloyl derivatives, acylating agents, or other thiol groups by way of oxidation and the formation of mixed disulfides. Carboxy groups can be reacted with diazoalkanes, diazoacetyl compounds, carbonyldiimidazole, carbodiimides. Hydroxyl groups can be reacted with epoxides, oxiranes, carbonyldiimidazole, N,N′-disuccinimidyl carbonate, N-hydroxysuccinimidyl chloroformate, periodate (for oxidation), alkyl halogens, or isocyanates. Aldehyde and ketone groups can react with hydrazines, reagents forming Schiff bases, and other groups in reductive amination reactions or Mannich condensation reactions. Still other reactions suitable for cross-linking reactions are known in the art. Such cross-linking reagents and reactions are described in G. T. Hermanson, “Bioconjugate Techniques” (Academic Press, San Diego, 1996), incorporated herein by this reference.

When the improvement is made by use of a compound analog, the compound analog can be, but is not limited to, a compound analog selected from the group consisting of:
(a) alteration of side chains to increase or decrease lipophilicity;
(b) addition of an additional chemical functionality to alter a property selected from the group consisting of reactivity, electron affinity, and binding capacity; and
(c) alteration of salt form.

When the improvement is made by use of a prodrug system, the prodrug system can be, but is not limited to, a prodrug system selected from the group consisting of:
(a) the use of enzyme sensitive esters;
(b) the use of dimers;
(c) the use of Schiff bases;
(d) the use of pyridoxal complexes; and
(e) the use of caffeine complexes.

The use of prodrug systems is described in T. Järvinen et al., “Design and Pharmaceutical Applications of Prodrugs” in Drug Discovery Handbook (S. C. Gad, ed., Wiley-Interscience, Hoboken, N.J., 2005), ch. 17, pp. 733-796, incorporated herein by this reference. This publication describes the use of enzyme sensitive esters as prodrugs. The use of dimers as prodrugs is described in U.S. Pat. No. 7,879,896 to Allegretti et al., incorporated herein by this reference. The use of peptides in prodrugs is described in S. Prasad et al., “Delivering Multiple Anticancer Peptides as a Single Prodrug Using Lysyl-Lysine as a Facile Linker,” J. Peptide Sci. 13: 458-467 (2007), incorporated herein by this reference. The use of Schiff bases as prodrugs is described in U.S. Pat. No. 7,619,005 to Epstein et al., incorporated herein by this reference. The use of caffeine complexes as prodrugs is described in U.S. Pat. No. 6,443,898 to Unger et al., incorporated herein by this reference.

When the improvement is made by use of a multiple drug system, the multiple drug system can be, but is not limited to, a multiple drug system selected from the group consisting of:
(a) use of multi-drug resistance inhibitors;
(b) use of specific drug resistance inhibitors;
(c) use of specific inhibitors of selective enzymes;
(d) use of signal transduction inhibitors;
(e) use of repair inhibition; and
(f) use of topoisomerase inhibitors with non-overlapping side effects.

Multi-drug resistance inhibitors are described in U.S. Pat. No. 6,011,069 to Inomata et al., incorporated herein by this reference.

Specific drug resistance inhibitors are described in T. Hideshima et al., “The Proteasome Inhibitor PS-341 Inhibits Growth, Induces Apoptosis, and Overcomes Drug Resistance in Human Multiple Myeloma Cells,” Cancer Res. 61:3071-3076 (2001), incorporated herein by this reference.

Repair inhibition is described in N. M. Martin, “DNA Repair Inhibition and Cancer Therapy,” J. Photochem. Photobiol. B 63: 62-170 (2001), incorporated herein by this reference.

When the improvement is made by biotherapeutic enhancement, the biotherapeutic enhancement can be performed by use in combination as sensitizers/potentiators with a therapeutic agent or technique that can be, but is not limited to, a therapeutic agent or technique selected from the group consisting of:
(a) cytokines;
(b) lymphokines;
(c) therapeutic antibodies;
(d) antisense therapies;
(e) gene therapies;
(f) ribozymes;
(g) RNA interference; and
(h) vaccines.

Antisense therapies are described, for example, in B. Weiss et al., “Antisense RNA Gene Therapy for Studying and Modulating Biological Processes,” Cell. Mol. Life Sci. 55:334-358 (1999), incorporated herein by this reference.

Ribozymes are described, for example, in S. Pascolo, “RNA-Based Therapies” in Drug Discovery Handbook (S. C. Gad, ed., Wiley-Interscience, Hoboken, N.J., 2005), ch. 27, pp. 1273-1278, incorporated herein by this reference.

RNA interference is described, for example, in S. Pascolo, “RNA-Based Therapies” in Drug Discovery Handbook (S. C. Gad, ed., Wiley-Interscience, Hoboken, N.J., 2005), ch. 27, pp. 1278-1283, incorporated herein by this reference.

As described above, typically, cancer vaccines are based on an immune response to a protein or proteins occurring in cancer cells that does not occur in normal cells. Cancer vaccines include Provenge™ for metastatic hormone-refractory prostate cancer, Oncophage™ for kidney cancer, CimaVax-EGF™ for lung cancer, MOBILAN, Neuvenge for Her2/neu expressing cancers such as breast cancer, colon cancer, bladder cancer, and ovarian cancer, Stimuvax™ for breast cancer, and others. Cancer vaccines are described in S. Pejawar-Gaddy & O. Finn, (2008), supra.

When the biotherapeutic enhancement is use in combination as sensitizers/potentiators with a therapeutic antibody, the therapeutic antibody can be, but is not limited to, a therapeutic antibody selected from the group consisting of bevacizumab (Avastin™), rituximab (Rituxan™), trastuzumab (Herceptin™), and cetuximab (Erbitux™).

When the improvement is made by use of biotherapeutic resistance modulation, the biotherapeutic resistance modulation can be, but is not limited to, use against NSCLC or GBM resistant to a therapeutic agent or technique selected from the group consisting of:
(a) biological response modifiers;
(b) cytokines;
(c) lymphokines;
(d) therapeutic antibodies;
(e) antisense therapies;
(f) gene therapies;
(g) ribozymes;
(h) RNA interference; and
(i) vaccines.

When the biotherapeutic resistance modulation is use against tumors resistant to therapeutic antibodies, the therapeutic antibody can be, but is not limited to, a therapeutic antibody selected from the group consisting of bevacizumab (Avastin™), rituximab (Rituxan™), trastuzumab (Herceptin™), and cetuximab (Erbitux™).

When the improvement is made by radiation therapy enhancement, the radiation therapy enhancement can be, but is not limited to, a radiation therapy enhancement agent or technique selected from the group consisting of:
(a) hypoxic cell sensitizers;
(b) radiation sensitizers/protectors;
(c) photosensitizers;
(d) radiation repair inhibitors;
(e) thiol depleters;
(f) vaso-targeted agents;
(g) DNA repair inhibitors;
(h) radioactive seeds;
(i) radionuclides;
(j) radiolabeled antibodies; and
(k) brachytherapy.

A substituted hexitol derivative such as dianhydrogalactitol can be used in combination with radiation for the treatment of NSCLC, as described above.

Hypoxic cell sensitizers are described in C. C. Ling et al., “The Effect of Hypoxic Cell Sensitizers at Different Irradiation Dose Rates,” Radiation Res. 109:396-406 (1987), incorporated herein by this reference. Radiation sensitizers are described in T. S. Lawrence, “Radiation Sensitizers and Targeted Therapies,” Oncology 17 (Suppl. 13):23-28 (2003), incorporated herein by this reference. Radiation protectors are described in S. B. Vuyyuri et al., “Evaluation of D-Methionine as a Novel Oral Radiation Protector for Prevention of Mucositis,” Clin. Cancer Res. 14:2161-2170 (2008), incorporated herein by this reference. Photosensitizers are described in R. R. Allison & C. H. Sibata, “Oncologic Photodynamic Therapy Photosensitizers: A Clinical Review,” Photodiagnosis Photodynamic Ther. 7:61-75 (2010), incorporated herein by this reference. Radiation repair inhibitors and DNA repair inhibitors are described in M. Hingorani et al., “Evaluation of Repair of Radiation-Induced DNA Damage Enhances Expression from Replication-Defective Adenoviral Vectors,” Cancer Res. 68:9771-9778 (2008), incorporated herein by this reference. Thiol depleters are described in K. D. Held et al., “Postirradiation Sensitization of Mammalian Cells by the Thiol-Depleting Agent Dimethyl Fumarate,” Radiation Res. 127:75-80 (1991), incorporated herein by this reference. Vaso-targeted agents are described in A. L. Seynhaeve et al., “Tumor Necrosis Factor α Mediates Homogeneous Distribution of Liposomes in Murine Melanoma that Contributes to a Better Tumor Response,” Cancer Res. 67:9455-9462 (2007). As described above, radiation therapy is employed for the treatment of NSCLC, so radiation therapy enhancement is significant for this malignancy. Also as described above, radiation therapy enhancement is significant for the treatment of GBM, as radiation therapy is frequently employed for this malignancy; hypoxic cell sensitizers are frequently employed for the treatment of GBM.

When the improvement is by use of a novel mechanism of action, the novel mechanism of action can be, but is not limited to, a novel mechanism of action that is a therapeutic interaction with a target or mechanism selected from the group consisting of:
(a) inhibitors of poly-ADP ribose polymerase;
(b) agents that affect vasculature or vasodilation;
(c) oncogenic targeted agents;
(d) signal transduction inhibitors;
(e) EGFR inhibition;
(f) protein kinase C inhibition;
(g) phospholipase C downregulation;
(h) Jun downregulation;
(i) histone genes;
(j) VEGF;
(k) ornithine decarboxylase;
(l) ubiquitin C;
(m) Jun D;
(n) v-Jun;
(o) GPCRs;
(p) protein kinase A;
(q) protein kinases other than protein kinase A;
(r) prostate specific genes;
(s) telomerase;
(t) histone deacetylase; and
(u) tyrosine kinase inhibitors.

EGFR inhibition is described in G. Giaccone & J. A. Rodriguez, “EGFR Inhibitors: What Have We Learned from the Treatment of Lung Cancer,” Nat. Clin. Pract. Oncol. 11:554-561 (2005), incorporated herein by this reference. Protein kinase C inhibition is described in H. C. Swannie & S. B. Kaye, “Protein Kinase C Inhibitors,” Curr. Oncol. Rep. 4:37-46 (2002), incorporated herein by this reference. Phospholipase C downregulation is described in A. M. Martelli et al., “Phosphoinositide Signaling in Nuclei of Friend Cells: Phospholipase C β Downregulation Is Related to Cell Differentiation,” Cancer Res. 54:2536-2540 (1994), incorporated herein by this reference. Downregulation of Jun (specifically, c-Jun) is described in A. A. P. Zada et al., “Downregulation of c-Jun Expression and Cell Cycle Regulatory Molecules in Acute Myeloid Leukemia Cells Upon CD44 Ligation,” Oncogene 22:2296-2308 (2003), incorporated herein by this reference. The role of histone genes as a target for therapeutic intervention is described in B. Calabretta et al., “Altered Expression of G1-Specific Genes in Human Malignant Myeloid Cells,” Proc. Natl. Acad. Sci. USA 83:1495-1498 (1986). The role of VEGF as a target for therapeutic intervention is described in A. Zielke et al., “VEGF-Mediated Angiogenesis of Human Pheochromocytomas Is Associated to Malignancy and Inhibited by anti-VEGF Antibodies in Experimental Tumors,” Surgery 132:1056-1063 (2002), incorporated herein by this reference. The role of ornithine decarboxylase as a target for therapeutic intervention is described in J. A. Nilsson et al., “Targeting Ornithine Decarboxylase in Myc-Induced Lymphomagenesis Prevents Tumor Formation,” Cancer Cell 7:433-444 (2005), incorporated herein by this reference. The role of ubiquitin C as a target for therapeutic intervention is described in C. Aghajanian et al., “A Phase I Trial of the Novel Proteasome Inhibitor PS341 in Advanced Solid Tumor Malignancies,” Clin. Cancer Res. 8:2505-2511 (2002), incorporated herein by this reference. The role of Jun D as a target for therapeutic intervention is described in M. M. Caffarel et al., “JunD Is Involved in the Antiproliferative Effect of Δ⁹-Tetrahydrocannibinol on Human Breast Cancer Cells,” Oncogene 27:5033-5044 (2008), incorporated herein by this reference. The role of v-Jun as a target for therapeutic intervention is described in M. Gao et al., “Differential and Antagonistic Effects of v-Jun and c-Jun,” Cancer Res. 56:4229-4235 (1996), incorporated herein by this reference. The role of protein kinase A as a target for therapeutic intervention is described in P. C. Gordge et al., “Elevation of Protein Kinase A and Protein Kinase C in Malignant as Compared With Normal Breast Tissue,” Eur. J. Cancer 12:2120-2126 (1996), incorporated herein by this reference. The role of telomerase as a target for therapeutic intervention is described in E. K. Parkinson et al., “Telomerase as a Novel and Potentially Selective Target for Cancer Chemotherapy,” Ann. Med. 35:466-475 (2003), incorporated herein by this reference. The role of histone deacetylase as a target for therapeutic intervention is described in A. Melnick & J. D. Licht, “Histone Deacetylases as Therapeutic Targets in Hematologic Malignancies,” Curr. Opin. Hematol. 9:322-332 (2002), incorporated herein by this reference.

When the improvement is made by use of selective target cell population therapeutics, the use of selective target cell population therapeutics can be, but is not limited to, a use selected from the group consisting of:
(a) use against radiation sensitive cells;
(b) use against radiation resistant cells; and
(c) use against energy depleted cells.

The improvement can also be made by use of a substituted hexitol derivative in combination with ionizing radiation as described above, particularly with respect to the use of ionizing radiation for the treatment of NSCLC or GBM as described above.

When the improvement is made by use of an agent that counteracts myelosuppression, the agent that counteracts myelosuppression can be, but is not limited to, a dithiocarbamate.

U.S. Pat. No. 5,035,878 to Borch et al., incorporated herein by this reference, discloses dithiocarbamates for treatment of myelosuppression; the dithiocarbamates are compounds of the formula R¹R²NCS(S)M or R¹R²NCSS—SC(S)NR³R⁴, wherein R¹, R², R³, and R⁴ are the same or different, and R¹, R², R³, and R⁴ are aliphatic, cycloaliphatic, or heterocycloaliphatic groups that are unsubstituted or substituted by hydroxyl; or wherein one of R¹ and R² and one of R³ and R⁴ can be hydrogen; or wherein R¹, R², R³, and R⁴ taken together with the nitrogen atom upon which the pair of R groups is substituted, can be a 5-membered or 6-membered N-heterocyclic ring which is aliphatic or aliphatic interrupted by a ring oxygen or a second ring nitrogen, and M is hydrogen or one equivalent or a pharmaceutically acceptable cation, in which case the rest of the molecule is negatively charged.

U.S. Pat. No. 5,294,430 to Borch et al., incorporated herein by this reference, discloses additional dithiocarbamates for treatment of myelosuppression. In general, these are compounds of Formula (D-I):

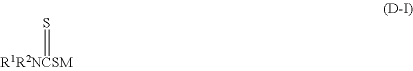

wherein:

(i) R¹ and R² are the same or different C₁-C₆ alkyl groups, C₃-C₆ cycloalkyl groups, or C₅-C₆ heterocycloalkyl groups; or

(ii) one of R¹ and R², but not both, can be H; or

(iii) R¹ and R² taken together with the nitrogen atom can be a 5-membered or 6-membered N-heterocyclic ring which is aliphatic or aliphatic interrupted by a ring oxygen or a second ring nitrogen; and

(iv) M is hydrogen or one equivalent of a pharmaceutically acceptable cation, in which case the rest of the molecule is negatively charged; or

(v) M is a moiety of Formula (D-II):

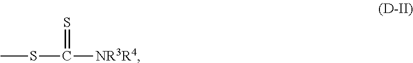

wherein R³ and R⁴ are defined in the same manner as R¹ and R². Where the group defined by Formula (D-I) is an anion, the cation can be an ammonium cation or can be derived from a monovalent or divalent metal such as an alkali metal or an alkaline earth metal, such as Na⁺, K⁺, or Zn⁺². In the case of the dithiocarbamic acids, the group defined by Formula (D-I) is linked to an ionizable hydrogen atom; typically, the hydrogen atom will dissociate at a pH above about 5.0. Among dithiocarbamates that can be used are: N-methyl,N-ethyldithiocarbamates, hexamethylenedithiocarbamic acid, sodium di(β-hydroxyethyl)dithiocarbamate, various dipropyl, dibutyl and diamyl dithiocarbamates, sodium N-methyl,N-cyclobutylmethyl dithiocarbamate, sodium N-allyl-N-cyclopropylmethyldithiocarbamate, cyclohexylamyldithiocarbamates, dibenzyl-dithiocarbamates, sodium dimethylene-dithiocarbamate, various pentamethylene dithiocarbamate salts, sodium pyrrolidine-N-carbodithioate, sodium piperidine-N-carbodithioate, sodium morpholine-N-carbo-dithioate, α-furfuryl dithiocarbamates and imidazoline dithiocarbamates. Another alternative is a compound where R¹ of Formula (D-I) is a hydroxy-substituted or, preferably, a (bis to penta) polyhydroxy-substituted lower alkyl group having up to 6 carbon atoms. For example, R¹ can be HO—CH₂—CHOH—CHOH—CHOH—CHOH—CH₂—. In such compounds, R² can be H or lower alkyl (unsubstituted or substituted with one or more hydroxyl groups). Steric problems can be minimized when R² is H, methyl, or ethyl. Accordingly, a particularly preferred compound of this type is an N-methyl-glucamine dithiocarbamate salt, the most preferred cations of these salts being sodium or potassium. Other preferred dithiocarbamates include the alkali or alkaline earth metal salts wherein the anion is di-n-butyldithiocarbamate, di-n-propyldithiocarbamate, pentamethylenedithiocarbamate, or tetramethylene dithiocarbamate.

When the improvement is made by use with an agent that increases the ability of the substituted hexitol to pass through the blood-brain barrier to treat brain metastases of NSCLC or to treat GBM, the agent that increases the ability of the substituted hexitol to pass through the blood-brain barrier can be, but is not limited to, an agent selected from the group consisting of:
(a) a chimeric peptide of the structure of Formula (D-III):

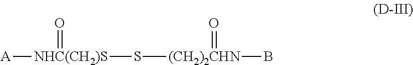

wherein: (A) A is somatostatin, thyrotropin releasing hormone (TRH), vasopressin, alpha interferon, endorphin, muramyl dipeptide or ACTH 4-9 analogue; and (B) B is insulin, IGF-I, IGF-II, transferrin, cationized (basic) albumin or prolactin; or a chimeric peptide of the structure of Formula (D-III) wherein the disulfide conjugating bridge between A and B is replaced with a bridge of Subformula (D-III(a)):

A-NH(CH₂)₂S—S—B(cleavable linkage) (D-III(a)),

wherein the bridge is formed using cysteamine and EDAC as the bridge reagents; or a chimeric peptide of the structure of Formula (D-III) wherein the disulfide conjugating bridge between A and B is replaced with a bridge of Subformula (D-III(b)):

A-NH═CH(CH₂)₃CH═NH—B(non-cleavable linkage) (D-III(b)),

wherein the bridge is formed using glutaraldehyde as the bridge reagent;
(b) a composition comprising either avidin or an avidin fusion protein bonded to a biotinylated substituted hexitol derivative to form an avidin-biotin-agent complex including therein a protein selected from the group consisting of insulin, transferrin, an anti-receptor monoclonal antibody, a cationized protein, and a lectin;
(c) a neutral liposome that is pegylated and incorporates the substituted hexitol derivative, wherein the polyethylene glycol strands are conjugated to at least one transportable peptide or targeting agent;
(d) a humanized murine antibody that binds to the human insulin receptor linked to the substituted hexitol derivative through an avidin-biotin linkage; and
(e) a fusion protein comprising a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative, wherein the fusion protein is linked to the substituted hexitol by a covalent link to biotin.

Agents that improve penetration of the blood-brain barrier are disclosed in W. M. Pardridge, “The Blood-Brain Barrier: Bottleneck in Brain Drug Development,” NeuroRx 2:3-14 (2005), incorporated herein by this reference.

One class of these agents is disclosed in U.S. Pat. No. 4,801,575 to Pardridge, incorporated herein by this reference, which discloses chimeric peptides for delivery of agents across the blood-brain barrier. These chimeric peptides include peptides of the general structure of Formula (D-IV):

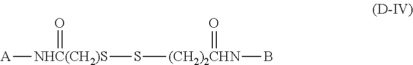

wherein:
(i) A is somatostatin, thyrotropin releasing hormone (TRH), vasopressin, alpha interferon, endorphin, muramyl dipeptide or ACTH 4-9 analogue; and
(ii) B is insulin, IGF-I, IGF-II, transferrin, cationized (basic) albumin or prolactin. In another alternative, the disulfide conjugating bridge between A and B is replaced with a bridge of Subformula (D-IV(a)):

A-NH(CH₂)₂S—S—B(cleavable linkage) (D-IV(a));

the bridge of Subformula (D-III(a)) is formed when cysteamine and EDAC are employed as the bridge reagents. In yet another alternative, the disulfide conjugating bridge between A and B is replaced with a bridge of Subformula (D-IV(b)):

A-NH═CH(CH₂)₃CH═NH—B(non-cleavable linkage) (D-IV(b));

the bridge of Subformula (D-III(b)) is formed when glutaraldehyde is employed as the bridge reagent.

U.S. Pat. No. 6,287,792 to Pardridge et al., incorporated herein by this reference, discloses methods and compositions for delivery of agents across the blood-brain barrier comprising either avidin or an avidin fusion protein bonded to a biotinylated agent to form an avidin-biotin-agent complex. The avidin fusion protein can include the amino acid sequences of proteins such as insulin or transferrin, an anti-receptor monoclonal antibody, a cationized protein, or a lectin.

U.S. Pat. No. 6,372,250 to Pardridge, incorporated herein by this reference, discloses methods and compositions for delivery of agents across the blood-brain barrier employing liposomes. The liposomes are neutral liposomes. The surface of the neutral liposomes is pegylated. The polyethylene glycol strands are conjugated to transportable peptides or other targeting agents. Suitable targeting agents include insulin, transferrin, insulin-like growth factor, or leptin. Alternatively, the surface of the liposome could be conjugated with 2 different transportable peptides, one peptide targeting an endogenous BBB receptor and the other targeting an endogenous BCM (brain cell plasma membrane) peptide. The latter could be specific for particular cells within the brain, such as neurons, glial cells, pericytes, smooth muscle cells, or microglia. Targeting peptides may be endogenous peptide ligands of the receptors, analogues of the endogenous ligand, or peptidomimetic MAbs that bind the same receptor of the endogenous ligand. Transferrin receptor-specific peptidomimetic monoclonal antibodies can be used as transportable peptides. Monoclonal antibodies to the human insulin receptor can be used as transportable peptides. The conjugation agents which are used to conjugate the blood-barrier targeting agents to the surface of the liposome can be any of the well-known polymeric conjugation agents such as sphingomyelin, polyethylene glycol (PEG) or other organic polymers, with PEG preferred. The liposomes preferably have diameters of less than 200 nanometers. Liposomes having diameters of between 50 and 150 nanometers are preferred. Especially preferred are liposomes or other nanocontainers having external diameters of about 80 nanometers. Suitable types of liposomes are made with neutral phospholipids such as 1-palmitoyl-2-oleoyl-sn-glycerol-3-phosphocholine (POPC), diphosphatidyl phosphocholine, distearoylphosphatidylethanolamine (DSPE), or cholesterol. The transportable peptide is linked to the liposome as follows: A transportable peptide such as insulin or an HIRMAb is thiolated and conjugated to a maleimide group on the tip of a small fraction of the PEG strands; or, surface carboxyl groups on a transportable peptide such as transferrin or a TfRMAb are conjugated to a hydrazide (Hz) moiety on the tip of the PEG strand with a carboxyl activator group such as N-methyl-N′-3(dimethylaminopropyl)carbodiimide hydrochloride (EDAC); a transportable peptide is thiolated and conjugated via a disulfide linker to the liposome that has been reacted with N-succinimidyl 3-(2-pyridylthio)propionate (SPDP); or a transportable peptide is conjugated to the surface of the liposome with avidin-biotin technology, e.g., the transportable peptide is mono-biotinylated and is bound to avidin or streptavidin (SA), which is attached to the surface of the PEG strand.

U.S. Pat. No. 7,388,079 to Pardridge et al., incorporated herein by this reference, discloses the use of a humanized murine antibody that binds to the human insulin receptor; the humanized murine antibody can be linked to the agent to be delivered through an avidin-biotin linkage.

U.S. Pat. No. 8,124,095 to Pardridge et al., incorporated herein by this reference, discloses monoclonal antibodies that are capable of binding to an endogenous blood-brain barrier receptor-mediated transport system and are thus capable of serving as a vector for transport of a therapeutic agent across the BBB. The monoclonal antibody can be, for example, an antibody specifically binding the human insulin receptor on the human BBB.

United States Patent Application Publication No. 2005/0085419 by Morrison et al., incorporated herein by this reference, discloses a fusion protein for delivery of a wide variety of agents to a cell via antibody-receptor-mediated endocytosis comprises a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative. Typically, the antigen is a protein. Typically, the protein antigen on the surface of the cell is a receptor such as a transferrin receptor- or an insulin receptor. The invention also includes an antibody construct incorporating the fusion protein that is either a heavy chain or a light chain together with a complementary light chain or heavy chain to form an intact antibody molecule. The therapeutic agent can be a non-protein molecule and can be linked covalently to biotin.

When the improvement is an improvement in the therapeutic employment of a substituted hexitol derivative such as dianhydrogalactitol for treatment of brain metastases of NSCLC or for the treatment of GBM made by use of an agent that suppresses the growth of cancer stem cells (CSCs), the agent that suppresses the growth of cancer stem cells can be, but is not limited to: (1) naphthoquinones; (2) VEGF-DLL4 bispecific antibodies; (3) farnesyl transferase inhibitors; (4) gamma-secretase inhibitors; (5) anti-TIM3 antibodies; (6) tankyrase inhibitors; (7) Wnt pathway inhibitors other than tankyrase inhibitors; (8) camptothecin-binding moiety conjugates; (9) Notch1 binding agents, including antibodies; (10) oxabicycloheptanes and oxabicycloheptenes; (11) inhibitors of the mitochondrial electron transport chains or the mitochondrial tricarboxylic acid cycle; (12) Axl inhibitors; (13) dopamine receptor antagonists; (14) anti-RSPO1 antibodies; (15) inhibitors or modulators of the Hedgehog pathway; (16) caffeic acid analogs and derivatives; (17) Stat3 inhibitors; (18) GRP-94-binding antibodies; (19) Frizzled receptor polypeptides; (20) immunoconjugates with cleavable linkages; (21) human prolactin, growth hormone, or placental lactogen; (22) anti-prominin-1 antibody; (23) antibodies specifically binding N-cadherin; (24) DR5 agonists; (25) anti-DLL4 antibodies or binding fragments thereof; (26) antibodies specifically binding GPR49; (27) DDR1 binding agents; (28) LGR5 binding agents; (29) telomerase-activating compounds; (30) fingolimod plus anti-CD74 antibodies or fragments thereof; (31) an antibody that prevents the binding of CD47 to SIPRα or a CD47 mimetic; (32) thienopyranone kinase inhibitors for inhibition of PI-3 kinases; (33) cancer-stem-cell-binding peptides; (34) diphtheria toxin-interleukin 3 conjugates; (35) inhibitors of histone deacetylase; (36) progesterone or analogs thereof; (37) antibodies binding the negative regulatory region (NRR) of Notch2; (38) inhibitors of HGFIN; (39) immunotherapeutic peptides; (40) inhibitors of CSCPK or related kinases; (41) imidazo[1,2-a]pyrazine derivatives as α-helix mimetics; (42) antibodies directed to an epitope of variant Heterogeneous Ribonucleoprotein G (HnRNPG); (43) antibodies binding TES7 antigen; (44) antibodies binding the ILR3α subunit; (45) ifenprodil tartrate and other compounds with a similar activity; (46) antibodies binding SALL4; (47) antibodies binding Notch4; (48) bispecific antibodies binding both NBR1 and Cep55; (49) Smo inhibitors; (50) peptides blocking or inhibiting interleukin-1 receptor 1; (51) antibodies specific for CD47 or CD19; (52) histone methyltransferase inhibitors; (53) antibodies specifically binding Lg5; (54) antibodies specifically binding EFNA1; (55) phenothiazine derivatives; (56) HDAC inhibitors plus AKT inhibitors; (57) ligands binding to cancer-stem-line-specific cell surface antigen stem cell markers; (58) Notch receptor agonists; (59) binding agents binding human MET; (60) PDGFR-β inhibitors; (61) pyrazolo compounds with histone demethylase activity; (62) heterocyclic substituted 3-heteroaryidenyl-2-indolinone derivatives; (63) albumin-binding arginine deiminase fusion proteins; (64) hydrogen-bond surrogate peptides and peptidomimetics that reactivate p53; (65) prodrugs of 2-pyrrolinodoxorubicin conjugated to antibodies; (66) targeted cargo proteins; (67) bisacodyl and analogs thereof; (68) N¹-cyclic amine-N⁵-substituted phenyl biguanide derivative; (69) fibulin-3 protein; (70) modulators of SCFSkp2; (71) inhibitors of Slingshot-2; (72) monoclonal antibodies specifically binding DCLK1 protein; (73) antibodies or soluble receptors that modulate the Hippo pathway; (74) selective inhibitors of CDK8 and CDK19; (75) antibodies and antibody fragments specifically binding IL-17; (76) antibodies specifically binding FRMD4A; (77) monoclonal antibodies specifically binding the ErbB-3 receptor; (78) antibodies that specifically bind human RSPO3 and modulate 3-catenin activity; (79) esters of 4,9-dihydroxy-naphtho[2,3-b]furans; (80) CCR5 antagonists; (81) antibodies that specifically bind the extracellular domain of human C-type lectin-like molecule (CLL-1); (82) anti-hypertension compounds; (83) anthraquinone radiosensitizer agents plus ionizing radiation; (84) CDK-inhibiting pyrrolopyrimidinone derivatives; (85) analogs of CC-1065 and conjugates thereof; (86) antibodies specifically binding to the protein Notum; (87) CDK8 antagonists; (88) bHLH proteins and nucleic acids encoding them; (89) inhibitors of the histone methyltransferase EZH2; (90) sulfonamides inhibiting carbonic anhydrase isoforms; (91) antibodies specifically binding DEspR; (92) antibodies specifically binding human leukemia inhibitory factor (LIF); (93) doxovir; (94) inhibitors of mTOR; (95) antibodies specifically binding FZD10; (96) napthofurans; (97) death receptor agonists; (98) tigecycline; (99) strigolactones and strigolactone analogs; and (100) compounds inducing methuosis. Other compounds and methods capable of suppression of stem cell proliferation are known in the art.

Increasing importance has been placed on the existence and role of cancer stem cells with respect to metastasis, drug resistance, and other aspects of cancer proliferation. Cancer stem cells were first identified in acute myeloid leukemia but since have been identified in many other types of malignancies. Cancer stem cells possess many of the characteristics associated with normal stem cells, in particular the ability to give rise to all cell types found in a particular cancer sample, as well as possibly other cell types. Cancer stem cells are therefore tumorigenic, and may generate tumors through the stem cell processes of self-renewal and differentiation into multiple cell types. Cancer stem cells can also undergo clonal evolution through the occurrence of mutations that confer more aggressive properties and their selection.

Cancer stem cells are described in G. H. Heppner et al., “Tumor Heterogeneity: Biological Implications and Therapeutic Consequences,” Cancer Metastasis Rev. 2:5-23 (1983); T. Reya et al., “Stem Cells, Cancer, and Cancer Stem Cells,” Nature 414:105-111 (2001); P. B. Gupta et al., “Cancer Stem Cells: Mirage or Reality,” Nature Med. 15:1010-1012 (2009); S. K. Singh et al., “Identification of a Cancer Stem Cell in Human Brain Tumors,” Cancer Res. 63:5821-5828 (2003); M. Al-Hajj et al., “Prospective Identification of Tumorigenic Breast Cancer Cells,” Proc. Natl. Acad. Sci. USA 100:3983-3988 (2003); S. Zhang et al., “Identification and Characterization of Ovarian Cancer-Initiating Cells from Primary Human Tumors,” Cancer Res. 68:4311-4320 (2008); A. B. Alvero et al., “Molecular Phenotyping of Human Ovarian Cancer Stem Cells Unravels the Mechanisms for Repair and Chemoresistance,” Cell Cycle 8:158-166 (2009); J. P. Sullivan et al., “Aldehyde Dehydrogenase Activity Selects for Lung Adenocarcinoma Stem Cells Dependent on Notch Signaling,” Cancer Res. 70:9937-9948 (2010); and L. Jin et al., “Monoclonal Antibody-Mediated Targeting of CD123, IL-3 Receptor Chain α, Eliminates Human Acute Myeloid Leukemic Stem Cells,” Cell Stem Cell 5:31-42 (2009), all of which are incorporated herein by this reference.

U.S. Pat. No. 8,871,802 to Jiang et al., incorporated herein by this reference, discloses naphthoquinones for suppression of cancer stem cell proliferation, including, but not limited to: 2-sulfinyl substituted naphtho[2,3-b]furan-4,9-diones; 2-sulfonyl substituted naphtho[2,3-b]furan-4,9-diones; 2-(1-hydroxy-2-nitroethenyl) substituted naphtho[2,3-b]furan-4,9-diones; 2-(1-hydroxy-2-methylsulfinylethenyl) substituted naphtho[2,3-b]furan-4,9-diones; 2-(1-hydroxy-2-methylsulfonylethenyl) substituted naphtho[2,3-b]furan-4,9-diones; 2-(1-methyl-2-methylsulfinylethenyl) substituted naphtho[2,3-b]furan-4,9-diones; 2-sulfonyl substituted naphtho[2,3-b]thiophene-4,9-diones; and 2-sulfinyl substituted naphtho[2,3-b]thiophene-4,9-diones.

U.S. Pat. No. 8,858,941 to Gurney et al., incorporated herein by this reference, discloses VEGF-DLL4 bispecific antibodies.

U.S. Pat. No. 8,853,274 to Wang, incorporated herein by this reference, discloses the use of farnesyl transferase inhibitors and gamma-secretase inhibitors to suppress cancer stem cell proliferation. The use of gamma-secretase inhibitors to suppress cancer stem cell proliferation is also disclosed in United States Patent Application Publication No. 2014/0227173 by Eberhart et al., incorporated herein by this reference. The gamma-secretase inhibitors include compounds of Formula (IV)

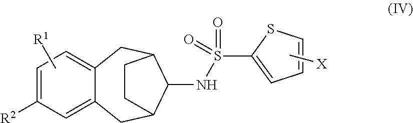

wherein:

(1) X is halogen;

(2) R¹ is hydrogen, halogen, hydroxy, (C₁-C₆)alkyl, or (C₁-C₄)alkoxy; and

(3) R² is a moiety of Subformula (IV(a))

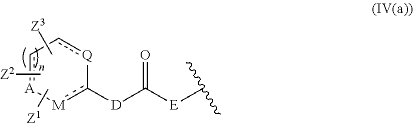

wherein: (a) E is CH₂ or NH; (b) D is (CH₂)ₘ, O(CH₂)ₘ, HN(CH₂)ₘ, or CH═CH, wherein m is 0, 1, or 2; (c) A and Q are independently N, NCH₃, or C; (d) M is C or C═O; (e) n is 1 or 2; (f) Z¹ and Z² are independently hydrogen, halogen, halo(C₁-C₄)alkyl or phenyl; or Z¹ and Z², when attached to carbon atoms, form a 6-membered aryl ring with the carbon atoms to which they are attached; and (g) Z³ is hydrogen, halogen, halo(C₁-C₄)alkyl or phenyl.

U.S. Pat. No. 8,841,418 to Karsunky et al., incorporated herein by this reference, discloses the use of anti-TIM3 antibodies to suppress CSC proliferation. The use of anti-TIM3 antibodies is also disclosed in U.S. Pat. No. 8,647,623 to Takayanagi et al., incorporated herein by this reference.

U.S. Pat. No. 8,841,299 to Hermann et al., incorporated herein by this reference, discloses tankyrase inhibitors useful for modulation of the Wnt pathway, including substituted pyrrolo[1,2-a]pyrazines such as, but not limited to, 6-bromo-3-(4-methoxy-phenyl)-2H-pyrrolo[1,2-a]pyrazin-1-one, 1-oxo-3-(4-trifluoromethyl-phenyl)-1,2-dihydro-pyrrolo[1,2-a]pyrazine-6-carbonitrile, N-hydroxy-1-oxo-3-(4-trifluoromethyl-phenyl)-1,2-dihydro-pyrrolo[1,2-a]pyrazine-6-carboxamidine, 1-oxo-3-(4-trifluoromethyl-phenyl)-1,2-dihydro-pyrrolo[1,2-a]pyrazine-6-c-arboxamidine, 6-(4,5-dihydro-1H-imidazol-2-yl)-3-(4-trifluoromethyl-phenyl)-2H-pyrrolo[1,2-a]pyrazin-1-one, 6-methyl-3-(4-trifluoromethyl-phenyl)-2H-pyrrolo[1,2-a]pyrazin-1-one, 6-hydroxymethyl-3-(4-trifluoromethyl-phenyl)-2H-pyrrolo[1,2-a]pyrazin-1-one, 3-[4-(2-fluoro-phenyl)-piperazin-1-yl]-6-methyl-2H-pyrrolo[1,2-a]pyrazin-1-one, and 6-bromo-3-(4-trifluoromethyl-phenyl)-2H-pyrrolo[1,2-a]pyrazin-1-one. U.S. Pat. No. 8,722,661 to Haynes et al., incorporated herein by this reference, also discloses tankyrase inhibitors, such as, but not limited to, 7-methyl-2-(4-pyridin-4-yl-piperazin-1-yl)-3,7-dihydro-pyrrolo[2,3-d]pyrimidin-4-one, 4-[4-(7-methyl-4-oxo-4,7-dihydro-3H-pyrrolo[2,3-d]pyrimidin-2-yl)-piperazin-1-yl]-benzoic acid ethyl ester, 2-[4-(4-chloro-phenyl)-piperazin-1-yl]-7-methyl-3,7-dihydro-pyrrolo[2,3-d]pyrimidin-4-one, 7-methyl-2-(4-pyridin-2-yl-piperazin-1-yl)-3,7-dihydro-pyrrolo[2,3-d]pyrimidin-4-one, 2-[4-(4-fluoro-2-methanesulfonyl-phenyl)-piperazin-1-yl]-7-methyl-3,7-dihydro-pyrrolo[2,3-d]pyrimidin-4-one, 7-methyl-2-[4-(3-trifluoromethyl-pyridin-2-yl)-piperazin-1-yl]-3,7-dihydro-pyrrolo[2,3-d]pyrimidin-4-one, 2-[4-(3,5-dichloro-phenyl)-piperazin-1-yl]-7-methyl-3,7-dihydro-pyrrolo[2,3-d]pyrimidin-4-one, 7-methyl-2-(4-pyrimidin-2-yl-piperazin-1-yl)-3,7-dihydro-pyrrolo[2,3-d]pyrimidin-4-one, 2-[4-(7-methyl-4-oxo-4,7-dihydro-3H-pyrrolo[2,3-d]pyrimidin-2-yl)-piperazin-1-yl]-nicotinonitrile, 4-(7-methyl-4-oxo-4,7-dihydro-3H-pyrrolo[2,3-d]pyrimidin-2-yl)-3,4,5,6-tetrahydro-2H-[1,2′]bipyrazinyl-3′-carbonitrile, and 7-methyl-2-(4-methyl-piperazin-1-yl)-3,7-dihydro-pyrrolo[2,3-d]pyrimidin-4-one. United States Patent Application Publication No. 2014/0121231 by Bolin et al., incorporated herein by this reference, discloses pyranopyridone inhibitors of tankyrase. Other Wnt pathway inhibitors are disclosed in U.S. Pat. No. 8,445,491 to Lum et al., incorporated herein by this reference, and in U.S. Pat. No. 8,304,408 to Wrasidlo et al., incorporated herein by this reference. The compounds of U.S. Pat. No. 8,445,491 to Lum et al. include compounds of Formula (V) or Formula (VI), wherein Formula (V) is

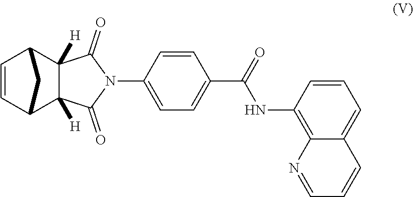 [SMILES: O=C(Nc1cccc2cccnc12)c1ccc(N2C(=O)[C@@H]3[C@H](C2=O)[C@@H]2C=C[C@H]3C2)cc1]

### and Formula (VI) is

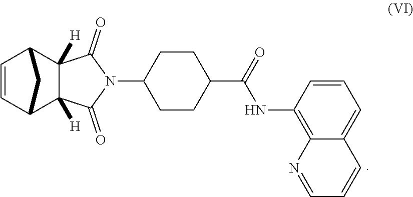 [SMILES: O=C(Nc1cccc2cccnc12)C1CCC(N2C(=O)[C@@H]3[C@H](C2=O)[C@@H]2C=C[C@H]3C2)CC1]

The compounds of U.S. Pat. No. 8,304,408 to Wrasidlo et al. are debromohymenialdesine or debromohymenialdesine analogs, including compounds of Formula (VII)

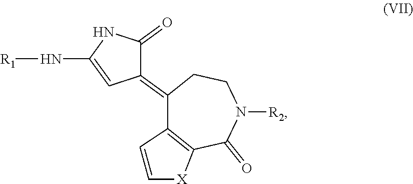

wherein X is selected from the group consisting of NH, O, S and CH₂, and the R₁ and/or the R₂ group are independently selected from the group consisting of hydrogen, halo, hydroxy, mercapto, cyano, formyl, alkyl, heteroalkyl, heteroalkenyl, heteroalkynyl, haloalkyl, alkenyl, alkynyl, aryl, substituted alkyl, substituted alkenyl. substituted alkynyl, amino, nitro, alkoxy, haloalkoxy, thioalkoxy, alkanoyl, haloalkanoyl and carboxy, wherein the “hetero” term refers to groups that contain one or more heteroatoms selected from the group consisting of O, S, N and combinations thereof. Still other tankyrase inhibitors are disclosed in United States Patent Application Publication No. 2014/0121231 by Bolin et al., incorporated herein by this reference, including pyranopyridone inhibitors of Formula (VIII)

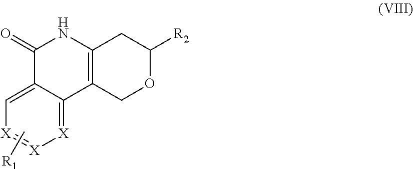

wherein:

(1) X is independently in each occurrence N or CH;

(2) Y is S, O, CH or NCH₃;

(3) M is S or CH;

(4) R₁ is H, C₁-C₆ alkyl, C₃-C₇ cycloalkyl, C(CH₃)₂OH, CN, NO₂, CO₂CH₃, CONH₂, NH₂, or halogen; and

(5) R₂ is selected from the group consisting of H, optionally substituted C₁-C₆ alkyl, C₅-C₁₂ spiroalkyl, C₁-C₆ alkoxy, C₃-C₇ cycloalkyl, heterocycloalkyl, and substituted heterocycloalkyl, wherein the heterocycloalkyl is optionally substituted by C₁-C₆ alkyl, C₁-C₆ hydroxyalkyl, C₁-C₃ alkoxy-C₁-C₆ alkyl, oxetanyl, tetrahydrofuranyl, pyranyl, or SO₂R₃ wherein R₃ is C₁-C₆ alkyl, C₁-C₆ hydroxyalkyl, oxetanyl, tetrahydrofuranyl, or pyranyl.

U.S. Pat. No. 8,834,886 to Govindan et al. and U.S. Pat. No. 8,268,317 to Govindan et al., both incorporated herein by this reference, discloses camptothecin-binding moiety conjugates that can target cancer stem cell antigens such as CD133 or CD44; the conjugates can include a monoclonal antibody as targeting moiety.

U.S. Pat. No. 8,834,875 to Van Der Horst, incorporated herein by this reference, discloses Notch1 binding agents, specifically antibodies that specifically bind to a non-ligand binding membrane proximal region of the extracellular domain of human Notch1. Other anti-Notch1 antibodies that can be used for suppression of proliferation of cancer stem cells are disclosed in U.S. Pat. No. 8,784,811 to Lewicki et al., U.S. Pat. No. 8,460,661 to Gurney et al., U.S. Pat. No. 8,435,513 to Gurney et al., and U.S. Pat. No. 8,226,943 to Gurney et al., U.S. Pat. No. 8,088,617 to Gurney et al., U.S. Pat. No. 7,919,092 to Lewicki, all of which are incorporated herein by this reference.

U.S. Pat. No. 8,822,461 to Kovach et al., U.S. Pat. No. 8,541,458 to Kovach et al., U.S. Pat. No. 8,426,444 to Kovach et al., U.S. Pat. No. 7,998,957 to Kovach et al., all incorporated herein by this reference, discloses oxabicycloheptanes and oxabicycloheptenes that can suppress cancer stem cell proliferation. These compounds are inhibitors of protein phosphorylation and interact with N—CoR.

U.S. Pat. No. 8,815,844 to Clement et al., incorporated herein by this reference, discloses inhibitors of the mitochondrial electron transport chains or the mitochondrial tricarboxylic acid cycle for suppression of cancer stem cell proliferation; the inhibitors include rotenone, myxothiazole, stigmatellin, and piericidin.

Inhibitors of the receptor protein tyrosine kinase Axl are usable for suppression of cancer stem cell proliferation. Inhibitors of Axl are disclosed in U.S. Pat. No. 8,839,364 to Singh et al., including polycyclic aryl and polycyclic heteroaryl substituted triazoles; U.S. Pat. No. 8,839,347 to Goff et al., including bicyclic aryl substituted triazoles or heteroaryl substituted triazoles such as N³-(3-(bicyclo[2.2.1]heptan-2-yl)-1,2,3,4,5,6-hexahydrobenzo[d]azocin-8-yl)-1-(2-chloro-7-methylthieno[3,2-d]pyrimidin-4-yl)-1H-1,2,4-triazole-3,5-diamine; U.S. Pat. No. 8,796,259 to Ding et al., including N³-heteroaryl substituted triazoles and N⁵-heteroaryl substituted triazoles; U.S. Pat. No. 8,741,898 to Goff et al., including polycyclic heteroaryl substituted triazoles such as 1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-2-yl)-1H-1,2,4-triazole-3,5-diamine; U.S. Pat. No. 8,618,331 to Goff et al., including polycyclic heteroaryl substituted triazoles such as N³-(4-(4-cyclohexanylpiperazin-1-yl)phenyl)-1-(6,7-dihydro-5H-benzo[6,7]cyclohepta[1,2-c]pyridazin-3-yl)-1H-1,2,4-triazole-3,5-diamine; 1-(6,7-dihydro-5H-benzo[6,7]cyclohepta[1,2-c]pyridazin-3-yl)-N³-(3-fluoro-4-(4-(pyrrolidin-1-yl)piperidin-1-yl)-1H-1,2,4-triazole-3,5-diamine; 1-(6,7-dihydro-5H-benzo[6,7]cyclohepta[1,2-c]pyridazin-3-yl)-N³-(3-fluoro-4-(4-methyl-3-phenylpiperazin-1-yl)phenyl)-1H-1,2,4-triazole-3,5-diamine; 1-(6,7-dihydro-5H-benzo[6,7]cyclohepta[1,2-c]pyridazin-3-yl)-N³-(3-fluoro-(4-(4-piperidin-1-yl)piperidin-1-yl)phenyl)-1H-1,2,4-triazole-3,5-diamine; 1-(6,7-dihydro-5H-benzo[6,7]cyclohepta[1,2-c]pyridazin-3-yl)-N³-(3-fluoro-4-(4-(indolin-2-on-1-yl)piperidin-1-yl)phenyl)-1H-1,2,4-triazole-3,5-diamine; 1-(6,7-dihydro-5H-benzo[6,7]cyclohepta[1,2-c]pyridazin-3-yl)-N³-(3-fluoro-4-(4-(morpholin-4-yl)piperidin-1-yl)phenyl)-1H-1,2,4-triazole-3,5-diamine; 1-(6,7-dihydro-5H-benzo[6,7]cyclohepta[1,2-c]pyridazin-3-yl)-N³-(4-(4-cyclopentyl-2-methylpiperazin-1-yl)phenyl)-1H-1,2,4-triazole-3,5-diamine; and 1-(6,7-dihydro-5H-benzo[6,7]cyclohepta[1,2-c]pyridazin-3-yl)-N³-(4-(3,5-dimethylpiperazin-1-yl)phenyl)-1H-1,2,4-triazole-3,5-diamine; U.S. Pat. No. 8,609,650 to Goff et al., including bridged bicyclic aryl and bridged bicyclic heteroaryl substituted triazoles, such as 1-(1,4-ethano-8-phenyl-1,2,3,4-tetrahydro-1,5-naphthyridin-6-yl)-N³-(3-fluoro-4-(4-(pyrrolidin-1-yl)piperidin-1-yl)phenyl)-1H-1,2,4-triazole-3,5-diamine; 1-(1,4-ethano-8-thiophen-2-yl-1,2,3,4-tetrahydro-1,5-naphthyridin-6-yl)-N³-(3-fluoro-4-(4-(pyrrolidin-1-yl)piperidin-1-yl)phenyl)-1H-1,2,4-triazole-3,5-diamine; 1-(1,4-ethano-8-pyridin-4-yl-1,2,3,4-tetrahydro-1,5-naphthyridin-6-yl)-N³-(3-fluoro-4-(4-(pyrrolidin-1-yl)piperidin-1-yl)phenyl)-1H-1,2,4-triazole-3,5-diamine; 1-(1,4-ethano-8-phenyl-1,2,3,4-tetrahydro-1,5-naphthyridin-6-yl)-N³-(3-fluoro-4-(3-carboxypiperazin-1-yl)phenyl)-1H-1,2,4-triazole-3,5-diamine; 1-(1,4-ethano-8-phenyl-1,2,3,4-tetrahydro-1,5-naphthyridin-6-yl)-N³-(4-(4-methylpiperazin-1-yl)phenyl)-1H-1,2,4-triazole-3,5-diamine; 1-(1,4-ethano-8-phenyl-1,2,3,4-tetrahydro-1,5-naphthyridin-6-yl)-N³-(3-fluoro-4-(4-bicyclo[2.2.1]heptan-2-ylpiperazin-1-yl)phenyl)-1H-1,2,4-triazole-3,5-diamine; 1-(1,4-ethano-8-phenyl-1,2,3,4-tetrahydro-1,5-naphthyridin-6-yl)-N³-(3-fluoro-4-(4-cyclohexyl piperazin-1-yl)phenyl)-1H-1,2,4-triazole-3,5-diamine; 1-(1,4-ethano-8-phenyl-1,2,3,4-tetrahydro-1,5-naphthyridin-6-yl)-N³-(3-fluoro-4-(4-(4-methylpiperazin-1-yl)piperidin-lyl)phenyl)-1H-1,2,4-triazole-3,5-diamine; 1-(1,4-ethano-8-phenyl-1,2,3,4-tetrahydro-1,5-naphthyridin-6-yl)-N³-(3-fluoro-4-(4-ethyloxycarbonylmethylpiperazin-1-yl)phenyl)-1H-1,2,4-triazole-3,5-diamine; 1-(1,4-ethano-8-phenyl-1,2,3,4-tetrahydro-1,5-naphthyridin-6-yl)-N³-(3-fluoro-4-(4-carboxymethylpiperazin-1-yl)phenyl)-1H-1,2,4-triazole-3,5-diamine; and 1-(1,4-ethano-8-(4-trifluoromethylphenyl)-1-1,2,3,4-tetrahydro-1,5-naphthyridin-6-yl)-N³-(3-fluoro-4-(4-(pyrrolidin-1-yl)piperidin-1-yl)phenyl)-1H-1,2,4-triazole-3,5-diamine; U.S. Pat. No. 8,492,373 to Goff et al., including bicyclic aryl and bicyclic heteroaryl substituted triazoles, including N³-(3-cyclopentyl-1,2,3,4,5,6-hexahydrobenzo[d]azocin-8-yl)-1-(6-fluoroquinazolin-4-yl)-1H-1,2,4-triazole-3,5-diamine; 1-(benzo[d]thiazol-2-yl)-N³-(3-cyclopentyl-1,2,3,4,5,6-hexahydrobenzo[d]azocin-8-yl)-1H-1,2,4-triazole-3,5-diamine; 1-(benzo[d]thiazol-2-yl)-N³-(3-cyclopentyl-1,2,3,4,5,6-hexahydrobenzo[d]azocin-9-yl)-1H-1,2,4-triazole-3,5-diamine; 1-(2-chloro-7-methylthieno[3,2-d]pyrimidin-4-yl)-N³-(2,3,4,5-tetrahydrobenzo[b][1,4]dioxocin-8-yl)-1H-1,2,4-triazole-3,5-diamine; N³-(3-cyclopentyl-1,2,3,4,5,6-hexahydro-benzo[d]azocin-8-yl)-1-(6,7-dimethoxyquinazolin-4-yl)-1H-[1,2,4]triazole-3,5-diamine; 1-(2-chloro-7-methylthieno[3,2-d]pyrimidin-4-yl)-N³-(3-cyclopentyl-1,2,3,4,5,6-hexahydrobenzo[d]azocin-8-yl)-1H-1,2,4-triazole-3,5-diamine; N³-(3-(bicyclo[2.2.1]heptan-2-yl)-1,2,3,4,5,6-hexahydrobenzo[d]azocin-8-yl)-1-(2-chloro-7-methylthieno[3,2-c]pyrimidin-4-yl)-1H-1,2,4-triazole-3,5-diamine; N-3 (bicyclo[2.2.1]heptan-2-yl)-1,2,3,4,5,6-hexahydrobenzo[d]azocin-8-yl)-1-(6,7-dimethoxyquinazolin-4-yl)-1H-1,2,4-triazole-3,5-diamine; N³-(3-cyclopentyl-1,2,3,4,5,6-hexahydrobenzo[d]azocin-8-yl)-1-(7-methylthieno[3,2-d]pyrimidin-4-yl)-1H-1,2,4-triazole-3,5-diamine; N³-(3-(bicyclo[2.2.1]heptan-2-yl)-1,2,3,4,5,6-hexahydrobenzo[d]azocin-8-yl)-1-(7-methylthieno[3,2-d]pyrimidin-4-yl)-1H-1,2,4-triazole-3,5-diamine; N³-(1-oxo-1,2,3,4,5,6-hexahydrobenzo[c]azocin-9-yl)-1-(2-chloro-7-methylthieno[3,2-d]pyrimidin-4-yl)-1H-1,2,4-triazole-3,5-diamine; N³-(1-oxo-1,2,3,4,5,6-hexahydrobenzo[c]azocin-9-yl)-1-(7-methylthieno [3,2-d]pyrimidin-4-yl)-1H-1,2,4-triazole-3,5-diamine; and N³-(1-oxo-1,2,3,4,5,6-hexahydrobenzo[c]azocin-9-yl)-1-(6,7-dimethoxyquinazolin-4-yl)-1H-1,2,4-triazole-3,5-diamine; U.S. Pat. No. 8,431,594 to Singh et al., including bridged bicyclic heteroaryl substituted triazoles, such as (7S)-1-(1,4-ethano-8-phenyl-1,2,3,4-tetrahydro-1,5-naphthyridin-6-yl)-N³-(7-(t-butoxycarbonylamino)-6,7,8,9-tetrahydro-5H-benzo[7]annulene-2-yl)-1H-1,2,4-triazole-3,5-diamine; (7S)-1-(1,4-ethano-8-phenyl-1,2,3,4-tetrahydro-1,5-naphthyridin-6-yl)-N³-(7-(diethylamino)-6,7,8,9-tetrahydro-5H-benzo[7]annulene-2-yl)-1H-1,2,4-triazole-3,5-diamine; (7S)-1-(1,4-ethano-8-phenyl-1,2,3,4-tetrahydro-1,5-naphthyridin-6-yl)-N³-(7-(dimethylamino)-6,7,8,9-tetrahydro-5H-benzo[7]annulene-2-yl)-1H-1,2,4-triazole-3,5-diamine; (7S)-1-(1,4-ethano-8-phenyl-1,2,3,4-tetrahydro-1,5-naphthyridin-6-yl)-N³-(7-(isopropylamino)-6,7,8,9-tetrahydro-5H-benzo[7]annulene-2-yl)-1H-1,2,4-triazole-3,5-diamine; (7S)-1-(1,4-ethano-8-phenyl-1,2,3,4-tetrahydro-1,5-naphthyridin-6-yl)-N³-(7-(cyclobutylamino)-6,7,8,9-tetrahydro-5H-benzo[7]annulene-2-yl)-1H-1,2,4-triazole-3,5-diamine; (7S)-1-(1,4-ethano-8-phenyl-1,2,3,4-tetrahydro-1,5-naphthyridin-6-yl)-N³-(7-(dipropylamino)-6,7,8,9-tetrahydro-5H-benzo[7]annulene-2-yl)-1H-1,2,4-triazole-3,5-diamine; (7S)-1-(1,4-ethano-8-phenyl-1,2,3,4-tetrahydro-1,5-naphthyridin-6-yl)-N³-(7-(isobutylamino)-6,7,8,9-tetrahydro-5H-benzo[7]annulene-2-yl)-1H-1,2,4-triazole-3,5-diamine; and (7S)-1-(1,4-ethano-8-phenyl-1,2,3,4-tetrahydro-1,5-naphthyridin-6-yl)-N³-(7-(diisobutylamino)-6,7,8,9-tetrahydro-5H-benzo[7]annulene-2-yl)-1H-1,2,4-triazole-3,5-diamine; U.S. Pat. No. 8,348,838 to Singh et al., including polycyclic heteroaryl substituted triazoles such as 1-(6,7-dihydro-5H-benzo[6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine; 1-(6,7-dihydro-5H-benzo[6,7]cyclohepta[1,2-c]pyridazin-3-yl)-N³-((7S)-7-((2-methylpropyl)amino)-6,7,8,9-tetrahydro-5H-benzo[7]annulene-2-yl)-1H-1,2,4-triazole-3,5-diamine; 1-(6,7-dihydro-5H-benzo[6,7]cyclohepta[1,2-c]pyridazin-3-yl)-N³-((7S)-7-((propyl)amino)-6,7,8,9-tetrahydro-5H-benzo[7]annulene-2-yl)-1H-1,2,4-triazole-3,5-diamine; 1-(6,7-dihydro-5H-benzo[6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine; 1-(6,7-dihydro-5H-benzo[6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine; 1-(6,7-dihydro-5H-benzo[6,7]cyclohepta[1,2-c]pyridazin-3-yl)-N³-((7S)-7-(2-propylamino)-6,7,8,9-tetrahydro-5H-benzo[7]annulene-2-yl)-1H-1,2,4-triazole-3,5-diamine; and 1-(6,7-dihydro-5H-benzo[6,7]cyclohepta[1,2-c]pyridazin-3-yl)-N³-((7S)-7-((3,3-dimethylbut-2-yl)amino)-6,7,8,9-tetrahydro-5H-benzo[7]annulene-2-yl)-1H-1,2,4-triazole-3,5-diamine; U.S. Pat. No. 8,288,382 to Goff et al., including diaminothiazoles including 5-(quinoxalin-2-yl)-N²-(3,4,5-trimethoxyphenyl)thiazole-2,4-diamine; N²-(4-(2-(pyrrolidin-1-yl)ethoxy)phenyl)-5-(quinoxalin-2-yl)thiazole-2,4-diamine; N²-(4-(2-(pyrrolidin-1-yl)ethoxy)phenyl)-5-(quinazolin-4-yl)thiazole-2,4-diamine; 5-(quinazolin-4-yl)-N²-(3,4,5-trimethoxyphenyl)thiazole-2,4-diamine; 5-(isoquinolin-1-yl)-N²-(4-(2-(pyrrolidin-1-yl)ethoxy)phenyl)thiazole-2,4-diamine; 5-(benzo[d]thiazol-2-yl)-N²-(4-(2-(pyrrolidin-1-yl)ethoxy)phenyl)thiazole-2,4-diamine; 5-(6,7-dimethoxyquinazolin-4-yl)-N²-(4-(2-(pyrrolidin-1-yl)ethoxy)phenyl)thiazole-2,4-diamine; and N²-(3-chloro-4-(2-(pyrrolidin-1-yl)ethoxy)phenyl)-5-(6,7-dimethoxyquinazolin-4-yl)thiazole-2,4-diamine; U.S. Pat. No. 8,012,965 to Goff et al., including bridged bicyclic aryl and bridged bicyclic heteroaryl substituted triazoles such as 1-((6R,8R)-6,8-dimethylmethano-5,6,7,8-tetrahydroquinoline-2-yl)-N³-(3-fluoro-4-(4-(pyrrolidin-1-yl)piperidin-1-yl)phenyl)-1H-1,2,4-triazole-3,5-diamine; 1-(7,7-dimethyl-(6R,8R)6,8-methano-5,6,7,8-tetrahydroquinoline-2-yl)-N³-(4-(4-methylpiperazin-1-yl)phenyl)-1H-1,2,4-triazole-3,5-diamine; 1-(7,7-dimethyl-(6R,8R)6,8-methano-5,6,7,8-tetrahydroquinoline-2-yl)-N³-(3-fluoro-4-(4-bicyclo[2.2.1]heptan-2-yl piperazin-1-yl)phenyl)-1H-1,2,4-triazole-3,5-diamine; 1-(5,8-methano-4-phenyl-5,6,7,8-tetrahydroquinoline-2-yl)-N³-(3-fluoro-4-(4-(pyrrolidin-1-yl)piperidin-1-yl)phenyl)-1H-1,2,4-triazole-3,5-diamine; 1-(5,8-methano-4-phenyl-5,6,7,8-tetrahydroquinoline-2-yl)-N³-(3-fluoro-4-(4-cyclohexyl piperazin-1-yl)phenyl)-1H-1,2,4-thiazole-3,5-diamine; 1-(5,8-methano-4-phenyl-5,6,7,8-tetrahydroquinoline-2-yl)-N³-(4-(4-methylpiperazin-1-yl)phenyl)-1H-1,2,4-triazole-3,5-diamine; 1-(5,8-methano-4-phenyl-5,6,7,8-tetrahydroquinoline-2-yl)-N³-(3-methyl-4-(4-(4-methylpiperazin-1-yl)piperidin-1 yl)phenyl)-1H-1,2,4-thiazole-3,5-diamine; 1-(5,8-methano-4-thiophen-2-yl-5,6,7,8-tetrahydroquinoline-2-yl)-N³-(3-fluoro-4-(4-(4-methylpiperazin-1-yl)piperidin-1 yl)phenyl)-1H-1,2,4-thiazole-3,5-diamine; 1-(5,8-methano-4-thiophen-2-yl-5,6,7,8-tetrahydroquinoline-2-yl)-N.sup.3-(3-fluoro-4-(4-(pyrrolidin-1-yl)piperidin-1-yl)phenyl)-1H-1,2,4-triazole-3,5-diamine; and 1-(5,8-methano-4-thiophen-2-yl-5,6,7,8-tetrahydroquinoline-2-yl)-N³-(3-fluoro-4-(4-dimethylaminopiperidin-1-yl)phenyl)-1H-1,2,4-triazole-3,5-diamine; U.S. Pat. No. 7,879,856 to Goff et al., including diaminothiazoles such as 5-(quinoxalin-2-yl)-N.sup.2-(3,4,5-trimethoxyphenyl)thiazole-2,4-diamine; N²-(4-(2-(pyrrolidin-1-yl)ethoxy)phenyl)-5-(quinoxalin-2-yl)thiazole-2,4-diamine; N²-(4-(2-(pyrrolidin-1-yl)ethoxy)phenyl)-5-(quinolin-4-yl)thiazole-2,4-diamine; 5-(quinazolin-4-yl)-N²-(3,4,5-trimethoxyphenyl)thiazole-2,4-diamine; 5-(isoquinolin-1-yl)-N²-(4-(2-(pyrrolidin-1-yl)ethoxy)phenyl)thiazole-2,4-diamine; 5-(benzo[d]thiazol-2-yl)-N²-(4-(2-(pyrrolidin-1-yl)ethoxy)phenyl)thiazole-2,4-diamine; 5-(6,7-dimethoxyquinazolin-4-yl)-N²-(4-(2-(pyrrolidin-1-yl)ethoxy)phenyl)thiazole-2,4-diamine; and N²-(3-chloro-4-(2-(pyrrolidin-1-yl)ethoxy)phenyl)-5-(6,7-dimethoxyquinazolin-4-yl)thiazole-2,4-diamine; U.S. Pat. No. 7,872,000 to Goff et al., including bicyclic aryl and bicyclic heteroaryl substituted triazoles such as N³-(3-cyclopentyl-1,2,3,4,5,6-hexahydrobenzo[d]azocin-8-yl)-1-(6-fluoroquinazolin-4-yl)-1H-1,2,4-thiazole-3,5-diamine; 1-(benzo[d]thiazol-2-yl)-N³-(3-cyclopentyl-1,2,3,4,5,6-hexahydrobenzo[d]azocin-8-yl)-1H-1,2,4-triazole-3,5-diamine; 1-(benzo[d]thiazol-2-yl)-N³-(3-cyclopentyl-1,2,3,4,5,6-hexahydrobenzo[d]azocin-9-yl)-1H-1,2,4-triazole-3,5-diamine; 1-(2-chloro-7-methylthieno[3,2-d]pyrimidin-4-yl)-N′-(2,3,4,5-tetrahydrobe-nzo[b][1,4]dioxocin-8-yl)-1H-1,2,4-thiazole-3,5-diamine; N³-(3-cyclopentyl-1,2,3,4,5,6-hexahydro-benzo[d]azocin-8-yl)-1-(6,7-dimethoxyquinazolin-4-yl)-1H-[1,2,4]triazole-3,5-diamine; 1-(2-chloro-7-methylthieno[3,2-d]pyrimidin-4-yl)-N³-(3-cyclopentyl-1,2,3,4,5,6-hexahydrobenzo[d]azocin-8-yl)-1H-1,2,4-triazole-3,5-diamine; N³-(3-(bicyclo[2.2.1]heptan-2-yl)-1,2,3,4,5,6-hexahydrobenzo[d]azocin-8-yl)-1-(2-chloro-7-methylthieno[3,2-d]pyrimidin-4-yl)-1H-1,2,4-triazole-3,5-diamine; N³-(3-(bicyclo[2.2.1]heptan-2-yl)-1,2,3,4,5,6-hexahydrobenzo[d]azocin-8-yl)-1-(6,7-dimethoxyquinazolin-4-yl)-1H-1,2,4-triazole-3,5-diamine; N³-(3-cyclopentyl-1,2,3,4,5,6-hexahydrobenzo[d]azocin-8-yl)-1-(7-methylthieno[3,2-d]pyrimidin-4-yl)-1H-1,2,4-triazole-3,5-diamine; N³-(3-(bicyclo[2.2.1]heptan-2-yl)-1,2,3,4,5,6-hexahydrobenzo[d]azocin-8-yl)-1-(7-methylthieno[3,2-d]pyrimidin-4-yl)-1H-1,2,4-triazole-3,5-diam-ine; N.sup.3-(1-oxo-1,2,3,4,5,6-hexahydrobenzo[c]azocin-9-yl)-1-(2-chloro-7-methylthieno[3,2-d]pyrimidin-4-yl)-1H-1,2,4-triazole-3,5-diamine; N³-(1-oxo-1,2,3,4,5,6-hexahydrobenzo[c]azocin-9-yl)-1-(7-methylthieno[3,2-d]pyrimidin-4-yl)-1H-1,2,4-triazole-3,5-diamine; and N³-(1-oxo-1,2,3,4,5,6-hexahydrobenzo[c]azocin-9-yl)-1-(6,7-dimethoxyquinazolin-4-yl)-1H-1,2,4-triazole-3,5-diamine; and U.S. Pat. No. 7,709,482 to Goff et al., including polycyclic heteroaryl substituted triazoles such as 1-(6,7-dimethoxy-quinazolin-4-yl)-N³-(5,7,8,9-tetrahydrospiro[cyclohepta[b]pyridine-6,2′-[1,3]dioxolane]-3-yl)-1H-1,2,4-triazole-3,5-diamine; 1-(2-chloro-7-methylthieno[3,2-d]pyrimidin-4-yl)-N³-(5,7,8,9-tetrahydrospiro[cyclohepta[b]pyridine-6,2′-[1,3]dioxolane]-3-yl)-1H-1,2,4-triazole-3,5-diamine; 1-(2-chloro-7-methylthieno[3,2-d]pyrimidin-4-yl)-N³-(5,6,8,9-tetrahydrospiro[cyclohepta[b]pyridine-7,2′-[1,3]dioxolane]-3-yl)-1H-1,2,4-triazole-3,5-diamine; and 1-(2-chloro-7-methylthieno[3,2-d]pyrimidin-4-yl)-N³-(5′,5′-dimethyl-6,8,9,10-tetrahydro-5H-spiro[cycloocta[b]pyridine-7,2′-[1,3]dioxane]-3-yl)-1H-1,2,4-triazole-3,5-diamine, all of which patents are incorporated herein by this reference.

U.S. Pat. No. 8,809,299 by Bhatia et al., incorporated herein by this reference, discloses a method of suppression of proliferation of cancer stem cells comprising administration of a dopamine receptor antagonist such as thioridazine and a chemotherapeutic agent, such as a DNA synthesis inhibitor such as cytarabine, or a microtubule inhibitor such as paclitaxel or docetaxel.

U.S. Pat. No. 8,802,097 to Gurney et al., incorporated herein by this reference, discloses anti-RSPO1 antibodies that can suppress proliferation of cancer stem cells by modulating β-catenin activity and thus the Wnt pathway.

Inhibitors or modulators of the Hedgehog pathway are also useful for suppression of proliferation of cancer stem cells. Such inhibitors or modulators are disclosed in U.S. Pat. No. 8,785,635 to Austad et al., including cyclopamine analogs; U.S. Pat. No. 8,669,243 to Dahmane et al., including steroid-derived cyclopamine analogs; U.S. Pat. No. 8,575,141 to Dahmane et al., including steroid-derived cyclopamine analogs; U.S. Pat. No. 8,431,566 to Castro et al., including cyclopamine lactam analogs; U.S. Pat. No. 8,426,436 to Castro et al., including heterocyclic cyclopamine analogs; U.S. Pat. No. 8,293,760 to Castro et al., including cyclopamine lactam analogs; U.S. Pat. No. 8,236,956 to Adams et al., including cyclopamine analogs; U.S. Pat. No. 8,017,648 to Castro et al., including cyclopamine analogs; and U.S. Pat. No. 7,994,191 to Castro et al., including heterocyclic cyclopamine analogs, all of which patents are incorporated herein by this reference. Additional Hedgehog pathway inhibitors are disclosed in U.S. Pat. No. 5,807,491 to Cheng et al., incorporated herein by this reference, such as 4-(5-{[4-chloro-3-(5-phenyl-1H-imidazol-2-yl)phenyl]amino)-1,2,3,4-tetrahydroisoquinolin-2-yl)-1-1 (4}-thian-1-one; 1-(5-{[4-chloro-3-(5-phenyl-1H-imidazol-2-yl)phenyl]amino}-1,2,3,4-tetrahydroisoquinolin-2-yl)-3-hydroxy-2-(hydroxymethyl)-2-methylpropan-1-one; 4-(5-{[4-chloro-3-(5-phenyl-1H-imidazol-2-yl)phenyl]amino}-1,2,3,4-tetrahydroisoquinolin-2-yl)-thiane-1,1-dione; N-[4-chloro-3-(5-phenyl-1H-imidazol-2-yl)phenyl]-2-methanesulfonyl-1,2,3,4-tetrahydroisoquinolin-5-amine; N-[3-(1H-1,3-benzodiazol-2-yl)-4-methylphenyl]-2-methanesulfonyl-1,2,3,4-tetrahydroisoquinolin-5-amine; N-[4-chloro-3-(5-phenyl-1H-imidazol-2-yl)phenyl]-2-(1-ethylpiperidin-4-yl)-1,2,3,4-tetrahydroisoquinolin-5-amine; 1-(5-{[4-chloro-3-(5-phenyl-1H-imidazol-2-yl)phenyl]amino}-1,2,3,4-tetrahydroisoquinolin-2-yl)-2-methanesulfonylethan-1-one; (2R)-1-(5-{[4-chloro-3-(5-phenyl-1H-imidazol-2-yl)phenyl]amino}-1,2,3,4-tetrahydroisoquinolin-2-yl)-2-hydroxypropan-1-one; 1-(5-{[4-chloro-3-(5-phenyl-1H-imidazol-2-yl)phenyl]amino}-1,2,3,4-tetrahydroisoquinolin-2-yl)-2,3-dihydroxypropan-1-one; 1-(5-{[4-chloro-3-(5-phenyl-1H-imidazol-2-yl)phenyl]amino}-1,2,3,4-tetrahydroisoquinolin-2-yl)-2-hydroxypropan-1-one; 1-[4-(5-{[4-chloro-3-(5-phenyl-1H-imidazol-2-yl)phenyl]amino}-1,2,3,4-tetrahydroisoquinolin-2-yl)piperidin-1-yl]ethan-1-one; 4-(5-{[4-chloro-3-(5-phenyl-1H-imidazol-2-yl)phenyl]amino}-1,2,3,4-tetrahydroisoquinolin-2-yl)-thian-1-one; 5-{[4-chloro-3-(5-phenyl-1H-imidazol-2-yl)phenyl]amino}-1,2,3,4-tetrahydroisoquinoline-2-sulfonamide; 1-(5-{[4-chloro-3-(5-phenyl-1H-imidazol-2-yl)phenyl]amino}-1,2,3,4-tetrahydroisoquinolin-2-yl)-3-hydroxy-2,2-dimethylpropan-1-one; 2-methanesulfonyl-N-[3-(5-methoxy-1H-1,3-benzodiazol-2-yl)-4-methylphenyl]-1,2,3,4-tetrahydroisoquinolin-5-amine; N-{4-chloro-3-[6-(dimethylamino)-1H-1,3-benzodiazol-2-yl]phenyl}-2-methanesulfonyl-1,2,3,4-tetrahydroisoquinolin-5-amine; 2-[(5-{[4-chloro-3-(5-phenyl-1H-imidazol-2-yl)phenyl]amino}-1,2,3,4-tetrahydroisoquinoline-2-sulfonyl)amino]ethan-1-ol; (2R)-3-(5-{[4-chloro-3-(5-phenyl-1H-imidazol-2-yl)phenyl]amino}-1,2,3,4-tetrahydroisoquinolin-2-yl)propane-1,2-diol; and 1-(5-{[4-chloro-3-(5-phenyl-1H-imidazol-2-yl)phenyl]amino}-1,2,3,4-tetrahydroisoquinolin-2-yl)-2-methanesulfinylethan-1-one. Additional Hedgehog pathway inhibitors are also disclosed in U.S. Pat. No. 8,507,471 to Dierks et al., incorporated herein by this reference, including biphenylcarboxamide derivatives such as N-(6-((2R,6S)-2,6-dimethylmorpholino)pyridin-3-yl)-2-methyl-4′-(trifluoromethoxy)biphenyl-3-carboxamide. The transmembrane protein Smoothened (Smo) acts as a positive regulator of Hedgehog signaling, and thus inhibitors of Smo also act to inhibit signaling by the Hedgehog pathway. Inhibitors of Smo are disclosed in U.S. Pat. No. 8,481,542 to He et al., including pyridazinyl derivatives such as 2-[(R)-4-(4,5-dimethyl-6-phenoxy-pyridazin-3-yl)-2-methyl-3,4,5,6-tetra-hydro-2H-[1,2′]bipyrazinyl-5′-yl]-propan-2-ol; 2-[(R)-4-(6-(hydroxyl-phenyl-methyl)-4,5-dimethyl-pyridazin-3-yl)-2-methyl-3,4,5,6-tetrahydro-2H-[1,2]bipyrazinyl-5′-yl]-propan-2-ol; 2-[(R)-4-(4,5-dimethyl-6-pyridin-4-ylmethyl-pyridazin-3-yl)-2-methyl-3,4,5,6-tetrahydro-2H-[1,2′]bipyrazinyl-5′-yl]-propan-2-ol; 2-[(R)-4-(4,5-dimethyl-6-pyridin-2-ylmethyl-pyridazin-3-yl)-2-methyl-3,4,5,6-tetrahydro-2H-[1,2′]bipyrazinyl-5′-yl]-propan-2-ol; 2-[(R)-4-(6-benzyl-4,5-dimethyl-pyridazin-3-yl)-2-methyl-3,4,5,6-tetrahydro-2H-[1,2′]bipyrazinyl-5′-yl]-propan-2-ol; 2-[4-(6-benzyl-4,5-dimethyl-pyridazin-3-yl)-3,4,5,6-tetrahydro-2H-[1,2′]bipyrazinyl-5′-yl]-propan-2-ol; 2-[(S)-4-(6-benzyl-4,5-dimethyl-pyridazin-3-yl)-2-methyl-3,4,5,6-tetrahydro-2H-[1,2′]bipyrazinyl-5′-yl]-propan-2-ol; 2-[(R)-4-6-benzyl-4,5-dimethyl-pyridazin-3-yl)-2-ethyl-3,4,5,6-tetrahydro-2H-[1,2′]bipyrazinyl-5′-yl]-propan-2-ol; 1-[(R)-4-(6-benzyl-4,5-dimethyl-pyridazin-3-yl)-2-methyl-3,4,5,6-tetrahydro-2H-[1,2′]bipyrazinyl-5′-yl]-ethanone; and 2-[(R)-4-(6-benzyl-4,5-dimethyl-pyridazin-3-yl)-2-methyl-3,4,5,6-tetrahydro-2H-[1,2′]bipyrazinyl-5′-yl]-propane-1,2-diol. United States Patent Application Publication No. 2013/0261299 by He et al., incorporated herein by this reference, discloses including pyridazinyl derivatives as Smo inhibitors, such as (R)-4-(4,5-dimethyl-6-phenoxy-pyridazin-3-yl)-2-methyl-3,4,5,6-tetrahydro-2H-[1,2′]bipyrazinyl-5′-carboxylic methyl ester; (R)-4-(4,5-dimethyl-6-phenylamino-pyridazin-3-yl)-2-methyl-3,4,5,6-tetrahydro-2H-[1,2′]bipyrazinyl-5′-carboxylic acid methyl ester; (R)-4-(4,5-dimethyl-6-phenylamino-pyridazin-3-yl)-2-methyl-3,4,5,6-tetrahydro-2H-[1,2′]bipyrazinyl-5′-carboxylic acid phenylamide; 2-[(R)-4-(4,5-dimethyl-6-phenylamino-pyridazin-3-yl)-2-methyl-3,4,5,6-tetrahydro-2H-[1,2′]bipyrazinyl-5′-yl]-propan-2-01; (R)-4-[6-(4-fluoro-phenyl)-4,5-dimethyl-pyridazin-3-yl]-2-methyl-3,4,5,6 tetrahydro-2H-[1,2′]bipyrazinyl-5′-carboxylic acid methyl ester; (R)-4-[6-(4-trifluoromethyl-phenyl)-4,5-dimethyl-pyridazin-3-yl]-2-methyl-3,4,5,6-tetrahydro-2H-[1,2′]bipyrazinyl-5′-carboxylic acid methyl ester, (R)-4-[6-(4-trifluoromethyl-phenyl)-4,5-dimethyl-pyridazin-3-yl]-2-methyl-3,4,5,6-tetrahydro-2H-[1,2′]bipyrazinyl-5′-carboxylic acid; (R)-4-[6-(4-fluoro-phenyl)-4,5-dimethyl-pyridazin-3-yl]-2-methyl-3,4,5,6-tetrahydro-2H-[1,2′]bipyrazinyl-5′-carboxylic acid; methyl 5-(4-(6-benzyl-4,5-dimethylpyridazin-3-yl)piperidin-1-yl)pyrazine-2-carboxylate; 2-{5-[4-(6-benzyl-4,5-dimethyl-pyridazin-3-yl)-piperidin-1-yl]-pyrazin-2-yl}-propan-2-ol; 3-benzyl-6-{1-[5-(1-methoxy-1-methyl-ethyl)-pyrazin-2-yl]-piperidin-4-yl}-4,5-dimethyl-pyridazine; 3-benzyl-6-{1-[5-(trifluoromethyl)pyridin-2-yl]-piperidin-4-yl}-4,5-dimethyl-pyridazine; (R)-4-(6-benzoyl-4,5-dimethyl-pyridazin-3-yl)-2-methyl-3,4,5,6-tetrahydro-2H-[1,2′]bipyrazinyl-5′-carboxylic acid methyl ester; (6-{(R)-4-[4-(1-Hydroxy-1-methyl-ethyl)-phenyl]-3-methyl-piperazin-1-yl}-4,5-dimethyl-pyridazin-3-yl)-phenyl-methanone; (R)-4[6-(hydroxyl-phenyl-methyl)-4,5-dimethyl-pyridazin-3-yl]-2-methyl-3,4,5,6-tetrahydro-2H-[1,2′]bipyrazinyl-5′-carboxylic acid methyl ester; (R)-4-(4,5-dimethyl-6-pyridin-4-ylmethyl-pyridazin-3-yl)-2-methyl-3,4,5,6-tetrahydro-2H-[1,2′]bipyrazinyl-5′-carboxylic acid methyl ester; (R)-4-(4,5-dimethyl-6-pyridin-3-ylmethyl-pyridazin-3-yl)-2-methyl-3,4,5,6-tetrahydro-2H-[1,2′]bipyrazinyl-5′-carboxylic acid methyl ester; 2-[(R)-4-(4,5-dimethyl-6-pyridin-3-ylmethyl-pyridazin-3-yl)-2-methyl-3,4,5,6-tetrahydro-2H-[1,2′]bipyrazinyl-5′-yl]-propan-2-ol; (R)-4-(4,5-dimethyl-6-pyridin-2-ylmethyl-pyridazin-3-yl)-2-methyl-3,4,5,6-tetrahydro-2H-[1,2′]bipyrazinyl-5′-carboxylic acid methyl ester; 2-{(R)-4-[6-(difluoro-phenyl-methyl)-4,5-dimethyl-pyridazin-3-yl]-2-methyl-3,4,5,6-tetrahydro-2H-[1,2′]bipyrazinyl-5′-yl}-propan-2-ol; 3-benzyl-6-[4-(5-chloro-1H-imidazol-2-yl)piperidin-1-yl]-4,5-dimethyl-pyridazine; 1′-(6-benzyl-4,5-dimethyl-pyridazin-3-yl)-5-(1-hydroxy-1-methyl-ethyl)-2′,3′,5′,6′-tetrahydro-1′H-[2,4]bipyridinyl-4′-carbonitrile; 3-benzyl-4,5-dimethyl-6-[4-(4-trifluoromethyl-1H-imidazol-2-yl)-piperidin-1-yl]-pyridazine; 1′-(6-benzyl-4,5-dimethyl-pyridazin-3-yl)-5-(1-hydroxy-1-methyl-ethyl)-2′,3′,5′,6′-tetrahydro-1′H-[2,4]bipyridinyl-4′-ol; 2-[1′-(6-benzyl-4,5-dimethyl-pyridazin-3-yl)-4-fluoro-1′,2′,3′,4′,5′,6′-hexahydro-[2,4]bipyridinyl-5-yl]-propan-2-ol; 2-(6-{(S)-4-[4-(2-chloro-benzyl)-6,7-dihydro-5H-cyclopenta[d]pyridazin-1-yl]-3-methyl-piperazin-1-yl}-pyridin-3-yl)-propan-2-ol; (R)-4-(6-benzyl-4,5-dimethyl-pyridazin-3-yl)-2-methyl-3,4,5,6-tetrahydro-2H-[1,2′]bipyrazinyl-5′-carboxylic acid methyl ester; 3-benzyl-4,5-dimethyl-6-[(R)-3-methyl-4-(4-trifluoro-methanesulfonylpheny-l)-piperazin-1-yl]-pyridazine; and 2-[(R)-4-(6-benzyl-4,5-dimethyl-pyridazin-3-yl)-2-methyl-3,4,5,6-tetrahyd-ro-2H-[1,2′]bipyrazinyl-5′-yl]-2,2-dimethoxy-ethanol.

U.S. Pat. No. 8,779,151 to Priebe et al., incorporated herein by this reference, discloses caffeic acid analogs and derivatives that can suppress proliferation of cancer stem cells.

U.S. Pat. No. 8,779,001 to Tweardy et al., incorporated herein by this reference, discloses Stat3 inhibitors that can suppress proliferation of cancer stem cells, such as 4-[3-(2,3-dihydro-1,4-benzodioxin-6-yl)-3-oxo-1-propen-1-yl]benzoic acid; 4{5-[(3-ethyl-4-oxo-2-thioxo-1,3-thiazolidin-5-ylidene)methyl]-2-furyl}benzoic acid; 4-[({3-[(carboxymethyl)thio]-4-hydroxy-1-naphthyl}amino)sulfonyl]benzoic acid; 3-({2-chloro-4-[(1,3-dioxo-1,3-dihydro-2H-inden-2-ylidene)methyl]-6-ethoxyphenoxy}methyl)benzoic acid; methyl 4-({[3-(2-methyoxy-2-oxoethyl)-4,8-dimethyl-2-oxo-2H-chromen-7-yl]oxy}methyl)benzoate; and 4-chloro-3-{5-[(1,3-diethyl-4,6-dioxo-2-thioxotetrahydro-5(2H)-pyrimidinylidene)methyl]-2-furyl}benzoic acid. Other inhibitors of Stat3 are disclosed in U.S. Pat. No. 8,445,517 to Frank, incorporated herein by this reference, including pyrimethamine, pimozide, guanabenz acetate, alprenolol hydrochloride, nifuroxazide, solanine alpha, fluoxetine hydrochloride, ifosfamide, pyrvinium pamoate, moricizine hydrochloride, 3-(1,3-benzodioxol-5-yl)-1,6-dimethyl-pyrimido[5,4-e]-1,2,4-triazine-5,7(1H,6H)-dione and 3-(2-hydroxyphenyl)-3-phenyl-N,N-dipropylpropanamide.

Antibodies that bind GRP94 can also be used to suppress cancer stem cell proliferation. Such antibodies are disclosed in U.S. Pat. No. 8,771,687 to Ferrone et al., incorporated herein by this reference, and can be used together with a BRAF inhibitor such as vemurafenib or PLX4720 (N-(3-(5-chloro-1H-pyrrolo[2,3-b]pyridine-3-carbonyl)-2,4-difluorophenyl)propane-1-sulfonamide).

Frizzled receptor polypeptides can also be used to suppress cancer stem cell proliferation. Such Frizzled receptor polypeptides can comprise a soluble receptor that comprises a Fri domain of a FZD receptor that binds a ligand of a human FZD receptor and is capable of inhibiting tumor growth, and are disclosed in U.S. Pat. No. 8,765,913 to Gurney et al., incorporated herein by this reference. Similarly, anti-frizzled receptor antibodies can be used to suppress cancer stem cell proliferation, and are disclosed in U.S. Pat. No. 8,507,442 to Gurney et al., incorporated herein by this reference.

Immunoconjugates with cleavable linkages capable of targeting a stem cell antigen are disclosed in U.S. Pat. No. 8,759,496 to Govindan et al., U.S. Pat. No. 8,741,300 to Govindan et al., U.S. Pat. No. 7,999,083 to Govindan et al., United States Patent Application Publication No. 2014/0286860 by Govindan et al., all of which are incorporated herein by this reference.

The use of human prolactin, growth hormone, or placental lactogen for sensitizing cancer stem cells to chemotherapeutic agents is disclosed in U.S. Pat. No. 8,759,289 to Chen et al., incorporated herein by this reference.

The use of anti-prominin-1 antibody having ADCC activity or CDC activity to suppress cancer stem cell proliferation is disclosed in U.S. Pat. No. 8,722,858 to Yoshida, incorporated herein by this reference.

The use of antibodies specifically binding N-cadherin to suppress cancer stem cell proliferation is disclosed in U.S. Pat. No. 8,703,920 to Reiter et al., incorporated herein by this reference. The antibodies can be fully human antibodies.

The use of DR5 agonists to suppress cancer stem cell proliferation is disclosed in U.S. Pat. No. 8,703,712 to Buchsbaum et al., incorporated herein by this reference. The DR5 agonist can be a DR5 antibody.

The use of anti-DLL4 antibodies or binding fragments thereof to suppress cancer stem cell proliferation is disclosed in U.S. Pat. No. 8,685,401 to Harris et al., incorporated herein by this reference. The antibodies or binding fragments can be used together with radiation. DLL4 is a Notch ligand. The use of anti-DLL4 antibodies is also disclosed in U.S. Pat. No. 8,663,636 to Foltz et al., incorporated herein by this reference; the antibodies include fully human antibodies. The use of anti-DLL4 antibodies is also disclosed in U.S. Pat. No. 8,192,738 to Bedian et al., incorporated herein by this reference; the antibodies can include fully human antibodies.

The use of antibodies specifically binding GPR49 to suppress cancer stem cell proliferation is disclosed in U.S. Pat. No. 8,680,243 to Funahashi et al., incorporated herein by this reference. GPR49 is a member of the LGR family and is a hormone receptor. Anti-GPR49 antibodies are also disclosed in United States Patent Application Publication No. 2014/0302054 by Reyes et al. and in United States Patent Application Publication No. 2014/0256041 by Reyes et al., both incorporated herein by this reference. These antibodies can be monoclonal, humanized, or fully human antibodies.

U.S. Pat. No. 8,652,843 to Gurney et al., incorporated herein by this reference, discloses DDR1 binding agents, including antibodies, that can be used to suppress cancer stem cell proliferation. The antibodies bind to an extracellular domain of DDR1 and modulate DDR1 activity.

U.S. Pat. No. 8,628,774 to Gurney et al., incorporated herein by this reference, discloses LGR5 binding agents, including antibodies, that can be used to suppress cancer stem cell proliferation.

U.S. Pat. No. 8,609,736 to Gazit et al., incorporated herein by this reference, discloses the use of telomerase-activating compounds of Formula (IX)

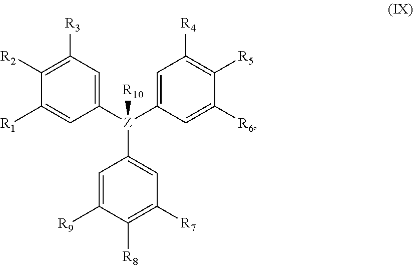

wherein Z is carbon, nitrogen, phosphorus, arsenic, silicon or germanium; R₁ to R₉ are the same or different, H, D, OH, halogen, nitro, CN, nitrileamido, amidosulfide, amino, aldehyde, substituted ketone, —COOH, ester, trifluoromethyl, amide, substituted or unsubstituted alkyl, alkenyl, alkynyl, aryl, arylalkyl, alkylaryl, arylsulfonyl, arylalkylenesulfonyl, alkoxy, alkylalkoxy, haloalkyl, alkylhaloalkyl, haloaryl, aryloxy, amino, monoalkylamino, dialkylamino, alkylamido, arylamino, arylamido, alkylthio, arylthio, heterocycloalkyl, alkylheterocycloalkyl, heterocycloalkylalkyl, heteroaryl, hetroarylalkyl, alkylheteroaryl; or R₃, R₄, or R₇ forms a fused cycloalkyl, heterocycloalkyl, aromatic or heteroaromatic ring with the main aromatic ring; and R₁₀ is absent, H, D, OH, halogen, oxo, nitro, CN, nitrileamido, amidosulfide, amino, aldehyde, substituted ketone, —COOH, ester, trifluoromethyl, amide, substituted or unsubstituted alkyl, alkenyl, alkynyl, aryl, arylalkyl, alkylaryl, arylsulfonyl, arylalkylenesulfonyl, alkoxy, haloalkyl, haloaryl, cycloalkyl, alkylcycloalkyl, aryloxy, monoalkylamino, dialkylamino, alkylamido, arylamino, arylamido, alkylthio, arylthio, heterocycloalkyl, alkylheterocycloalkyl, heterocycloalkylalkyl, heteroaryl, hetroarylalkyl, alkylheteroaryl; or its isomer, pharmaceutically acceptable salt, pharmaceutical product, hydrate, N-oxide, crystal or any combination thereof.

U.S. Pat. No. 8,591,892 to Alinari et al., incorporated herein by this reference, discloses methods for suppression of proliferation of cancer stem cells by administration of fingolimod and anti-CD74 antibodies or fragments thereof. The use of anti-CD74 antibodies to suppress cancer stem cell proliferation is also disclosed in U.S. Pat. No. 8,367,037 to Byrd et al. and in U.S. Pat. No. 8,119,101 to Byrd et al., both incorporated herein by this reference.

U.S. Pat. No. 8,562,997 to Jaiswal et al., incorporated herein by this reference, discloses methods for suppression of proliferation of cancer stem cells by administration of an antibody that prevents the binding of CD47 to SIPRα or administration of a CD47 mimetic.

U.S. Pat. No. 8,557,807 to Morales et al., incorporated herein by this reference, discloses thienopyranone kinase inhibitors for inhibition of PI-3 kinases that can be used to suppress cancer stem cell proliferation. In general, the kinase inhibitors are compounds of Formula (X)

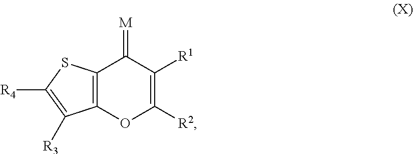

wherein:

(1) M is O or S;

(2) R¹ is selected from H, F, Cl, Br, I, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O-substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;

(3) R² is selected from the group consisting of Subformulas (X(a)) and (X(b))

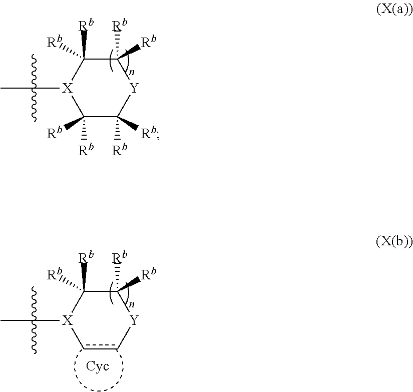

wherein:

(4) X is N;

(5) n is 1;

(6) Y is O;

(7) R^{b} is hydrogen or independently at each instance any group selected from F, Cl, Br, I, alkyl, alkenyl, alkynyl, carbocycle, aryl, heterocycle, heteroaryl, formyl, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O-substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;

(8) R₃ is selected from H, F, Cl, Br, I, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O-substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;

(9) R₄ is selected from the group consisting of from H, F, Cl, Br, I, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O-substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate; and

(10) Cyc is selected from the group consisting of aryl, substituted aryl, heterocycle, substituted heterocycle, carbocycle, and substituted carbocycle.

U.S. Pat. No. 8,530,429 to Robbins et al., incorporated herein by this reference, discloses a method for suppression of cancer stem cell proliferation, particularly for glioblastoma multiforme, comprising administration of peptides that bind to cancer stem cells. The peptides are between 12 and 20 amino acids, and are conjugated to an anti-tumor agent. The peptides can be comprised of L-amino acids, D-amino acids, a mixture of L- and D-amino acids, or a retro-inverso peptide formed of D-amino acids arranged in reverse order.

U.S. Pat. No. 8,470,307 to Frankel, incorporated herein by this reference, discloses the use of a diphtheria toxin-interleukin 3 conjugate to suppress cancer stem cell proliferation. Preferably, the conjugate is a fusion protein comprising amino acids 1-388 of diphtheria toxin fused via a peptide linker to full-length, human interleukin-3.

U.S. Pat. No. 8,455,688 to Kovach et al., incorporated herein by this reference, discloses inhibitors of histone deacetylase (HDAC) useful for suppression of cancer stem cell proliferation, including compounds of Formula (XI)

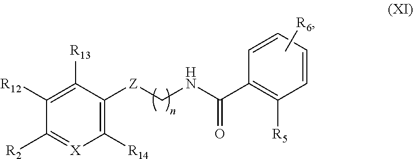

wherein:

(1) n is 1-10;

(2) X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;

(3) R₂ is H or NR₃R₄, wherein R₃ and R₄ are each independently H or C₂-C₆ alkyl;

(4) R₅ is SH; and

(5) R₆, R₁₂, R₁₃, and R₁₄ are each independently H, OH, SH, F, Cl, SO₂R₁₅, NO₂, trifluoromethyl, methoxy, or CO—R₁₅, wherein R₁₅ is alkyl, alkenyl, alkynyl, C₃-C₈ cycloalkyl, or aryl, or a salt of the compound of Formula (XI).

U.S. Pat. No. 8,435,972 to Stein et al., incorporated herein by this reference, discloses the use of progesterone and analogs and derivatives thereof to suppress cancer stem cell proliferation, including pregnenolone, dehydroepiandrosterone, allopregnanolone tetrahydrodeoxycorticosterone, alphaxolone, alphadolone, hydroxydione, minaxolone, ganaxolone, and 3α-hydroxy-5α-pregnane-20-one, and their sulfates.

U.S. Pat. No. 8,404,239 to Siebel et al., incorporated herein by this reference, discloses antibodies that bind the negative regulatory region (NRR) of Notch2. The antibodies can be monoclonal antibodies. The antibodies can be used to suppress cancer stem cell proliferation. Antibodies that bind other regions of Notch2, such as a non-ligand binding region, are disclosed in U.S. Pat. No. 8,206,713 to Lewicki et al., incorporated herein by this reference, and can be used to suppress cancer stem cell proliferation. The antibodies can be monoclonal antibodies, chimeric antibodies, humanized antibodies, or human antibodies. Still other antibodies that bind Notch2 are disclosed in United States Patent Application Publication No. 2014/0314782 by Christian et al., incorporated herein by this reference, and can be used to suppress cancer stem cell proliferation.

U.S. Pat. No. 8,383,806 to Rameshwar, incorporated herein by this reference, discloses a protein receptor, HGFIN, and inhibitors thereof, including siRNA specific for HGFIN. The inhibitors of HGFIN can be used to suppress cancer stem cell proliferation and can also be used to reverse carboplatin resistance.

U.S. Pat. No. 8,318,677 to Weinschenk et al., incorporated herein by this reference, discloses immunotherapeutic peptides that can be used to suppress cancer stem cell proliferation.

U.S. Pat. No. 8,299,106 to Li et al., incorporated herein by this reference, discloses thiazole-substituted indolin-2-ones that are inhibitors of CSCPK and related kinases, and that can be used to suppress cancer stem cell proliferation. Additional inhibitors of CSCPK and related kinases are disclosed in United States Patent Application Publication No. 2014/0275033 by Li et al., incorporated herein by this reference.

U.S. Pat. No. 8,293,743 to Kahn, incorporated herein by this reference, discloses substituted imidazo[1,2-a]pyrazine derivatives as α-helix mimetics that can be used to suppress cancer stem cell proliferation.

U.S. Pat. No. 8,273,550 by Cizeau et al., incorporated herein by this reference, discloses antibodies directed to an epitope of variant Heterogeneous Ribonucleoprotein G (HnRNPG), including monoclonal, chimeric, and humanized antibodies, that can be used to suppress cancer stem cell proliferation.

U.S. Pat. No. 8,216,570 to Mather et al. and U.S. Pat. No. 7,778,714 to Mather et al., both incorporated herein by this reference, discloses antibodies, including monoclonal antibodies, that bind TES7 antigen, and that can be used to suppress cancer stem cell proliferation.

U.S. Pat. No. 8,163,279 to Bergstein, incorporated herein by this reference, discloses antibodies binding to the ILR3α subunit that can be used to suppress cancer stem cell proliferation. The antibodies can be conjugated to a cytotoxic agent.

U.S. Pat. No. 8,058,243 to Tyers et al., incorporated herein by this reference, discloses the use of a compound selected from the group consisting of (±)butaclamol, R(−) propylnorapomorphine, apomorphine, cis-(Z) flupenthixol, hexahydro-sila-difenidol, ifenprodil tartrate, carbetapentane citrate, fenretinide, WHI-P131, SB 202190, p-aminophenethyl-m-trifluoromethylphenyl piperazine (PAPP), and dihydrocapsaicin to suppress cancer stem cell proliferation. A particularly preferred compound is ifenprodil tartrate.

U.S. Pat. No. 7,790,407 to Ma, incorporated herein by this reference, discloses antibodies specific for SALL4, including isoforms SALL4A, SALL4B, and SALL4C. SALL4 is a zinc finger transcription factor. The antibodies can be used to suppress cancer stem cell proliferation.

U.S. Pat. No. 7,754,206 to Clarke et al., incorporated herein by this reference, discloses antibodies specifically binding Notch4 that modulate the activity of a Notch4 ligand, such as Delta 1, Delta 2, Delta-like ligand 4 (D114), Jagged 1 or Jagged 2. The antibodies can be used to suppress cancer stem cell proliferation.

United States Patent Application Publication No. 2014/0314836 by Doxsey et al., incorporated herein by this reference, discloses a method of suppressing cancer stem cell proliferation by inducing degradation of a midbody derivative in cells by increasing the amount of Neighbor of BRCA1 (NBR1) in the cell or potentiating binding between NBR1 and Centrosomal Protein of 55 kDa (Cep55) in the cell. This can be done by employing a bispecific antibody that binds to both NBR1 and Cep55.

United States Patent Application Publication No. 2014/0309184 by Rocconi et al., incorporated herein by this reference, discloses a method for suppressing cancer stem cell proliferation by administration of a Smo inhibitor, such as N-[2-methyl-5-[(methylamino)methyl]phenyl]-4-[(4-phenyl-2-quinazolinyl)amino]-benzamide (BMS-833923), and a chemotherapeutic agent such as a platinum-based therapeutic agent.

United States Patent Application Publication No. 2014/0308294 by Seshire et al., incorporated herein by this reference, discloses peptides that block or inhibit the interleukin-1 receptor 1, and can be used to suppress proliferation of cancer stem cells.

United States Patent Application Publication No. 2014/0303354 by Masternak et al., incorporated herein by this reference, discloses antibodies specific for CD47 or CD19 that can be used to suppress proliferation of cancer stem cells. The antibodies can be bispecific.

United States Patent Application Publication No. 2014/0303106 by Zheng et al., incorporated herein by this reference, discloses histone methyltransferase inhibitors that can be used to suppress proliferation of cancer stem cells. The compounds include compounds of Formula (XII) and Formula (XIII)

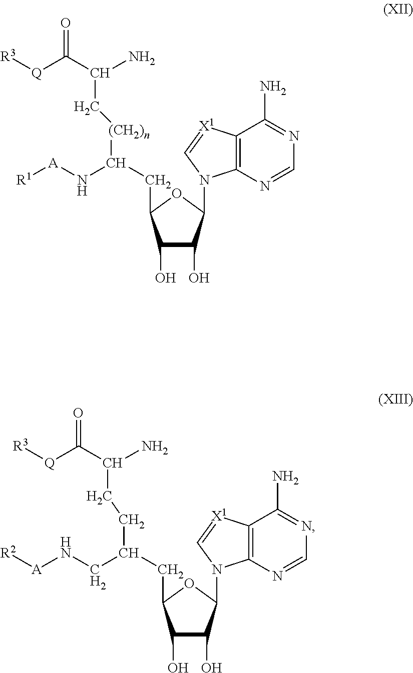

wherein:

(1) X¹ is N or CH;

(2) Q is NH or O;

(3) A is selected from the group consisting of a valence bond, (C₁-C₂₀) hydrocarbyl, (C₁-C₂₀) oxaalkyl, and (C₁-C₂₀) azaalkyl;

(4) R¹ is selected from the group consisting of hydrogen, —C(═NH)NH₂, —C(═NH)NH(C₁-C₁₀)hydrocarbyl, fluoro(C₁-C₆)hydrocarbyl, and —CH(NH₂)COOH, with the provisos that: (a) when A is a valence bond, R¹ cannot be H; and (b) when QR³ is OH, R¹ cannot be fluoro(C₁-C₆)hydrocarbyl;

(5) R² is selected from the group consisting of hydrogen, —C(═NH)NH₂, —C(═NH)NH(C₁-C₁₀)hydrocarbyl, fluoro(C₁-C₆)hydrocarbyl, and —CH(NH₂)COOH;

(6) R³ is selected from the group consisting of hydrogen and (C₁-C₂₀) hydrocarbyl; and

(7) n is 1 or 2.

United States Patent Application Publication No. 2014/0302511 by Yamazaki et al., incorporated herein by this reference, discloses antibodies to a stem cell surface marker Lg5, which can be used to suppress proliferation of cancer stem cells.

United States Patent Application Publication No. 2014/0302034 by Bankovich et al., incorporated herein by this reference, discloses antibodies that specifically bind to EFNA1; the antibodies can include multispecific antibodies and can be humanized. The antibodies can be used to suppress proliferation of cancer stem cells.

United States Patent Application Publication No. 2014/0294994 by Huang, incorporated herein by this reference, discloses antipsychotic phenothiazine derivatives for suppression of cancer stem cell proliferation. The derivatives can be, but are not limited to, trifluoperazine, chlorpromazine, thioridazine, perphenazine, triflupromazine, or promazine. The derivatives can be used with another antineoplastic agent such as gefitinib or cisplatin.

United States Patent Application Publication No. 2014/0294856 by Aboagye et al., incorporated herein by this reference, discloses methods for suppression of proliferation of cancer stem cells employing a HDAC6 inhibitor and an AKT inhibitor. Suitable HDAC6 inhibitors include tubacin, tubastatin A, and cyclic tetrapeptide hydroxamic acids. Suitable AKT inhibitors include BEZ-235, PI-103, API-2, LY294002, Wortmannin, AKT VIII, BKM120, BGT226, Everolimus, Choline kinase inhibitors, bcl-2 inhibitors, Hsp-90 inhibitors, multi-kinase inhibitors, mTOR kinase inhibitors, proteasome inhibitors, and TORC1/TORC2 inhibitors.

United States Patent Application Publication No. 2014/0286961 by Bergstein, incorporated herein by this reference, discloses a method of suppressing proliferation of cancer stem cells employing administration of a ligand that binds to a cancer-stem-line-specific cell surface antigen stem cell marker, wherein the antigen is selected from the group consisting of CD34, Scl/Tal-1, Flk-1/KDR, Tie-1, Tie-2, c-Kit, AC133, PU.1, ikaros, beta-1 alpha (2,3,5) integrin, cytokeratin 19, basonuclin, skin 1a-i/Epoc-1/Oct11, cytokeratin 14, LEF-1, SP-1, SP-2, EGF-R, MUC-1, c-Kit, SCF, Ag/s270.38, 374.3, 18.11, AFP, IGF-2, TGF-alpha/beta, GGT, Isl-1, FA-1, TRA-1-60, SSEA (1,3,4), BCL-2, Muc-1, ESA, HMWCk (5,14), pp32, CD44, notch, numb, nestin, and p75.

United States Patent Application Publication No. 2014/0286955 by Aifantis et al., incorporated herein by this reference, discloses methods for suppressing proliferation of cancer stem cells by administration of a Notch receptor agonist, such as a Notch1 receptor agonist and a Notch2 receptor agonist.

United States Patent Application Publication No. 2014/0286951 by Gurney et al., incorporated herein by this reference, discloses binding agents, including antibodies, that bind human MET. The antibodies can be bispecific, with a second binding site binding one or more components of the Wnt pathway; the second binding site can be a soluble human frizzled 8 (FZD8) FZD8 receptor. The binding agents can be used for suppression of proliferation of cancer stem cells.

United States Patent Application Publication No. 2014/0275201 by Mani et al., incorporated herein by this reference, disclose the use of a PDGFR-β inhibitor to suppress proliferation of cancer stem cells. The PDGFR-β inhibitor can be sunitinib, axitinib, BIBF1120, MK-2461, dovitinib, pazopanib, telatinib, CP 673451, or TSU-68.

United States Patent Application Publication No. 2014/0275092 by Albrecht et al., incorporated herein by this reference, discloses pyrazolo compounds that have histone demethylase activity and are useful for suppressing proliferation of cancer stem cells.

United States Patent Application Publication No. 2014/0275076 by Tsuboi et al., incorporated herein by this reference, discloses heterocyclic substituted 3-heteroaryidenyl-2-indolinone derivatives that can be used to suppress proliferation of cancer stem cells.

United States Patent Application Publication No. 2014/0255377 by Wong et al., incorporated herein by this reference, discloses albumin-binding arginine deiminase fusion proteins that can be used to suppress proliferation of cancer stem cells.

United States Patent Application Publication No. 2014/0220159 by Arora et al., incorporated herein by this reference, discloses hydrogen-bond surrogate peptides and peptidomimetics that reactivate p53 and that can be used for suppressing proliferation of cancer stem cells. United States Patent Application Publication No. 2014/0205655 by Arora et al., incorporated herein by this reference, similarly discloses oligooxopiperazines for reactivating p53, such as oligooxopiperazines that substantially mimic helix αB of the C-terminal transactivation domain of Hypoxia-Inducible Factor 1α and that can be used for suppressing proliferation of cancer stem cells.

United States Patent Application Publication No. 2014/0219956 by Govindan et al., incorporated herein by this reference, discloses prodrugs of 2-pyrrolinodoxorubicin conjugated to antibodies that can be used for suppressing proliferation of cancer stem cells.

United States Patent Application Publication No. 2014/0193358 by Merchant, incorporated herein by this reference, discloses a method for targeting cancer stem cells comprising administering to the subject a targeted cargo protein, wherein the targeted cargo protein comprises: (a) one or more cargo moieties; and (b) one or more targeting moieties that bind to a target displayed by a cancer stem cell, wherein the targeting moiety is derived from a natural ligand to the target. The cargo moiety can comprise a toxin, and the targeting moiety can comprise a pro-apoptosis member of the BCL-2 family selected from BAX, BAD, BAT, BAK, BIK, BOK, BID BIM, BMF and BOK.

United States Patent Application Publication No. 2014/0186872 by Feve et al., incorporated herein by this reference, discloses bisacodyl and analogs thereof as useful for suppression of proliferation of cancer stem cells.

United States Patent Application Publication No. 2014/0179660 by Kim et al., incorporated herein by this reference, discloses N¹-cyclic amine-N⁵-substituted phenyl biguanide derivatives useful for suppression of proliferation of cancer stem cells. The biguanide derivatives include N¹-piperidine-N⁵-(3-bromo)phenyl biguanide; N¹-piperidine-N⁵-phenyl biguanide; N¹-piperidine-N⁵-(3-methyl)phenyl biguanide; N¹-piperidine-N⁵-(3-ethyl)phenyl biguanide; N¹-piperidine-N⁵-(3-hydroxy)phenyl biguanide; N¹-piperidine-N⁵-(3-hydroxymethyl)phenyl biguanide; N¹-piperidine-N⁵-(3-methoxy)phenyl biguanide; N¹-piperidine-N⁵-(4-fluoro)phenyl biguanide; N¹-piperidine-N⁵-(2-fluoro)phenyl biguanide; N¹-piperidine-N⁵-(3-fluoro)phenyl biguanide; N¹-pyrrolidine-N⁵-(4-chloro)phenyl biguanide; N¹-piperidine-N⁵-(4-chloro)phenyl biguanide; N¹-pyrrolidine-N⁵-(3-chloro)phenyl biguanide; N¹-piperidine-N⁵-(3-chloro)phenyl biguanide; N¹-azepane-N⁵-(3-chloro)phenyl biguanide; N¹-morpholine-N5-(3-bromo)phenyl biguanide; N¹-pyrrolidine-N⁵-(3-trifluoromethyl)phenyl biguanide; N¹-piperidine-N⁵-(3-trifluoromethyl)phenyl biguanide; N¹-azetidine-N⁵-(4-trifluoromethyl)phenyl biguanide; N1-pyrrolidine-N5-(4-trifluoromethyl)phenyl biguanide; N¹-piperidine-N⁵-(4-trifluoromethyl)phenyl biguanide; N¹-pyrrolidine-N⁵-(3-trifluoromethoxy)phenyl biguanide; N¹-piperidine-N⁵-(3-trifluoromethoxy)phenyl biguanide; N¹-piperidine-N⁵-(3-difluoromethoxy)phenyl biguanide; N¹-azetidine-N⁵-(4-trifluoromethoxy)phenyl biguanide; N¹-pyrrolidine-N⁵-(4-trifluoromethoxy)phenyl biguanide; N¹-piperidine-N⁵-(4-trifluoromethoxy)phenyl biguanide; N¹-morpholine-N⁵-(4-trifluoromethoxy)phenyl biguanide; N¹-(4-methyl)piperazine-N⁵-(4-trifluoromethoxy)phenyl biguanide; N¹-piperidine-N⁵-(3-amino)phenyl biguanide; N¹-piperidine-N⁵-(4-dimethylamino)phenyl biguanide; N¹-piperidine-N⁵-(4-acetamide)phenyl biguanide; N¹-piperidine-N⁵-(3-acetamide)phenyl biguanide; N¹-piperidine-N⁵-(4-(1H-tetrazole-5-yl))phenyl biguanide; N¹-piperidine-N⁵-(3-methylsulfonamide)phenyl biguanide; N¹-piperidine-N⁵-(4-sulfonic acid)phenyl biguanide; N¹-piperidine-N⁵-(4-methylthio)phenyl biguanide; N¹-piperidine-N⁵-(4-sulfamoyl)phenyl biguanide; N¹-piperidine-N⁵-(3,5-dimethoxy)phenyl biguanide; N¹-piperidine-N⁵-(4-fluoro-3-trifluoromethyl)phenyl biguanide; N¹-piperidine-N⁵-(4-chloro-3-trifluoromethyl)phenyl biguanide; and N¹-pyrrolidine-N⁵-(3-fluoro-4-trifluoromethyl)phenyl biguanide.

United States Patent Application Publication No. 2014/0147423 by Kim et al., incorporated herein by this reference, discloses the use of fibulin-3 protein to induce the reduction of activity of Wnt/β-catenin, MMP2, and MMP7. The fibulin-3 protein can be used to suppress proliferation of cancer stem cells.

United States Patent Application Publication No. 2014/0142120 by Cardozo et al., incorporated herein by this reference, discloses modulators of SCFSkp2, a protein that is part of the ubiquitin proteasome system. The modulators are useful for suppression of cancer stem cell proliferation. The modulators include compounds of Formula (XIV) and Formula (XV)

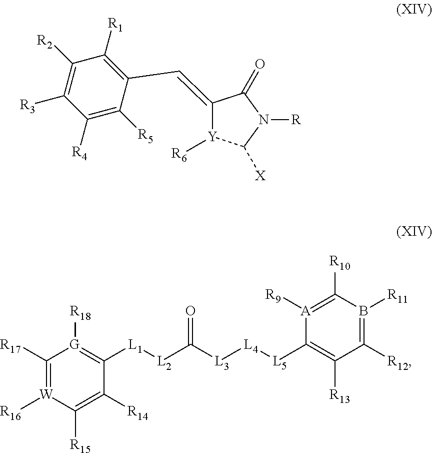

wherein, in Formula (XIV):

(1) [symbol] is a single or double bond;

(2) R is H, C₁-C₆ alkyl, C₂-C₆ alkenyl, C₂-C₆ alkynyl, R₇, CH₂R₇, CH₂C(O)R₇, or CH₂C(O)NHR₇;

(3) R₁ is H, OR₈, or OCH₂COOR₈;

(4) R₂ is H, OR₈, or OCH₂COOR₈;

(5) R₃ is H, C₁-C₆ alkyl, C₂-C₆ alkenyl, C₂-C₆ alkynyl, OCH₂COOR₈, or OS(O)₂R₇NHC(O)R₈; or R₂ and R₃ can combine to form —OCH₂O—;

(6) R₄ is H or halogen;

(7) R₅ is H or OR₈, or R₄ and R₅ can combine to form a 6-membered aryl ring;

(8) R₆ is optional, and, if present, is COOR₈;

(9) R₇ is a monocyclic or polycyclic aryl, or a monocyclic or polycyclic heterocyclyl or heteroaryl containing 1-5 heteroatoms selected from the group consisting of nitrogen, oxygen, and sulfur, each R₇ being optionally substituted from 1-3 times with substituents selected from the group consisting of halogen, COOR₈, C₁-C₆ alkyl, C₂-C₆ alkenyl, and C₂-C₆ alkynyl;

(10) R₈ is hydrogen, C₁-C₆ alkyl, C₂-C₆ alkenyl, or C₂-C₆ alkynyl;

(11) X is S, O, C₁-C₆ alkyl, C₂-C₆ alkenyl, or C₂-C₆ alkynyl; and

(12) Y is S or C.

In Formula (XV),

(1) A is O or C;

(2) B is C or absent;

(3) G is C or S;

(4) W is C or absent;

(5) L₁ is independently selected from the group consisting of: (a) absent; (b) —C(S)NH—, and (c) a moiety of Subformula (XV(a))

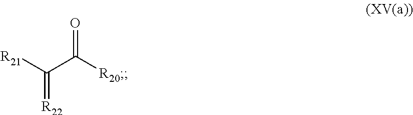

(6) L₂ is NH or O;

(7) L₃ is absent or —CH₂—;

(8) L₄ is absent or —R₂₄═N—N═CH—;

(9) L₅ is absent or —C(O)—;

(10) R₉ is H;

(11) R₁₀ is H, halogen, C₁-C₆ alkyl, C₂-C₆ alkenyl, or C₂-C₆ alkynyl;

(12) R₁₁ is H, halogen, NO₂, OCH₂COOR₂₃, OC(O)R₂₃, or OR₂₃;

(13) R₁₂ is H or OR₂₃;

(14) R₁₃ is H;

(15) R₁₄ is H, OR₂₃, C(O)NH₂, or COOR₂₃;

(16) R₁₅ is H, halogen, C₁-C₆ alkyl, C₂-C₆ alkenyl, C₂-C₆ alkynyl, or COOR₂₃;

(17) R₁₆ is H, halogen, C₁-C₆ alkyl, C₂-C₆ alkenyl, C₂-C₆ alkynyl, —CH═R₂₄, or COOR₂₃;

(18) R₁₇ is H, halogen, C₁-C₆ alkyl, C₂-C₆ alkenyl, C₂-C₆ alkynyl, or COOR₂₃;

(19) R₁₈ is H, halogen, C₁-C₆ alkyl, C₂-C₆ alkenyl, C₂-C₆ alkynyl, OR₂₃, or COOR₂₃;

(20) R₂₀ is —NH—, —NH—N—CH—, or NH₂;

(21) R₂₁ is —(CH2)ₙ—, where n is 0 to 6;

(22) R₂₂ is —CH— or —CHR₂₄;

(23) R₂₃ is H, C₁-C₆ alkyl, C₂-C₆ alkenyl, or C₂-C₆ alkynyl; and

(24) R₂₄ is a monocyclic or polycyclic aryl, or a monocyclic or polycyclic heterocyclyl or heteroaryl containing 1-5 heteroatoms selected from the group consisting of nitrogen, oxygen, and sulfur, each R₂₄ being optionally substituted from 1-3 times with substituents selected from the group consisting of OH, C₁-C₆ alkyl, C₂-C₆ alkenyl, C₂-C₆ alkynyl, ═O, ═NH, NH₂, halogen, and COOR₂₃.

United States Patent Application Publication No. 2014/0094466 by Haga et al., incorporated herein by this reference, discloses inhibitors of Slingshot-2 that can be used to suppress proliferation of cancer stem cells. The inhibitors include 3-[(4,5-dimethoxy-3-oxo-1H-isobenzofuran-1-yl)amino]-4-methylbenzoic acid; 2-ethoxy-5-(4-phenylpiperidine-1-sulfonyl)benzoic acid; and 3-[bis(2-methoxyethyl)sulfamoyl]benzoic acid.

United States Patent Application Publication No. 2014/0056972 by Houchen et al., incorporated herein by this reference, discloses monoclonal antibodies specifically binding DCLK1 protein. The monoclonal antibodies can be incorporated into drug conjugates, and are useful for suppression of proliferation of cancer stem cells.

United States Patent Application Publication No. 2014/0056890 by Gurney et al., incorporated herein by this reference, discloses antibodies and soluble receptors that modulate the Hippo pathway and that can be used for suppression of proliferation of cancer stem cells.

United States Patent Application Publication No. 2014/0038958 by Ronnison et al., incorporated herein by this reference, discloses selective inhibitors of CDK8 and CDK19 that can be used for suppression of proliferation of cancer stem cells. The selective inhibitors can be compounds of Formula (XVI) or (XVII)

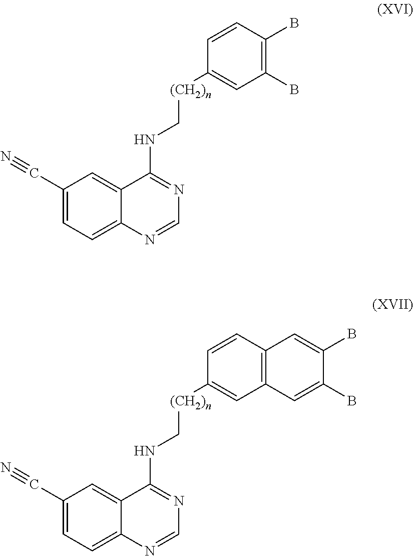

wherein:

(1) each B is independently hydrogen or a moiety of Subformula (XVI(a))

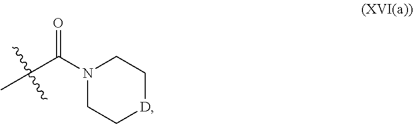

provided that at least one B is hydrogen and not more than one B is hydrogen; D is selected from —NH, —N-lower alkyl, or O; and n is 0-2.

United States Patent Application Publication No. 2014/0023650 by Bastid et al., incorporated herein by this reference, discloses antibodies and antibody fragments specifically binding IL-17 that can be used for suppressing proliferation of cancer stem cells.

United States Patent Application Publication No. 2014/0023589 by Watt et al., incorporated herein by this reference, discloses antibodies that specifically bind to FRMD4A and that can be used for suppressing proliferation of cancer stem cells.

United States Patent Application Publication No. 2014/0017259 by Aurisicchio et al., incorporated herein by this reference, discloses monoclonal antibodies that specifically bind the ErbB-3 receptor and that can be used for suppressing proliferation of cancer stem cells.

United States Patent Application Publication No. 2014/0017253 by Gurney et al., incorporated herein by this reference, discloses antibodies that specifically bind human RSPO3 and modulate β-catenin activity; the antibodies can be used for suppressing proliferation of cancer stem cells.

United States Patent Application Publication No. 2013/0345176 to Jiang et al., incorporated herein by this reference, discloses esters of 4,9-dihydroxy-naphtho[2,3-b]furans that are converted into 4,9-dihydroxy-naphtho[2,3-b]furans in vivo and that can be used for suppressing proliferation of cancer stem cells.

United States Patent Application Publication No. 2013/0303512 by Pestell, incorporated herein by this reference, discloses the use of CCR5 antagonists that can be used for suppressing proliferation of cancer stem cells. The CCR5 antagonists include 4,4-difluoro-N-[(1S)-3-[(1R,5S)-3-(3-methyl-5-propan-2-yl-1,2,4-triazol-4-yl)-8-azabicyclo[3.2.1]octan-8-yl]-1-phenylpropyl]cyclohexane-1-carboxamide; (4,6-dimethylpyrimidin-5-yl)-[4-[(3S)-4-[(1R)-2-methoxy-1-[4-(trifluoromethyl)phenyl]ethyl]-3-methylpiperazin-1-yl]-4-methylpiperidin-1-yl]methanone; 4,4-difluoro-N-[(1 S)-3-[(1R,5S)-3-(3-methyl-5-propan-2-yl-1,2,4-triazol-4-yl)-8-azabicyclo[3.2.1]octan-8-yl]-1-phenylpropyl]cyclohexane-1-carboxamide; N-(1S)-3-3-(3-isopropyl-5-methyl-4H-1,2,4-triazol-4-yl)-exo-8-azabicyclo[3.2.1]oct-8-yl-1-phenylpropylcyclobutanecarboxamide; N-(1S)-3-3-(3-isopropyl-5-methyl-4H-1,2,4-triazol-4-yl)-exo-8-azabicyclo[3.2.1]oct-8-yl-1-phenylpropylcyclopentanecarboxamide; N-(1S)-3-3-(3-isopropyl-5-methyl-4H-1,2,4-triazol-4-yl)-exo-8-azabicyclo[3.2.1]oct-8-yl-1-phenylpropyl-4,4,4-trifluorobutanamide; N-(1S)-3-3-(3-isopropyl-5-methyl-4H-1,2,4-triazol-4-yl)-exo-8-azabicyclo[3.2.1]oct-8-yl-1-phenylpropyl-4,4-difluorocyclohexanecarboxamide; and N-(1S)-3-3-(3-isopropyl-5-methyl-4H-1,2,4-triazol-4-yl)-exo-8-azabicyclo[3.2.1]oct-8-yl-1-(3-fluorophenyl)propyl-4,4-difluorocyclohexanecarboxamide.

United States Patent Application Publication No. 2013/0295118 by Jiang et al., incorporated herein by this reference, discloses antibodies that specifically bind the extracellular domain of human C-type lectin-like molecule (CLL-1). The antibodies can be used for suppression of cancer stem cell proliferation. The antibodies can be humanized and can be conjugated to a therapeutic compound.

United States Patent Application Publication No. 2013/0287688 by Jain et al., incorporated herein by this reference, discloses the use of anti-hypertension compounds for suppression of cancer stem cell proliferation. The anti-hypertension compounds include losartan, candesartan, eprosartan mesylate, EXP 3174, irbesartan, L158,809, olmesartan, saralasin, telmisartin, valsartan, aliskiren, remikiren, enalkiren, SPP635, benazepril, captopril, enalapril, fosinopril, lisinopril, moexipril, perindopril, quinapril, ramipril, trandolapril, ABT-510, CVX-045, LSKL, DN-9693, and FG-3019. Particular classes of anti-hypertension compounds include an angiotensin II receptor blocker, an antagonist of renin angiotensin aldosterone system, an angiotensin converting enzyme (ACE) inhibitor, a thrombospondin 1 (TSP-1) inhibitor, a transforming growth factor 31 inhibitor, a stromal cell-derived growth factor 1α inhibitor, or a connective tissue growth factor (CTGF) inhibitor.

United States Patent Application Publication No. 2013/0267757 to Schaffer et al., incorporated herein by this reference, discloses anthraquinone radiosensitizer agents that can be used together with ionizing radiation to suppress cancer stem cell proliferation. The anthraquinone radiosensitizer agents include hexamethyl hypericin, hypericin tetrasulfonic acid, and tetrabromohypericin.

United States Patent Application Publication No. 2013/0237495 by Lee et al., incorporated herein by this reference, discloses CDK-inhibiting pyrrolopyrimidinone derivatives that can be used for suppression of cancer stem cell proliferation. The derivatives are CDK1 or CDK2 inhibitors. The derivatives include 4-amino-6-bromo-1-((2S,3R,4R,5S)-3,4-dihydroxy-5-(hydroxymethyl)-tetrahydrofuran-2-yl)-1H-pyrrolo[2,3-d]pyrimidinone-5-carboxamide; ((2S,3R,4R,5S)-5-(4-amino-6-bromo-5-carbamoyl-1H-pyrrolo[2,3-d]pyrimidinone-1-yl)-3,4-dihydroxy-tetrahydrofuran-2-yl)methyl isobutylate; ((2S,3R,4R,5S)-5-(4-amino-6-bromo-5-carbamoyl-1H-pyrrolo[2,3-d]pyrimidinone-1-yl)-3,4-dihydroxy-tetrahydrofuran-2-yl)methyl pivalate; (2S,3R,4S,5S)-2-(4-amino-6-bromo-5-carbamoyl-1H-pyrrolo[2,3-d]pyrimidinone-1-yl)-5-(isobutyryloxymethyl)-tetrahydrofuran-3,4-diyl diacetate; ((2S,3R,4R,5S)-5-(4-amino-6-bromo-5-carbamoyl-1H-pyrrolo[2,3-d]pyrimidinone-1-yl)-3,4-dihydroxy-tetrahydrofuran-2-yl)methyl benzoate; ((2S,3R,4R,5S)-5-(4-amino-6-bromo-5-carbamoyl-1H-pyrrolo[2,3-d]pyrimidinone-1-yl)-3,4-dihydroxy-tetrahydrofuran-2-yl)methyl propionate; and ((2S,3R,4R,5S)-5-(4-amino-6-bromo-5-carbamoyl-1H-pyrrolo[2,3-d]pyrimidinone-1-yl)-3,4-dihydroxy-tetrahydrofuran-2-yl)methyl cyclohexanecarboxylate.

United States Patent Application Publication No. 2013/0224227 by Beusker et al., incorporated herein by this reference, discloses analogs of the DNA alkylating agent CC-1065 and their conjugates; the conjugates can include bifunctional linkers. The analogs and conjugates can be used to suppress cancer stem cell proliferation.

United States Patent Application Publication No. 2013/0224191 to Stull et al., incorporated herein by this reference, discloses antibodies specifically binding to the protein Notum that can be used to suppress cancer stem cell proliferation.

United States Patent Application Publication No. 2013/0217014 by Firestein et al., incorporated herein by this reference, discloses CDK8 antagonists that can be used to suppress cancer stem cell proliferation. The CDK8 antagonists include flavopiridol, ABT-869, AST-487, BMS-387032/SNS032, BIRB-796, sorafenib, staurosporine, cortistatin, cortistatin A, and a steroidal alkaloid or derivative thereof.

United States Patent Application Publication No. 2013/0210739 by Hugnot et al., incorporated herein by this reference, discloses bHLH proteins and nucleic acids encoding them that can be used to suppress cancer stem cell proliferation.

United States Patent Application Publication No. 2013/0210024 by Yu et al., incorporated herein by this reference, discloses a method of cancer treatment, including suppression of proliferation of cancer stem cells, by activating FBOX32 expression through the inhibition of the histone methyltransferase EZH2. The EZH2 inhibitor can be isoliquiritigenin or 3-Deazaneplanocin A.

United States Patent Application Publication No. 2013/0190396 by Supuran et al., incorporated herein by this reference, discloses sulfonamides that inhibit carbonic anhydrase isoforms and can be used for suppression of proliferation of cancer stem cells. The sulfonamides include 4-{[(benzylamino)carbonyl]amino}benzenesulfonamide; 4-{[(benzhydrylamino) carbonyl]amino}benzenesulfonamide; 4-{[(4′-fluorophenyl)carbamoyl]amino}benzenesulfonamide; 4-{[(4′-bromophenyl)carbamoyl]amino}benzenesulfonamide; 4-{[(2′-methoxyphenyl)carbamoyl]amino}benzenesulfonamide; 4-{[(2′-isopropylphenyl)carbamoyl]amino}benzenesulfonamide; 4-{[(4′-isopropylphenyl)carbamoyl]amino}benzenesulfonamide; 4-{[(4′-n-butylphenyl)carbamoyl]amino}benzenesulfonamide; 4-{[(4′-butoxyphenyl)carbamoyl]amino}benzenesulfonamide; 4-{[(4′-n-octylphenyl)carbamoyl]amino}benzenesulfonamide; 4-{[(4′-cyanophenyl)carbamoyl]amino}benzenesulfonamide; 4-{[(2′-cyanophenyl)carbamoyl]amino}benzenesulfonamide; 4-{[(4′-phenoxyphenyl)carbamoyl]amino}benzenesulfonamide; 4-{[(biphenyl-2′-yl)carbamoyl]amino}benzenesulfonamide; 4-{[(3′-nitrophenyl)carbamoyl]amino}benzenesulfonamide; 4-{[(4′-Methoxy-2′-methylphenyl)carbamoyl]amino}benzenesulfonamide; 4-[(cyclopentylcarbamoyl)amino]benzenesulfonamide; 4-{([(3′,5′-dimethylphenyl)amino]carbonylamino)}benzenesulfonamide; 4-{[(2′,3′-dihydro-1H-inden-5′-ylamino]carbonylamino)}benzenesulfonamide; 4-{[([3′,5′-bis(trifluoromethyl)phenyl]aminocarbonyl)amino]}benzenesulfonamide; 3-(3-(4′-Iodophenyl) ureido)benzenesulfonamide; 3-(3-(4′-fluorophenyl)ureido)benzenesulfonamide; 3-(3-(3′-nitrophenyl)ureido)benzenesulfonamide; 3-(3-(4′-acetylphenyl)ureido)benzenesulfonamide; 3-(3-(2′-isopropylphenyl)ureido)benzenesulfonamide; 3-(3-(perfluorophenyl)ureido)benzenesulfonamide; 4-(3-(4′-chloro-2-fluorophenyl)ureido)benzenesulfonamide; 4-(3-(4′-bromo-2′-fluorophenyl)ureido)benzenesulfonamide; 4-(3-(2′-fluoro-5′-nitrophenyl)ureido)benzenesulfonamide; and 4-(3-(2′,4′,5′-trifluorophenyl)ureido)benzenesulfonamide.

United States Patent Application Publication No. 2013/0177500 by Ruiz-Opazo et al., incorporated herein by this reference, discloses antibodies specifically binding DEspR and fragments thereof, including fully human, composite engineered human, humanized, monoclonal, and polyclonal antibodies that can be used for suppression of cancer stem cell proliferation.

United States Patent Application Publication No. 2013/0142808 by Suarez et al., incorporated herein by this reference, discloses antibodies specifically binding human leukemia inhibitory factor (LIF); the antibodies specifically bind full-length LIF but do not bind fragments of LIF, and can be used for suppression of cancer stem cell proliferation.

United States Patent Application Publication No. 2013/0116224 by Gershon, incorporated herein by this reference, discloses the use of doxovir to suppress cancer stem cell proliferation.

United States Patent Application Publication No. 2013/0102613 by Xu et al., incorporated herein by this reference, discloses the use of an inhibitor of mTOR to suppress cancer stem cell proliferation. Inhibitors of mTOR are well known in the art, and include, but are not limited to: sirolimus: temsirolimus, everolimus; rapamune; ridaforolimus; AP23573 (deforolimus); CCI-779 (rapamycin 42-ester with 3-hydroxy-2-(hydroxymethyl)-2-methylpropionic acid); AZD8055 ((5-(2,4-bis((S)-3-methylmorpholino)pyrido[2,3-d]pyrimidin-7-yl)-2-methoxyphenyl)methanol); PKI-587 (1-(4-(4-(dimethylamino)piperidine-1-carbonyl)phenyl)-3-(4-(4,6-dimorpholino-1,3,5-triazin-2-yl)phenyl)urea); NVP-BEZ235 (2-methyl-2-{4-[3-methyl-2-oxo-8-(quinolin-3-yl)-2,3-dihydro-1H-imidazo[4,5-c]quinolin-1-yl]phenyl}propanenitrile); LY294002 ((2-(4-morpholinyl)-8-phenyl-4H-1-benzopyran-4-one); 40-O-(2-hydroxyethyl)-rapamycin; ABT578 (zotarolimus); biolimus-7; biolimus-9; AP23675; AP23841; TAFA-93; 42-O-(methyl-D-glucosylcarbonyl)rapamycin; 42-O-[2-(methyl-D-glucosylcarbonyloxy)ethyl]rapamycin; 31-O-(methyl-D-glucosylcarbonyl)rapamycin; 42-O-(2-hydroxyethyl)-31-O-(methyl-D-glucosylcarbonyl)rapamycin; 42-O-(2-O-methyl-D-fructosylcarbonyl)rapamycin; 42-O-[2-(2-O-methyl-D-fructosylcarbonyloxy)ethyl]rapamycin; 42-O-(2-O-methyl-L-fructosylcarbonyl)rapamycin; 42-O-[2-(2-O-methyl-L-fructosylcarbonyloxy)ethyl]rapamycin; 31-O-(2-O-methyl-D-fructosylcarbonyl)rapamycin; 42-O-(2-hydroxyethyl)-31-O-(2-O-methyl-D-fructosylcarbonyl)rapamycin; 31-O-(2-O-methyl-L-fructosylcarbonyl)rapamycin; 42-O-(2-hydroxyethyl)-31-O-(2-O-methyl-L-fructosylcarbonyl)rapamycin; 42-O-(D-allosylcarbonyl)rapamycin; 42-O-[2-(D-allosylcarbonyloxy)ethyl]rapamycin; 42-O-(L-allosylcarbonyl)rapamycin; 42-O-[2-(L-allosylcarbonyloxy)ethyl]rapamycin; 31-O-(D-allosylcarbonyl)rapamycin; 42-O-(2-hydroxyethyl)-31-O-(D-allosylcarbonyl)rapamycin; 31-O-(L-allosylcarbonyl)rapamycin; 42-O-(2-hydroxyethyl)-31-O-(L-allosylcarbonyl)rapamycin; 42-O-(D-fructosylcarbonyl)rapamycin; 42-O-[2-(D-fructosylcatfionyloxy)ethyl]rapamycin; 42-O-(L-fructosylcarbonyl)rapamycin; 42-O-[2-(L-fructosylcarbonyloxy)ethyl]rapamycin; 31-O-(D-fructosylcarbonyl)rapamycin; 42-O-(2-hydroxyethyl)-31-O-(D-fructosylcarbonyl)rapamycin; 31-O-(L-fructosylcarbonyl)rapamycin; 42-O-(2-hydroxyethyl)-31-O-(L-fructosylcarbonyl)rapamycin; 42-O-(D-fucitolylcarbonyl)rapamycin; 42-O-[2-(D-fucitolylcarbonyloxy)ethyl]rapamycin; 42-O-(L-fucitolylcarbonyl)rapamycin; 42-O-[2-(L-fucitolylcarbonyloxy)ethyl]rapamycin; 31-O-(D-fucitolylcarbonyl)rapamycin; 42-O-(2-hydroxyethyl)-31-O-(D-fucitolylcarbonyl)rapamycin; 31-O-(L-fucitolylcarbonyl)rapamycin; 42-O-(2-hydroxyethyl)-31-O-(L-fucitolylcarbonyl)rapamycin; 42-O-(D-glucalylcarbonyl)rapamycin; 42-O-[2-(D-glucalylcarbonyloxy)ethyl]rapamycin; 42-O-(D-glucosylcarbonyl)rapamycin; 42-O-[2-(D-glucosylcarbonyloxy)ethyl]rapamycin; 42-O-(L-glucosylcarbonyl)rapamycin; 42-O-[2-(L-glucosylcarbonyloxy)ethyl]rapamycin; 31-O-(D-glucalylcarbonyl)rapamycin; 42-O-(2-hydroxyethyl)-31-O-(D-glucalylcarbonyl)rapamycin; 31-O-(D-glucosylcarbonyl)rapamycin; 42-O-(2-hydroxyethyl)-31-O-(D-glucosylcarbonyl)rapamycin; 31-O-(L-glucosylcarbonyl) rapamycin; 42-O-(2-hydroxyethyl)-31-O-(L-glucosylcarbonyl)rapamycin; 42-O-(L-sorbosylcarbonyl)rapamycin; 42-O-(D-sorbosylcarbonyl)rapamycin; 31-O-(L-sorbosylcarbonyl) rapamycin; 31-O-(D-sorbosylcarbonyl)rapamycin; 42-O-[2-(L-sorbosylcarbonyloxy) ethyl]rapamycin; 42-O-[2-(D-sorbosylcarbonyloxy)ethyl]rapamycin; 42-O-(2-hydroxyethyl)-31-O-(D-sorbosylcarbonyl)rapamycin; 42-O-(2-hydroxyethyl)-31-O-(L-sothosylcarbonyl) rapamycin; 42-O-(D-lactalylcarbonyl)rapamycin; 42-O-[2-(D-lactalylcarbonyloxy) ethyl]rapamycin; 31-O-(D-lactalylcarbonyl)rapamycin; 42-O-(2-hydroxyethyl)-31-O-(D-lactalylcarbonyl)rapamycin; 42-O-(D-sucrosylcarbonyl)rapamycin; 42-O-[2-(D-sucrosylcarbonyloxy)ethyl]rapamycin; 31-O-(D-sucrosylcarbonyl)rapamycin; 42-O-(2-hydroxyethyl)-31-O-(D-sucrosylcarbonyl)rapamycin; 42-O-(D-gentobiosylcarbonyl)rapamycin 42-O-[2-(D-gentobiosylcarbonyloxy)ethyl]rapamycin; 31-O-(D-gentobiosylcarbonyl)rapamycin 42-O-(2-hydroxyethyl)-31-O-(D-gentobiosylcarbonyl)rapamycin 42-O-(D-cellobiosylcarbonyl)rapamycin; 42-O-[2-(D-cellobiosylcarbonyloxy)ethyl]rapamycin; 31-O-(D-cellobiosylcarbonyl)rapamycin; 42-O-(2-hydroxyethyl)-31-O-(D-cellobiosylcarbonyl) rapamycin; 42-O-(D-turanosylcarbonyl)rapamycin; 42-O-[2-(D-turanosylcarbonyloxy) ethyl]rapamycin; 31-O-(D-turanosylcarbonyl)rapamycin; 42-O-(2-hydroxyethyl)-31-O-(D-turanosylcarbonyl)rapamycin; 42-O-(D-palatinosylcarbonyl)rapamycin; 42-O-[2-(D-palatinosylcarbonyloxy)ethyl]rapamycin; 31-O-(D-palatinosylcarbonyl)rapamycin; 42-O-(2-hydroxyethyl)-31-O-(D-palatinosylcarbonyl)rapamycin; 42-O-(D-isomaltosylcarbonyl) rapamycin; 42-O-[2-(D-isomaltosylcarbonyloxy)ethyl]rapamycin; 31-O-(D-isomaltosylcarbonyl) rapamycin; 42-O-(2-hydroxyethyl)-31-O-(D-isomaltosylcarbonyl)rapamycin; 42-O-(D-maltulosylcarbonyl)rapamycin; 42-O-[2-(D-maltulosylcarbonyloxy)ethyl]rapamycin; 42-O-(D-maltosylcarbonyl)rapamycin; 42-O-[2-(D-maltosylcathonyloxy)ethyl]rapamycin; 31-O-(D-maltulosylcarbonyl)rapamycin; 42-O-(2-hydroxyethyl)-31-O-(D-maltulosylcarbonyl)rapamycin; 31-O-(D-maltosylcarbonyl)rapamycin; 42-O-(2-hydroxyethyl)-31-O-(D-maltosylcarbonyl) rapamycin; 42-O-(D-lactosylcarbonyl)rapamycin; 42-O-[2-(D-lactosylcarbonyloxy) ethyl]rapamycin; 31-O-(methyl-D-lactosylcarbonyl)rapamycin; 42-O-(2-hydroxyethyl)-31-O-(methyl-D-lactosylcarbonyl)rapamycin; 42-O-(D-melibiosylcarbonyl)rapamycin; 31-O-(D-melibiosylcarbonyl)rapamycin; 42-O-(2-hydroxyethyl)-31-O-(D-melibiosylcarbonyl)rapamycin; 42-O-(D-leucrosylcarbonyl)rapamycin; 42-O-[2-(D-leucrosylcarbonyloxy)ethyl]rapamycin; 31-O-(D-leucrosylcarbonyl)rapamycin; 42-O-(2-hydroxyethyl)-31-O-(D-leucrosylcarbonyl) rapamycin; 42-O-(D-raffinosylcarbonyl)rapamycin; 42-O-[2-(D-raffinosylcarbonyloxy) ethyl]rapamycin; 31-O-(D-raffinosylcarbonyl)rapamycin; 42-O-(2-hydroxyethyl)-31-O-(D-raffinosylcarbonyl)rapamycin; 42-O-(D-isomaltotriosylcarbonyl)rapamycin; 42-O-[2-(D-isomaltosylcarbonyloxy)ethyl]rapamycin; 31-O-(D-isomaltotriosylcarbonyl)rapamycin; 42-O-(2-hydroxyethyl)-31-O-(D-isomaltotriosylcarbonyl)rapamycin; 42-O-(D-cellotetraosylcarbonyl) rapamycin; 42-O-[2-(D-cellotetraosylcarbonyloxy)ethyl]rapamycin; 31-O-(D-cellotetraosylcarbonyl)rapamycin; 42-O-(2-hydroxyethyl)-31-O-(D-cellotetraosylcarbonyl) rapamycin; 42-O-(valiolylcarbonyl)rapamycin; 42-O-[2-(D-valiolylcarbonyloxy) ethyl]rapamycin; 31-O-(valiolylcarbonyl)rapamycin; 42-O-(2-hydroxyethyl)-31-O-(valiolylcarbonyl)rapamycin; 42-O-(valiolonylcarbonyl)rapamycin; 42-O-[2-(D-valiolonylcarbonyloxy)ethyl]rapamycin; 31-O-(valiolonylcarbonyl)rapamycin; 42-O-(2-hydroxyethyl)-31-O-(valiolonylcarbonyl)rapamycin; 42-O-(valienolylcarbonyl)rapamycin 42-O-[2-(D-valienolylcarbonyloxy)ethyl]rapamycin; 31-O-(valienolylcarbonyl)rapamycin; 42-O-(2-hydroxyethyl)-31-O-(valienolylcarbonyl)rapamycin; 42-O-(valienoneylcarbonyl)rapamycin; 42-O-[2-(D-valienoneylcarbonyloxy)ethyl]rapamycin; 31-O-(valienoneylcarbonyl)rapamycin; 42-O-(2-hydroxyethyl)-31-O-(valienoneylcarbonyl)rapamycin; PI-103 (3-[4-(4-morpholinyl) pyrido[3′,2′:4,5]furo[3,2-d]pyrimidin-2-yl]-phenol); KU-0063794 ((5-(2-((2R,6S)-2,6-dimethylmorpholino)-4-morpholinopyrido[2,3-d]pyrimidin-7-yl)-2-methoxyphenyl)methanol); PF-04691502 (2-amino-8-((1r,4r)-4-(2-hydroxyethoxy)cyclohexyl)-6-(6-methoxypyridin-3-yl)-4-methylpyrido[2,3-d]pyrimidin-7(8H)-one); CH132799; RG7422 ((5)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one); Palomid 529 (3-(4-methoxybenzyloxy)-8-(1-hydroxyethyl)-2-methoxy-6H-benzo[c]chromen-6-one); PP242 (2-(4-amino-1-isopropyl-1H-pyrazolo[3,4-d]pyrimidin-3-yl)-1H-indol-5-ol); XL765 (N-[4-[[[3-[(3,5-dimethoxyphenyl)amino]-2-quinoxalinyl]amino]sulfonyl]phenyl]-3-methoxy-4-methyl-benzamide); GSK1059615 ((Z)-5-((4-(pyridin-4-yl)quinolin-6-yl)methylene)thiazolidine-2,4-dione); PKI-587 (1-(4-(4-(dimethylamino)piperidine-1-carbonyl)phenyl)-3-(4-(4,6-dimorpholino-1,3,5-triazin-2-yl)phenyl)urea); WAY-600 (6-(1H-indol-5-yl)-4-morpholino-1-(1-(pyridin-3-ylmethyl)piperidin-4-yl)-1H-pyrazolo[3,4-d]pyrimidine); WYE-687 (methyl 4-(4-morpholino-1-(1-(pyridin-3-ylmethyl)piperidin-4-yl)-1H-pyrazolo[3,4-d]pyrimidin-6-yl)phenylcarbamate); WYE-125132 (N-[4-[1-(1,4-dioxaspiro[4.5]dec-8-yl)-4-(8-oxa-3-azabicyclo[3.2.1]oct-3-yl)-1H-pyrazolo[3,4-d]pyrimidin-6-yl]phenyl]-N′-methyl-urea); and WYE-354.

United States Patent Application Publication No. 2013/0095104 by Cummings et al., incorporated herein by this reference, discloses antibodies, including monoclonal antibodies or antigen-binding fragments thereof, specifically binding to FZD10. The antibodies can be conjugated to an antineoplastic agent.

United States Patent Application Publication No. 2013/0034591 by Li et al., incorporated herein by this reference, discloses the use of napthofuran compounds for suppression of stem cell proliferation. The compounds include 2-acetyl-4H,9H-naphtho[2,3-b]furan-4,9-dione.

United States Patent Application Publication No. 2013/0004521 by Buchsbaum et al., incorporated herein by this reference, discloses the use of death receptor agonists, such as death receptor antibodies such as DR4 antibodies or DR5 antibodies, for suppression of cancer stem cell proliferation.

United States Patent Application Publication No. 2012/0329721 by Schimmer et al., incorporated herein by this reference, discloses the use of tigecycline for suppression of cancer stem cell proliferation.

United States Patent Application Publication No. 2014/0323563 by Kapulnik et al., incorporated herein by this reference, discloses the use of strigolactones and strigolactone analogs for suppression of cancer stem cell proliferation.

United States Patent Application Publication No. 2014/0322128 by Maltese et al., incorporated herein by this reference, discloses compounds useful for suppression of cancer stem cell proliferation by induction of methuosis. The compounds include trans-3-(2-methyl-1H-indol-3-yl)-1-(4-pyridinyl)-2-propen-1-one; trans-3-(1H-indol-3-yl)-1-phenyl-2-propen-1-one; trans-3-(1H-indol-3-yl)-1-(2-pyridinyl)-2-propen-1-one; trans-3-(1H-indol-3-yl)-1-(3-pyridinyl)-2-propen-1-one; trans-3-(1H-indol-3-yl)-1-(4-pyridinyl)-2-propen-1-one; trans-3-(5-methoxy-1H-indol-3-yl)-1-(4-pyridinyl)-2-propen-1-one; trans-3-(5-phenylmethoxy-1H-indol-3-yl)-1-(4-pyridinyl)-2-propen-1-one; trans-3-(5-Hydroxy-1H-indol-3-yl)-1-(4-pyridinyl)-2-propen-1-one; trans-3-(5-methoxy-1H-indol-3-yl)-1-(3-pyridinyl)-2-propen-1-one; trans-3-(5-methoxy-1H-indol-3-yl)-1-(pyrazine)-2-propen-1-one; trans-3-(5-methoxy-2-methyl-1H-indol-3-yl)-1-(4-pyridinyl)-2-propen-1-one; trans-3-(5-methoxy-1-methyl-indol-3-yl)-1-(4-pyridinyl)-2-propen-1-one; trans-3-(5-hydroxy-1H-indol-3-yl)-1-(4-pyridinyl)-2-propen-1-one; trans-3-[5-((4-methylbenzoate)methoxy)-1H-indol-3-yl)]-1-(4-pyridinyl)-2-propen-1-one; and trans-3-[5-((4-carboxyphenyl)-methoxy)-1H-indol-3-yl)]-1-(4-pyridinyl)-2-propen-1-one.

Another aspect of the present invention is a composition to improve the efficacy and/or reduce the side effects of suboptimally administered drug therapy employing a substituted hexitol derivative for the treatment of NSCLC or GBM comprising an alternative selected from the group consisting of:

(i) a therapeutically effective quantity of a modified substituted hexitol derivative or a derivative, analog, or prodrug of a substituted hexitol derivative or a modified substituted hexitol derivative, wherein the modified substituted hexitol derivative or the derivative, analog or prodrug of the substituted hexitol derivative or modified substituted hexitol derivative possesses increased therapeutic efficacy or reduced side effects for treatment of NSCLC or GBM as compared with an unmodified substituted hexitol derivative;

(ii) a composition comprising:
(a) a therapeutically effective quantity of a substituted hexitol derivative, a modified substituted hexitol derivative, or a derivative, analog, or prodrug of a substituted hexitol derivative or a modified substituted hexitol derivative; and
(b) at least one additional therapeutic agent, therapeutic agent subject to chemosensitization, therapeutic agent subject to chemopotentiation, diluent, excipient, solvent system, drug delivery system, agent to counteract myelosuppression, or agent that increases the ability of the substituted hexitol to pass through the blood-brain barrier, wherein the composition possesses increased therapeutic efficacy or reduced side effects for treatment of NSCLC or GBM as compared with an unmodified substituted hexitol derivative;

(iii) a therapeutically effective quantity of a substituted hexitol derivative, a modified substituted hexitol derivative or a derivative, analog, or prodrug of a substituted hexitol derivative or a modified substituted hexitol derivative that is incorporated into a dosage form, wherein the substituted hexitol derivative, the modified substituted hexitol derivative or the derivative, analog, or prodrug of a substituted hexitol derivative or a modified substituted hexitol derivative incorporated into the dosage form possesses increased therapeutic efficacy or reduced side effects for treatment of NSCLC or GBM as compared with an unmodified substituted hexitol derivative;

(iv) a therapeutically effective quantity of a substituted hexitol derivative, a modified substituted hexitol derivative or a derivative, analog, or prodrug of a substituted hexitol derivative or a modified substituted hexitol derivative that is incorporated into a dosage kit and packaging, wherein the substituted hexitol derivative, the modified substituted hexitol derivative or the derivative, analog, or prodrug of a substituted hexitol derivative or a modified substituted hexitol derivative incorporated into the dosage kit and packaging possesses increased therapeutic efficacy or reduced side effects for treatment of NSCLC or GBM as compared with an unmodified substituted hexitol derivative; and

(v) a therapeutically effective quantity of a substituted hexitol derivative, a modified substituted hexitol derivative or a derivative, analog, or prodrug of a substituted hexitol derivative or a modified substituted hexitol derivative that is subjected to a bulk drug product improvement, wherein substituted hexitol derivative, a modified substituted hexitol derivative or a derivative, analog, or prodrug of a substituted hexitol derivative or a modified substituted hexitol derivative subjected to the bulk drug product improvement possesses increased therapeutic efficacy or reduced side effects for treatment of NSCLC or GBM as compared with an unmodified substituted hexitol derivative.

As detailed above, typically the unmodified substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the unmodified substituted hexitol derivative is dianhydrogalactitol.

In one alternative, the composition comprises a drug combination comprising:

(i) a substituted hexitol derivative; and

(ii) an additional therapeutic agent selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) monofunctional alkylating agents;
(h) bifunctional alkylating agents;
(i) alkylating agents that damage DNA at a different place than does dianhydrogalactitol;
(j) anti-tubulin agents;
(k) antimetabolites;
(l) berberine;
(m) apigenin;
(n) amonafide;
(o) colchicine or analogs;
(p) genistein;
(q) etoposide;
(r) cytarabine;
(s) camptothecins;
(t) vinca alkaloids;
(u) 5-fluorouracil;
(v) curcumin;
(w) NF-κB inhibitors;
(x) rosmarinic acid;
(y) mitoguazone;
(z) tetrandrine;
(aa) temozolomide;
(ab) VEGF inhibitors;
(ac) cancer vaccines;
(ad) EGFR inhibitors;
(ae) tyrosine kinase inhibitors;
(af) poly (ADP-ribose) polymerase (PARP) inhibitors;
(ag) ALK inhibitors; and
(ah) agents that suppress proliferation of cancer stem cells.

In another alternative, the composition comprises:

(i) a substituted hexitol derivative; and

(ii) a therapeutic agent subject to chemosensitization selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(j) berberine;
(k) apigenin;
(l) amonafide;
(m) colchicine or analogs;
(n) genistein;
(o) etoposide;
(p) cytarabine;
(q) camptothecins;
(r) vinca alkaloids;
(s) topoisomerase inhibitors;
(t) 5-fluorouracil;
(u) curcumin;
(v) NF-κB inhibitors;
(w) rosmarinic acid;
(x) mitoguazone;
(y) tetrandrine;
(z) a tyrosine kinase inhibitor;
(aa) an inhibitor of EGFR; and
(ab) an inhibitor of PARP;
wherein the substituted hexitol derivative acts as a chemosensitizer.

In still another alternative, the composition comprises:

(i) a substituted hexitol derivative; and

(ii) a therapeutic agent subject to chemopotentiation selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(j) berberine;
(k) apigenin;
(l) amonafide;
(m) colchicine or analogs;
(n) genistein;
(o) etoposide;
(p) cytarabine;
(q) camptothecins;
(r) vinca alkaloids;
(s) 5-fluorouracil;
(t) curcumin;
(u) NF-κB inhibitors;
(v) rosmarinic acid;
(w) mitoguazone;
(x) tetrandrine;
(y) a tyrosine kinase inhibitor;
(z) an inhibitor of EGFR; and
(aa) an inhibitor of PARP;
   wherein the substituted hexitol derivative acts as a chemopotentiator.

In yet another alternative, the substituted hexitol derivative is subjected to a bulk drug product improvement, wherein the bulk drug product improvement is selected from the group consisting of:
(a) salt formation;
(b) preparation as a homogeneous crystal structure;
(c) preparation as a pure isomer;
(d) increased purity;
(e) preparation with lower residual solvent content; and
(f) preparation with lower residual heavy metal content.

In still another alternative, the composition comprises a substituted hexitol derivative and a diluent, wherein the diluent is selected from the group consisting of:
(a) an emulsion;
(b) dimethylsulfoxide (DMSO);
(c) N-methylformamide (NMF)
(d) DMF;
(e) ethanol;
(f) benzyl alcohol;
(g) dextrose-containing water for injection;
(h) Cremophor™;
(i) cyclodextrin; and
(j) PEG.

In still another alternative, the composition comprises a substituted hexitol derivative and a solvent system, wherein the solvent system is selected from the group consisting of:
(a) an emulsion;
(b) dimethylsulfoxide (DMSO);
(c) N-methylformamide (NMF)
(d) DMF;
(e) ethanol;
(f) benzyl alcohol;
(g) dextrose-containing water for injection;
(h) Cremophor™;
(i) cyclodextrin; and
(j) PEG.

In yet another alternative, the composition comprises a substituted hexitol derivative and an excipient, wherein the excipient is selected from the group consisting of:
(a) mannitol;
(b) albumin;
(c) EDTA;
(d) sodium bisulfite;
(e) benzyl alcohol;
(f) a carbonate buffer; and
(g) a phosphate buffer.

In still another alternative, the substituted hexitol derivative is incorporated into a dosage form selected from the group consisting of:
(a) tablets;
(b) capsules;
(c) topical gels;
(d) topical creams;
(e) patches;
(f) suppositories; and
(g) lyophilized dosage fills.

In yet another alternative, the substituted hexitol derivative is incorporated into a dosage kit and packaging selected from the group consisting of amber vials to protect from light and stoppers with specialized coatings to improve shelf-life stability. As indicated above, the dosage kit and packaging can be labeled to indicate details of use and may contain one or more than one therapeutically active agent; if more than one therapeutic agent is included, the two or more therapeutic agents can be combined or separately packaged.

In still another alternative, the composition comprises a substituted hexitol derivative and a drug delivery system selected from the group consisting of:
(a) nanocrystals;
(b) bioerodible polymers;
(c) liposomes;
(d) slow release injectable gels; and
(e) microspheres.

In still another alternative, the substituted hexitol derivative is present in the composition in a drug conjugate form selected from the group consisting of:
(a) a polymer system;
(b) polylactides;
(c) polyglycolides;
(d) amino acids;
(e) peptides; and
(f) multivalent linkers.

In yet another alternative, the therapeutic agent is a modified substituted hexitol derivative and the modification is selected from the group consisting of:
(a) alteration of side chains to increase or decrease lipophilicity;
(b) addition of an additional chemical functionality to alter a property selected from the group consisting of reactivity, electron affinity, and binding capacity; and
(c) alteration of salt form.

In still another alternative, the substituted hexitol derivative is in the form of a prodrug system, wherein the prodrug system is selected from the group consisting of:
(a) the use of enzyme sensitive esters;
(b) the use of dimers;
(c) the use of Schiff bases;
(d) the use of pyridoxal complexes; and
(e) the use of caffeine complexes.

In yet another alternative, the composition comprises a substituted hexitol derivative and at least one additional therapeutic agent to form a multiple drug system, wherein the at least one additional therapeutic agent is selected from the group consisting of:
(a) an inhibitor of multi-drug resistance;
(b) a specific drug resistance inhibitor;
(c) a specific inhibitor of a selective enzyme;
(d) a signal transduction inhibitor,
(e) an inhibitor of a repair enzyme; and
(f) a topoisomerase inhibitor with non-overlapping side effects.

In yet another alternative, the composition comprises a substituted hexitol derivative and an agent to counteract myelosuppression as described above. Typically, the agent to counteract myelosuppression is a dithiocarbamate.

In yet another alternative, the composition comprises a substituted hexitol derivative and an agent that increases the ability of the substituted hexitol to pass through the blood-brain barrier as described above. Typically, the agent that increases the ability of the substituted hexitol to pass through the blood-brain barrier is an agent selected from the group consisting of:
(a) a chimeric peptide of the structure of Formula (D-III):

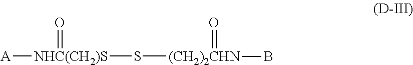

wherein: (A) A is somatostatin, thyrotropin releasing hormone (TRH), vasopressin, alpha interferon, endorphin, muramyl dipeptide or ACTH 4-9 analogue; and (B) B is insulin, IGF-I, IGF-II, transferrin, cationized (basic) albumin or prolactin; or a chimeric peptide of the structure of Formula (D-III) wherein the disulfide conjugating bridge between A and B is replaced with a bridge of Subformula (D-III(a)):

A-NH(CH₂)₂S—S—B(cleavable linkage) (D-III(a)),

wherein the bridge is formed using cysteamine and EDAC as the bridge reagents; or a chimeric peptide of the structure of Formula (D-III) wherein the disulfide conjugating bridge between A and B is replaced with a bridge of Subformula (D-III(b)):

A-NH═CH(CH₂)₃CH═NH—B(non-cleavable linkage) (D-III(b)),

wherein the bridge is formed using glutaraldehyde as the bridge reagent;
(b) a composition comprising either avidin or an avidin fusion protein bonded to a biotinylated substituted hexitol derivative to form an avidin-biotin-agent complex including therein a protein selected from the group consisting of insulin, transferrin, an anti-receptor monoclonal antibody, a cationized protein, and a lectin;
(c) a neutral liposome that is pegylated and incorporates the substituted hexitol derivative, wherein the polyethylene glycol strands are conjugated to at least one transportable peptide or targeting agent;
(d) a humanized murine antibody that binds to the human insulin receptor linked to the substituted hexitol derivative through an avidin-biotin linkage; and
(e) a fusion protein comprising a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative, wherein the fusion protein is linked to the substituted hexitol by a covalent link to biotin.

In still another alternative, the composition comprises a substituted hexitol derivative and an agent that suppresses proliferation of cancer stem cells, wherein the agent that suppresses proliferation of cancer stem cells is selected from the group consisting of: (1) naphthoquinones; (2) VEGF-DLL4 bispecific antibodies; (3) farnesyl transferase inhibitors; (4) gamma-secretase inhibitors; (5) anti-TIM3 antibodies; (6) tankyrase inhibitors; (7) Wnt pathway inhibitors other than tankyrase inhibitors; (8) camptothecin-binding moiety conjugates; (9) Notch1 binding agents, including antibodies; (10) oxabicycloheptanes and oxabicycloheptenes; (11) inhibitors of the mitochondrial electron transport chains or the mitochondrial tricarboxylic acid cycle; (12) Axl inhibitors; (13) dopamine receptor antagonists; (14) anti-RSPO1 antibodies; (15) inhibitors or modulators of the Hedgehog pathway; (16) caffeic acid analogs and derivatives; (17) Stat3 inhibitors; (18) GRP-94-binding antibodies; (19) Frizzled receptor polypeptides; (20) immunoconjugates with cleavable linkages; (21) human prolactin, growth hormone, or placental lactogen; (22) anti-prominin-1 antibody; (23) antibodies specifically binding N-cadherin; (24) DR5 agonists; (25) anti-DLL4 antibodies or binding fragments thereof; (26) antibodies specifically binding GPR49; (27) DDR1 binding agents; (28) LGR5 binding agents; (29) telomerase-activating compounds; (30) fingolimod plus anti-CD74 antibodies or fragments thereof; (31) an antibody that prevents the binding of CD47 to SIPRα or a CD47 mimetic; (32) thienopyranone kinase inhibitors for inhibition of PI-3 kinases; (33) cancer-stem-cell-binding peptides; (34) diphtheria toxin-interleukin 3 conjugates; (35) inhibitors of histone deacetylase; (36) progesterone or analogs thereof; (37) antibodies binding the negative regulatory region (NRR) of Notch2; (38) inhibitors of HGFIN; (39) immunotherapeutic peptides; (40) inhibitors of CSCPK or related kinases; (41) imidazo[1,2-a]pyrazine derivatives as α-helix mimetics; (42) antibodies directed to an epitope of variant Heterogeneous Ribonucleoprotein G (HnRNPG); (43) antibodies binding TES7 antigen; (44) antibodies binding the ILR3α subunit; (45) ifenprodil tartrate and other compounds with a similar activity; (46) antibodies binding SALL4; (47) antibodies binding Notch4; (48) bispecific antibodies binding both NBR1 and Cep55; (49) Smo inhibitors; (50) peptides blocking or inhibiting interleukin-1 receptor 1; (51) antibodies specific for CD47 or CD19; (52) histone methyltransferase inhibitors; (53) antibodies specifically binding Lg5; (54) antibodies specifically binding EFNA1; (55) phenothiazine derivatives; (56) HDAC inhibitors plus AKT inhibitors; (57) ligands binding to cancer-stem-line-specific cell surface antigen stem cell markers; (58) Notch receptor agonists; (59) binding agents binding human MET; (60) PDGFR-3 inhibitors; (61) pyrazolo compounds with histone demethylase activity; (62) heterocyclic substituted 3-heteroaryidenyl-2-indolinone derivatives; (63) albumin-binding arginine deiminase fusion proteins; (64) hydrogen-bond surrogate peptides and peptidomimetics that reactivate p53; (65) prodrugs of 2-pyrrolinodoxorubicin conjugated to antibodies; (66) targeted cargo proteins; (67) bisacodyl and analogs thereof; (68) N¹-cyclic amine-N⁵-substituted phenyl biguanide derivative; (69) fibulin-3 protein; (70) modulators of SCFSkp2; (71) inhibitors of Slingshot-2; (72) monoclonal antibodies specifically binding DCLK1 protein; (73) antibodies or soluble receptors that modulate the Hippo pathway; (74) selective inhibitors of CDK8 and CDK19; (75) antibodies and antibody fragments specifically binding IL-17; (76) antibodies specifically binding FRMD4A; (77) monoclonal antibodies specifically binding the ErbB-3 receptor; (78) antibodies that specifically bind human RSPO3 and modulate β-catenin activity; (79) esters of 4,9-dihydroxy-naphtho[2,3-b]furans; (80) CCR5 antagonists; (81) antibodies that specifically bind the extracellular domain of human C-type lectin-like molecule (CLL-1); (82) anti-hypertension compounds; (83) anthraquinone radiosensitizer agents plus ionizing radiation; (84) CDK inhibiting pyrrolopyrimidinone derivatives; (85) analogs of CC-1065 and conjugates thereof; (86) antibodies specifically binding to the protein Notum; (87) CDK8 antagonists; (88) bHLH proteins and nucleic acids encoding them; (89) inhibitors of the histone methyltransferase EZH2; (90) sulfonamides inhibiting carbonic anhydrase isoforms; (91) antibodies specifically binding DEspR; (92) antibodies specifically binding human leukemia inhibitory factor (LIF); (93) doxovir; (94) inhibitors of mTOR; (95) antibodies specifically binding FZD10; (96) napthofurans; (97) death receptor agonists; (98) tigecycline; (99) strigolactones and strigolactone analogs; and (100) compounds inducing methuosis.

When a pharmaceutical composition according to the present invention includes a prodrug, prodrugs and active metabolites of a compound may be identified using routine techniques known in the art. See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

When the pharmacologically active compound in a pharmaceutical composition according to the present invention possesses a sufficiently acidic, a sufficiently basic, or both a sufficiently acidic and a sufficiently basic functional group, these group or groups can accordingly react with any of a number of inorganic or organic bases, and inorganic and organic acids, to form a pharmaceutically acceptable salt. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, β-hydroxybutyrates, glycolates, tartrates, methane-sulfonates, propanesulfonates, naphthalene-1-sulfonates, naphthalene-2-sulfonates, and mandelates. If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

The amount of a given pharmacologically active agent, such as a substituted hexitol derivative such as dianhydrogalactitol or an analog or derivative of dianhydrogalactitol as described above, that is included in a unit dose of a pharmaceutical composition according to the present invention will vary depending upon factors such as the particular compound, disease condition and its severity, the identity (e.g., weight) of the subject in need of treatment, but can nevertheless be routinely determined by one skilled in the art. Typically, such pharmaceutical compositions include a therapeutically effective quantity of the pharmacologically active agent and an inert pharmaceutically acceptable carrier or diluent. Typically, these compositions are prepared in unit dosage form appropriate for the chosen route of administration, such as oral administration or parenteral administration. A pharmacologically active agent as described above can be administered in conventional dosage form prepared by combining a therapeutically effective amount of such a pharmacologically active agent as an active ingredient with appropriate pharmaceutical carriers or diluents according to conventional procedures. These procedures may involve mixing, granulating and compressing or dissolving the ingredients as appropriate to the desired preparation. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like. A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of a pharmacologically active agent as described above is dissolved in an aqueous solution of an organic or inorganic acid, such as 0.3 M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the particular complex being used, the particular composition formulated, the mode of administration and the particular site, host and disease and/or condition being treated. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular therapeutic agent, the route of administration, the time of administration, the rate of excretion of the particular compound being employed, the severity of the condition, other health considerations affecting the subject, and the status of liver and kidney function of the subject. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Optimal dosages for a given set of conditions can be ascertained by those skilled in the art using conventional dosage-determination tests in view of the experimental data for an agent. For oral administration, an exemplary daily dose generally employed is from about 0.001 to about 3000 mg/kg of body weight, with courses of treatment repeated at appropriate intervals. In some embodiments, the daily dose is from about 1 to 3000 mg/kg of body weight. Other dosages are as described above.

Typical daily doses in a patient may be anywhere between about 500 mg to about 3000 mg, given once or twice daily, e.g., 3000 mg can be given twice daily for a total dose of 6000 mg. In one embodiment, the dose is between about 1000 to about 3000 mg. In another embodiment, the dose is between about 1500 to about 2800 mg. In other embodiments, the dose is between about 2000 to about 3000 mg. Typically, doses are from about 1 mg/m² to about 40 mg/m². Preferably, doses are from about 5 mg/m² to about 25 mg/m². Additional alternatives for dosages are as described above with respect to schedules of administration and dose modification. Dosages can be varied according to the therapeutic response.

Plasma concentrations in the subjects may be between about 100 μM to about 1000 μM. In some embodiments, the plasma concentration may be between about 200 μM to about 800 μM. In other embodiments, the concentration is about 300 μM to about 600 μM. In still other embodiments the plasma concentration may be between about 400 to about 800 μM. In another alternative, the plasma concentration can be between about 0.5 μM to about 20 μM, typically 1 μM to about 10 μM. Administration of prodrugs is typically dosed at weight levels, which are chemically equivalent to the weight levels of the fully active form.

The compositions of the invention may be manufactured using techniques generally known for preparing pharmaceutical compositions, e.g., by conventional techniques such as mixing, dissolving, granulating, dragee-making, levitating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations, which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, solutions, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Pharmaceutical formulations for parenteral administration can include aqueous solutions or suspensions. Suitable lipophilic solvents or vehicles include fatty oils such as sesame oil or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility or dispersibility of the composition to allow for the preparation of highly concentrated solutions, or can contain suspending or dispersing agents. Pharmaceutical preparations for oral use can be obtained by combining the pharmacologically active agent with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate.

Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-α-naphthylamine, or lecithin can be used. Also, chelators such as EDTA can be used. Other ingredients that are conventional in the area of pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, particularly as described above. For administration of any of the compounds used in the present invention, preparations should meet sterility, pyrogenicity, general safety, and purity standards as required by the FDA Office of Biologics Standards or by other regulatory organizations regulating drugs.

For administration intranasally or by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days; in other alternatives, depending on the therapeutic agent and the formulation employed, release may occur over hours, days, weeks, or months. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

A pharmaceutical composition can be administered by a variety of methods known in the art. The routes and/or modes of administration vary depending upon the desired results. Depending on the route of administration, the pharmacologically active agent may be coated in a material to protect the targeting composition or other therapeutic agent from the action of acids and other compounds that may inactivate the agent. Conventional pharmaceutical practice can be employed to provide suitable formulations or compositions for the administration of such pharmaceutical compositions to subjects. Any appropriate route of administration can be employed, for example, but not limited to, intravenous, parenteral, intraperitoneal, intravenous, transcutaneous, subcutaneous, intramuscular, intraurethral, or oral administration. Depending on the severity of the malignancy or other disease, disorder, or condition to be treated, as well as other conditions affecting the subject to be treated, either systemic or localized delivery of the pharmaceutical composition can be used in the course of treatment. The pharmaceutical composition as described above can be administered together with additional therapeutic agents intended to treat a particular disease or condition, which may be the same disease or condition that the pharmaceutical composition is intended to treat, which may be a related disease or condition, or which even may be an unrelated disease or condition.

Pharmaceutical compositions according to the present invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymers, lactide/glycolide copolymers, or polyoxyethylene-polyoxypropylene copolymers may be used to control the release of the compounds. Other potentially useful parenteral delivery systems for molecules of the invention include ethylene-vinyl acetate copolymer particles, osmotic pumps, and implantable infusion systems. Formulations for inhalation may contain excipients, for example, lactose, or may be aqueous solutions containing, e.g., polyoxyethylene-9-lauryl ether, glycocholate and deoxycholate, or can be oily solutions for administration or gels.

Pharmaceutical compositions according to the present invention are usually administered to the subjects on multiple occasions. Intervals between single dosages can be weekly, monthly or yearly. Intervals can also be irregular as indicated by therapeutic response or other parameters well known in the art. Alternatively, the pharmaceutical composition can be administered as a sustained release formulation, in which case less frequent administration is required. Dosage and frequency vary depending on the half-life in the subject of the pharmacologically active agent included in a pharmaceutical composition. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regime.

For the purposes of the present application, treatment can be monitored by observing one or more of the improving symptoms associated with the disease, disorder, or condition being treated, or by observing one or more of the improving clinical parameters associated with the disease, disorder, or condition being treated. In the case of NSCLC, the clinical parameters can include, but are not limited to, reduction in tumor burden, reduction in pain, improvement in lung function, improvement in Karnofsky Performance Score, and reduction in occurrence of tumor spread or metastasis. As used herein, the terms “treatment,” “treating,” or equivalent terminology are not intended to imply a permanent cure for the disease, disorder, or condition being treated. Compositions and methods according to the present invention are not limited to treatment of humans, but are applicable to treatment of socially or economically important animals, such as dogs, cats, horses, cows, sheep, goats, pigs, and other animal species of social or economic importance. Unless specifically stated, compositions and methods according to the present invention are not limited to the treatment of humans.

Sustained-release formulations or controlled-release formulations are well-known in the art. For example, the sustained-release or controlled-release formulation can be (1) an oral matrix sustained-release or controlled-release formulation; (2) an oral multilayered sustained-release or controlled-release tablet formulation; (3) an oral multiparticulate sustained-release or controlled-release formulation; (4) an oral osmotic sustained-release or controlled-release formulation; (5) an oral chewable sustained-release or controlled-release formulation; or (6) a dermal sustained-release or controlled-release patch formulation.

The pharmacokinetic principles of controlled drug delivery are described, for example, in B. M. Silber et al., “Pharmacokinetic/Pharmacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251, incorporated herein by this reference.

One of ordinary skill in the art can readily prepare formulations for controlled release or sustained release comprising a pharmacologically active agent according to the present invention by modifying the formulations described above, such as according to principles disclosed in V. H. K. Li et al, “Influence of Drug Properties and Routes of Drug Administration on the Design of Sustained and Controlled Release Systems” in Controlled Drug Delivery: Fundamentals and Applications (J. R. Robinson & V. H. L. Lee, eds, 2d ed., Marcel Dekker, New York, 1987), ch. 1, pp. 3-94, incorporated herein by this reference. This process of preparation typically takes into account physicochemical properties of the pharmacologically active agent, such as aqueous solubility, partition coefficient, molecular size, stability, and nonspecific binding to proteins and other biological macromolecules. This process of preparation also takes into account biological factors, such as absorption, distribution, metabolism, duration of action, the possible existence of side effects, and margin of safety, for the pharmacologically active agent. Accordingly, one of ordinary skill in the art could modify the formulations into a formulation having the desirable properties described above for a particular application.

U.S. Pat. No. 6,573,292 by Nardella, U.S. Pat. No. 6,921,722 by Nardella, U.S. Pat. No. 7,314,886 to Chao et al., and U.S. Pat. No. 7,446,122 by Chao et al., which disclose methods of use of various pharmacologically active agents and pharmaceutical compositions in treating a number of diseases and conditions, including cancer, and methods of determining the therapeutic effectiveness of such pharmacologically active agents and pharmaceutical compositions, are all incorporated herein by this reference.

In view of the results reported in the Examples below, another aspect of the present invention is a method of treating NSCLC or GBM comprising the step of administering a therapeutically effective quantity of a substituted hexitol derivative such as dianhydrogalactitol to a patient suffering from the malignancy.

In this method, the substituted hexitol derivative can be selected from the group consisting of galactitols, substituted galacitols, dulcitols, and substituted dulcitols. Typically, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the substituted hexitol derivative is dianhydrogalactitol.

Typically, when the substituted hexitol derivative is dianhydrogalactitol, the therapeutically effective quantity of dianhydrogalactitol is from about 1 mg/m² to about 40 mg/m². Preferably, the therapeutically effective quantity of dianhydrogalactitol is from about 5 mg/m² to about 25 mg/m². Therapeutically active quantities of substituted hexitol derivatives other than dianhydrogalactitol can be determined by one of ordinary skill in the art by using the molecular weight of the particular substituted hexitol derivative and the activity of the particular substituted hexitol derivative, such as the in vitro activity of the substituted hexitol derivative against a standard cell line. Other suitable dosages are described above with respect to dose modification and schedule of administration and also in the Examples.

Typically, the substituted hexitol derivative such as dianhydrogalactitol is administered by a route selected from the group consisting of intravenous and oral. Preferably, the substituted hexitol derivative such as dianhydrogalactitol is administered intravenously.

The method can further comprise the step of administering a therapeutically effective dose of ionizing radiation. The method can further comprise the step of administering a therapeutically effective dose of an additional chemotherapeutic agent selected from the group consisting of cisplatin, carboplatin, bevacizumab, paclitaxel, Abraxane™ (paclitaxel bound to albumin as a delivery vehicle), docetaxel, etoposide, gemcitabine, vinorelbine tartrate, and pemetrexed. Suitable methods for administration of these agents and suitable dosages are well known in the art. The method can also further comprise the step of administering a therapeutically effective quantity of a corticosteroid. The method can also further comprise the step of administering a therapeutically effective quantity of at least one chemotherapeutic agent selected from the group consisting of lomustine, a platinum-containing chemotherapeutic agent, vincristine, and cyclophosphamide. The method can also further comprise administering a therapeutically effective quantity of a tyrosine kinase inhibitor or an EGFR inhibitor.

When the method further comprises the step of administering a therapeutically effective dose of ionizing radiation, suitable parameters for administration of the ionizing radiation are as described above, including dosages, administration of the ionizing radiation in a single dose or in fractionated doses, and the specific type of ionizing radiation administered.

In another significant alternative, the method can further comprise administering to the patient a therapeutically effective quantity of an agent that suppresses the growth of cancer stem cells. Suitable agents that suppress the growth of cancer stem cells are described above.

Typically, the substituted hexitol derivative such as dianhydrogalactitol substantially suppresses the growth of cancer stem cells (CSCs). Typically, the suppression of the growth of cancer stem cells is at least 50%. Preferably, the suppression of the growth of cancer stem cells is at least 99%.

Typically, the substituted hexitol derivative such as dianhydrogalactitol is effective in suppressing the growth of cancer cells possessing O⁶-methylguanine-DNA methyltransferase (MGMT)-driven drug resistance. Typically, the substituted hexitol derivative such as dianhydrogalactitol is also effective in suppressing the growth of cancer cells resistant to temozolomide.

The method can further comprise the administration of a therapeutically effective quantity of a tyrosine kinase inhibitor as described above.

The method can further comprise the administration of a therapeutically effective quantity of an epidermal growth factor receptor (EGFR) inhibitor as described above. The EGFR inhibitor can affect either wild-type binding sites or mutated binding sites, including EGFR Variant III, as described above.

Additionally, to treat brain metastases of NSCLC, the method can further comprise administering to the patient a therapeutically effective quantity of an agent that increases the ability of the substituted hexitol to pass through the blood-brain barrier. Alternatively, the method can further comprise administering to the patient a therapeutically effective quantity of an agent to counteract myelosuppression.

The invention is illustrated by the following Examples. These Examples are included for illustrative purposes only, and are not intended to limit the invention.

### Example 1

### In Vivo Efficacy of Dianhydrogalactitol in the Treatment of Non-Small-Cell Lung Cancer Employing a Mouse Xenograft Model

### Background

The median overall survival time for patients with stage IV non-small cell lung cancer (NSCLC) is 4 months, and 1- and 5-year survival is less than 16% and 2%, respectively. NSCLC is usually treated with surgery followed by treatment with either Tyrosine Kinase Inhibitors (TKIs) (e.g., erlotinib, gefitinib) or platinum-based regimens (e.g. cisplatin). TKIs have resulted in vastly improved outcomes for patients with EGFR mutations; however, TKI resistance has emerged as a significant unmet medical need, and long-term prognosis with platinum-based therapies is poor. Additionally, the incidence of brain metastases is high in patients with NSCLC with a poor prognosis.

Dianhydrogalactitol is a structurally unique bi-functional alkylating agent mediating interstrand DNA crosslinks at targeting N⁷ of guanine, thus differing in mechanism of action from TKIs and cisplatin. Dianhydrogalactitol further crosses the blood-brain barrier and accumulates in tumor tissue. Dianhydrogalactitol has demonstrated activity against NSCLC in preclinical and clinical trials, both as a single agent and in combination with other treatment regimens, suggesting dianhydrogalactitol may be a therapeutic option for drug-resistant NSCLC and NSCLC patients with brain metastasis.

The purpose of the study reported in this Example is to evaluate the activity of dianhydrogalactitol in in vivo models of drug-resistant NSCLC in comparison to other drugs, including cisplatin. Rag2 mice bearing subcutaneous human lung adenocarcinoma xenograft tumors of either TKI-resistant (H1975) or TKI-sensitive (A549) origin were treated.

Cell Lines and Animals

Two human NSCLC cell lines, A549 (TKI-sensitive) and H1975 (TKI-resistant), were used as xenograft tumor models in female Rag2 mice. The mice were 6 to 8 weeks of age and weighed 18-23 grams. 10 mice were used per group. The results reported below are for the A549 NSCLC cell line.

Drugs

Cisplatin was used in normal saline at a dose of 5 mg/kg. Administration was intravenous.

Dianhydrogalactitol was used in 0.9% sodium chloride for injection at 1.5 mg/kg to 6 mg/kg. Administration was intraperitoneal.

The study grouping was as shown in Table 1, below (“VAL-083” is dianhydrogalactitol).

TABLE 1
Study Grouping

| | | | TA/CA* | | | |
|---|---|---|---|---|---|---|
| | Group | No. | Dose | Admin. | Volume | Timepoint/ |
| Gp# | Name | mice | (mg/kg) | Route | (uL/20 g) | Schedule |
| 1 | Untreated | 10 | — | | | |
| | control | | | | | |
| 2 | Cisplatin | 10 | 5 | i.v. | 200 | Q7D X 3 |
| | control | | | | | |
| 3 | VAL-083 | 10 | 1.5 | i.p. | 200 | M, W, F X 3 |
| | dose 1 | | | | | |
| 4 | VAL-083 | 10 | 3 | i.p. | 200 | M, W, F X 3 |
| | dose 2 | | | | | |
| 5 | VAL-083 | 10 | 6 | i.p. | 200 | M, W, F X 3 |
| | dose 3 | | | | | |

*TA: Test Article; CA: Control Article

Treatment was initiated at a tumor volume of 100 mm³ to 150 mm³.

Experimental Design

Cell Preparation and Tissue Culture.

The A549 human lung carcinoma cell line had been obtained from the American Type Culture Collection (Cat. # CCL-185). The cells were started from a frozen vial of lab stock that were frozen down from the ATCC original vial and kept in liquid nitrogen. Cell cultures with a passage number of 3 to 10 and a confluence of 80%-90% were used. Cells were grown in RPMI 1640 supplemented with 10% fetal bovine serum and 2 mL L-glutamine at 37° C. in 5% CO₂ environment. Cells were subcultured once weekly with a split ratio 1:3 to 1:8 and expanded.

For cell preparation and harvesting for subcutaneous (s.c.) inoculation, the cells were rinsed briefly once with Hanks Balanced Salt Solution without calcium or magnesium. Fresh trypsin/EDTA solution (0.25% trypsin with tetrasodium EDTA) was added, the flask was laid horizontally to ensure that the cells were covered by trypsin/EDTA, and the extra trypsin/EDTA was aspirated. The cells were allowed to sit at 37° C. for a few minutes. The cells were observed under an inverted microscope until the cell layer was dispersed, fresh medium was added, 50 μL of cell suspension was taken and mixed with trypan blue (1:1), and the cells were counted and cell viability assessed by using Cellometer Auto T4. The cells were centrifuged at 200×g for 7 minutes and the supernatant was aspirated. The cells were resuspended in growth medium to obtain a concentration of 100×10⁶ cells/mL. For inoculation, 5×10⁶ cells were used in an injection volume of 50 μL per mouse in 1:1 Matrigel™.

Tumor Cell Implantation

On day 0, tumor cells were implanted subcutaneously into mice in a volume of 50 μL in Matrigel™ using a 28-gauge needle; injection of the tumor cells was in the back of the mice. Mice were randomly assigned to groups based on tumor volume. The means of the tumor volumes prior at the time of randomization were 89.15 mm³, 86.08 mm³, 95.49 mm³, 87.15 mm³ and 81.76 mm³ for groups 1-5, respectively.

Dose Administration

Dianhydrogalactitol (DAG) was provided as a lyophilized product at 40 mg of DAG per vial. For administration, 5 mL of 0.9% sodium chloride for injection, USP (saline) was added to yield a DAG solution with a concentration of 8 mg/mL. This stock solution was stable for 4 hours at room temperature or for 24 hours at 4° C. Further dilutions were made to prepare solutions of injection of 0.9 mg/mL (for administration of 0.18 mg/mouse in 0.2 mL; diluted from the 8 mg/mL reconstituted solution); of 0.45 mg/mL (for administration of 0.09 mg/mouse in 0.2 mL; a 1 to 2 dilution of the 0.9 mg/mL solution); and of 0.225 mg/mL (for administration of 0.045 mg/mouse in 0.2 mL; a 1 to 2 dilution of the 0.45 mg/mL solution).

Intravenous Injections

Mice were injected with the required volume to administer the prescribed dose (mg/kg) to the animals based on individual mouse weights using a 28-gauge needle. The injection volume was 200 μL for a 20-g mouse. The mice were briefly (less than 30 seconds) restrained during intravenous injections. Dilation of the vein for intravenous injections was achieved by holding the animals under a heat lamp for a period of between 1-2 minutes.

Intraperitoneal Injections

Mice were individually weighed and injected intraperitoneally according to body weight at the specified injection concentration (see Table 1). The injection volume was based on 200 μL per 20-g mouse. The abdominal surface was wiped down with 70% isopropyl alcohol to clean the injection site.

Data Collection

Tumor Monitoring

Tumor growth was monitored by measuring tumor dimensions with calipers beginning on the first day of treatment. Tumor length and width measurements were obtained each Monday, Wednesday, and Friday. Tumor volumes were calculated according to the equation L×W²/2 with the length (in mm) being defined as the longer axis of the tumor. Animals were weighed at the time of tumor measurement. Tumors were allowed to grow to a maximum of 800 mm³ before termination.

All animals had blood collected by cardiac puncture at termination for CBC (complete blood count) with differentiation. Statistical significance (p<0.05) between untreated control and groups 4 or 5 (dianhydrogalactitol-treated groups) was found for hemoglobin (g/L) for CBC analysis. Differential analysis was performed; however, it is noted that even in control mice there are low white blood cell (WBC) numbers (due to the fact that the strain is immunocompromised, which would affect WBC production). For WBC, statistical significance (p<0.05) was observed for lymphocytes and eosinophils. There were no differences between control non-tumor bearing animals (mouse ID # control 1 and control 2) and untreated control tumor-bearing animals (group 1; mouse ID #1-10) for CBC/differential analyses.

Observations of Animals

Clinical Observations

All animals were observed post-administration, and at least once per day, more frequently if deemed necessary, during the pre-treatment and treatment periods for morbidity and mortality. In particular, signs of ill-health were based on body weight loss, change in appetite, and behavioral signs such as altered gait, lethargy, and gross manifestations of stress. If signs of severe toxicity or tumor-related illness were seen, the animals were terminated by isoflurane overdose followed by CO₂ asphyxiation, and a necropsy was performed to assess other signs of toxicity. The following organs were examined: liver, gall bladder, spleen, lung, kidney, heart, intestine, lymph nodes, and bladder. Any unusual findings were noted.

The methodology was reviewed and approved by the Institutional Animal Care Committee (IACC) at the University of British Columbia. The housing and use of animals were performed in accordance with the Canadian Council on Animal Care Guidelines.

Summaries for the administration of dianhydrogalactitol (“VAL-083”) and cisplatin are shown in Tables 2-3, below:

TABLE 2
Administration of Dianhydrogalactitol

| GROUP# | | DOSE | | | | | | | | |
|---|---|---|---|---|---|---|---|---|---|---|
| VAL-083 | TREATMENT | mg/kg | MICE/ | AVR. | CONC. | INJECTED | TOTAL | TOTAL | STOCK | Saline |
| Stock conc. | 0.80 * | mg/ml | group | WT g | mg/ml | ml/20 g | ml | mg | ml | ml |
| 3 | VAL-083 | 1.5 | 10 | 20.0 | 0.150 | 0.200 | 3.00 | 0.450 | 0.563 | 2.438 |
| 4 | VAL-083 | 3.0 | 10 | 20.0 | 0.300 | 0.200 | 3.00 | 0.900 | 1.125 | 1.875 |
| 5 | VAL-083 | 6.0 | 10 | 20.0 | 0.600 | 0.200 | 3.00 | 1.800 | 2.250 | 0.750 |
| | | | | | | Total: | 9.00 | 3.150 | 3.938 | |

TABLE 3
Administration of Cisplatin

| | | Dose, | Mice/ | Average | Conc., | Injected, | Total, | Total, | Stock. | Saline, |
|---|---|---|---|---|---|---|---|---|---|---|
| Group # | Treatment | mg/kg | Group | Weight, g | mg/mL | ml/20 g | mL | mg | mL | mL |
| Cisplatin | Cisplatin | 5.0 | 10 | 20.0 | 0.500 | 0.200 | 3.00 | 1.500 | 1.500 | 1.500 |
| Control | | | | | | | | | | |

Results and Conclusion

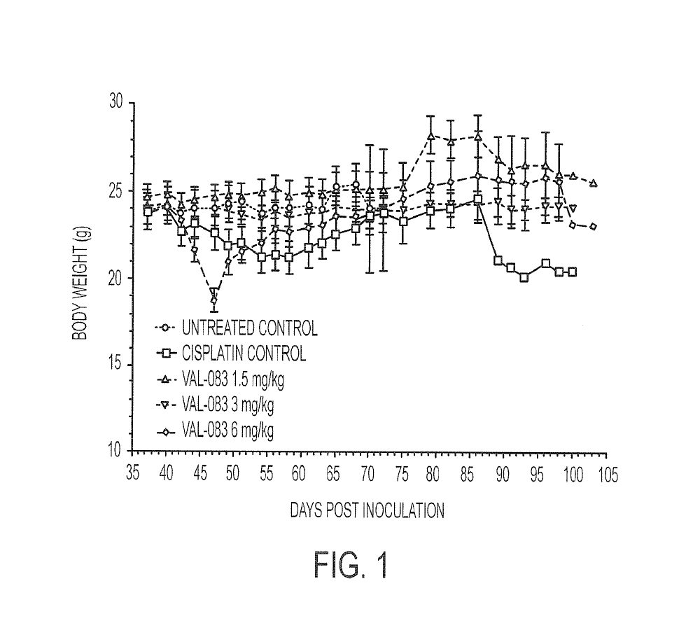

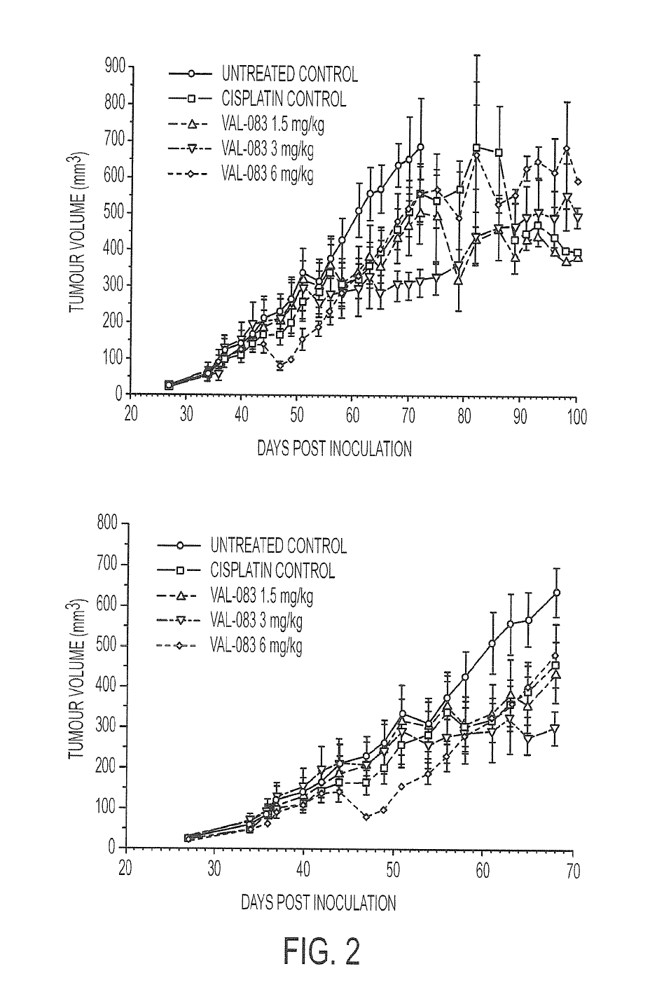

The results are shown in FIGS. 1-2.

FIG. 1 shows body weight on the y-axis versus days post-inoculation on the x-axis. In FIGS. 1-2, • is the untreated control; ▪ is the cisplatin control; ▴ is dianhydrogalactitol at 1.5 mg/kg; ▴ is dianhydrogalactitol at 3.0 mg/kg; and □ is dianhydrogalactitol at 6.0 mg/kg.

According to the results of FIG. 1, body weight loss was observed in mice treated with 5 mg/kg cisplatin (group 2) and 6 mg/kg dianhydrogalactitol (group 5). Group 5 treatment was stopped after 3 doses due to significant body weight loss. Body weights are shown as means±S.D.

FIG. 2 shows the tumor volume (means±S.E.M.) for the A549 tumor-bearing female Rag2 mice with tumor volume on the y axis versus days post-inoculation on the x-axis. The top panel of FIG. 2 represents all mice for the complete duration of the study. The bottom panel of FIG. 2 represents all mice until day 70 (last day for untreated control group).

To summarize the results, mice were administered with untreated control (group 1), Cisplatin at 5 mg/kg Q7D×3 i.v. (group 2) or dianhydrogalactitol at 1.5 mg/kg i.p. (group 3), 3 mg/kg (group 4), and 6 mg/kg (group 5) Monday, Wednesday, Friday for 3 weeks and tumor volume was measured 3×weekly and summarized in FIG. 2. The top panel indicates tumor volume for all animals and the bottom panel shows results for animal until day 70. Note that the number of animals remaining on study on day 70 was 2/10 (group 1), 6/10 (group 2), 7/10 (group 3), 6/10 (group 4) and 8/10 (group 5). For groups 1-5, a mean tumor volume of 200 mm³ was observed on days 43, 49, 45, 42 and 54, respectively. For groups 1-4, a mean tumor volume of 400 mm³ was reached on days 56, 66, 67 and 81 respectively. The doubling times for groups 1-4 were 13, 17, 22 and 39, respectively. A tumor growth delay of 26 days was observed in animals administered 3 mg/kg dianhydrogalactitol compared to untreated controls. The positive control of 5 mg/kg cisplatin had a tumor growth delay of only 4 days in comparison.

In terms of the tolerability of the dosages, dianhydrogalactitol at 6 mg/kg resulted in significant weight loss and morbidity of the mice and only 3 of the 9 scheduled doses were administered. The 5 mg/kg dose of cisplatin may also be near the MTD as 1 mouse was unable to receive the last dose.

In conclusion, administration of dianhydrogalactitol at a dose of 3 mg/kg resulted in a significant tumor growth delay as compared to cisplatin at 5 mg/kg.

### Example 2

### Response to Dianhydrogalactitol With or Without Radiation Therapy in Primary Glioblastoma Multiforme Cultures

The standard of care for glioblastoma multiforme (GBM) patients is surgical resection followed by temozolomide (TMZ) and radiation (XRT). TMZ is most effective for a minority of patients that exhibit epigenetic inactivation of O⁶-methylguanine DNA methyltransferase (MGMT), a DNA repair enzyme that removes the methyl-group adducts that are caused by TMZ. Thus, adducts that are not subject to the DNA repair mechanism of MGMT might provide additional benefit to GBM patients, the majority of which express MGMT and are TMZ-resistant, or acquire resistance after TMZ administration. The N7 alkylating agent, dianhydrogalactitol (“VAL-083”), is not subject to MGMT mediated repair and might therefore be a more potent chemotherapeutic. Dianhydrogalactitol is a first-in-class alkylating agent that crosses the blood brain barrier and is currently in clinical trials for glioma patients with recurrent disease. We have recently shown that cancer stem cells (CSC) and their paired non-CSC cultures derived from primary GBM tissues exhibit similar responses to TMZ, with this response dependent on the presence or absence of MGMT expression. We sought to investigate how our panel of stem and non-stem cultures responds to dianhydrogalactitol alone or in combination with XRT, and how the response would compare to TMZ.

A summary of the cultures tested is shown in Table 4. “VAL” refers to dianhydrogalactitol and “XRT” refers to radiation. “CSC” refers to cancer stem cells, while “non-CSC” refers to non-cancer-stem cell cultures.

TABLE 4

| | | | | | Cell | Cell |
|---|---|---|---|---|---|---|
| | | | FACS | FACS | Viability | Viability |
| | FACS | FACS | VAL/ | VAL/ | VAL/ | VAL/ |
| Cell Line | Val#1 | Val#2 | XRT#1 | XRT#2 | XRT#1 | XRT#2 |
| 7996 CSC | X | X | X | | X | |
| 7996 Non- | X | X | X | | X | |
| CSC | | | | | | |
| 8161 CSC | X | X | X | | X | |
| 8161 Non- | X | X | | | | |
| CSC | | | | | | |
| 8279 CSC | X | | | | | |
| 8565 CSC | X | | X | | X | |
| 8565 Non- | X | X | | | | |
| CSC | | | | | | |
| 9030 CSC | X | | X | | X | |
| U251 | X | X | X | | X | |

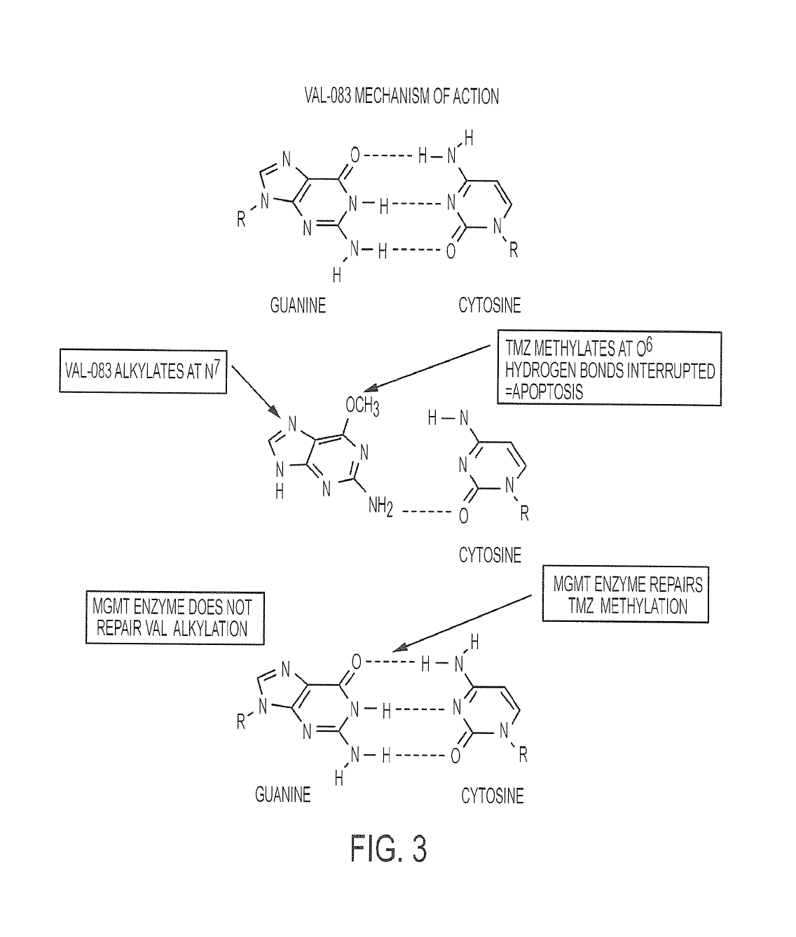

The mechanism of action for dianhydrogalactitol (“VAL-083”) is shown in FIG. 3.

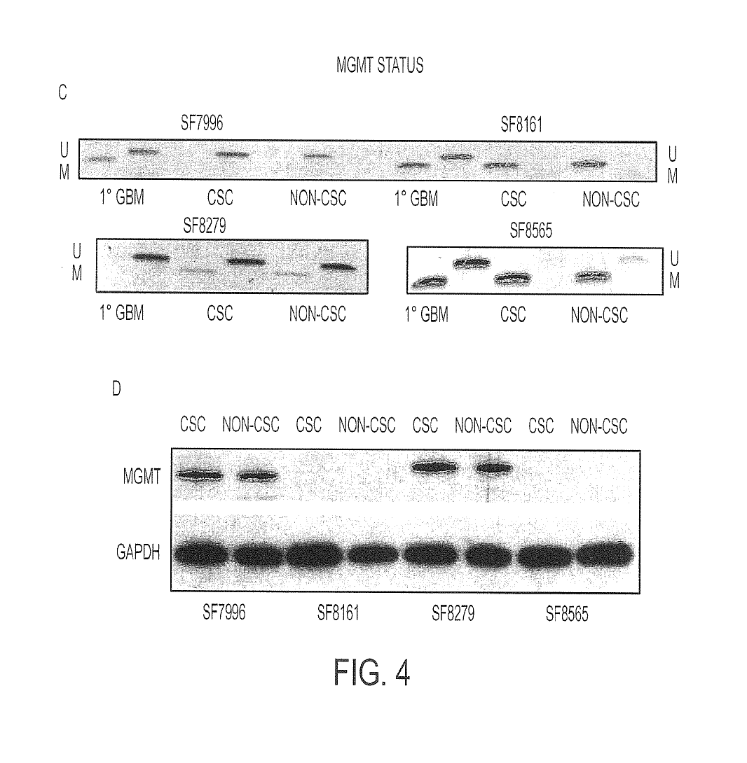

FIG. 4 shows the MGMT status of the cultures. “GAPDH” refers to glyceraldehyde-3-phosphate dehydrogenase as a control. For the cell cultures, CSCs were cultured in NSA media supplemented with B27, EGF and bFGF. Non-CSCs were grown in DMEM:F12 with 10% FBS. MGMT methylation and protein expression analysis of each culture was characterized. TMZ or VAL-083 was added to the cultures in the indicated concentrations. Depending on the experiment, cells were also irradiated with 2 Gy in a Cesium irradiator. For assays, cell cycle analysis was performed with Propidium Iodide staining and FACs analysis. Cell viability was analyzed with CellTiter-Glo™ and read on a Promega GloMax™. In FIG. 4, Panel C shows the methylation status of MGMT for cell lines SF7996, SF8161, SF8279, and SF8565; “U” refers to unmethylated and “M” refers to methylated. In FIG. 4, “1° GBM” refers to primary glioblastoma multiforme cell cultures. FIG. 4 shows MGMT western blot analysis of protein extracts from 4 pairs of CSC and non-CSC cultures derived from primary GBM tissue.

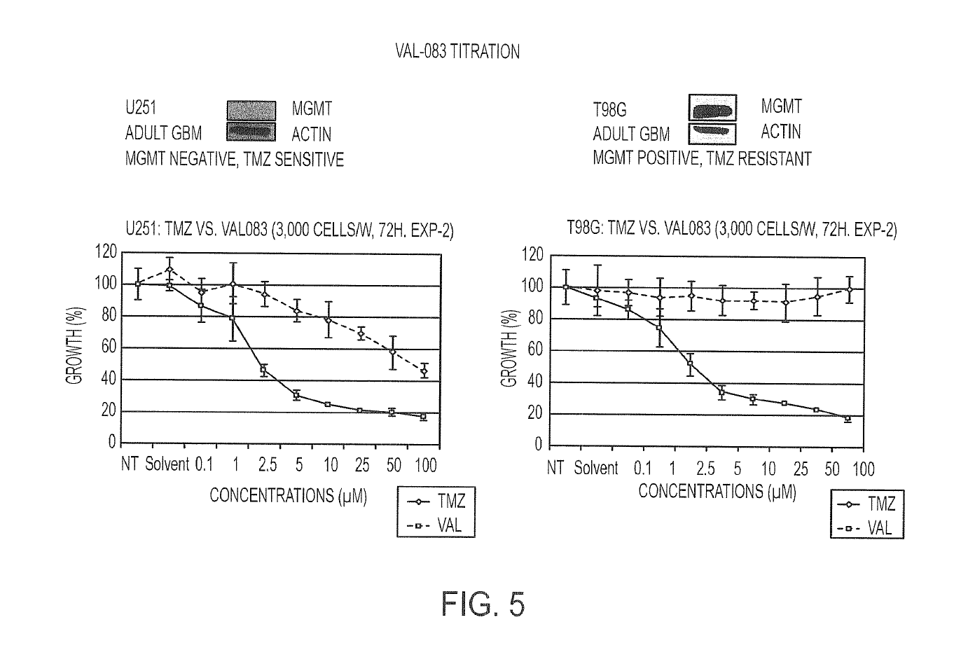

FIG. 5 shows that dianhydrogalactitol (“VAL-083”) was better than TMZ for inhibiting tumor cell growth and that this occurred in an MGMT-independent manner.

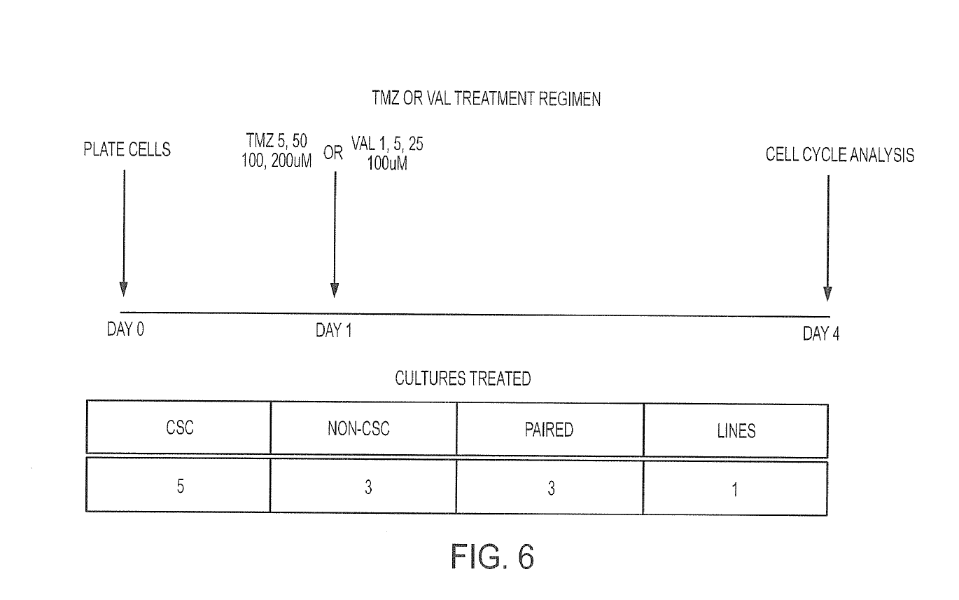

FIG. 6 shows schematics of various treatment regimens for temozolomide (“TMZ”) or dianhydrogalactitol (“VAL”), with or without radiation (“XRT”).

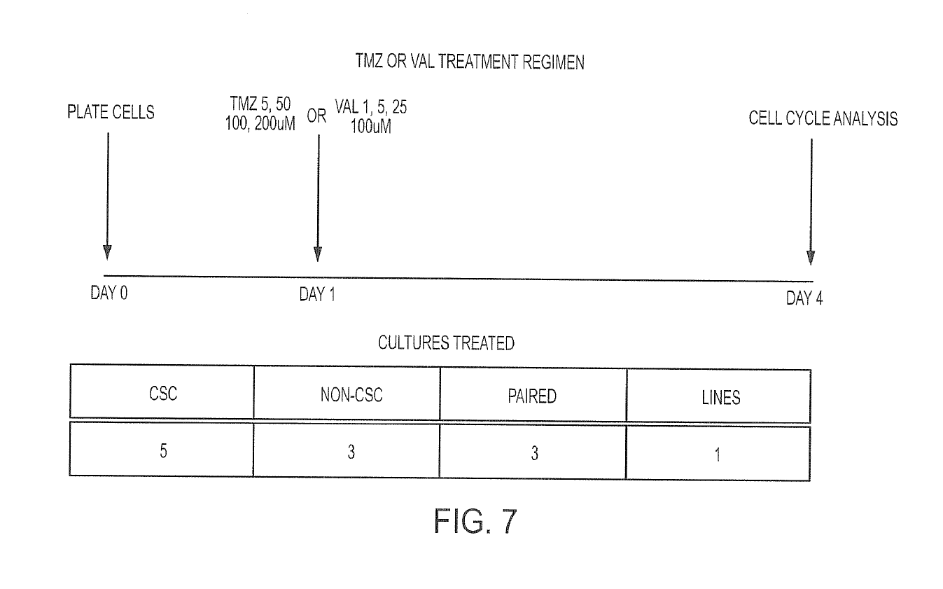

FIG. 7 shows cell cycle analyses for cancer stem cells (CSC) treated with TMZ or dianhydrogalactitol (“VAL-083”), for 7996 CSC, 8161 CSC, 8565 CSC, and 8279 CSC. In these cell cycle analyses, G2 is shown at the top, S in the middle, and G1 at the bottom.

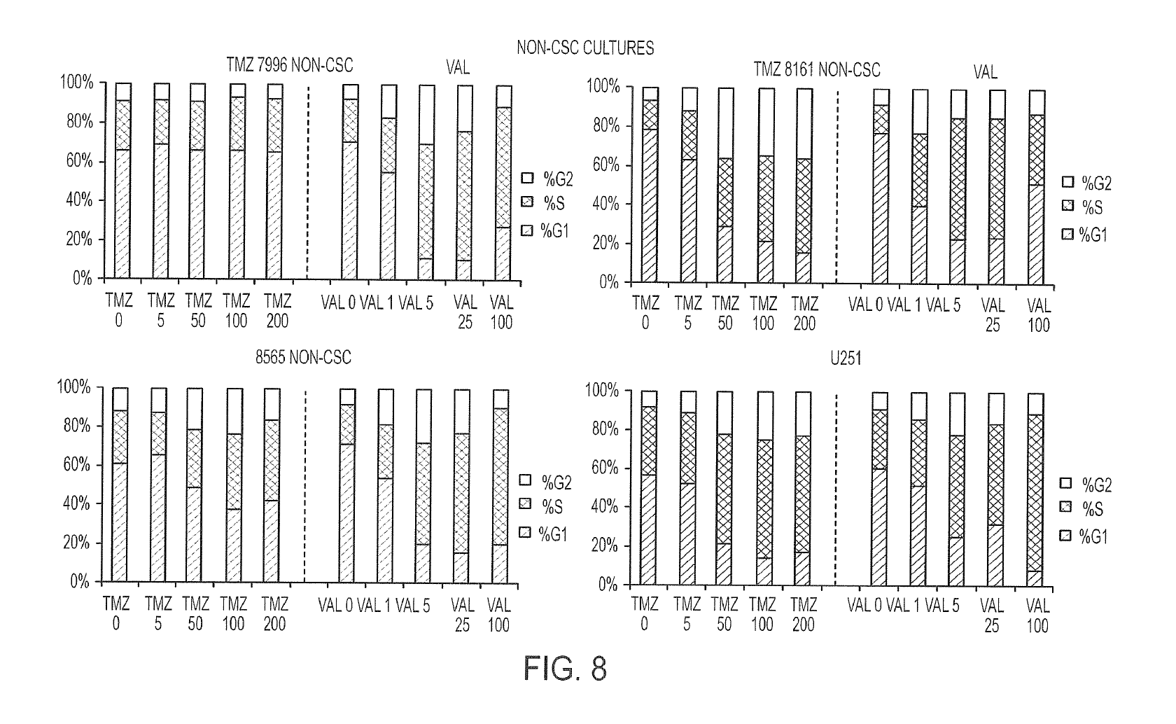

FIG. 8 shows cell cycle analyses for non-stem-cell cultures treated with TMZ or dianhydrogalactitol (“VAL-083”), for 7996 non-CSC, 8161 non-CSC, 8565 non-CSC, and U251. In these cell cycle analyses, G2 is shown at the top, S in the middle, and G1 at the bottom.

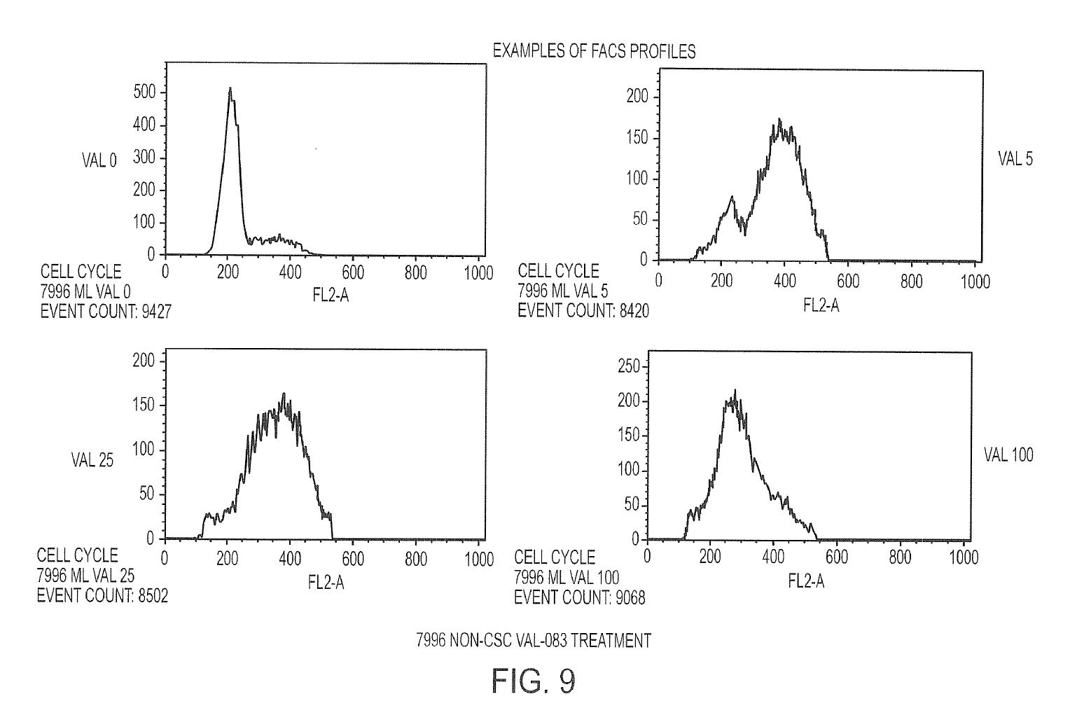

FIG. 9 shows examples of FACS profiles for 7996 non-CSC dianhydrogalactitol (“VAL”) treatment.

Regarding these results, dianhydrogalactitol appears to cause cell death at lower concentrations than temozolomide. Odd cell cycle profiles appear in some cultures; in some cases, there is a dip in G1 at a small dianhydrogalactitol dose (1-5 μM) and then G1 appears to recover at a larger dose (100 μM). The activity of dianhydrogalactitol is not affected by MGMT status or the stem-cell or non-stem-cell status of the culture.

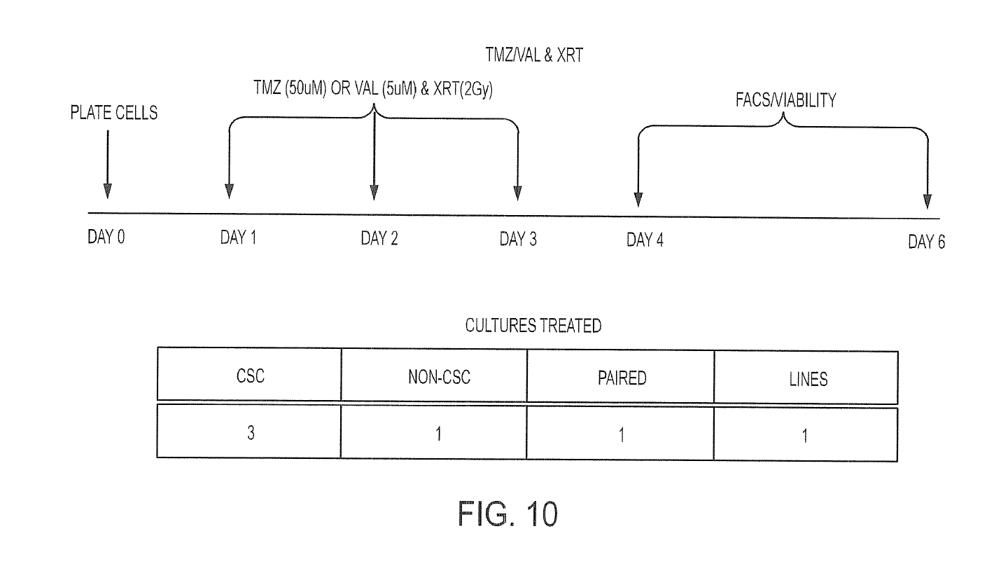

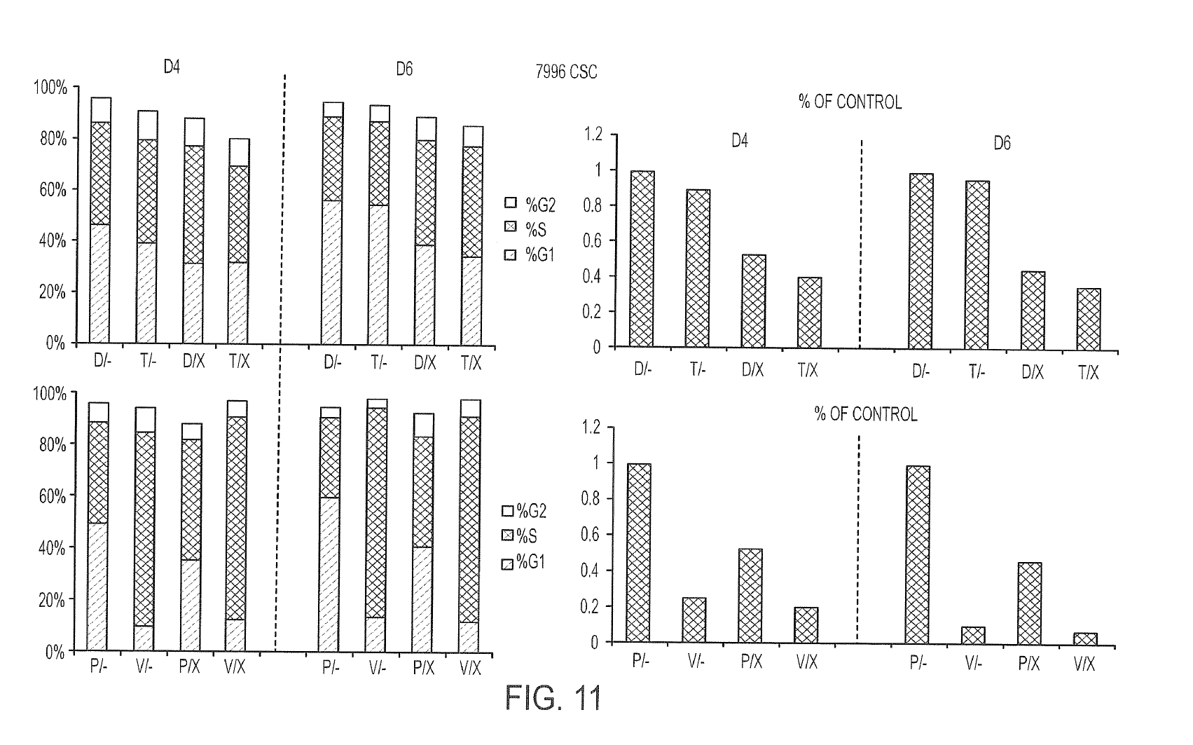

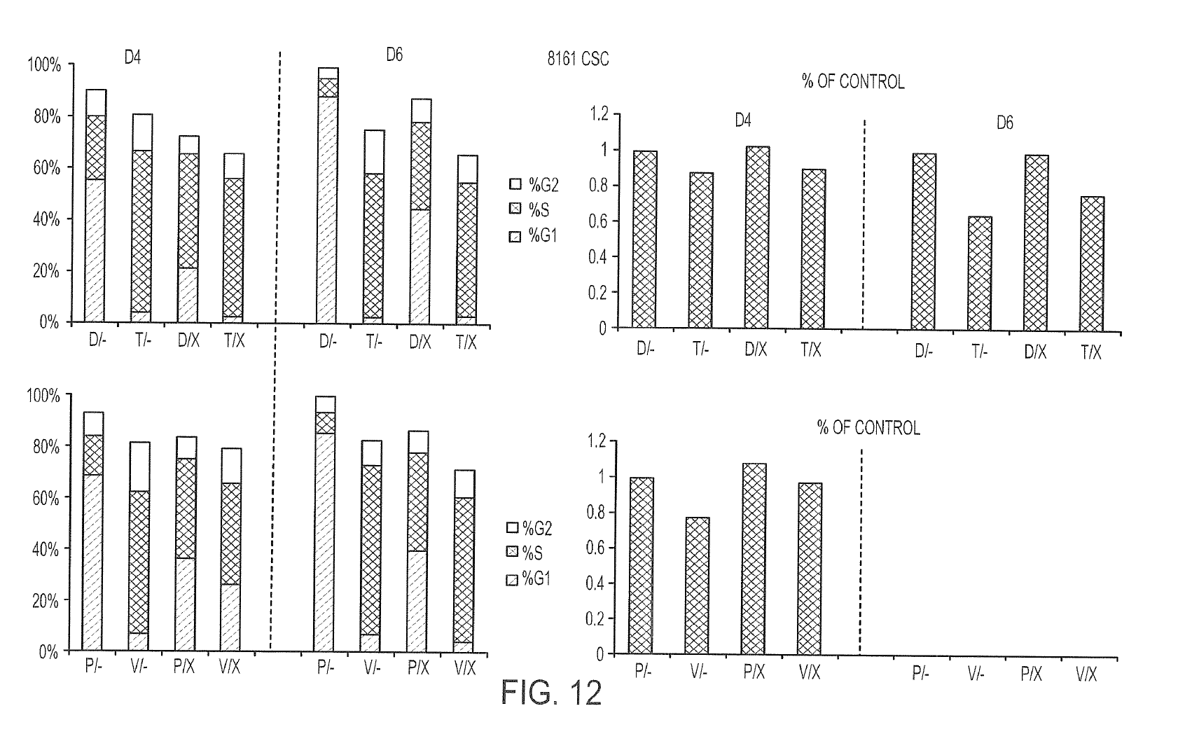

FIG. 10 shows a schematic of the treatment regimen using either temozolomide (“TMZ”) or dianhydrogalactitol (“VAL”) and radiation (“XRT”).

FIG. 11 shows results for 7996 CSC for TMZ only, VAL only, and TMZ or VAL with XRT. In FIG. 11, for TMZ “-D/-” indicates DMSO only (vehicle), “-T/-” indicates TMZ only, and “-D/X” or “-T/X” indicate DMSO or TMZ with XRT. Similarly, for VAL, “—P/-” indicates phosphate buffered saline (PBS) only (vehicle), “—V/-” indicates VAL only, and “—P/X” or “—V/X” indicate PBS or VAL with XRT. The left side of FIG. 11 shows cell cycle analysis where G2 is shown at the top, S in the middle, and G1 at the bottom; both 4- and 6-day results are shown, with the 4-day results (“D4”) presented to the left of the 6-day results (“D6”). The right side of FIG. 11 shows the results for cell viability as a percentage of control for D4 and D6.

FIG. 12 shows results for 8161 CSC depicted as in FIG. 11.

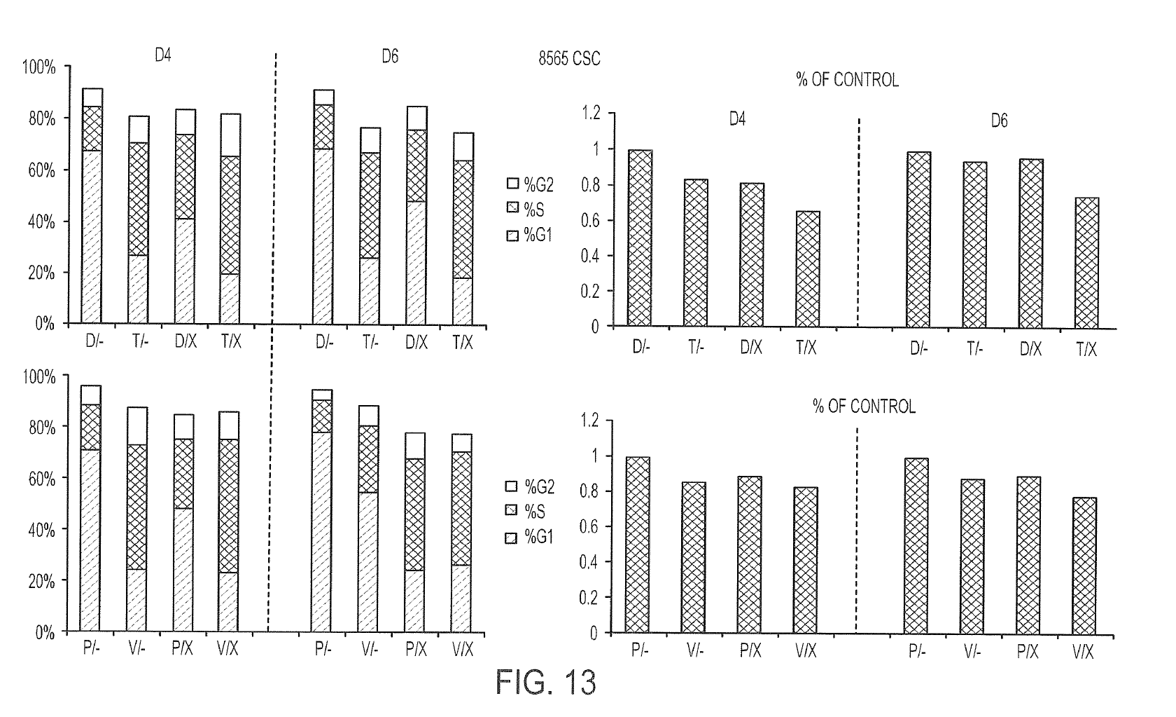

FIG. 13 shows results for 8565 CSC depicted as in FIG. 11.

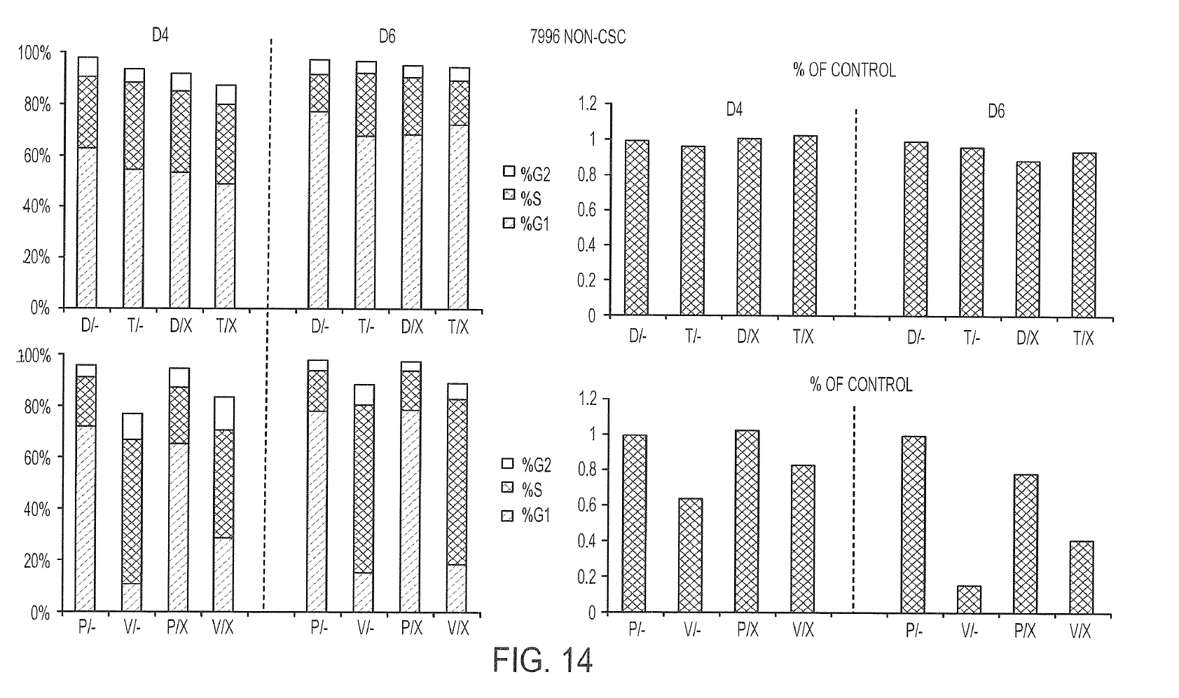

FIG. 14 shows results for 7996 non-CSC depicted as in FIG. 11.

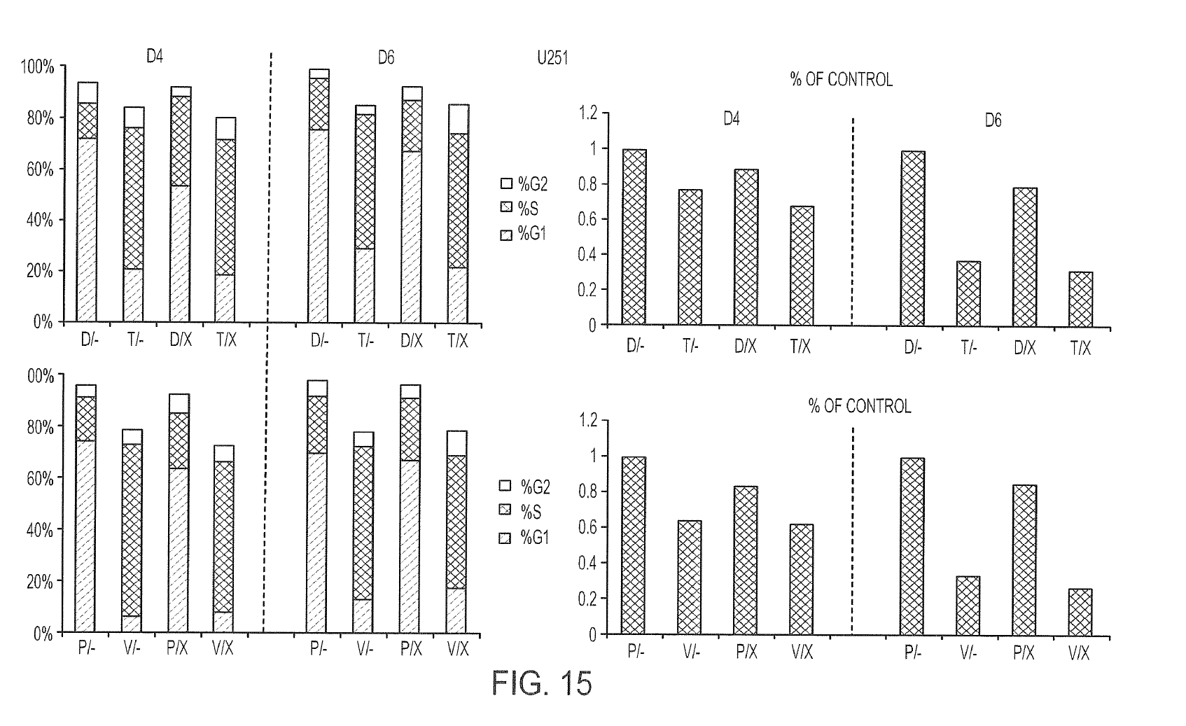

FIG. 15 shows results for U251 depicted as in FIG. 11.

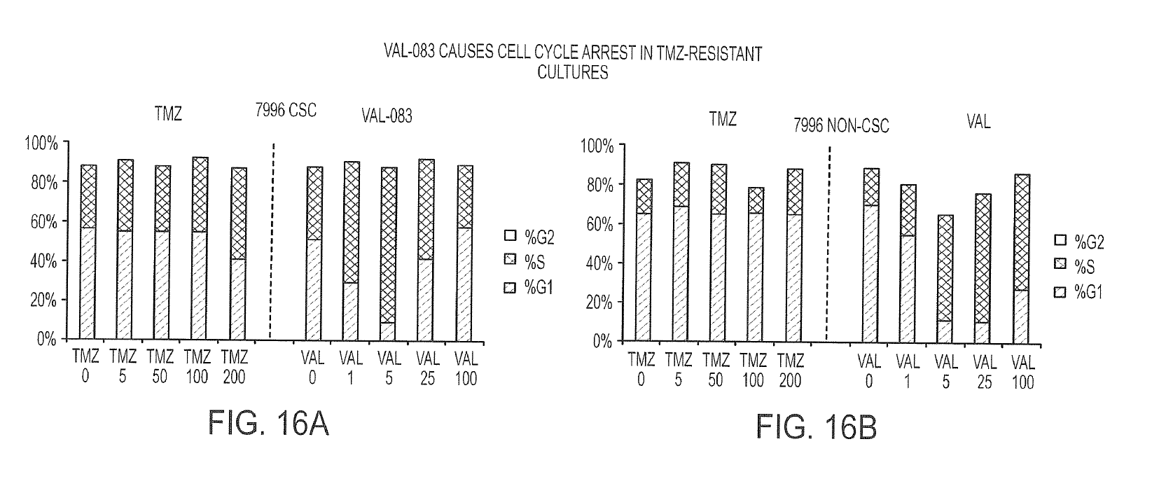

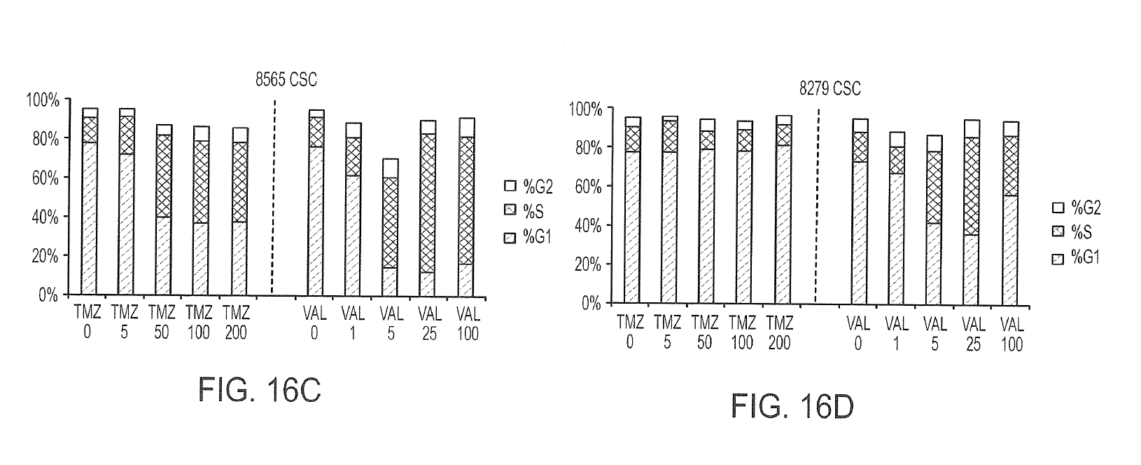

FIG. 16 shows that dianhydrogalactitol causes cell cycle arrest in TMZ-resistant cultures. In FIG. 16, cells were treated with either increasing doses of TMZ (5, 50 100 and 200 μM) or dianhydrogalactitol (“VAL-083”) (1, 5, 25 and 100 μM) and cell cycle analysis was performed 4 days post treatment. TMZ resistant cultures (A, B, D) exhibited sensitivity to VAL-083, even at single-micromolar doses. Furthermore, this response was not dependent on culture type as paired CSC (A) and non-CSC (B) both exhibit sensitivity to VAL-083.

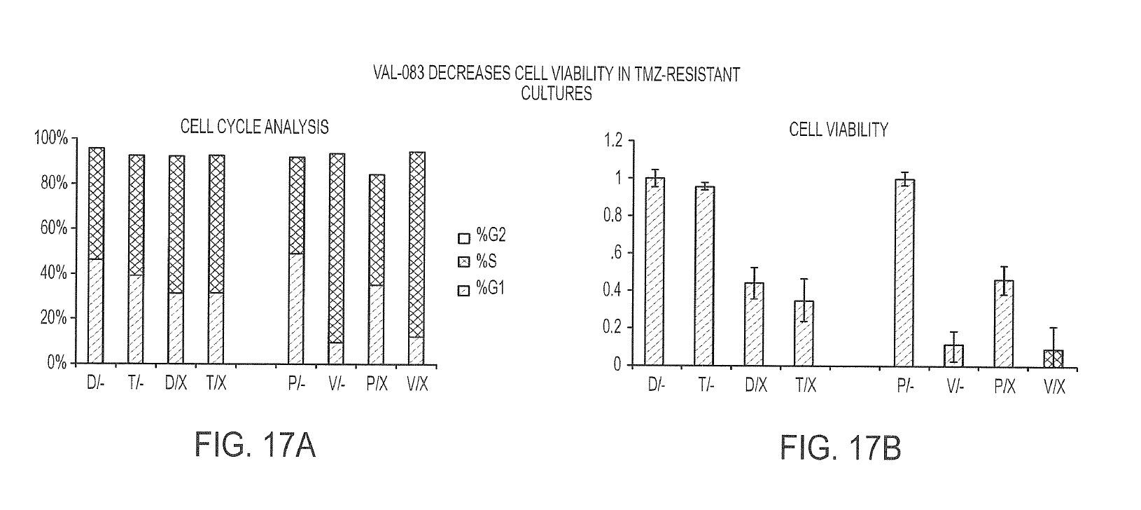

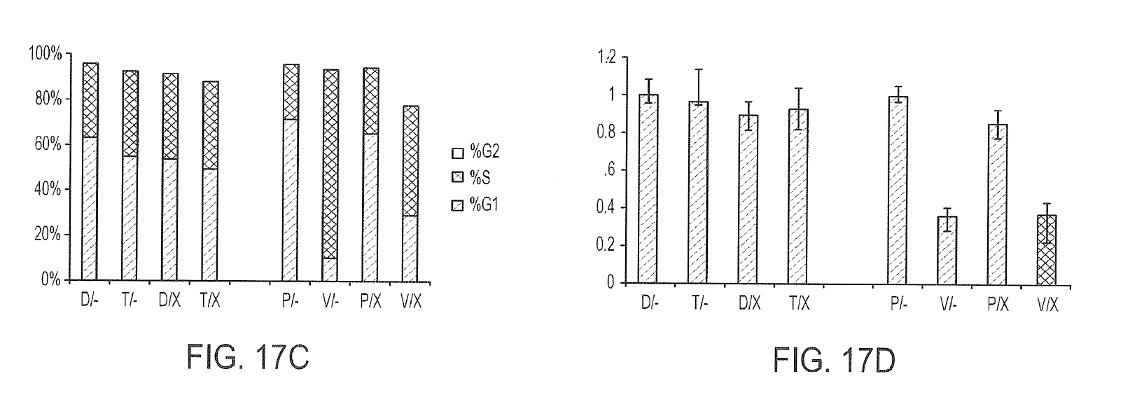

FIG. 17 shows that dianhydrogalactitol decreases cell viability in TMZ-resistant cultures. In FIG. 17, TMZ (50 μM) or dianhydrogalactitol (“VAL-083”) (5 μM) were added to primary CSC cultures at various doses with or without irradiation (2 Gy). Shown are cell cycle profile analysis at day 4 post treatment (A,C) and cell viability analysis at day 6 post treatment (B,D) for the paired CSC (A,B) and non-CSC (C,D) 7996 culture. Whereas these cultures are not very sensitive to TMZ, they are to VAL-083. However, the addition of radiation (XRT) in both cases does not result in increased sensitivity (D=DMSO, T=TMZ, X=XRT, P=PBS).

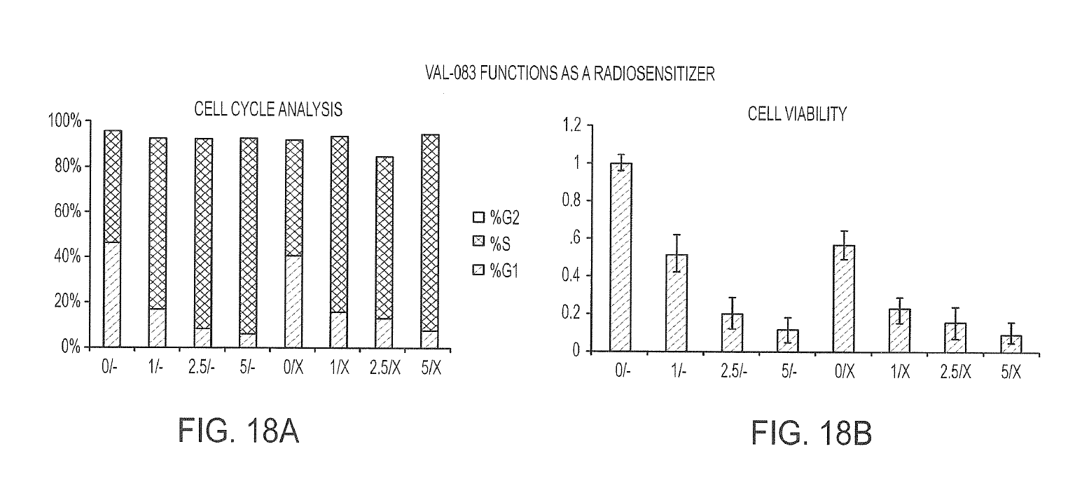

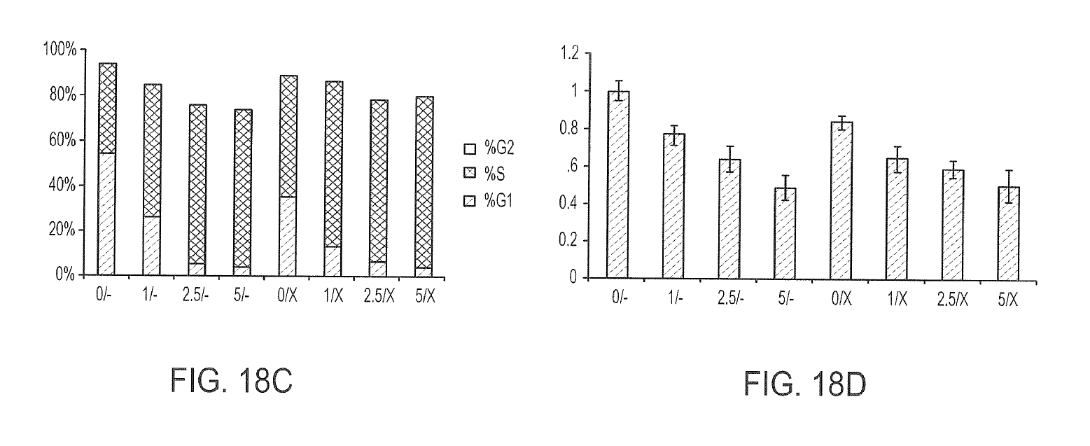

FIG. 18 shows that dianhydrogalactitol acts as a radiosensitizer in primary CSC cultures. In FIG. 18, dianhydrogalactitol (“VAL-083”) was added to primary CSC cultures at various doses (1, 2.5 and 5 μM) with or without irradiation (2 Gy). Shown are cell cycle profile analysis at day 4 post treatment (A,C) and cell viability analysis at day 6 post treatment (B,D) for two different patient-derived CSC cultures, 7996 (A,B) and 8565 (C,D).

Additional experiments were performed to test the effect of the duration of drug administration. Temozolomide was added for 3 hours and then washed out. Dianhydrogalactitol was left on for the duration of the treatment. These experiments were performed to determine the results if temozolomide was left on indefinitely or if dianhydrogalactitol was washed out after 3 hours.

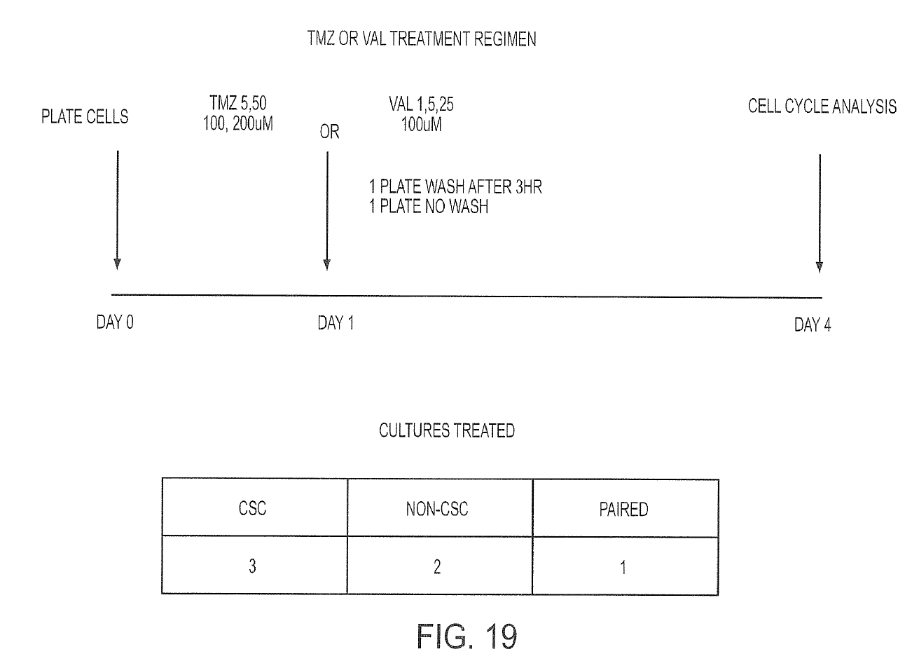

FIG. 19 shows the treatment regimens with a wash or no wash for both dianhydrogalactitol and temozolomide.

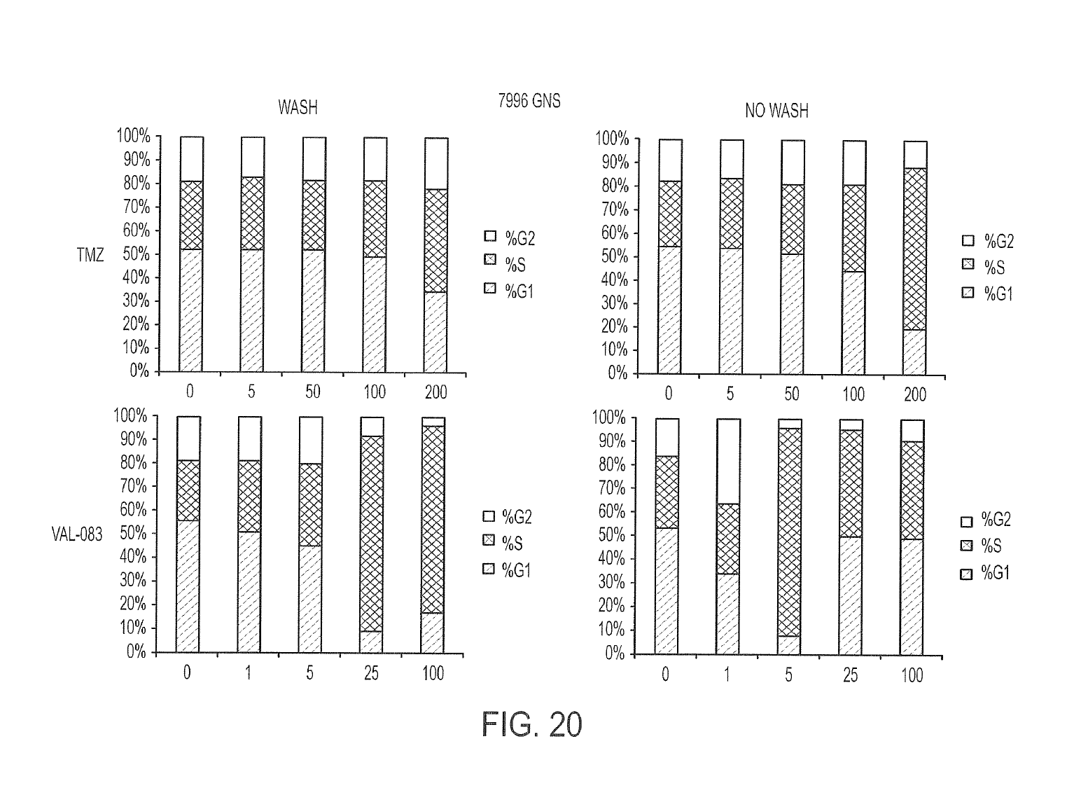

FIG. 20 shows the results for 7996 GNS, showing cell cycle analysis where G2 is shown at the top, S in the middle, and G1 at the bottom. Results for TMZ are shown on the top and results for dianhydrogalactitol on the bottom. Results with a wash are shown on the left and results without a wash are shown on the right.

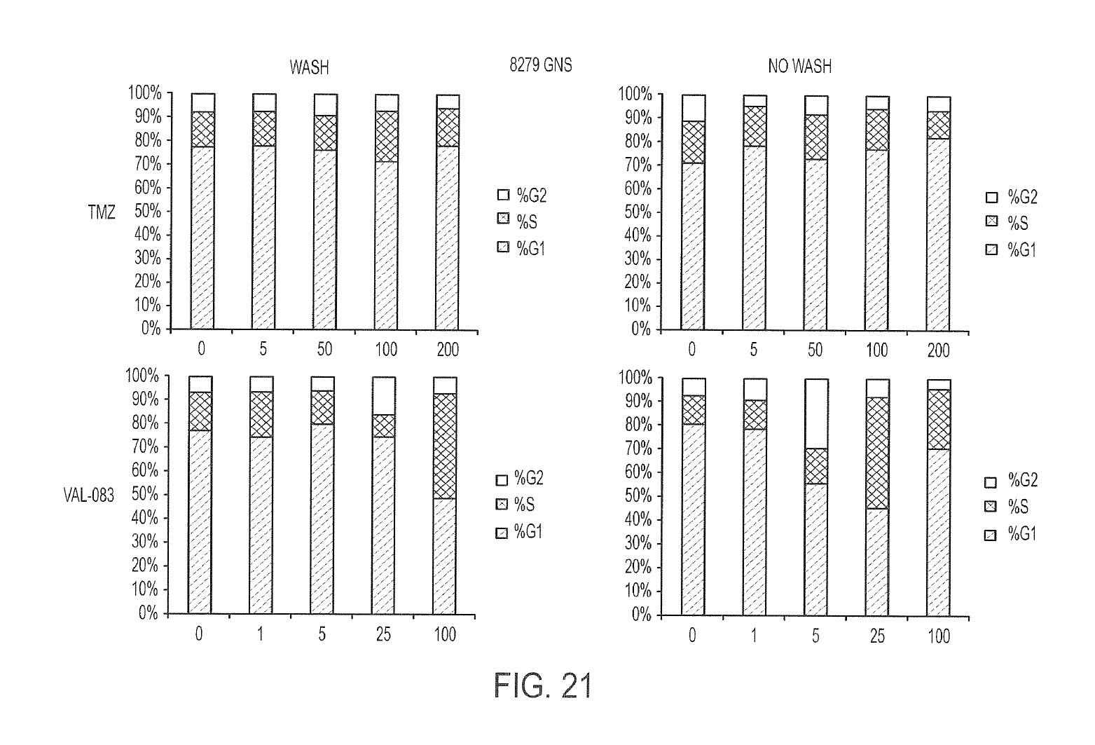

FIG. 21 shows the results for 8279 GNS, depicted as in FIG. 20.

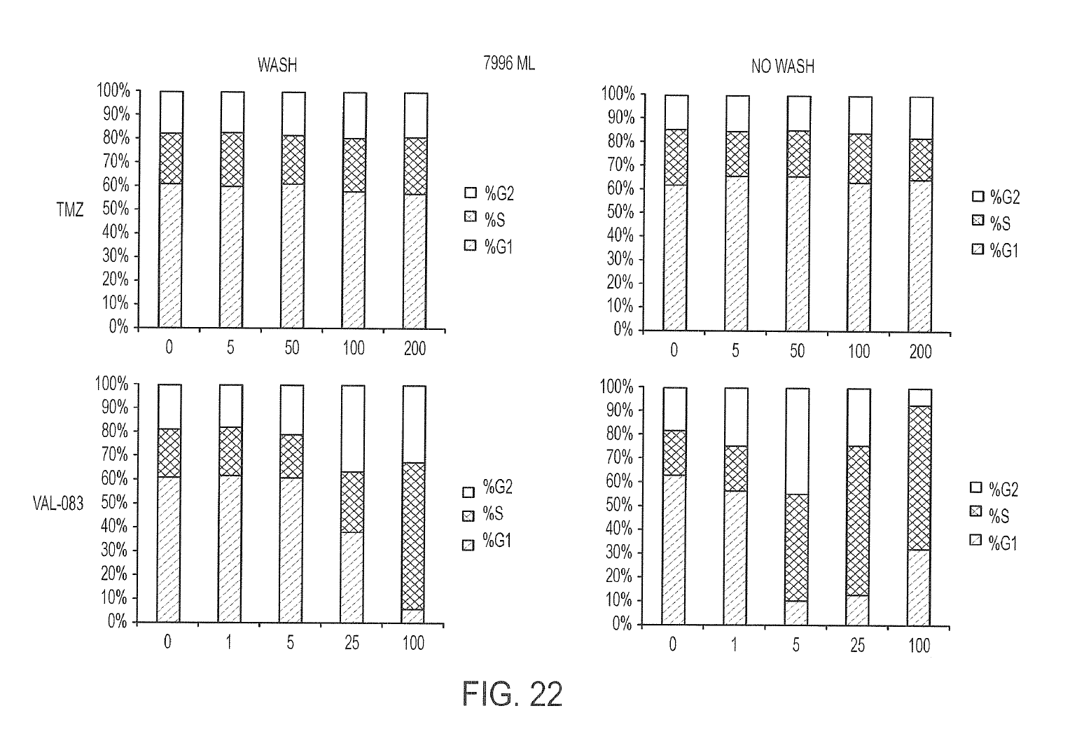

FIG. 22 shows the results for 7996 ML, depicted as in FIG. 20.

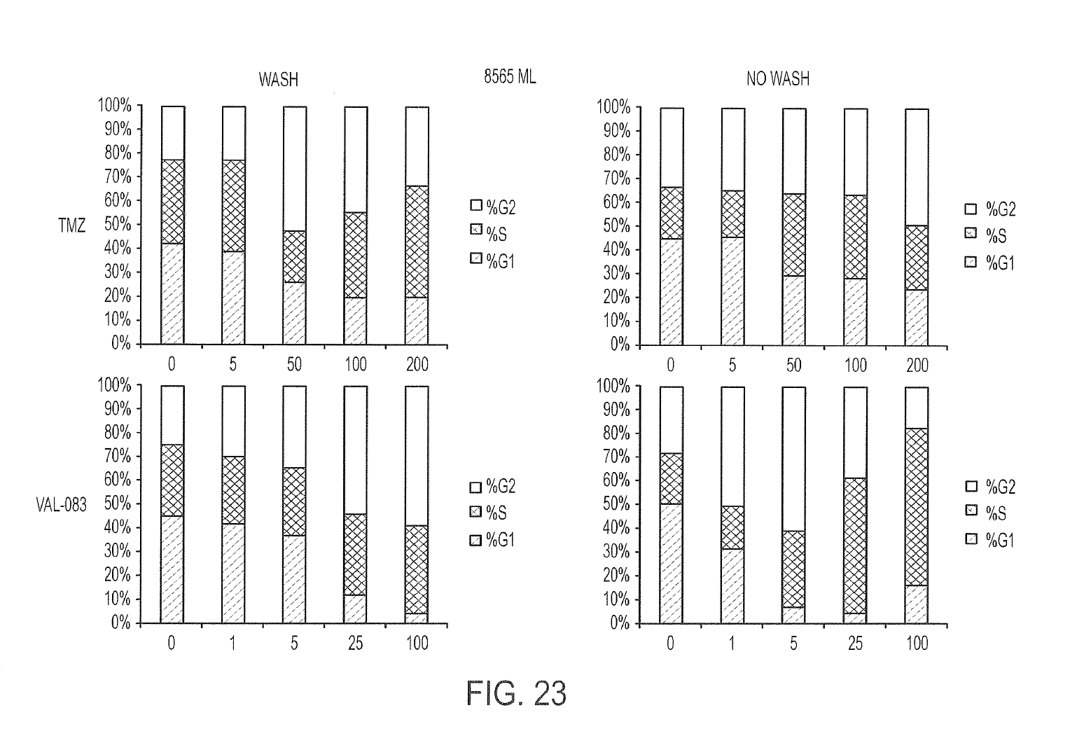

FIG. 23 shows the results for 8565 ML, depicted as in FIG. 20.

In these experiments, temozolomide did not appear to have any more effect if left on for longer than 3 hours. Dianhydrogalactitol had less effect when washed out after 3 hours.

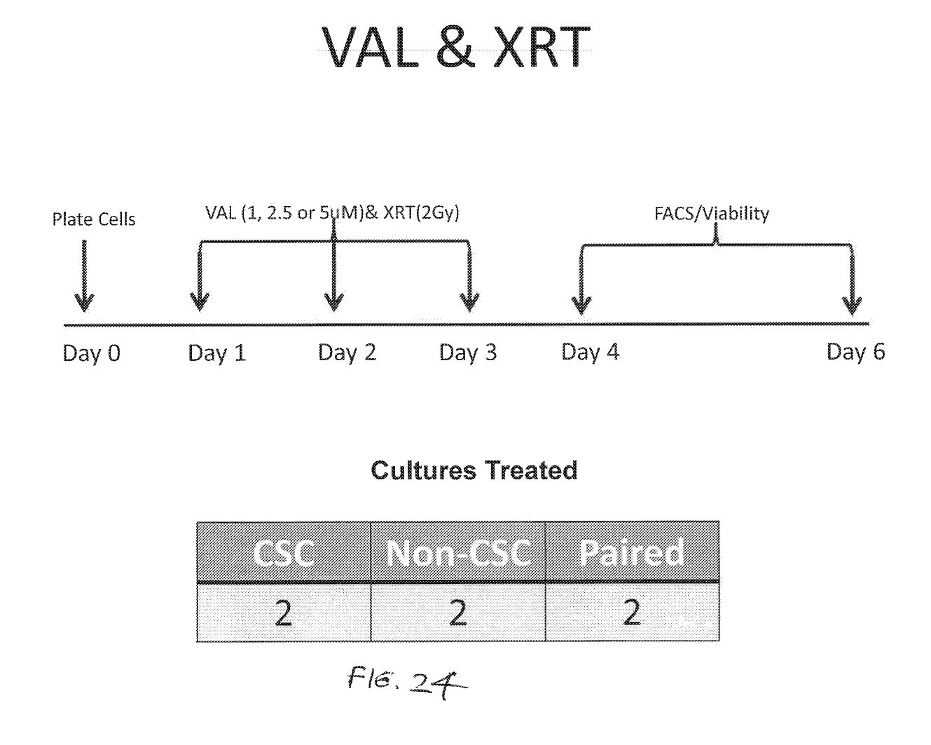

FIG. 24 shows the treatment regimens for combining dianhydrogalactitol (“VAL”) and radiation (“XRT”).

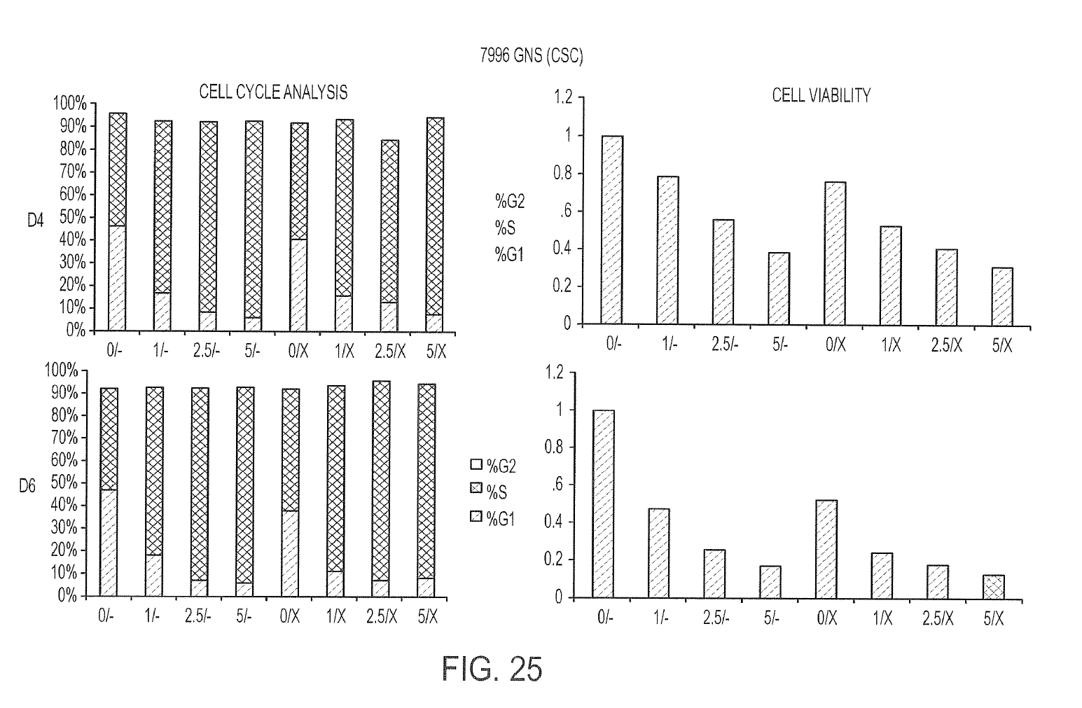

FIG. 25 shows the results for 7996 GNS (CSC) when dianhydrogalactitol is combined with radiation. Results are shown at day 4 (“D4”) on the top and day 6 (“D6”) on the bottom. The left side shows cell cycle analysis where G2 is shown at the top, S in the middle, and G1 at the bottom. The right side shows cell viability at D4 and D6.

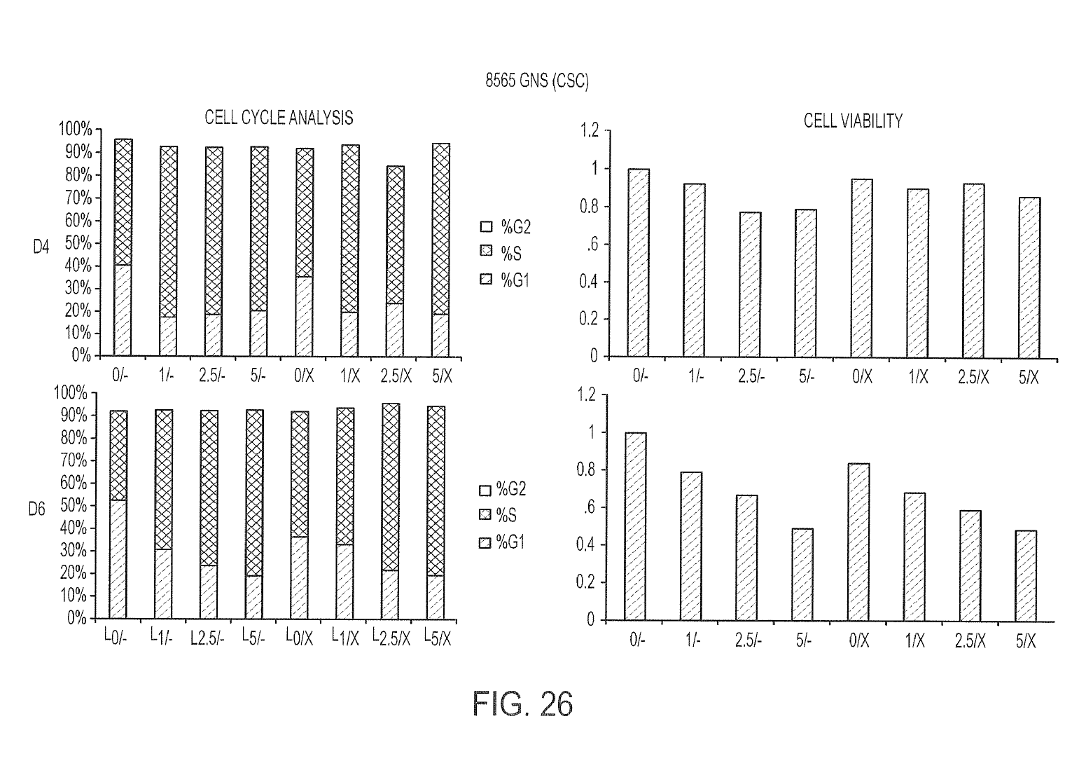

FIG. 26 shows the results for 8565 GNS (CSC) as depicted in FIG. 25.

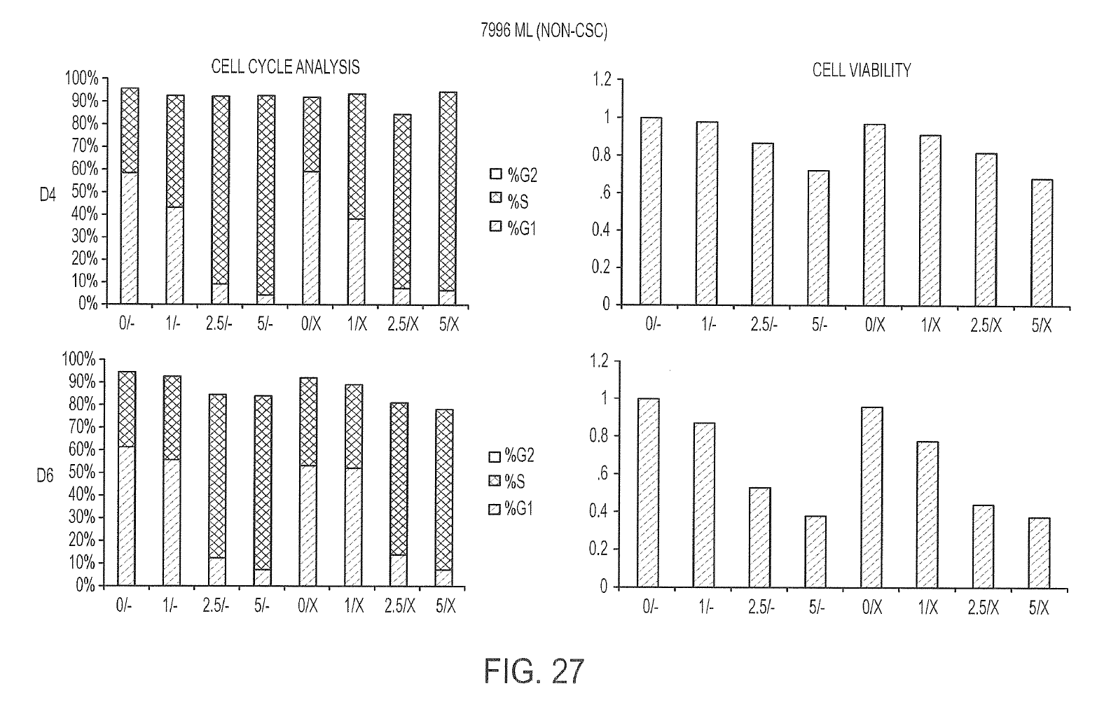

FIG. 27 shows the results for 7996 ML (non-CSC) as depicted in FIG. 25.

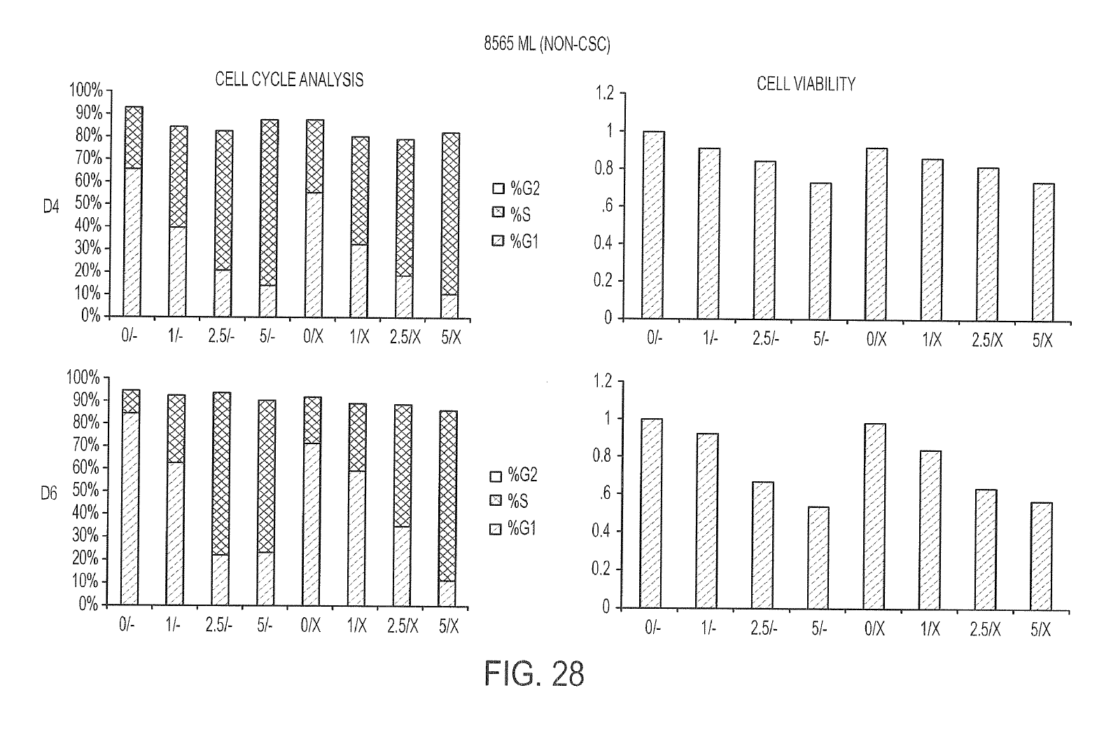

FIG. 28 shows the results for 8565 ML (non-CSC) as depicted in FIG. 25.

In summary, dianhydrogalactitol results in cell cycle arrest and loss of cell viability in nearly all cultures tested. Dianhydrogalactitol appears to cause cell cycle arrest and loss of cell viability at lower concentrations than temozolomide. Furthermore, the efficacy of dianhydrogalactitol is not affected by MGMT status or cell culture condition (stem versus non-stem) as all primary cultures tested were sensitive to dianhydrogalactitol exposure. For all cultures tested, a potential additive effect of dianhydrogalactitol with radiation was seen, particularly at low concentrations of dianhydrogalactitol, such as 1 μL. This was most pronounced in 7996 GNS (CSC) with 20% reduction in cell viability. These results suggest that dianhydrogalactitol may provide a greater clinical benefit to glioma patients compared to the standard of care chemotherapy, temozolomide.

### Example 3

### Use of Dianhydrogalactitol to Treat Patients with Recurrent Malignant Glioma or Progressive Secondary Brain Tumor

Tumors of the brain are among the most challenging malignancies to treat. Median survival for patients with recurrent disease is <6 months for glioblastoma multiforme (GBM). Central Nervous System (CNS) metastases have evolved as a major contributor to cancer mortality based on improvements in systemic therapies that cannot reach tumors spreading to the brain.

Front-line systemic therapy is temozolomide but resistance due to O⁶-methylguanine-DNA-methyltransferase (MGMT) activity is implicated in poor outcomes. Such resistance vastly reduces survival.

Dianhydrogalactitol is a first-in-class bifunctional N⁷ DNA-alkylating agent that readily crosses the blood-brain barrier and accumulates in brain tissue. Dianhydrogalactitol causes interstrand DNA crosslinks at the N⁷-guanine (E. Institóris et al., “Absence of Cross-Resistance Between Two Alkylating Agents: BCNU vs. Bifunctional Galactitol,” Cancer Chemother. Pharmacol. 24:311-313 (1989), incorporated herein by this reference), which is distinct from the mechanisms of other alkylating agents used in GBM. The use of dianhydrogalactitol as an antineoplastic agent has been described in L. Németh et al., “Pharmacologic and Antitumor Effects of 1,2:5,6-Dianhydrogalactitol (NSC-132313),” Cancer Chemother. Rep. 56:593-602 (1972), incorporated herein by this reference. Historical clinical data further suggest comparable or enhanced survival and improved safety compared to TMZ and BCNU and reported absence of cross-resistance between dianhydrogalactitol and both TMZ and BCNU, supports the potential efficacy of dianhydrogalactitol in the treatment of GBM patients failing other agents. Dianhydrogalactitol has been granted orphan drug status by FDA and EMA for the treatment of gliomas. Previous clinical studies suggest that dianhydrogalactitol has anti-tumor activity against a range of cancers including GBM.

In in vitro studies, dianhydrogalactitol demonstrated activity in pediatric and adult GBM cell lines, as well as GBM cancer stem cells. In particular, dianhydrogalactitol can overcome resistance attributable to MGMT activity in vitro.

In light of extensive safety data from clinical trials and promising efficacy in central nervous system (CNS) tumors, we have initiated a new clinical study to establish the maximum tolerated dose (MTD) and identify a dose and dosing regimen for future efficacy trials in GBM.

Dose limiting toxicity is expected to be myelosuppression, the management of which has improved in recent years.

Early in the development of dianhydrogalactitol, a cumulative IV dose of 125 mg/m² delivered in a 35 day cycle in combination with radiation was shown superior to radiation alone in brain cancer (R. T. Eagan et al., “Dianhydrogalactitol and Radiation Therapy. Treatment of Supratentorial Glioma,” JAMA 241:2046-2050 (1979), incorporated herein by this reference).

As indicated above, expression of O⁶-methylguanine methyltransferase (MGMT) has been linked to poor patient outcome in GBM patients treated with temozolomide (TMZ). The cytotoxic activity of dianhydrogalactitol is independent of the MGMT associated chemotherapeutic resistance in vitro (FIG. 1) and thus has potential to be effective in TMZ-resistant GBM.

In the present study, the cumulative dose in a 33 day cycle ranges from 9 mg/m² (cohort 1) to 240 mg/m² (cohort 7). Five dose cohorts, with the highest 33 day cycle cumulative dose of 120 mg/m², have completed the trial with no drug-related serious adverse events: MTD was not yet reached. Enrollment for cohort 6 (33 day cumulative dose: 180 mg/m²) has been initiated. The final cohort of this study, cohort 7 (33 day cumulative dose: 240 mg/m²), will be initiated subject to no dose-limiting toxicity (DLT) in cohort 6; the results will determine the design of the safety and efficacy registration trial.

The methodology of the study reported in this Example is as follows: An open-label, single arm Phase I/II dose-escalation study designed to evaluate the safety, tolerability, pharmacokinetics and anti-tumor activity of dianhydrogalactitol in patients with: (i) histologically confirmed initial diagnosis of primary WHO Grade IV malignant GBM, now recurrent, or (ii) progressive secondary brain tumor, having failed standard brain radiotherapy, and with brain tumor progression after at least one line of systemic therapy. The study utilizes a 3+3 dose escalation design, until the MTD or the maximum specified dose is reached. Patients receive dianhydrogalactitol intravenously at the assigned dose on days 1, 2, and 3 of each 21-day treatment cycle. In Phase II, additional patients will be treated at the MTD (or other selected optimum Phase II dose) to measure tumor responses. All patients enrolled have previously been treated with surgery and/or radiation, if appropriate, and must have failed both bevacizumab and TMZ, unless contraindicated. For these studies, the following is a summary of the inclusion criteria: (1) Patients must be greater than or equal to 18 years old. (2) There is a histologically confirmed initial diagnosis of primary WHO Grade IV malignant glioma (glioblastoma), now recurrent, or progressive secondary brain tumor, the patient has failed standard brain radiotherapy, and the patient has brain tumor progression after at least one line of systemic therapy. (3) If GBM, the patient has been previously treated for GBM with surgery and/or radiation, if appropriate, and the patient must have failed both bevacizumab (Avastin®) and temozolomide (Temodar®), unless either or both are contraindicated. (4) The patient must have a predicted life expectancy of at least 12 weeks. The following is a summary of the exclusion criteria: (1) There is a current history of neoplasm other than the entry diagnosis. Patients with previous cancers treated and cured with local therapy alone may be considered. (2) There is evidence of leptomeningeal spread of disease. (3) The patient had undergone prior treatment with prolifeprospan 20 with carmustine wafer (Gliadel® wafer) within 60 days prior to first treatment (Day 0). (4) The patient had undergone prior treatment with intracerebral agents. (5) The patient shows evidence of recent hemorrhage on baseline MRI of the brain. (6) The patient is being administered concomitant medications that are strong inhibitors of cytochrome P450 and CYP3A up to 14 days before Cycle 1, Day 1 (pimozide, diltiazem, erythromycin, clarithromycin, and quinidine, and amiodarone up to 90 days before.

The results are as follows: No drug-related serious adverse events have been detected, and maximum tolerated dose (MTD) has not been reached at doses up to 30 mg/m². Enrollment and evaluation of Cohort 7 (40 mg/m²) is ongoing. Higher doses may be enrolled subject to completion of mandated safety observation period with Cohort 6 (30 mg/m²). Patients enrolled present with refractory progressive GBM and a dire prognosis. All GBM patients enrolled to date have failed front-line temozolomide and all except one had failed second-line bevacizumab therapy. The primary endpoint of this portion of the study is to determine a modernized dosing regimen for advancement to registration-directed clinical trials. Tumor volume is measured after every second cycle and patients exhibiting any evidence of continued progression at any time during the study are discontinued, but cycle 1 toxicity is captured for MTD determination. In this design, it is not possible to perform a rigorous assessment of patient benefit due to slowed tumor growth. Tumor volume is assessed during the study based on RANO criteria. Two patients exhibiting a response (stable disease or partial response) reported in early cohorts improved clinical signs with a maximum response of 28 cycles (84 weeks) prior to discontinuing due to adverse events unrelated to study. To date, one of two patients in cohort 6 (30 mg/m²) exhibited stable disease after 1 cycle of treatment. Outcomes analysis of cohort 6 is ongoing. These preliminary data support continued exploration of higher dose cohorts.

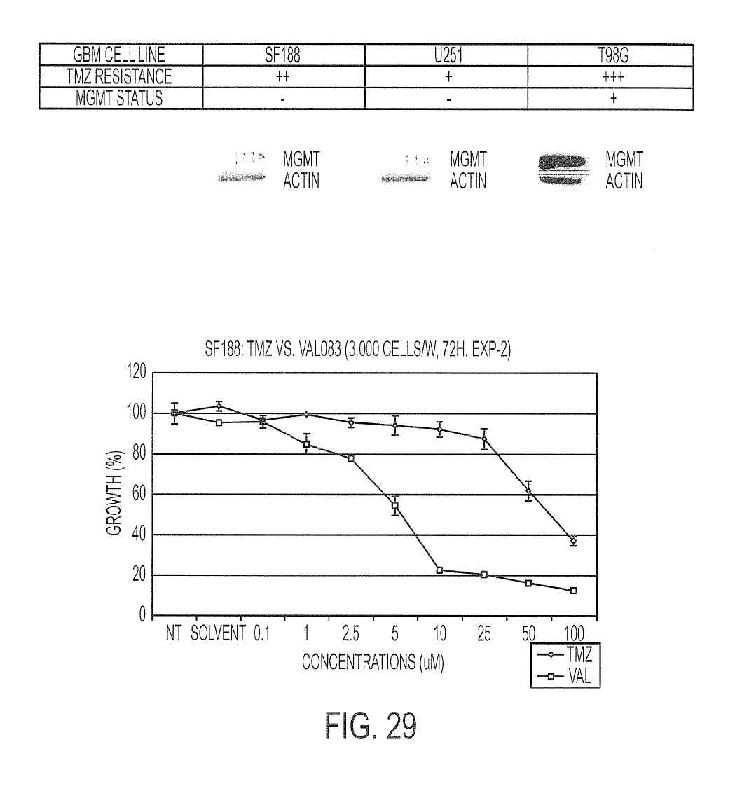

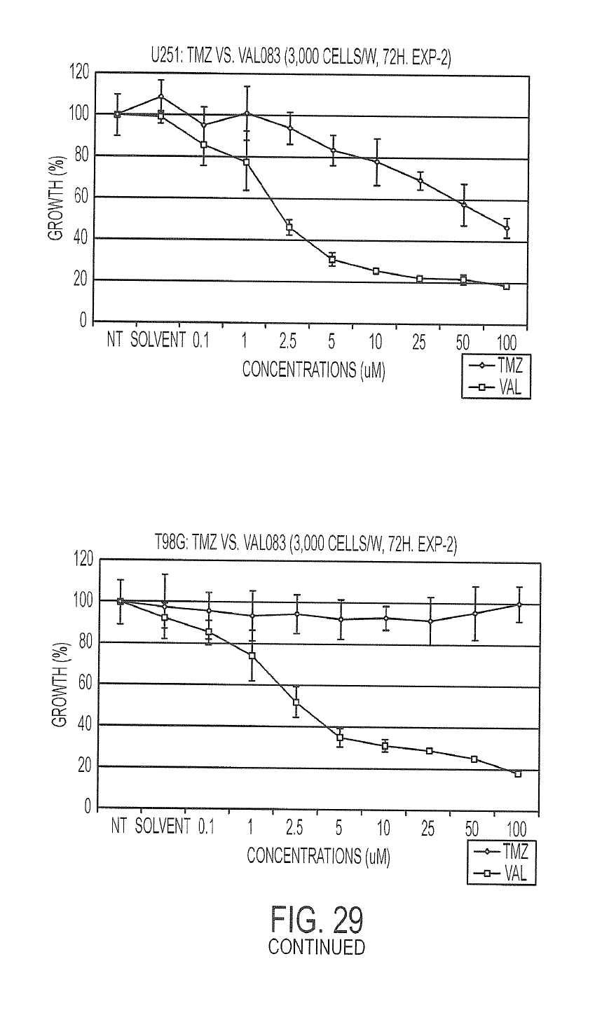

FIG. 29 shows the activity of dianhydrogalactitol (VAL-083) and temozolomide (TMZ) in MGMT negative pediatric human GBM cell line SF188 (first panel), MGMT negative human GBM cell line U251 (second panel) and MGMT positive human GBM cell line T98G (third panel); immunoblots showing detection of MGMT and actin (as a control) in the individual cell lines are shown under the table providing the properties of the cell lines.

Dianhydrogalactitol was better than TMZ for inhibiting tumor growth in GBM cell lines SF188, U251, and T98G, activity independent of MGMT (FIG. 29). Dianhydrogalactitol furthermore inhibited the growth of cancer stem cells (BT74, GBM4 and GBM8) by 80-100% in neurosphere growth assays, with minimal effect on normal human neural stem cells (K. Hu et al., “VAL083, a Novel N7 Alkylating Agent, Surpasses Temozolomide Activity and Inhibits Cancer Stem Cells Providing a New Potential Treatment Option for Glioblastoma Multiforme,” Cancer Res. 72(8) Suppl. 1: 1538 (2012), incorporated herein by this reference).

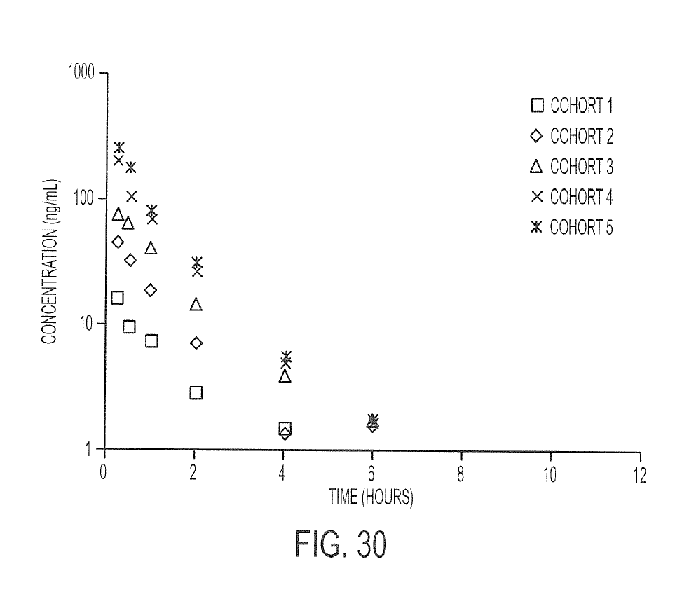

Pharmacokinetic analyses show dose-dependent systemic exposure with a short plasma 1-2 h half-life; average Cₘₐₓ at 20 mg/m² is 266 ng/mL (0.18 μg/mL or ˜1.8 μM). Pharmacokinetic analyses of cohort 6 (30 mg/m²) are ongoing. In previous clinical trials using less sensitive bioanalytical methods than today's LC-MS-MS method (R. T. Eagan et al., “Clinical and Pharmacologic Evaluation of Split-Dose Intermittent Therapy with Dianhydrogalactitol,” Cancer Treat. Rep. 66:283-287 (1982), incorporated herein by this reference), iv infusion of approximately 3-4 times higher doses (60-72 mg/m²) led to Cₘₐₓ ranging from 1.9 to 5.6 μg/mL, and the concentration-time curve was bi-exponential, similar to the finding in the current trial. Pharmacokinetics are linear and consistent with previous published data suggesting higher levels can be achieved at higher doses in the current trial. In vitro studies indicate that μM concentrations of dianhydrogalactitol), as obtained in cohorts 4, 5 and 6, are effective against various glioma cell lines (as shown in FIG. 29). FIG. 30 shows the plasma concentration-time profiles of dianhydrogalactitol showing dose-dependent systemic exposure (mean of 3 subjects per cohort).

TABLE 5
Prior Therapy, Serious Adverse Events (SAE), Dose-Limiting Toxicities
(DLT) and Tumor Response of the Patients Evaluated

| Tumor | | | | | Tumor |
|---|---|---|---|---|---|
| Type | n | Prior Therapy | DLT | SAE | Response |
| GBM | 8 | Surgery/XRT/ | None | None (n = 6) | Overall = 25% |
| | | TMZ/BEV | | Not related to | PR (1); SD (1) |
| | | | | study drug | |
| | | | | (n = 2)* | |
| | 6** | Standard of | None | None (n = 5) | Overall = 17% |
| | | care*** | | | SD (1) |

*Three events in two patients;
**Breast adenocarcinoma (2); small-cell lung carcinoma (3); melanoma (1);
***Whole-brain radiotherapy and stereotactic radiosurgery when appropriate, plus at least one line of systemic therapy.

Table 6 shows a comparison of historical clinical data for dianhydrogalactitol in comparison with other therapies.

TABLE 6
Historical Clinical Data with Dianhydrogalactitol
Support the Potential for Comparable or Enhanced
Survival Similar to Standard Chemotherapy with an
Improved Safety Profile in the Treatment of GBM

| GBM | Dianhydrogalactitol | Temozolomide | Carmustine |
|---|---|---|---|
| Chemotherapy | (Eagan (1979)) | (Stupp (2005)) | (BCNU) |
| Median O.S. | 67 weeks | 58 weeks | 40-50 weeks |
| (XRT + Chemo) | | | |
| DLT | Hematologic | Hematologic | Hematologic |
| Nadir | 18-21 days | 21-28 days | 21-35 days |
| Recovery | Within 7-8 days | Within 14 days | 42-56 days |
| Other Severe | None | Nausea, | Pulmonary, |
| Toxicities | | vomiting, | nausea, |
| Reported (>2%) | | fatigue, | vomiting, |
| | | asthenia, | encephalop- |
| | | neuropathy | athy, renal |

The references for Table 6 are as follows: “Eagan (1979)” is R. T. Eagan et al., “Dianhydrogalactitol and Radiation Therapy. Treatment of Supratentorial Glioma,” JAMA 241:2046-2050 (1979); “Stupp (2005)” is R. Stupp et al., “Radiotherapy Plus Concomitant and Adjuvant Temozolomide for Glioblastoma,” New. Engl. J. Med. 352:987-996 (2005), both of which are incorporated herein by this reference.

Table 7 is a table summarizing the dosing schedule for the trial reported in this Example.

TABLE 7

| | | | | Cumulative dose | |
|---|---|---|---|---|---|
| | | | | in 33-day cycle | |
| Dose Escalation | | | | (comparison to | |
| Scheme | | | | NCI historical | |
| (mg/m²) | | Patients | | regimen of 125 | |
| Original | Revised | Treated | Status | mg/m² per cycle) | |
| 1.5 | 1.5 | 3 | Completed - No DLT | 9 | mg/m² |
| 3.0 | 3.0 | 4 | Completed - No DLT | 18 | mg/m² |
| 5.0 | 5.0 | 10* | Completed - No DLT | 30 | mg/m² |
| 10.0 | 10.0 | 3 | Completed - NO DLT | 60 | mg/m² |
| 15.0 | 20.0 | 4 | Completed - NO DLT | 120 | mg/m² |
| 20.0 | | | | | |
| 25.0 | 30.0 | 3 | Completed - No DLT | 180 | mg/m² |
| 30.0 | | | Analysis ongoing | | |
| n/a | 40.0 | 3 | Enrolling | 240 | mg/m² |
| | | (planned) | | | |

*Cohorts 2 and 3 were expanded to allow for patient demand and to gather additional data on CNS metastases patients.

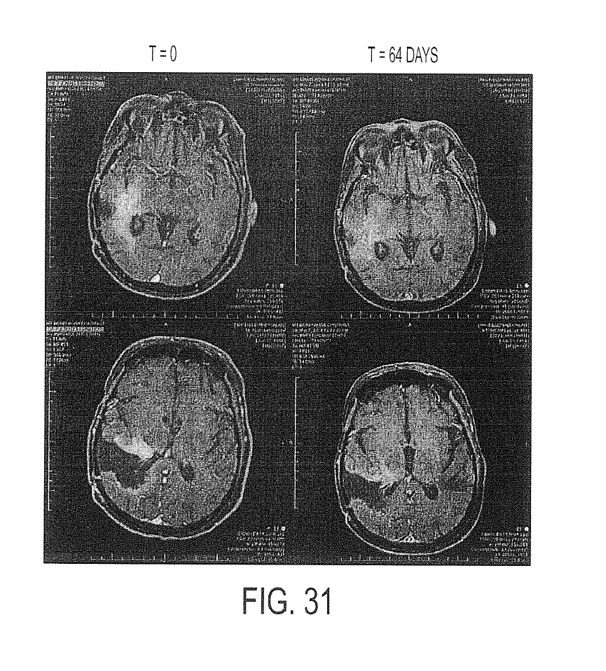

FIG. 31 shows MRI scans of a patient (Patient #26) before (at T=0 days) on the left and after (at T=64 days) on the right after two cycles of dianhydrogalactitol treatment. Thick confluent regions of abnormal enhancement have diminished, now appearing more heterogeneous.

In summary, dianhydrogalactitol shows activity against recurrent glioblastoma multiforme that has proven resistant to previous treatment with temozolomide or bevacizumab. Dianhydrogalactitol also shows activity against progressive secondary brain tumors, including tumors that arise from metastases of breast adenocarcinoma, small-cell lung carcinoma, or melanoma. Dianhydrogalactitol therefore provides a new treatment modality for treatment of these malignancies of the central nervous system, especially in circumstances where the malignancies have proven resistant to therapeutic agents such as temozolomide or bevacizumab.

In particular, dianhydrogalactitol had previously demonstrated promising clinical activity against newly-diagnosed and recurrent GBM in historical NCI-sponsored clinical trials. Dianhydrogalactitol has potent MGMT-independent cytotoxic activity against GBM cell lines in vitro. Pharmacokinetic analyses show dose-dependent increase in exposure with a short plasma 1-2 h half-life and a Cₘₐₓ of <265 ng/mL (1.8 μM) at 20 mg/m² (see FIG. 2). The pharmacokinetic data is consistent with literature from previous trials, suggesting activity of dianhydrogalactitol in brain tumors; plasma concentration achieved in the 20 mg/m² cohort is sufficient to inhibit glioma cell growth in vitro. Dianhydrogalactitol therapy is well tolerated to date; no drug-related serious adverse events have been detected. The maximum tolerate dose (MTD) has not been reached after completion of cohort 6 (30 mg/m²); enrollment and analysis of cohort 7 (40 mg/m²) is ongoing.

Due to prior chemotherapy and radiation therapy, patients with secondary brain tumors are likely more prone to myelosuppression and may have a different MTD (maximum tolerated dose) than patients with GBM. This can be determined by assessing function of the immune system and monitoring possible myelosuppression.

### ADVANTAGES OF THE INVENTION

The present invention provides improved methods and compositions employing dianhydrogalactitol for the treatment of non-small-cell lung carcinoma (NSCLC), a type of lung cancer that has proven resistant to chemotherapy by conventional means. The present invention also provides improved methods and compositions employing dianhydrogalactitol for the treatment of glioblastoma multiforme (GBM).

The use of dianhydrogalactitol to treat NSCLC or GBM is expected to be well tolerated and not to result in additional side effects. Dianhydrogalactitol can be used together with radiation or other chemotherapeutic agents. Additionally, dianhydrogalactitol can be used to treat brain metastases of NSCLC and can be used to treat NSCLC in patients who have developed resistance to platinum-based therapeutic agents such as cisplatin or to tyrosine

Methods according to the present invention possess industrial applicability for the preparation of a medicament for the treatment of NSCLC or GBM. Compositions according to the present invention possess industrial applicability as pharmaceutical compositions, particularly for the treatment of NSCLC or GBM.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

What is claimed is:

1. A method to improve the efficacy and/or reduce the side effects of the administration of a substituted hexitol derivative for treatment of non-small-cell lung carcinoma (NSCLC) or glioblastoma multiforme (GBM) comprising the steps of:
(a) identifying at least one factor or parameter associated with the efficacy and/or occurrence of side effects of the administration of the substituted hexitol derivative for treatment of NSCLC or GBM; and
(b) modifying the factor or parameter to improve the efficacy and/or reduce the side effects of the administration of the substituted hexitol derivative for treatment of NSCLC or GBM.

2. The method of claim 1 wherein the substituted hexitol derivative is selected from the group consisting of galactitols, substituted galacitols, dulcitols, and substituted dulcitols.

3. The method of claim 2 wherein the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol.

4. The method of claim 3 wherein the substituted hexitol derivative is dianhydrogalactitol.

5. The method of claim 1 wherein the method improves the efficacy and/or reduces the side effects of the administration of a substituted hexitol derivative for treatment of non-small-cell lung carcinoma (NSCLC).

6. The method of claim 1 wherein the method improves the efficacy and/or reduces the side effects of the administration of a substituted hexitol derivative for treatment of glioblastoma multiforme (GBM).

7. The method of claim 1 wherein the factor or parameter is selected from the group consisting of:
(a) dose modification;
(b) route of administration;
(c) schedule of administration;
(d) administration to promote preferential accumulation in brain tissue;
(e) selection of disease stage;
(f) patient selection;
(g) patient/disease phenotype;
(h) patient/disease genotype;
(i) pre/post-treatment preparation
(j) toxicity management;
(k) pharmacokinetic/pharmacodynamic monitoring;
(l) drug combinations;
(m) chemosensitization;
(n) chemopotentiation;
(o) post-treatment patient management;
(p) alternative medicine/therapeutic support;
(q) bulk drug product improvements;
(r) diluent systems;
(s) solvent systems;
(t) excipients;
(u) dosage forms;
(v) dosage kits and packaging;
(w) drug delivery systems;
(x) drug conjugate forms;
(y) compound analogs;
(z) prodrugs;
(aa) multiple drug systems;
(ab) biotherapeutic enhancement;
(ac) biotherapeutic resistance modulation;
(ad) radiation therapy enhancement;
(ae) novel mechanisms of action;
(af) selective target cell population therapeutics;
(ag) use with ionizing radiation;
(ah) use with an agent that counteracts myelosuppression; and
(aj) use with an agent that increases the ability of the substituted hexitol to pass through the blood-brain barrier to treat brain metastases of NSCLC.

8. The method of claim 7 wherein the substituted hexitol derivative is dianhydrogalactitol.

9. The method of claim 7 wherein the improvement is made by dose modification and the dose modification is at least one dose modification selected from the group consisting of:
(i) continuous i.v. infusion for hours to days;
(ii) biweekly administration;
(iii) doses greater than 5 mg/m²/day;
(iv) progressive escalation of dosing from 1 mg/m²/day based on patient tolerance;
(v) use of caffeine to modulate metabolism;
(vi) use of isoniazid to modulate metabolism;
(vii) selected and intermittent boosting of dosage administration;
(viii) administration of single and multiple doses escalating from 5 mg/m²/day via bolus;
(ix) oral dosages of below 30 mg/m²;
(x) oral dosages of above 130 mg/m²;
(xi) oral dosages up to 40 mg/m² for 3 days and then a nadir/recovery period of 18-21 days;
(xii) dosing at a lower level for an extended period;
(xiii) dosing at a higher level;
(xiv) dosing with a nadir/recovery period longer than 21 days;
(xv) the use of the substituted hexitol derivative as a single cytotoxic agent at 30 mg/m²/day×5 days, repeated monthly;
(xvi) dosing at 3 mg/kg;
(xvii) the use of a substituted hexitol derivative in combination therapy, at 30 mg/m²/day×5 days; and
(xviii) dosing at 40 mg/day×5 days in adult patients, repeated every two weeks.

10. The method of claim 9 wherein the substituted hexitol derivative is dianhydrogalactitol.

11. The method of claim 7 wherein the improvement is made by route of administration and the route of administration is at least one route of administration selected from the group consisting of:
(i) topical administration;
(ii) oral administration;
(iii) slow release oral delivery;
(iv) intrathecal administration;
(v) intraarterial administration;
(vi) continuous infusion;
(vii) intermittent infusion;
(viii) intravenous administration, such as intravenous administration for 30 minutes;
(ix) administration through a longer infusion; and
(x) administration through IV push.

12. The method of claim 10 wherein the substituted hexitol derivative is dianhydrogalactitol.

13. The method of claim 7 wherein the improvement is made by schedule of administration and the schedule of administration is at least one schedule of administration selected from the group consisting of:
(i) daily administration;
(ii) weekly administration;
(iii) weekly administration for three weeks;
(iv) biweekly administration;
(v) biweekly administration for three weeks with a 1-2 week rest period;
(vi) intermittent boost dose administration; and
(vii) daily administration for one week for multiple weeks.

14. The method of claim 13 wherein the substituted hexitol derivative is dianhydrogalactitol.

15. The method of claim 7 wherein the improvement is made by selection of disease stage and wherein the selection of disease stage is at least one selection of disease stage selected from the group consisting of:
(i) use in an appropriate disease stage for NSCLC or GBM;
(ii) use with an angiogenesis inhibitor to prevent or limit metastatic spread;
(iii) use for newly diagnosed disease;
(iv) use for recurrent disease; and
(v) use for resistant or refractory disease.

16. The method of claim 15 wherein the substituted hexitol derivative is dianhydrogalactitol.

17. The method of claim 7 wherein the improvement is made by patient selection and the patient selection is at least one patient selection carried out by a criterion selected from the group consisting of:
(i) selecting patients with a disease condition characterized by a high level of a metabolic enzyme selected from the group consisting of histone deacetylase and omithine decarboxylase;
(ii) selecting patients with a low or high susceptibility to a condition selected from the group consisting of thrombocytopenia and neutropenia;
(iii) selecting patients intolerant of GI toxicities;
(iv) selecting patients characterized by over- or under-expression of a gene selected from the group consisting of c-Jun, a GPCR, a signal transduction protein, VEGF, a prostate-specific gene, and a protein kinase.
(v) selecting patients characterized by carrying extra copies of the EGFR gene for NSCLC;
(vi) selecting patients characterized by methylation or lack of methylation of the promoter of the MGMT gene;
(vii) selecting patients characterized by an unmethylated promoter region of MGMT (O⁶-methylguanine methyltransferase);
(viii) selecting patients characterized by a methylated promoter region of MGMT;
(ix) selecting patients characterized by a high expression of MGMT;
(x) selecting patients characterized by a low expression of MGMT;
(xi) selecting patients characterized by a mutation in EGFR;
(xii) selecting patients being administered a platinum-based drug as combination therapy;
(xiii) selecting patients who do not have EGFR mutations and thus are less likely to respond to tyrosine kinase inhibitors (TKI);
(xiv) selecting patients who have become resistant to TKI treatment;
(xv) selecting patients who have the BIM co-deletion mutation and thus are less likely to respond to TKI treatment;
(xvi) selecting patients who have become resistant to platinum-based drug treatment; and
(xvii) selecting patients with brain metastases.

18. The method of claim 17 wherein the substituted hexitol derivative is dianhydrogalactitol.

19. The method of claim 17 wherein the criterion is selecting patients characterized by a mutation in EGFR and the mutation in EGFR is EGFR Variant III.

20. The method of claim 7 wherein the improvement is made by analysis of patient or disease phenotype and the analysis of patient or disease phenotype is a method of analysis of patient or disease phenotype carried out by a method selected from the group consisting of:
(a) use of a diagnostic tool, a diagnostic technique, a diagnostic kit, or a diagnostic assay to confirm a patient's particular phenotype;
(b) use of a method for measurement of a marker selected from the group consisting of histone deacetylase, omithine decarboxylase, VEGF, a protein that is a gene product of jun, and a protein kinase;
(c) surrogate compound dosing; and
(d) low dose pre-testing for enzymatic status.

21. The method of claim 20 wherein the substituted hexitol derivative is dianhydrogalactitol.

22. The method of claim 7 wherein the improvement is made by analysis of patient or disease genotype and wherein the method of analysis of patient or disease genotype is a method of analysis of patient or disease genotype carried out by a method selected from the group consisting of:
(i) use of a diagnostic tool, a diagnostic technique, a diagnostic kit, or a diagnostic assay to confirm a patient's particular genotype;
(ii) use of a gene chip;
(iii) use of gene expression analysis;
(iv) use of single nucleotide polymorphism (SNP) analysis;
(v) measurement of the level of a metabolite or a metabolic enzyme;
(vi) determination of copy number of the EGFR gene;
(vii) determination of status of methylation of promoter of MGMT gene;
(viii) determination of the existence of an unmethylated promoter region of the MGMT gene;
(ix) determination of the existence of a methylated promoter region of the MGMT gene;
(x) determination of the existence of high expression of MGMT; and
(xi) determination of the existence of low expression of MGMT.

23. The method of claim 22 wherein the method is use of single nucleotide polymorphism (SNP) analysis and wherein the SNP analysis is carried out on a gene selected from the group consisting of histone deacetylase, omithine decarboxylase, VEGF, a prostate specific gene, c-Jun, and a protein kinase.

24. The method of claim 22 wherein the substituted hexitol derivative is dianhydrogalactitol.

25. The method of claim 7 wherein the improvement is made by pre/post-treatment preparation and wherein the pre/post-treatment preparation is a method of pre/post treatment preparation selected from the group consisting of:
(i) the use of colchicine or an analog thereof;
(ii) the use of a diuretic;
(iii) the use of a uricosuric;
(iv) the use of uricase;
(v) the non-oral use of nicotinamide;
(vi) the use of a sustained-release form of nicotinamide;
(vii) the use of an inhibitor of poly-ADP ribose polymerase;
(viii) the use of caffeine;
(ix) the use of leucovorin rescue;
(x) infection control; and
(xi) the use of an anti-hypertensive agent.

26. The method of claim 25 wherein the substituted hexitol derivative is dianhydrogalactitol.

27. The method of claim 7 wherein the improvement is made by toxicity management and wherein the toxicity management is a method of toxicity management selected from the group consisting of:
(i) the use of colchicine or an analog thereof;
(ii) the use of a diuretic;
(iii) the use of a uricosuric;
(iv) the use of uricase;
(v) the non-oral use of nicotinamide;
(vi) the use of a sustained-release form of nicotinamide;
(vii) the use of an inhibitor of poly-ADP ribose polymerase;
(viii) the use of caffeine;
(ix) the use of leucovorin rescue;
(x) the use of sustained-release allopurinol;
(xi) the non-oral use of allopurinol;
(xii) the use of bone marrow transplants;
(xiii) the use of a blood cell stimulant;
(xiv) the use of blood or platelet infusions;
(xv) the administration of an agent selected from the group consisting of filgrastim, G-CSF, and GM-CSF;
(xvi) the application of a pain management technique;
(xvii) the administration of an anti-inflammatory agent;
(xviii) the administration of fluids;
(xix) the administration of a corticosteroid;
(xx) the administration of an insulin control medication;
(xxi) the administration of an antipyretic;
(xxii) the administration of an anti-nausea treatment;
(xxiii) the administration of an anti-diarrheal treatment;
(xxiv) the administration of N-acetylcysteine; and
(xxv) the administration of an antihistamine.

28. The method of claim 27 wherein the substituted hexitol derivative is dianhydrogalactitol.

29. The method of claim 7 wherein the improvement is made by pharmacokinetic/pharmacodynamic monitoring and wherein the pharmacokinetic/pharmacodynamic monitoring is a method selected from the group consisting of:
(i) multiple determinations of blood plasma levels; and
(ii) multiple determinations of at least one metabolite in blood or urine.

30. The method of claim 29 wherein the substituted hexitol derivative is dianhydrogalactitol.

31. The method of claim 7 wherein the improvement is made by drug combination and wherein the drug combination is a drug combination selected from the group consisting of:
(i) use with topoisomerase inhibitors;
(ii) use with fraudulent nucleosides;
(iii) use with fraudulent nucleotides;
(iv) use with thymidylate synthetase inhibitors;
(v) use with signal transduction inhibitors;
(vi) use with cisplatin or platinum analogs;
(vii) use with monofunctional alkylating agents;
(viii) use with bifunctional alkylating agents;
(ix) use with alkylating agents that damage DNA at a different place than does dianhydrogalactitol;
(x) use with anti-tubulin agents;
(xi) use with antimetabolites;
(xii) use with berberine;
(xiii) use with apigenin;
(xiv) use with amonafide;
(xv) use with colchicine or analogs;
(xvi) use with genistein;
(xvii) use with etoposide;
(xviii) use with cytarabine;
(xix) use with camptothecins
(xx) use with vinca alkaloids;
(xxi) use with 5-fluorouracil;
(xxii) use with curcumin;
(xxiii) use with NF-κB inhibitors;
(xxiv) use with rosmarinic acid;
(xxv) use with mitoguazone;
(xxvi) use with tetrandrine;
(xxvii) use with temozolomide;
(xxviii) use with VEGF inhibitors;
(xxix) use with cancer vaccines;
(xxx) use with EGFR inhibitors;
(xxxi) use with tyrosine kinase inhibitors;
(xxxii) use with poly (ADP-ribose) polymerase (PARP) inhibitors; and
(xxxiii) use with ALK inhibitors.

32. The method of claim 31 wherein the substituted hexitol derivative is dianhydrogalactitol.

33. The method of claim 7 wherein the improvement is made by chemosensitization and the chemosensitization is the use of a substituted hexitol derivative as a chemosensitizer in combination with an agent selected from the group consisting of:
(i) topoisomerase inhibitors;
(ii) fraudulent nucleosides;
(iii) fraudulent nucleotides;
(iv) thymidylate synthetase inhibitors;
(v) signal transduction inhibitors;
(vi) cisplatin or platinum analogs;
(vii) alkylating agents;
(viii) anti-tubulin agents;
(ix) antimetabolites;
(x) berberine;
(xi) apigenin;
(xii) amonafide;
(xiii) colchicine or analogs;
(xiv) genistein;
(xv) etoposide;
(xvi) cytarabine;
(xvii) camptothecins;
(xviii) vinca alkaloids;
(xix) topoisomerase inhibitors;
(xx) 5-fluorouracil;
(xxi) curcumin;
(xxii) NF-κB inhibitors;
(xxiii) rosmarinic acid;
(xxiv) mitoguazone;
(xxv) tetrandrine;
(xxvi) a tyrosine kinase inhibitor;
(xxvii) an inhibitor of EGFR; and
(xxviii) an inhibitor of PARP.

34. The method of claim 33 wherein the substituted hexitol derivative is dianhydrogalactitol.

35. The method of claim 7 wherein the improvement is made by chemopotentiation and the chemosensitization is the use of a substituted hexitol derivative as a chemopotentiator in combination with an agent selected from the group consisting of:
(i) topoisomerase inhibitors;
(ii) fraudulent nucleosides;
(iii) fraudulent nucleotides;
(iv) thymidylate synthetase inhibitors;
(v) signal transduction inhibitors;
(vi) cisplatin or platinum analogs;
(vii) alkylating agents;
(viii) anti-tubulin agents;
(ix) antimetabolites;
(x) berberine;
(xi) apigenin;
(xii) amonafide;
(xiii) colchicine or analogs;
(xiv) genistein;
(xv) etoposide;
(xvi) cytarabine;
(xvii) camptothecins;
(xviii) vinca alkaloids;
(xix) 5-fluorouracil;
(xx) curcumin;
(xxi) NF-κB inhibitors;
(xxii) rosmarinic acid;
(xxiii) mitoguazone;
(xxiv) tetrandrine;
(xxv) a tyrosine kinase inhibitor;
(xxvi) an inhibitor of EGFR; and
(xxvii) an inhibitor of PARP.

36. The method of claim 35 wherein the substituted hexitol derivative is dianhydrogalactitol.

37. The method of claim 7 wherein the improvement is made by post-treatment management and the post-treatment management is a method selected from the group consisting of:
(i) a therapy associated with pain management;
(ii) administration of an anti-emetic;
(iii) an anti-nausea therapy;
(iv) administration of an anti-inflammatory agent;
(v) administration of an anti-pyretic agent; and
(vi) administration of an immune stimulant.

38. The method of claim 37 wherein the substituted hexitol derivative is dianhydrogalactitol.

39. The method of claim 7 wherein the improvement is made by alternative medicine/post-treatment support and the alternative medicine/post-treatment support is a method selected from the group consisting of:
(i) hypnosis;
(ii) acupuncture;
(iii) meditation;
(iv) a herbal medication created either synthetically or through extraction; and
(v) applied kinesiology.

40. The method of claim 39 wherein the substituted hexitol derivative is dianhydrogalactitol.

41. The method of claim 7 wherein the improvement is made by a bulk drug product improvement and the bulk drug product improvement is a bulk drug product improvement selected from the group consisting of:
(i) salt formation;
(ii) preparation as a homogeneous crystal structure;
(iii) preparation as a pure isomer;
(iv) increased purity;
(v) preparation with lower residual solvent content; and
(vi) preparation with lower residual heavy metal content.

42. The method of claim 41 wherein the substituted hexitol derivative is dianhydrogalactitol.

43. The method of claim 7 wherein the improvement is made by use of a diluent and the diluent is a diluent selected from the group consisting of:
(i) an emulsion;
(ii) dimethylsulfoxide (DMSO);
(iii) N-methylformamide (NMF)
(iv) DMF;
(v) ethanol;
(vi) benzyl alcohol;
(vii) dextrose-containing water for injection;
(viii) Cremophor;
(ix) cyclodextrin; and
(x) PEG.

44. The method of claim 43 wherein the substituted hexitol derivative is dianhydrogalactitol.

45. The method of claim 7 wherein the improvement is made by use of a solvent system and the solvent system is a solvent system selected from the group consisting of:
(i) an emulsion;
(ii) dimethylsulfoxide (DMSO);
(iii) N-methylformamide (NMF)
(iv) DMF;
(v) ethanol;
(vi) benzyl alcohol;
(vii) dextrose-containing water for injection;
(viii) Cremophor;
(ix) cyclodextrin; and
(x) PEG.

46. The method of claim 45 wherein the substituted hexitol derivative is dianhydrogalactitol.

47. The method of claim 7 wherein the improvement is made by use of an excipient and the excipient is an excipient selected from the group consisting of:
(i) mannitol;
(ii) albumin;
(iii) EDTA;
(iv) sodium bisulfite;
(v) benzyl alcohol;
(vi) a carbonate buffer; and
(vii) a phosphate buffer.

48. The method of claim 47 wherein the substituted hexitol derivative is dianhydrogalactitol.

49. The method of claim 7 wherein the improvement is made by use of a dosage form and the dosage form is a dosage form selected from the group consisting of:
(i) tablets;
(ii) capsules;
(iii) topical gels;
(iv) topical creams;
(v) patches;
(vi) suppositories; and
(vii) lyophilized dosage fills.

50. The method of claim 49 wherein the substituted hexitol derivative is dianhydrogalactitol.

51. The method of claim 7 wherein the improvement is made by use of dosage kits and packaging and the dosage kits and packaging are selected from the group consisting of the use of amber vials to protect from light and the use of stoppers with specialized coatings to improve shelf-life stability.

52. The method of claim 51 wherein the substituted hexitol derivative is dianhydrogalactitol.

53. The method of claim 7 wherein the improvement is made by use of a drug delivery system and the drug delivery system is a drug delivery system selected from the group consisting of:
(i) nanocrystals;
(ii) bioerodible polymers;
(iii) liposomes;
(iv) slow release injectable gels; and
(v) microspheres.

54. The method of claim 53 wherein the substituted hexitol derivative is dianhydrogalactitol.

55. The method of claim 7 wherein the improvement is made by use of a drug conjugate form and the drug conjugate form is selected from the group consisting of:
(i) a polymer system;
(ii) polylactides;
(iii) polyglycolides;
(iv) amino acids;
(v) peptides; and
(vi) multivalent linkers.

56. The method of claim 55 wherein the substituted hexitol derivative is dianhydrogalactitol.

57. The method of claim 7 wherein the therapeutic agent is a modified substituted hexitol derivative and the modification is selected from the group consisting of:
(i) alteration of side chains to increase or decrease lipophilicity;
(ii) addition of an additional chemical functionality to alter a property selected from the group consisting of reactivity, electron affinity, and binding capacity; and
(iii) alteration of salt form.

58. The method of claim 57 wherein the modified substituted hexitol derivative is a modified dianhydrogalactitol.

59. The method of claim 7 wherein the improvement is made by use of a compound analog and the compound analog is a compound analog selected from the group consisting of:
(i) alteration of side chains to increase or decrease lipophilicity;
(ii) addition of an additional chemical functionality to alter a property selected from the group consisting of reactivity, electron affinity, and binding capacity; and
(iii) alteration of salt form.

60. The method of claim 59 wherein the compound analog is a compound analog of dianhydrogalactitol.

61. The method of claim 7 wherein the substituted hexitol derivative is in the form of a prodrug system and wherein the prodrug system is a prodrug system selected from the group consisting of:
(i) the use of enzyme sensitive esters;
(ii) the use of dimers;
(iii) the use of Schiff bases;
(iv) the use of pyridoxal complexes; and
(v) the use of caffeine complexes.

62. The method of claim 60 wherein the prodrug system is a prodrug system comprising a prodrug of dianhydrogalactitol.

63. The method of claim 7 wherein the improvement is made by use of a multiple drug system and the multiple drug system is a multiple drug system selected from the group consisting of:
(i) use of multi-drug resistance inhibitors;
(ii) use of specific drug resistance inhibitors;
(iii) use of specific inhibitors of selective enzymes;
(iv) use of signal transduction inhibitors;
(v) use of repair inhibition; and
(vi) use of topoisomerase inhibitors with non-overlapping side effects.

64. The method of claim 63 wherein the substituted hexitol derivative is dianhydrogalactitol.

65. The method of claim 7 wherein the improvement is made by biotherapeutic enhancement and the biotherapeutic enhancement is performed by use in combination as sensitizers/potentiators with a therapeutic agent or technique that is a therapeutic agent or technique selected from the group consisting of:
(i) cytokines;
(ii) lymphokines;
(iii) therapeutic antibodies;
(iv) antisense therapies;
(v) gene therapies;
(vi) ribozymes;
(vii) RNA interference; and
(viii) vaccines.

66. The method of claim 65 wherein the substituted hexitol derivative is dianhydrogalactitol.

67. The method of claim 7 wherein the improvement is made by biotherapeutic resistance modulation and the biotherapeutic resistance modulation is use against NSCLC resistant to a therapeutic agent or technique selected from the group consisting of:
(i) biological response modifiers;
(ii) cytokines;
(iii) lymphokines;
(iv) therapeutic antibodies;
(v) antisense therapies;
(vi) gene therapies;
(vii) ribozymes;
(viii) RNA interference; and
(ix) vaccines.

68. The method of claim 67 wherein the substituted hexitol derivative is dianhydrogalactitol.

69. The method of claim 7 wherein the improvement is made by radiation therapy enhancement and the radiation therapy enhancement is a radiation therapy enhancement agent or technique selected from the group consisting of:
(i) hypoxic cell sensitizers;
(ii) radiation sensitizers/protectors;
(iii) photosensitizers;
(iv) radiation repair inhibitors;
(e) thiol depleters;
(f) vaso-targeted agents;
(g) DNA repair inhibitors;
(h) radioactive seeds;
(i) radionuclides;
(j) radiolabeled antibodies; and
(k) brachytherapy.

70. The method of claim 69 wherein the substituted hexitol is dianhydrogalactitol.

71. The method of claim 7 wherein the improvement is made by use of a novel mechanism of action and the novel mechanism of action is a therapeutic interaction with a target or mechanism selected from the group consisting of:
(i) inhibitors of poly-ADP ribose polymerase;
(ii) agents that affect vasculature or vasodilation;
(iii) oncogenic targeted agents;
(iv) signal transduction inhibitors;
(v) EGFR inhibition;
(vi) protein kinase C inhibition;
(vii) phospholipase C downregulation;
(viii) Jun downregulation;
(ix) histone genes;
(x) VEGF;
(xi) omithine decarboxylase;
(xii) ubiquitin C;
(xiii) Jun D;
(xiv) v-Jun;
(xv) GPCRs;
(xvi) protein kinase A;
(xvii) protein kinases other than protein kinase A;
(xviii) prostate specific genes;
(xix) telomerase;
(xx) histone deacetylase; and
(xxi) tyrosine kinase inhibitors.

72. The method of claim 71 wherein the substituted hexitol derivative is dianhydrogalactitol.

73. The method of claim 7 wherein the improvement is made by use of selective target cell population therapeutics and the use of selective target cell population therapeutics is a use selected from the group consisting of:
(i) use against radiation sensitive cells;
(ii) use against radiation resistant cells; and
(iii) use against energy depleted cells.

74. The method of claim 73 wherein the substituted hexitol derivative is dianhydrogalactitol.

75. The method of claim 7 wherein the improvement is made by use of a substituted hexitol derivative in combination with ionizing radiation.

76. The method of claim 75 wherein the substituted hexitol derivative is dianhydrogalactitol.

77. The method of claim 75 wherein the ionizing radiation is administered concurrently with the substituted hexitol derivative.

78. The method of claim 75 wherein the ionizing radiation is administered separately from the substituted hexitol derivative.

79. The method of claim 75 wherein the ionizing radiation is administered in a single dose.

80. The method of claim 75 wherein the ionizing radiation is administered in fractionated doses.

81. The method of claim 75 wherein the radiation dosage is from about 40 Gy to about 79.2 Gy.

82. The method of claim 79 wherein the radiation dosage is about 60 Gy.

83. The method of claim 75 wherein the radiation is administered by a method selected from the group consisting of high-energy X-rays, high-energy electrons from a linear accelerator unit, and gamma rays from a cobalt-60-based device.

84. The method of claim 75 wherein the radiation is administered to treat NSCLC.

85. The method of claim 75 wherein the radiation is administered to treat GBM.

86. The method of claim 85 wherein the method further comprises administration of trans sodium crocetinate as a radiosensitizer.

87. The method of claim 7 wherein the improvement is made by use of an agent that counteracts myelosuppression and the agent that counteracts myelosuppression is a dithiocarbamate.

88. The method of claim 87 wherein the substituted hexitol derivative is dianhydrogalactitol.

89. The method of claim 7 wherein the improvement is made by use with an agent that increases the ability of the substituted hexitol to pass through the blood-brain barrier to treat brain metastases of NSCLC and the agent that increases the ability of the substituted hexitol to pass through the blood-brain barrier is an agent selected from the group consisting of:
(i) a chimeric peptide of the structure of Formula (D-III): 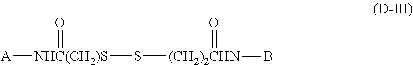
wherein: (A) A is somatostatin, thyrotropin releasing hormone (TRH), vasopressin, alpha interferon, endorphin, muramyl dipeptide or ACTH 4-9 analogue; and (B) B is insulin, IGF-I, IGF-II, transferrin, cationized (basic) albumin or prolactin; or a chimeric peptide of the structure of Formula (D-III) wherein the disulfide conjugating bridge between A and B is replaced with a bridge of Subformula (D-III(a)):
A-NH(CH₂)₂S—S—B(cleavable linkage) (D-III(a)),
wherein the bridge is formed using cysteamine and EDAC as the bridge reagents; or a chimeric peptide of the structure of Formula (D-III) wherein the disulfide conjugating bridge between A and B is replaced with a bridge of Subformula (D-III(b)):
A-NH═CH(CH₂)₃CH═NH—B(non-cleavable linkage) (D-III(b)),
wherein the bridge is formed using glutaraldehyde as the bridge reagent;
(ii) a composition comprising either avidin or an avidin fusion protein bonded to a biotinylated substituted hexitol derivative to form an avidin-biotin-agent complex including therein a protein selected from the group consisting of insulin, transferrin, an anti-receptor monoclonal antibody, a cationized protein, and a lectin;
(iii) a neutral liposome that is pegylated and incorporates the substituted hexitol derivative, wherein the polyethylene glycol strands are conjugated to at least one transportable peptide or targeting agent;
(iv) a humanized murine antibody that binds to the human insulin receptor linked to the substituted hexitol derivative through an avidin-biotin linkage; and
(v) a fusion protein comprising a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative, wherein the fusion protein is linked to the substituted hexitol by a covalent link to biotin.

90. The method of claim 89 wherein the substituted hexitol derivative is dianhydrogalactitol.

91. The method of claim 87 wherein the improvement is made by use of an agent that suppresses the growth of cancer stem cells.

92. The method of claim 91 wherein the agent that suppresses the growth of cancer stem cells is selected from the group consisting of: (1) naphthoquinones; (2) VEGF-DLL4 bispecific antibodies; (3) farnesyl transferase inhibitors; (4) gamma-secretase inhibitors; (5) anti-TIM3 antibodies; (6) tankyrase inhibitors; (7) Wnt pathway inhibitors other than tankyrase inhibitors; (8) camptothecin-binding moiety conjugates; (9) Notch1 binding agents, including antibodies; (10) oxabicycloheptanes and oxabicycloheptenes; (11) inhibitors of the mitochondrial electron transport chains or the mitochondrial tricarboxylic acid cycle; (12) Axl inhibitors; (13) dopamine receptor antagonists; (14) anti-RSPO1 antibodies; (15) inhibitors or modulators of the Hedgehog pathway; (16) caffeic acid analogs and derivatives; (17) Stat3 inhibitors; (18) GRP-94-binding antibodies; (19) Frizzled receptor polypeptides; (20) immunoconjugates with cleavable linkages; (21) human prolactin, growth hormone, or placental lactogen; (22) anti-prominin-1 antibody; (23) antibodies specifically binding N-cadherin; (24) DR5 agonists; (25) anti-DLL4 antibodies or binding fragments thereof; (26) antibodies specifically binding GPR49; (27) DDR1 binding agents; (28) LGR5 binding agents; (29) telomerase-activating compounds; (30) fingolimod plus anti-CD74 antibodies or fragments thereof; (31) an antibody that prevents the binding of CD47 to SIPRα or a CD47 mimetic; (32) thienopyranone kinase inhibitors for inhibition of PI-3 kinases; (33) cancer-stem-cell-binding peptides; (34) diphtheria toxin-interleukin 3 conjugates; (35) inhibitors of histone deacetylase; (36) progesterone or analogs thereof; (37) antibodies binding the negative regulatory region (NRR) of Notch2; (38) inhibitors of HGFIN; (39) immunotherapeutic peptides; (40) inhibitors of CSCPK or related kinases; (41) imidazo[1,2-a]pyrazine derivatives as α-helix mimetics; (42) antibodies directed to an epitope of variant Heterogeneous Ribonucleoprotein G (HnRNPG); (43) antibodies binding TES7 antigen; (44) antibodies binding the ILR3α subunit; (45) ifenprodil tartrate and other compounds with a similar activity; (46) antibodies binding SALL4; (47) antibodies binding Notch4; (48) bispecific antibodies binding both NBR1 and Cep55; (49) Smo inhibitors; (50) peptides blocking or inhibiting interleukin-1 receptor 1; (51) antibodies specific for CD47 or CD19; (52) histone methyltransferase inhibitors; (53) antibodies specifically binding Lg5; (54) antibodies specifically binding EFNA1; (55) phenothiazine derivatives; (56) HDAC inhibitors plus AKT inhibitors; (57) ligands binding to cancer-stem-line-specific cell surface antigen stem cell markers; (58) Notch receptor agonists; (59) binding agents binding human MET; (60) PDGFR-13 inhibitors; (61) pyrazolo compounds with histone demethylase activity; (62) heterocyclic substituted 3-heteroaryidenyl-2-indolinone derivatives; (63) albumin-binding arginine deiminase fusion proteins; (64) hydrogen-bond surrogate peptides and peptidomimetics that reactivate p53; (65) prodrugs of 2-pyrrolinodoxorubicin conjugated to antibodies; (66) targeted cargo proteins; (67) bisacodyl and analogs thereof; (68) N¹-cyclic amine-N⁵-substituted phenyl biguanide derivative; (69) fibulin-3 protein; (70) modulators of SCFSkp2; (71) inhibitors of Slingshot-2; (72) monoclonal antibodies specifically binding DCLK1 protein; (73) antibodies or soluble receptors that modulate the Hippo pathway; (74) selective inhibitors of CDK8 and CDK19; (75) antibodies and antibody fragments specifically binding IL-17; (76) antibodies specifically binding FRMD4A; (77) monoclonal antibodies specifically binding the ErbB-3 receptor; (78) antibodies that specifically bind human RSPO3 and modulate β-catenin activity; (79) esters of 4,9-dihydroxy-naphtho[2,3-b]furans; (80) CCR5 antagonists; (81) antibodies that specifically bind the extracellular domain of human C-type lectin-like molecule (CLL-1); (82) anti-hypertension compounds; (83) anthraquinone radiosensitizer agents plus ionizing radiation; (84) CDK-inhibiting pyrrolopyrimidinone derivatives; (85) analogs of CC-1065 and conjugates thereof; (86) antibodies specifically binding to the protein Notum; (87) CDK8 antagonists; (88) bHLH proteins and nucleic acids encoding them; (89) inhibitors of the histone methyltransferase EZH2; (90) sulfonamides inhibiting carbonic anhydrase isoforms; (91) antibodies specifically binding DEspR; (92) antibodies specifically binding human leukemia inhibitory factor (LIF); (93) doxovir; (94) inhibitors of mTOR; (95) antibodies specifically binding FZD10; (96) napthofurans; (97) death receptor agonists; (98) tigecycline; (99) strigolactones and strigolactone analogs; and (100) compounds inducing methuosis.

93. A composition to improve the efficacy and/or reduce the side effects of suboptimally administered drug therapy employing a substituted hexitol derivative for the treatment of NSCLC or GBM comprising an alternative selected from the group consisting of:
(a) a therapeutically effective quantity of a modified substituted hexitol derivative or a derivative, analog, or prodrug of a substituted hexitol derivative or a modified substituted hexitol derivative, wherein the modified substituted hexitol derivative or the derivative, analog or prodrug of the substituted hexitol derivative or modified substituted hexitol derivative possesses increased therapeutic efficacy or reduced side effects for treatment of NSCLC or GBM as compared with an unmodified substituted hexitol derivative;
(b) a composition comprising:
(i) a therapeutically effective quantity of a substituted hexitol derivative, a modified substituted hexitol derivative, or a derivative, analog, or prodrug of a substituted hexitol derivative or a modified substituted hexitol derivative; and
(ii) at least one additional therapeutic agent, therapeutic agent subject to chemosensitization, therapeutic agent subject to chemopotentiation, diluent, excipient, solvent system, drug delivery system, agent to counteract myelosuppression, or agent that increases the ability of the substituted hexitol to pass through the blood-brain barrier, wherein the composition possesses increased therapeutic efficacy or reduced side effects for treatment of NSCLC or GBM as compared with an unmodified substituted hexitol derivative;
(c) a therapeutically effective quantity of a substituted hexitol derivative, a modified substituted hexitol derivative or a derivative, analog, or prodrug of a substituted hexitol derivative or a modified substituted hexitol derivative that is incorporated into a dosage form, wherein the substituted hexitol derivative, the modified substituted hexitol derivative or the derivative, analog, or prodrug of a substituted hexitol derivative or a modified substituted hexitol derivative incorporated into the dosage form possesses increased therapeutic efficacy or reduced side effects for treatment of NSCLC or GBM as compared with an unmodified substituted hexitol derivative;
(d) a therapeutically effective quantity of a substituted hexitol derivative, a modified substituted hexitol derivative or a derivative, analog, or prodrug of a substituted hexitol derivative or a modified substituted hexitol derivative that is incorporated into a dosage kit and packaging, wherein the substituted hexitol derivative, the modified substituted hexitol derivative or the derivative, analog, or prodrug of a substituted hexitol derivative or a modified substituted hexitol derivative incorporated into the dosage kit and packaging possesses increased therapeutic efficacy or reduced side effects for treatment of NSCLC or GBM as compared with an unmodified substituted hexitol derivative; and
(e) a therapeutically effective quantity of a substituted hexitol derivative, a modified substituted hexitol derivative or a derivative, analog, or prodrug of a substituted hexitol derivative or a modified substituted hexitol derivative that is subjected to a bulk drug product improvement, wherein substituted hexitol derivative, a modified substituted hexitol derivative or a derivative, analog, or prodrug of a substituted hexitol derivative or a modified substituted hexitol derivative subjected to the bulk drug product improvement possesses increased therapeutic efficacy or reduced side effects for treatment of NSCLC or GBM as compared with an unmodified substituted hexitol derivative.

94. The composition of claim 93 wherein the composition possesses increased therapeutic efficacy or reduced side effects for treatment of NSCLC.

95. The composition of claim 93 wherein the composition possesses increased therapeutic efficacy or reduced side effects for treatment of GBM.

96. The composition of claim 93 wherein the unmodified substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol.

97. The composition of claim 96 wherein the unmodified substituted hexitol derivative is dianhydrogalactitol.

98. The composition of claim 93 wherein the composition comprises a drug combination comprising:
(a) a substituted hexitol derivative; and
(b) an additional therapeutic agent selected from the group consisting of:
(i) topoisomerase inhibitors;
(ii) fraudulent nucleosides;
(iii) fraudulent nucleotides;
(iv) thymidylate synthetase inhibitors;
(v) signal transduction inhibitors;
(vi) cisplatin or platinum analogs;
(vii) monofunctional alkylating agents;
(viii) bifunctional alkylating agents;
(ix) alkylating agents that damage DNA at a different place than does dianhydrogalactitol;
(x) anti-tubulin agents;
(xi) antimetabolites;
(xii) berberine;
(xiii) apigenin;
(xiv) amonafide;
(xv) colchicine or analogs;
(xvi) genistein;
(xvii) etoposide;
(xviii) cytarabine;
(xix) camptothecins;
(xx) vinca alkaloids;
(xxi) 5-fluorouracil;
(xxii) curcumin;
(xxiii) NF-κB inhibitors;
(xxiv) rosmarinic acid;
(xxv) mitoguazone;
(xxvi) tetrandrine;
(xxvii) temozolomide;
(xxviii) VEGF inhibitors;
(xxix) cancer vaccines;
(xxx) EGFR inhibitors;
(xxxi) tyrosine kinase inhibitors;
(xxxii) poly (ADP-ribose) polymerase (PARP) inhibitors;
(xxxiii) ALK inhibitors; and
(xxxiv) agents for the suppression of proliferation of cancer stem cells.

99. The composition of claim 98 wherein the substituted hexitol derivative is dianhydrogalactitol.

100. The composition of claim 93 wherein the composition comprises:
(a) a substituted hexitol derivative; and
(b) a therapeutic agent subject to chemosensitization selected from the group consisting of:
(i) topoisomerase inhibitors;
(ii) fraudulent nucleosides;
(iii) fraudulent nucleotides;
(iv) thymidylate synthetase inhibitors;
(v) signal transduction inhibitors;
(vi) cisplatin or platinum analogs;
(vii) alkylating agents;
(viii) anti-tubulin agents;
(ix) antimetabolites;
(x) berberine;
(xi) apigenin;
(xii) amonafide;
(xiii) colchicine or analogs;
(xiv) genistein;
(xv) etoposide;
(xvi) cytarabine;
(xvii) camptothecins;
(xviii) vinca alkaloids;
(xix) topoisomerase inhibitors;
(xx) 5-fluorouracil;
(xxi) curcumin;
(xxii) NF-κB inhibitors;
(xxiii) rosmarinic acid;
(xxiv) mitoguazone;
(xxv) tetrandrine;
(xxvi) a tyrosine kinase inhibitor;
(xxvii) an inhibitor of EGFR; and
(xxviii) an inhibitor of PARP;
wherein the substituted hexitol derivative acts as a chemosensitizer.

101. The composition of claim 100 wherein the substituted hexitol derivative is dianhydrogalactitol.

102. The composition of claim 93 wherein the composition comprises:
(a) a substituted hexitol derivative; and
(b) a therapeutic agent subject to chemopotentiation selected from the group consisting of:
(i) topoisomerase inhibitors;
(ii) fraudulent nucleosides;
(iii) fraudulent nucleotides;
(iv) thymidylate synthetase inhibitors;
(v) signal transduction inhibitors;
(vi) cisplatin or platinum analogs;
(vii) alkylating agents;
(viii) anti-tubulin agents;
(ix) antimetabolites;
(x) berberine;
(xi) apigenin;
(xii) amonafide;
(xiii) colchicine or analogs;
(xiv) genistein;
(xv) etoposide;
(xvi) cytarabine;
(xvii) camptothecins;
(xviii) vinca alkaloids;
(xix) 5-fluorouracil;
(xx) curcumin;
(xxi) NF-κB inhibitors;
(xxii) rosmarinic acid;
(xxiii) mitoguazone;
(xxiv) tetrandrine;
(xxv) a tyrosine kinase inhibitor;
(xxvi) an inhibitor of EGFR; and
(xxvii) an inhibitor of PARP;
wherein the substituted hexitol derivative acts as a chemopotentiator.

103. The composition of claim 102 wherein the substituted hexitol derivative is dianhydrogalactitol.

104. The composition of claim 93 wherein the substituted hexitol derivative is subjected to a bulk drug product improvement, wherein the bulk drug product improvement is selected from the group consisting of:
(a) salt formation;
(b) preparation as a homogeneous crystal structure;
(c) preparation as a pure isomer;
(d) increased purity;
(e) preparation with lower residual solvent content; and
(f) preparation with lower residual heavy metal content.

105. The composition of claim 104 wherein the substituted hexitol derivative is dianhydrogalactitol.

106. The composition of claim 93 wherein the composition comprises a substituted hexitol derivative and a diluent, wherein the diluent is selected from the group consisting of:
(a) an emulsion;
(b) dimethylsulfoxide (DMSO);
(c) N-methylformamide (NMF)
(d) DMF;
(e) ethanol;
(f) benzyl alcohol;
(g) dextrose-containing water for injection;
(h) Cremophor;
(i) cyclodextrin; and
(j) PEG.

107. The composition of claim 106 wherein the substituted hexitol derivative is dianhydrogalactitol.

108. The composition of claim 93 wherein the composition comprises a substituted hexitol derivative and a solvent system, wherein the solvent system is selected from the group consisting of:
(a) an emulsion;
(b) dimethylsulfoxide (DMSO);
(c) N-methylformamide (NMF)
(d) DMF;
(e) ethanol;
(f) benzyl alcohol;
(g) dextrose-containing water for injection;
(h) Cremophor;
(i) cyclodextrin; and
(j) PEG.

109. The composition of claim 108 wherein the substituted hexitol derivative is dianhydrogalactitol.

110. The composition of claim 93 wherein the composition comprises a substituted hexitol derivative and an excipient, wherein the excipient is selected from the group consisting of:
(a) mannitol;
(b) albumin;
(c) EDTA;
(d) sodium bisulfite;
(e) benzyl alcohol;
(f) a carbonate buffer; and
(g) a phosphate buffer.

111. The composition of claim 110 wherein the substituted hexitol derivative is dianhydrogalactitol.

112. The composition of claim 93 wherein the substituted hexitol derivative is incorporated into a dosage form selected from the group consisting of:
(a) tablets;
(b) capsules;
(c) topical gels;
(d) topical creams;
(e) patches;
(f) suppositories; and
(g) lyophilized dosage fills.

113. The composition of claim 112 wherein the substituted hexitol derivative is dianhydrogalactitol.

114. The composition of claim 93 wherein the substituted hexitol derivative is incorporated into a dosage kit and packaging selected from the group consisting of amber vials to protect from light and stoppers with specialized coatings to improve shelf-life stability.

115. The composition of claim 114 wherein the substituted hexitol derivative is dianhydrogalactitol.

116. The composition of claim 93 wherein the composition comprises a substituted hexitol derivative and a drug delivery system selected from the group consisting of:
(a) nanocrystals;
(b) bioerodible polymers;
(c) liposomes;
(d) slow release injectable gels; and
(e) microspheres.

117. The composition of claim 116 wherein the substituted hexitol derivative is dianhydrogalactitol.

118. The composition of claim 93 wherein the substituted hexitol derivative is present in the composition in a drug conjugate form selected from the group consisting of:
(a) a polymer system;
(b) polylactides;
(c) polyglycolides;
(d) amino acids;
(e) peptides; and
(f) multivalent linkers.

119. The composition of claim 118 wherein the substituted hexitol derivative is dianhydrogalactitol.

120. The composition of claim 93 wherein the therapeutic agent is a modified substituted hexitol derivative and the modification is selected from the group consisting of:
(a) alteration of side chains to increase or decrease lipophilicity;
(b) addition of an additional chemical functionality to alter a property selected from the group consisting of reactivity, electron affinity, and binding capacity; and
(c) alteration of salt form.

121. The composition of claim 120 wherein the modified substituted hexitol derivative is a modified dianhydrogalactitol.

122. The composition of claim 93 wherein the substituted hexitol derivative is in the form of a prodrug system, wherein the prodrug system is selected from the group consisting of:
(a) enzyme sensitive esters;
(b) dimers;
(c) Schiff bases;
(d) pyridoxal complexes; and
(e) caffeine complexes.

123. The composition of claim 122 wherein the substituted hexitol derivative is dianhydrogalactitol.

124. The composition of claim 93 wherein the composition comprises a substituted hexitol derivative and at least one additional therapeutic agent to form a multiple drug system, wherein the at least one additional therapeutic agent is selected from the group consisting of:
(a) an inhibitor of multi-drug resistance;
(b) a specific drug resistance inhibitor;
(c) a specific inhibitor of a selective enzyme;
(d) a signal transduction inhibitor;
(e) an inhibitor of a repair enzyme; and
(f) a topoisomerase inhibitor with non-overlapping side effects.

125. The composition of claim 124 wherein the substituted hexitol derivative is dianhydrogalactitol.

126. The composition of claim 93 wherein the composition comprises a substituted hexitol derivative and an agent to counteract myelosuppression, wherein the agent to counteract myelosuppression is a dithiocarbamate.

127. The composition of claim 126 wherein the substituted hexitol derivative is dianhydrogalactitol.

128. The composition of claim 93 wherein the composition comprises a substituted hexitol derivative and an agent that increases the ability of the substituted hexitol to pass through the blood-brain barrier, wherein the agent that increases the ability of the substituted hexitol to pass through the blood-brain barrier is selected from the group consisting of:
(a) a chimeric peptide of the structure of Formula (D-III): 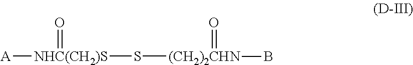
wherein: (A) A is somatostatin, thyrotropin releasing hormone (TRH), vasopressin, alpha interferon, endorphin, muramyl dipeptide or ACTH 4-9 analogue; and (B) B is insulin, IGF-I, IGF-II, transferrin, cationized (basic) albumin or prolactin; or a chimeric peptide of the structure of Formula (D-III) wherein the disulfide conjugating bridge between A and B is replaced with a bridge of Subformula (D-III(a)):
A-NH(CH₂)₂S—S—B(cleavable linkage) (D-III(a)),
wherein the bridge is formed using cysteamine and EDAC as the bridge reagents; or a chimeric peptide of the structure of Formula (D-III) wherein the disulfide conjugating bridge between A and B is replaced with a bridge of Subformula (D-III(b)):
A-NH═CH(CH₂)₃CH═NH—B(non-cleavable linkage) (D-III(b)),
wherein the bridge is formed using glutaraldehyde as the bridge reagent;
(b) a composition comprising either avidin or an avidin fusion protein bonded to a biotinylated substituted hexitol derivative to form an avidin-biotin-agent complex including therein a protein selected from the group consisting of insulin, transferrin, an anti-receptor monoclonal antibody, a cationized protein, and a lectin;
(c) a neutral liposome that is pegylated and incorporates the substituted hexitol derivative, wherein the polyethylene glycol strands are conjugated to at least one transportable peptide or targeting agent;
(d) a humanized murine antibody that binds to the human insulin receptor linked to the substituted hexitol derivative through an avidin-biotin linkage; and
(e) a fusion protein comprising a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative, wherein the fusion protein is linked to the substituted hexitol by a covalent link to biotin.

129. The composition of claim 128 wherein the substituted hexitol derivative is dianhydrogalactitol.

130. The composition of claim 93 wherein the composition comprises a substituted hexitol derivative and an agent that suppresses proliferation of cancer stem cells, wherein the agent that suppresses proliferation of cancer stem cells is selected from the group consisting of: (1) naphthoquinones; (2) VEGF-DLL4 bispecific antibodies; (3) farnesyl transferase inhibitors; (4) gamma-secretase inhibitors; (5) anti-TIM3 antibodies; (6) tankyrase inhibitors; (7) Wnt pathway inhibitors other than tankyrase inhibitors; (8) camptothecin-binding moiety conjugates; (9) Notch1 binding agents, including antibodies; (10) oxabicycloheptanes and oxabicycloheptenes; (11) inhibitors of the mitochondrial electron transport chains or the mitochondrial tricarboxylic acid cycle; (12) Ax inhibitors; (13) dopamine receptor antagonists; (14) anti-RSPO1 antibodies; (15) inhibitors or modulators of the Hedgehog pathway; (16) caffeic acid analogs and derivatives; (17) Stat3 inhibitors; (18) GRP-94-binding antibodies; (19) Frizzled receptor polypeptides; (20) immunoconjugates with cleavable linkages; (21) human prolactin, growth hormone, or placental lactogen; (22) anti-prominin-1 antibody; (23) antibodies specifically binding N-cadherin; (24) DR5 agonists; (25) anti-DLL4 antibodies or binding fragments thereof; (26) antibodies specifically binding GPR49; (27) DDR1 binding agents; (28) LGR5 binding agents; (29) telomerase-activating compounds; (30) fingolimod plus anti-CD74 antibodies or fragments thereof; (31) an antibody that prevents the binding of CD47 to SIPRα or a CD47 mimetic; (32) thienopyranone kinase inhibitors for inhibition of PI-3 kinases; (33) cancer-stem-cell-binding peptides; (34) diphtheria toxin-interleukin 3 conjugates; (35) inhibitors of histone deacetylase; (36) progesterone or analogs thereof; (37) antibodies binding the negative regulatory region (NRR) of Notch2; (38) inhibitors of HGFIN; (39) immunotherapeutic peptides; (40) inhibitors of CSCPK or related kinases; (41) imidazo[1,2-a]pyrazine derivatives as α-helix mimetics; (42) antibodies directed to an epitope of variant Heterogeneous Ribonucleoprotein G (HnRNPG); (43) antibodies binding TES7 antigen; (44) antibodies binding the ILR3α subunit; (45) ifenprodil tartrate and other compounds with a similar activity; (46) antibodies binding SALL4; (47) antibodies binding Notch4; (48) bispecific antibodies binding both NBR1 and Cep55; (49) Smo inhibitors; (50) peptides blocking or inhibiting interleukin-1 receptor 1; (51) antibodies specific for CD47 or CD19; (52) histone methyltransferase inhibitors; (53) antibodies specifically binding Lg5; (54) antibodies specifically binding EFNA1; (55) phenothiazine derivatives; (56) HDAC inhibitors plus AKT inhibitors; (57) ligands binding to cancer-stem-line-specific cell surface antigen stem cell markers; (58) Notch receptor agonists; (59) binding agents binding human MET; (60) PDGFR-β inhibitors; (61) pyrazolo compounds with histone demethylase activity; (62) heterocyclic substituted 3-heteroaryidenyl-2-indolinone derivatives; (63) albumin-binding arginine deiminase fusion proteins; (64) hydrogen-bond surrogate peptides and peptidomimetics that reactivate p53; (65) prodrugs of 2-pyrrolinodoxorubicin conjugated to antibodies; (66) targeted cargo proteins; (67) bisacodyl and analogs thereof; (68) N¹-cyclic amine-N⁵-substituted phenyl biguanide derivative; (69) fibulin-3 protein; (70) modulators of SCFSkp2; (71) inhibitors of Slingshot-2; (72) monoclonal antibodies specifically binding DCLK1 protein; (73) antibodies or soluble receptors that modulate the Hippo pathway; (74) selective inhibitors of CDK8 and CDK19; (75) antibodies and antibody fragments specifically binding IL-17; (76) antibodies specifically binding FRMD4A; (77) monoclonal antibodies specifically binding the ErbB-3 receptor; (78) antibodies that specifically bind human RSPO3 and modulate β-catenin activity; (79) esters of 4,9-dihydroxy-naphtho[2,3-b]furans; (80) CCR5 antagonists; (81) antibodies that specifically bind the extracellular domain of human C-type lectin-like molecule (CLL-1); (82) anti-hypertension compounds; (83) anthraquinone radiosensitizer agents plus ionizing radiation; (84) CDK-inhibiting pyrrolopyrimidinone derivatives; (85) analogs of CC-1065 and conjugates thereof; (86) antibodies specifically binding to the protein Notum; (87) CDK8 antagonists; (88) bHLH proteins and nucleic acids encoding them; (89) inhibitors of the histone methyltransferase EZH2; (90) sulfonamides inhibiting carbonic anhydrase isoforms; (91) antibodies specifically binding DEspR; (92) antibodies specifically binding human leukemia inhibitory factor (LIF); (93) doxovir; (94) inhibitors of mTOR; (95) antibodies specifically binding FZD10; (96) napthofurans; (97) death receptor agonists; (98) tigecycline; (99) strigolactones and strigolactone analogs; and (100) compounds inducing methuosis.

131. The method of claim 130 wherein the substituted hexitol derivative is dianhydrogalactitol.

132. A method of treating non-small-cell lung carcinoma (NSCLC) or glioblastoma multiforme (GBM) comprising the step of administering a therapeutically effective quantity of a substituted hexitol derivative to a patient suffering from NSCLC or GBM.

133. The method of claim 132 wherein the method is a method of treating NSCLC and comprises the step of administering a therapeutically effective quantity of a substituted hexitol derivative to a patients suffering from NSCLC.

134. The method of claim 132 wherein the method is a method of treating GBM and comprises the step of administering a therapeutically effective quantity of a substituted hexitol derivative to a patients suffering from GBM.

135. The method of claim 132 wherein the substituted hexitol derivative is selected from the group consisting of galactitols, substituted galacitols, dulcitols, and substituted dulcitols.

136. The method of claim 135 wherein the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol.

137. The method of claim 136 wherein the substituted hexitol derivative is dianhydrogalactitol.

138. The method of claim 137 wherein the therapeutically effective quantity of dianhydrogalactitol is a quantity of dianhydrogalactitol that results in a dosage of from about 1 mg/m² to about 40 mg/m².

139. The method of claim 138 wherein the therapeutically effective quantity of dianhydrogalactitol is a quantity of dianhydrogalactitol that results in a dosage of from about 5 mg/m² to about 25 mg/m².

140. The method of claim 137 wherein the dianhydrogalactitol is administered by a route selected from the group consisting of intravenous and oral.

141. The method of claim 132 further comprising a step selected from the group consisting of:
(a) administering a therapeutically effective dose of ionizing radiation;
(b) administering a therapeutically effective quantity of temozolomide;
(c) administering a therapeutically effective quantity of bevacizumab;
(d) administering a therapeutically effective quantity of a corticosteroid;
(e) administering a therapeutically effective quantity of at least one chemotherapeutic agent selected from the group consisting of lomustine, a platinum-containing chemotherapeutic agent, vincristine, and cyclophosphamide;
(f) administering a therapeutically effective quantity of a tyrosine kinase inhibitor;
(g) administering a therapeutically effective quantity of an EGFR inhibitor; and
(h) administering a therapeutically effective quantity of an agent that suppresses proliferation of cancer stem cells.

142. The method of claim 141 wherein the method further comprises the step of administering a therapeutically effective quantity of a platinum-containing chemotherapeutic agent and wherein the platinum-containing chemotherapeutic agent is selected from the group consisting of carboplatin, iproplatin, oxaliplatin, tetraplatin, satraplatin, picoplatin, nedaplatin, and triplatin.

143. The method of claim 142 wherein the administration of the substituted hexitol derivative together with the platinum-containing chemotherapeutic agent is a component of standard platinum doublet strategy.

144. The method of claim 141 wherein the method further comprises the step of administering a therapeutically effective dose of ionizing radiation, and wherein the ionizing radiation is administered concurrently with the substituted hexitol derivative.

145. The method of claim 141 wherein the method further comprises the step of administering a therapeutically effective dose of ionizing radiation, and wherein the ionizing radiation is administered separately from the substituted hexitol derivative.

146. The method of claim 141 wherein the method further comprises the step of administering a therapeutically effective dose of ionizing radiation, and wherein the ionizing radiation is administered in a single dose.

147. The method of claim 141 wherein the method further comprises the step of administering a therapeutically effective dose of ionizing radiation, and wherein the ionizing radiation is administered in fractionated doses.

148. The method of claim 141 wherein the method further comprises the step of administering a therapeutically effective dose of ionizing radiation, and wherein the radiation dosage is from about 40 Gy to about 79.2 Gy.

149. The method of claim 150 wherein the radiation dosage is about 60 Gy.

150. The method of claim 141 wherein the method further comprises the step of administering a therapeutically effective dose of ionizing radiation, and wherein the radiation is administered by a method selected from the group consisting of high-energy X-rays, high-energy electrons from a linear accelerator unit, and gamma rays from a cobalt-60-based device.

151. The method of claim 141 wherein the method is for the treatment of GBM, wherein the method further comprises the step of administering a therapeutically effective dose of ionizing radiation, and wherein the method also further comprises the step of administering trans sodium crocetinate as a radiosensitizer.

152. The method of claim 137 wherein the dianhydrogalactitol substantially suppresses the growth of cancer stem cells (CSCs).

153. The method of claim 137 wherein the dianhydrogalactitol is effective in suppressing the growth of cancer cells possessing O⁶-methylguanine-DNA methyltransferase (MGMT)-driven drug resistance.

154. The method of claim 137 wherein the dianhydrogalactitol is effective in suppressing the growth of cancer cells resistant to temozolomide.

155. The method of claim 134 wherein the method further comprises administering a therapeutically effective quantity of an EGFR inhibitor and wherein the EGFR inhibitor affects wild-type binding sites.

156. The method of claim 134 wherein the method comprises administering a therapeutically effective quantity of an EGFR inhibitor and wherein the EGFR inhibitor affects mutated binding sites.

157. The method of claim 156 wherein the EGFR inhibitor affects EGFR Variant III.

158. The method of claim 134 wherein the method further comprises administering to the patient a therapeutically effective quantity of an agent that increases the ability of the substituted hexitol to pass through the blood-brain barrier.

159. The method of claim 134 wherein the method further comprises administering to the patient a therapeutically effective quantity of an agent that counteracts myelosuppression.

160. The method of claim 134 wherein the method further comprises administering to the patient a therapeutically effective quantity of an agent that suppresses the growth of cancer stem cells.

161. The method of claim 160 wherein the agent that suppresses the growth of cancer stem cells is selected from the group consisting of: (1) naphthoquinones; (2) VEGF-DLL4 bispecific antibodies; (3) farnesyl transferase inhibitors; (4) gamma-secretase inhibitors; (5) anti-TIM3 antibodies; (6) tankyrase inhibitors; (7) Wnt pathway inhibitors other than tankyrase inhibitors; (8) camptothecin-binding moiety conjugates; (9) Notch1 binding agents, including antibodies; (10) oxabicycloheptanes and oxabicycloheptenes; (11) inhibitors of the mitochondrial electron transport chains or the mitochondrial tricarboxylic acid cycle; (12) Axl inhibitors; (13) dopamine receptor antagonists; (14) anti-RSPO1 antibodies; (15) inhibitors or modulators of the Hedgehog pathway; (16) caffeic acid analogs and derivatives; (17) Stat3 inhibitors; (18) GRP-94-binding antibodies; (19) Frizzled receptor polypeptides; (20) immunoconjugates with cleavable linkages; (21) human prolactin, growth hormone, or placental lactogen; (22) anti-prominin-1 antibody; (23) antibodies specifically binding N-cadherin; (24) DR5 agonists; (25) anti-DLL4 antibodies or binding fragments thereof; (26) antibodies specifically binding GPR49; (27) DDR1 binding agents; (28) LGR5 binding agents; (29) telomerase-activating compounds; (30) fingolimod plus anti-CD74 antibodies or fragments thereof; (31) an antibody that prevents the binding of CD47 to SIPRα or a CD47 mimetic; (32) thienopyranone kinase inhibitors for inhibition of PI-3 kinases; (33) cancer-stem-cell-binding peptides; (34) diphtheria toxin-interleukin 3 conjugates; (35) inhibitors of histone deacetylase; (36) progesterone or analogs thereof; (37) antibodies binding the negative regulatory region (NRR) of Notch2; (38) inhibitors of HGFIN; (39) immunotherapeutic peptides; (40) inhibitors of CSCPK or related kinases; (41) imidazo[1,2-a]pyrazine derivatives as α-helix mimetics; (42) antibodies directed to an epitope of variant Heterogeneous Ribonucleoprotein G (HnRNPG); (43) antibodies binding TES7 antigen; (44) antibodies binding the ILR3α subunit; (45) ifenprodil tartrate and other compounds with a similar activity; (46) antibodies binding SALL4; (47) antibodies binding Notch4; (48) bispecific antibodies binding both NBR1 and Cep55; (49) Smo inhibitors; (50) peptides blocking or inhibiting interleukin-1 receptor 1; (51) antibodies specific for CD47 or CD19; (52) histone methyltransferase inhibitors; (53) antibodies specifically binding Lg5; (54) antibodies specifically binding EFNA1; (55) phenothiazine derivatives; (56) HDAC inhibitors plus AKT inhibitors; (57) ligands binding to cancer-stem-line-specific cell surface antigen stem cell markers; (58) Notch receptor agonists; (59) binding agents binding human MET; (60) PDGFR-β inhibitors; (61) pyrazolo compounds with histone demethylase activity; (62) heterocyclic substituted 3-heteroaryidenyl-2-indolinone derivatives; (63) albumin-binding arginine deiminase fusion proteins; (64) hydrogen-bond surrogate peptides and peptidomimetics that reactivate p53; (65) prodrugs of 2-pyrrolinodoxorubicin conjugated to antibodies; (66) targeted cargo proteins; (67) bisacodyl and analogs thereof; (68) N¹-cyclic amine-N⁵-substituted phenyl biguanide derivative; (69) fibulin-3 protein; (70) modulators of SCFSkp2; (71) inhibitors of Slingshot-2; (72) monoclonal antibodies specifically binding DCLK1 protein; (73) antibodies or soluble receptors that modulate the Hippo pathway; (74) selective inhibitors of CDK8 and CDK19; (75) antibodies and antibody fragments specifically binding IL-17; (76) antibodies specifically binding FRMD4A; (77) monoclonal antibodies specifically binding the ErbB-3 receptor; (78) antibodies that specifically bind human RSPO3 and modulate β-catenin activity; (79) esters of 4,9-dihydroxy-naphtho[2,3-b]furans; (80) CCR5 antagonists; (81) antibodies that specifically bind the extracellular domain of human C-type lectin-like molecule (CLL-1); (82) anti-hypertension compounds; (83) anthraquinone radiosensitizer agents plus ionizing radiation; (84) CDK-inhibiting pyrrolopyrimidinone derivatives; (85) analogs of CC-1065 and conjugates thereof; (86) antibodies specifically binding to the protein Notum; (87) CDK8 antagonists; (88) bHLH proteins and nucleic acids encoding them; (89) inhibitors of the histone methyltransferase EZH2; (90) sulfonamides inhibiting carbonic anhydrase isoforms; (91) antibodies specifically binding DEspR; (92) antibodies specifically binding human leukemia inhibitory factor (LIF); (93) doxovir; (94) inhibitors of mTOR; (95) antibodies specifically binding FZD10; (96) napthofurans; (97) death receptor agonists; (98) tigecycline; (99) strigolactones and strigolactone analogs; and (100) compounds inducing methuosis.
